(19) United States
(12) Patent Application Publication
(10) Pub. No.: US 2026/0201408 A1
(43) Pub. Date: Jul. 16, 2026

(54) CORN EVENT 5307

(71) Applicant: Syngenta Crop Protection AG, Basel (CH)

(72) Inventors: Annick Jeanne de Frammond, Research Triangle Park, NC (US); Moez Rajabali Meghji, St. Louis, MO (US); Stephen L. New, Roseville, CA (US); Anna Underwood Prairie, Research Triangle Park, NC (US)

(73) Assignee: Syngenta Crop Protection AG, Basel (CH)

(21) Appl. No.: 19/311,785

(22) Filed: Aug. 27, 2025

Related U.S. Application Data
(63) Continuation of application No. 18/175,869, filed on Feb. 28, 2023, now Pat. No. 12,428,650.
Application No. 18/175,869 is a continuation of application No. 16/988,681, filed on Aug. 9, 2020, now abandoned.
Application No. 16/988,681 is a division of application No. 16/126,171, filed on Sep. 10, 2018, now Pat. No. 10,844,400.
Application No. 16/126,171 is a division of application No. 14/815,345, filed on Jul. 31, 2015, now Pat. No. 10,100,371.
Application No. 14/815,345 is a division of application No. 13/140,429, filed on Dec. 14, 2009, now Pat. No. 9,133,474.
(60) Provisional application No. 61/122,885, filed on Dec. 16, 2008.

(51) Int. Cl. C12N 15/82; C07K 14/415; C12Q 1/6895

(52) U.S. Cl. CPC C12N 15/8286; C07K 14/415; C12N 15/8241; C12Q 1/6895; Y02A 40/146

(57) ABSTRACT

A novel transgenic corn event designated 5307, is disclosed. The invention relates to DNA sequences of the recombinant constructs inserted into the corn genome and of genomic sequences flanking the insertion site that resulted in the 5307 event. The invention further relates to assays for detecting the presence of the DNA sequences of event 5307, to corn plants and corn seeds comprising the genotype of and to methods for producing a corn plant by crossing a corn plant comprising the event 5307 genotype with itself or another corn variety.

### RELATED APPLICATION INFORMATION

This application is a continuation of co-pending U.S. patent application Ser. No. 18/175,869, filed Feb. 28, 2023, which is a continuation of U.S. patent application Ser. No. 16/988,681 filed Aug. 9, 2020, which is a divisional of U.S. patent application Ser. No. 16/126,171 filed Sep. 10, 2018 (now U.S. Pat. No. 10,844,400 B2) which is a divisional of Ser. No. 14/815,345 filed Jul. 31, 2015 (now U.S. Pat. No. 10,100,371 B2), which is a divisional of U.S. patent application Ser. No. 13/140,429 (now U.S. Pat. No. 9,133,474 B2), which is a national stage filing under 35 U.S.C. § 371 of International Application No. PCT/US2009/067873, filed Dec. 14, 2009 and published Jul. 8, 2010 as WO 2010/077816, which claims the benefit of U.S. Provisional Application Ser. No. 61/122,885, filed Dec. 16, 2008, the contents of each of which are incorporated herein by reference in their entireties.

### STATEMENT REGARDING ELECTRONIC SUBMISSION OF A SEQUENCE LISTING

A sequence listing in XML format, submitted under 37 C.F.R. § 1.831 (a), entitled “71922 Sequence Listing.xml”, 482,567 bytes in size, generated on May 20, 2025, and filed via EFS-Web is provided in lieu of a paper copy. This sequence listing is hereby incorporated by reference into the specification for its disclosures.

### FIELD OF THE INVENTION

The invention relates generally to the field of plant molecular biology, plant transformation, and plant breeding. More specifically, the invention relates to insect resistant transgenic corn plants comprising a novel transgenic genotype and to methods of detecting the presence of the corn plant DNA in a sample and compositions thereof.

### BACKGROUND

Plant pests are a major factor in the loss of the world's important agricultural crops. About $8 billion are lost every year in the U.S. alone due to infestations of non-mammalian pests including insects. Species of corn rootworm are considered the most destructive corn pests. Important rootworm pest species include Diabrotica virgifera virgifera, the western corn rootworm; D. longicornis barberi, the northern corn rootworm, D. undecimpunctata howardi, the southern corn rootworm, and D. virgifera zeae, the Mexican corn rootworm.

Corn rootworm is mainly controlled by intensive applications of chemical pesticides. Good corn rootworm control can thus be reached, but these chemicals can sometimes also affect beneficial organisms. Another problem resulting from the wide use of chemical pesticides is the appearance of resistant insect varieties. This has been partially alleviated by various resistance management practices, but there is an increasing need for alternative pest control strategies. One such alternative includes the expression of foreign genes encoding insecticidal proteins in transgenic plants. This approach has provided an efficient means of protection against selected insect pests, and transgenic plants expressing insecticidal toxins have been commercialized, allowing farmers to reduce applications of chemical insecticides.

The expression of foreign genes in plants can to be influenced by their chromosomal position, perhaps due to chromatin structure or the proximity of transcriptional regulation elements close to the integration site (See for example, Weising et al., 1988, “Foreign Genes in Plants,” Ann. Rev. Genet. 22:421-477). Therefore, it is common to produce hundreds of different events and screen those events for a single event that has desired transgene expression levels and patterns for commercial purposes. An event that has desired levels or patterns of transgene expression is useful for introgressing the transgene into other genetic backgrounds by sexual outcrossing using conventional breeding methods. Progeny of such crosses maintain the transgene expression characteristics of the original transformant. This strategy is used to ensure reliable gene expression in a number of varieties that are well adapted to local growing conditions.

It would be advantageous to be able to detect the presence of a particular event in order to determine whether progeny of a sexual cross contain a transgene of interest. In addition, a method for detecting a particular event would be helpful for complying with regulations requiring the pre-market approval and labeling of foods derived from recombinant crop plants, for example. It is possible to detect the presence of a transgene by any well-known nucleic acid detection method including but not limited to thermal amplification (polymerase chain reaction (PCR)) using polynucleotide primers or DNA hybridization using nucleic acid probes. Typically, for the sake of simplicity and uniformity of reagents and methodologies for use in detecting a particular DNA construct that has been used for transforming various plant varieties, these detection methods generally focus on frequently used genetic elements, for example, promoters, terminators, and marker genes, because for many DNA constructs, the coding sequence region is interchangeable. As a result, such methods may not be useful for discriminating between constructs that differ only with reference to the coding sequence. In addition, such methods may not be useful for discriminating between different events, particularly those produced using the same DNA construct unless the sequence of chromosomal DNA adjacent to the inserted heterologous DNA (“flanking DNA”) is known.

The invention includes an insect resistant transgenic corn event that has incorporated into its genome a FR8a gene, disclosed in International Publication No. WO 08/121633, published Oct. 9, 2008, which is herein incorporated by reference, encoding a FR8a insecticidal toxin, useful in controlling Diabrotica spp. insect pests. The transgenic corn event also has incorporated in its genome a PMI gene, encoding a phosphomannose isomerase enzyme (PMI), disclosed in U.S. Pat. No. 5,767,378, which is herein incorporated by reference, useful as a selectable marker, which allows the plant to utilize mannose as a carbon source. The invention further includes novel isolated nucleic acid sequences which are unique to the transgenic corn event, useful for identifying the transgenic corn event and for detecting nucleic acids from the transgenic corn event in a biological sample, as well as kits comprising the reagents necessary for use in detecting these nucleic acids in a biological sample.

### SUMMARY

The invention is drawn to a transgenic corn event, designated 5307, comprising a novel transgenic genotype that comprises a FR8a gene and a PMI gene which confers insect resistance and the ability to utilize mannose as a carbon source, respectively, to the 5307 corn event and progeny thereof. The invention also provides transgenic corn plants comprising the genotype of the invention, seed from transgenic corn plants comprising the genotype of the invention, and to methods for producing a transgenic corn plant comprising the genotype of the invention by crossing a corn inbred comprising the genotype of the invention with itself or another corn line of a different genotype. The transgenic corn plants of the invention may have essentially all of the morphological and physiological characteristics of the corresponding isogenic non-transgenic corn plant in addition to those conferred upon the corn plant by the novel genotype of the invention. The invention also provides compositions and methods for detecting the presence of nucleic acids from event 5307 based on the DNA sequence of the recombinant expression cassettes inserted into the corn genome that resulted in the 5307 event and of genomic sequences flanking the insertion site. The 5307 event can be further characterized by analyzing expression levels of FR8a and PMI proteins as well as by testing efficacy against corn rootworm.

According to one aspect, the invention provides a preferably isolated nucleic acid molecule comprising at least 10 contiguous nucleotides of a heterologous DNA sequence inserted into the corn plant genome of corn event 5307 and at least 10 contiguous nucleotides of a corn plant genome DNA flanking the point of insertion of a heterologous DNA sequence inserted into the corn plant genome of corn event 5307. The preferably isolated nucleic acid molecule according to this aspect may comprise at least 20 or at least 50 contiguous nucleotides of a heterologous DNA sequence inserted into the corn plant genome of corn event 5307 and at least 20 or at least 50 contiguous nucleotides of a corn plant genome DNA flanking the point of insertion of a heterologous DNA sequence inserted into the corn plant genome of corn event 5307.

According to another aspect, the invention provides a preferably isolated nucleic acid molecule comprising at least one junction sequence of event 5307 selected from the group consisting of SEQ ID NO: 1 and SEQ ID NO: 2, and complements thereof. A junction sequence spans the junction between the heterologous DNA comprising the expression cassettes inserted into the corn genome and DNA from the corn genome flanking the insertion site and is diagnostic for the 5307 event.

According to another aspect, the invention provides a preferably isolated nucleic acid linking a heterologous DNA molecule to the corn plant genome in corn event 5307 comprising a sequence of from about 11 to about 20 contiguous nucleotides selected from the group consisting of SEQ ID NO: 1, SEQ ID NO: 2, and complements thereof.

According to another aspect, the invention provides a preferably isolated nucleic acid molecule comprising a nucleotide sequence selected from the group consisting of SEQ ID NO: 1, SEQ ID NO: 2, SEQ ID NO: 3, SEQ ID NO: 4, and complements thereof.

According to another aspect of the invention, an amplicon comprising a nucleic acid molecule of the invention is provided.

According to still another aspect of the invention, flanking sequence primers for detecting event 5307 are provided. Such flanking sequence primers comprise a preferably isolated nucleic acid sequence comprising at least 10-15 contiguous nucleotides from nucleotides 1-1348 as set forth in SEQ ID NO: 5 (arbitrarily designated herein as the 5′ flanking sequence), or the complements thereof, also disclosed as SEQ ID NO: 111. In one embodiment of this aspect the flanking sequence primers are selected from the group consisting of SEQ ID NO: 9 through SEQ ID NO: 14, and complements thereof.

In another aspect of the invention, the flanking sequences primers comprise a preferably isolated nucleic acid sequence comprising at least 10-15 contiguous nucleotides from nucleotides 1-1093 as set forth in SEQ ID NO: 6 (arbitrarily designated herein as the 3′ flanking sequence), or the complements thereof. In one embodiment of this aspect the flanking sequence primers are selected from the group consisting of SEQ ID NO: 69 through SEQ ID NO: 72, and complements thereof.

According to another aspect of the invention, primer pairs that are useful for nucleic acid amplification, for example, are provided. Such primer pairs comprise a first primer comprising a nucleotide sequence of at least 10-15 contiguous nucleotides in length which is or is complementary to one of the above-described genomic flanking sequences (SEQ ID NO: 5, or SEQ ID NO: 6) and a second primer comprising a nucleotide sequence of at least 10-15 contiguous nucleotides of heterologous DNA inserted into the event 5307 genome. The second primer preferably comprises a nucleotide sequence which is or is complementary to the insert sequence adjacent to the plant genomic flanking DNA sequence as set forth in SEQ ID NO: 7. In one embodiment of this aspect the insert sequence primers are selected from the group consisting of SEQ ID NO: 15 through SEQ ID NO: 68, and complements thereof.

According to another aspect of the invention, methods of detecting the presence of DNA corresponding to event 5307 in a biological sample are provided. Such methods comprise: (a) contacting the sample comprising DNA with a pair of primers that, when used in a nucleic acid amplification reaction with genomic DNA from corn event 5307; produces an amplicon that is diagnostic for corn event 5307; (b) performing a nucleic acid amplification reaction, thereby producing the amplicon; and (c) detecting the amplicon. In one embodiment of this aspect, the amplicon comprises a nucleotide sequence selected from the group consisting of SEQ ID NO: 1, SEQ ID NO: 2, SEQ ID NO: 3, SEQ ID NO: 4, SEQ ID NO: 5, SEQ ID NO: 6, and complements thereof.

According to another aspect, the invention provides methods of detecting the presence of a DNA corresponding to the 5307 event in a biological sample. Such methods comprise: (a) contacting the sample comprising DNA with a probe that hybridizes under high stringency conditions with genomic DNA from corn event 5307 and does not hybridize under high stringency conditions with DNA of a control corn plant; (b) subjecting the sample and probe to high stringency hybridization conditions; and (c) detecting hybridization of the probe to the DNA. The detected hybridized DNA sequence includes at least one polynucleotide sequence comprising SEQ ID NO: 1, SEQ ID NO: 2, SEQ ID NO: 3, SEQ ID NO: 4, SEQ ID NO: 5, SEQ ID NO: 6, and complements thereof.

According to another aspect of the invention, a kit is provided for the detection of event 5307 nucleic acids in a biological sample. The kit includes at least one DNA sequence comprising a sufficient length of polynucleotides which is or is complementary to SEQ ID NO: 1, SEQ ID NO: 2, SEQ ID NO: 3, SEQ ID NO: 4, SEQ ID NO: 5 or SEQ ID NO: 6, wherein the DNA sequences are useful as primers or probes that hybridize to isolated DNA from event 5307, and which, upon amplification of or hybridization to a nucleic acid sequence in a sample followed by detection of the amplicon or hybridization to the target sequence, are diagnostic for the presence of nucleic acid sequences from event 5307 in the sample. The kit further includes other materials necessary to enable nucleic acid hybridization or amplification methods.

In another aspect, the invention provides a method of detecting corn event 5307 protein in a biological sample comprising: (a) extracting protein from a sample of corn event 5307 tissue; (b) assaying the extracted protein using an immunological method comprising antibody specific for the insecticidal or selectable marker protein produced by the 5307 event; and (c) detecting the binding of said antibody to the insecticidal or selectable marker protein.

In another aspect, the invention provides a biological sample derived from a event 5307 corn plant, tissue, or seed, wherein the sample comprises a nucleic acid comprising a nucleotide sequence which is or is complementary to a sequence selected from the group consisting of SEQ ID NO: 1 and SEQ ID NO: 2, and wherein the sequence is detectable in the sample using a nucleic acid amplification or nucleic acid hybridization method. In one embodiment of this aspect, the sample is selected from the group consisting of corn flour, corn meal, corn syrup, corn oil, cornstarch, and cereals manufactured in whole or in part to contain corn by-products.

In another aspect, the invention provides an extract derived from a event 5307 corn plant, tissue, or seed comprising a nucleotide sequence which is or is complementary to a nucleotide sequence selected from the group consisting of SEQ ID NO: 1 and SEQ ID NO: 2. In one embodiment of this aspect, the sequence is detectable in the extract using a nucleic acid amplification or nucleic acid hybridization method. In another embodiment of this aspect, the sample is selected from the group consisting of corn flour, corn meal, corn syrup, corn oil, cornstarch, and cereals manufactured in whole or in part to contain corn by-products.

According to another aspect of the invention, corn plants and seeds comprising the nucleic acid molecules of the invention are provided. In one embodiment of the invention, a deposit of event 5307 corn seed was made to the American Type Culture Collection (ATCC) in accordance with the Budapest Treaty on 15 Oct. 2008. An example of said seed being deposited as ATCC Accession No: PTA-9561.

According to another aspect, the invention provides a method for producing a corn plant resistant to at least corn rootworm infestation comprising: (a) sexually crossing a first parent corn plant with a second parent corn plant, wherein first or second parent corn plant comprises corn event 5307 DNA, thereby producing a plurality of first generation progeny plants; (b) selecting a first generation progeny plant that is resistant to at least corn rootworm infestation; (c) selfing the first generation progeny plant, thereby producing a plurality of second generation progeny plants; (d) selecting from the second generation progeny plants, a plant that is at least resistant to corn rootworm infestation; wherein the second generation progeny plants comprise a nucleotide sequence selected from the group consisting of SEQ ID NO: 1, SEQ ID NO: 2, SEQ ID NO: 3 and SEQ ID NO: 4.

According to yet another aspect, the invention provides a method for producing corn seed comprising crossing a first parent corn plant with a second parent corn plant and harvesting the resultant first generation corn seed, wherein the first or second parent corn plant is an inbred corn plant of the invention.

According to another aspect, the invention provides a method of producing hybrid corn seeds comprising the steps of: (a) planting seeds of a first inbred corn line according to the invention and seeds of a second inbred corn line having a different genotype; (b) cultivating corn plants resulting from said planting until time of flowering; (c) emasculating flowers of corn plants of one of the corn inbred lines; (d) allowing pollination of the other inbred line to occur, and (e) harvesting the hybrid seed produced thereby.

According to another aspect of the invention, the invention provides a method of selecting corn plants and seeds comprising the nucleic acid molecules of event 5307 on chromosome 5. In one embodiment of the invention, polymorphic markers are used to select or track the sequences specific to the 5307 corn event. The invention provides a method of selecting sequences specific to the 5307 corn event comprising the steps of: (a) detecting a polymorphic marker sequence; (b) designing an assay for the purposes of detecting the marker; (c) running the assay on corn nucleic acid sequences from many corn lines, and (d) selecting corn lines based upon the sequences with nucleotides specific to corn event 5307.

According to another aspect of the invention, the invention provides a site on chromosome 5 for targeted integration of a heterologous nucleic acid. The invention provides a method of selecting sequences specific to the 5307 corn event for targeted integration comprising the steps of: (a) designing homologous sequences based on the insertion site or vector sequence; (b) using these homologous sequences at a target locus; (c) using zinc finger nucleases to create a break in the target locus, and (d) inserting a heterologous donor molecule within nucleotides specific to corn event 5307 or the vector sequence of pSYN12274. An example of this technique is demonstrated in Shukla et al. (Nature vol. 459, 21 May 2009).

The foregoing and other aspects of the invention will become more apparent from the following detailed description.

### DESCRIPTION OF THE SEQUENCES IN THE SEQUENCE LISTING

SEQ ID NO: 1 is the 5′ genome-insert junction.
SEQ ID NO: 2 is the 3′ insert-genome junction.
SEQ ID NO: 3 is the 5′ genome+insert sequence.
SEQ ID NO: 4 is the 3′ insert+genome sequence.
SEQ ID NO: 5 is the 5′ genome+insert sequence.
SEQ ID NO: 6 is the 3′ corn genome flanking sequence.
SEQ ID NO: 7 is the event 5307 full length insert.
SEQ ID Nos: 8-14 are 5′ flanking sequence primers useful in the invention.
SEQ ID Nos: 15-68 are 5307 transgene insert primers useful in the invention.
SEQ ID Nos: 69-72 are 3′ flanking sequence primers useful in the invention.
SEQ ID Nos: 73-75 are FR8a TAQMAN primers and probe.
SEQ ID Nos: 76-78 are PMI TAQMAN primers and probe.
SEQ ID Nos: 79-81 are ZmAdh TAQMAN primers and probe.
SEQ ID Nos: 82-90 are 5307 event specific primers and probes useful in the invention.
SEQ ID Nos: 91-102 are corn genomic primers and probes useful in the invention.
SEQ ID NO: 103 is the AC202955 Chromosome 5 Sequence, where Nis any base “A”, “T”, “G” or “C”.
SEQ ID NO: 104 is the umc1475 marker region.
SEQ ID Nos: 105-106 are umc1475 primers.
SEQ ID NO: 107 is the uaz190 marker region.
SEQ ID NOs: 108-109 are uaz190 primers
SEQ ID NO: 110 is the reverse complement of SEQ ID NO: 103, AC202955 Chromosome 5 Sequence, where N is any base “A”, “T”, “G” or “C”.
SEQ ID NO: 111 is the 5′ corn genome flanking sequence.

### BRIEF DESCRIPTION OF DRAWINGS

FIG. 1 illustrates a plant expression vector designated pSYN12274. The plasmid map identifies the SmaI and PmeI restriction sites used for Southern analysis.

FIG. 2 is a graphical map illustrating the organization of the elements comprising the heterologous nucleic acid sequences inserted into the genome of corn to create event 5307 and sets forth the relative positions at which the inserted nucleic acid sequences are linked to corn genomic DNA sequences which flank the ends of the inserted heterologous DNA sequences. 1=5′ flanking plant genome (SEQ ID NO: 5); 2=right border region; 3=CMP promoter; 4=FR8a gene; 5=NOS terminator; 6=ZmUbINT promoter; 7=PMI gene; 8=NOS terminator; 9=left border region (sections 2 through 9 are contained within SEQ ID NO: 7); and 10=3′ flanking plant genome (SEQ ID NO: 6).

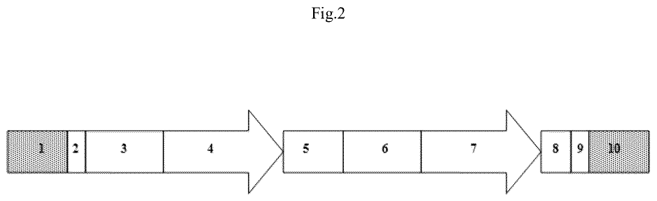

### DEFINITIONS

The following definitions and methods are provided to better define the invention and to guide those of ordinary skill in the art in the practice of the invention. Unless otherwise noted, terms used herein are to be understood according to conventional usage by those of ordinary skill in the relevant art. Definitions of common terms in molecular biology may also be found in Rieger et al., Glossary of Genetics: Classical and Molecular, 5^{th} edition, Springer-Verlag: New York, 1994.

As used herein, the term “amplified” means the construction of multiple copies of a nucleic acid molecule or multiple copies complementary to the nucleic acid molecule using at least one of the nucleic acid molecules as a template. Amplification systems include the polymerase chain reaction (PCR) system, ligase chain reaction (LCR) system, nucleic acid sequence based amplification (NASBA, Cangene, Mississauga, Ontario), Q-Beta Replicase systems, transcription-based amplification system (TAS), and strand displacement amplification (SDA). See, e.g., Diagnostic Molecular Microbiology: Principles and Applications, D. H. Persing et al., Ed., American Society for Microbiology, Washington, D.C. (1993). The product of amplification is termed an amplicon.

A “biological sample” is a plant, plant material or products comprising plant material. The term “plant” is intended to encompass corn (Zea mays) plant tissues, at any stage of maturity, as well as cells, tissues, organs taken from or derived from any such plant, including without limitation, any seeds, leaves, stems, flowers, roots, single cells, gametes, cell cultures, tissue cultures or protoplasts. “Plant material”, as used herein refers to material which is obtained or derived from a plant. Products comprising plant material relate to food, feed or other products which are produced using plant material or can be contaminated by plant material. It is understood that, in the context of the invention, such biological sample are tested for the presence of nucleic acids specific to corn event 5307, implying the presence of nucleic acids in the samples. Thus, the methods referred to herein for identifying corn event 5307 in biological samples, relate to the identification in biological samples of nucleic acids which from an event 5307 corn plant and are diagnostic for event 5307.

A “coding sequence” is a nucleic acid sequence that is transcribed into RNA such as mRNA, rRNA, tRNA, snRNA, sense RNA or antisense RNA. Preferably the RNA is then translated in an organism to produce a protein.

“Detection kit” as used herein refers to a kit used to detect the presence or absence of DNA from event 5307 corn plants in a sample comprising nucleic acid probes and primers of the invention, which hybridize specifically under high stringency conditions to a target DNA sequence, and other materials necessary to enable nucleic acid hybridization or amplification methods.

As used herein the term transgenic “event” refers to a recombinant plant produced by transformation and regeneration of a single plant cell with heterologous DNA, for example, an expression cassette that includes a gene of interest. The term “event” refers to the original transformant and/or progeny of the transformant that include the heterologous DNA. The term “event” also refers to progeny produced by a sexual outcross between the transformant and another corn line. Even after repeated backcrossing to a recurrent parent, the inserted DNA and the flanking DNA from the transformed parent is present in the progeny of the cross at the same chromosomal location. Normally, transformation of plant tissue produces multiple events, each of which represent insertion of a DNA construct into a different location in the genome of a plant cell. Based on the expression of the transgene or other desirable characteristics, a particular event is selected. Thus, “event 5307”, “5307 event” or “5307” as used herein, means the original 5307 transformant and/or progeny of the 5307 transformant, including any plant derived therefrom.

“Expression cassette” as used herein means a nucleic acid molecule capable of directing expression of a particular nucleotide sequence in an appropriate host cell, comprising a promoter operably linked to the nucleotide sequence of interest which is operably linked to termination signals. It also typically comprises sequences required for proper translation of the nucleotide sequence. The expression cassette may also comprise sequences not necessary in the direct expression of a nucleotide sequence of interest but which are present due to convenient restriction sites for removal of the cassette from an expression vector. The expression cassette comprising the nucleotide sequence of interest may be chimeric, meaning that at least one of its components is heterologous with respect to at least one of its other components. The expression cassette may also be one that is naturally occurring but has been obtained in a recombinant form useful for heterologous expression. Typically, however, the expression cassette is heterologous with respect to the host, i.e., the particular nucleic acid sequence of the expression cassette does not occur naturally in the host cell and must have been introduced into the host cell or an ancestor of the host cell by a transformation process known in the art. The expression of the nucleotide sequence in the expression cassette may be under the control of a constitutive promoter or of an inducible promoter that initiates transcription only when the host cell is exposed to some particular external stimulus. In the case of a multicellular organism, such as a plant, the promoter can also be specific to a particular tissue, or organ, or stage of development. An expression cassette, or fragment thereof, can also be referred to as “inserted sequence” or “insertion sequence” when transformed into a plant.

A “gene” is a defined region that is located within a genome and that, besides the aforementioned coding nucleic acid sequence, comprises other, primarily regulatory, nucleic acid sequences responsible for the control of the expression, that is to say the transcription and translation, of the coding portion. A gene may also comprise other 5′ and 3′ untranslated sequences and termination sequences. Further elements that may be present are, for example, introns.

“Gene of interest” refers to any gene which, when transferred to a plant, confers upon the plant a desired characteristic such as antibiotic resistance, virus resistance, insect resistance, disease resistance, or resistance to other pests, herbicide tolerance, improved nutritional value, improved performance in an industrial process or altered reproductive capability. The “gene of interest” may also be one that is transferred to plants for the production of commercially valuable enzymes or metabolites in the plant.

“Genotype” as used herein is the genetic material inherited from parent corn plants not all of which is necessarily expressed in the descendant corn plants. The 5307 genotype refers to the heterologous genetic material transformed into the genome of a plant as well as the genetic material flanking the inserted sequence.

A “heterologous” nucleic acid sequence is a nucleic acid sequence not naturally associated with a host cell into which it is introduced, including non-naturally occurring multiple copies of a naturally occurring nucleic acid sequence.

A “homologous” nucleic acid sequence is a nucleic acid sequence naturally associated with a host cell into which it is introduced.

The term “isolated” when used in relation to a nucleic acid refers to a nucleic acid sequence that is identified and separated from at least one contaminant nucleic acid with which it is ordinarily associated in its natural source. An isolated nucleic acid is present in a form or setting that is different from that in which it is found in nature. In contrast, a non-isolated nucleic acids such as DNA and RNA found in the state they exist in nature. An isolated nucleic acid may be in a transgenic plant and still be considered “isolated”.

“Operably-linked” refers to the association of nucleic acid sequences on a single nucleic acid fragment so that the function of one affects the function of the other. For example, a promoter is operably-linked with a coding sequence or functional RNA when it is capable of affecting the expression of that coding sequence or functional RNA (i.e., that the coding sequence or functional RNA is under the transcriptional control of the promoter). Coding sequences in sense or antisense orientation can be operably-linked to regulatory sequences.

“Primers” as used herein are isolated nucleic acids that are annealed to a complimentary target DNA strand by nucleic acid hybridization to form a hybrid between the primer and the target DNA strand, then extended along the target DNA strand by a polymerase, such as DNA polymerase. Primer pairs or sets can be used for amplification of a nucleic acid molecule, for example, by the polymerase chain reaction (PCR) or other conventional nucleic-acid amplification methods.

A “probe” is an isolated nucleic acid to which is attached a conventional detectable label or reporter molecule, such as a radioactive isotope, ligand, chemiluminescent agent, or enzyme. Such a probe is complimentary to a strand of a target nucleic acid, in the case of the invention, to a strand of genomic DNA from corn event, M5307. The genomic DNA of event 5307 can be from a corn plant or from a sample that includes DNA from the event. Probes according to the invention include not only deoxyribonucleic or ribonucleic acids but also polyamides and other probe materials that bind specifically to a target DNA sequence and can be used to detect the presence of that target DNA sequence.

Primers and probes are generally between 10 and 15 nucleotides or more in length, Primers and probes can also be at least 20 nucleotides or more in length, or at least 25 nucleotides or more, or at least 30 nucleotides or more in length. Such primers and probes hybridize specifically to a target sequence under high stringency hybridization conditions. Primers and probes according to the invention may have complete sequence complementarity with the target sequence, although probes differing from the target sequence and which retain the ability to hybridize to target sequences may be designed by conventional methods.

“Stringent conditions” or “stringent hybridization conditions” include reference to conditions under which a probe will hybridize to its target sequence, to a detectably greater degree than to other sequences. Stringent conditions are target-sequence-dependent and will differ depending on the structure of the polynucleotide. By controlling the stringency of the hybridization and/or wash conditions, target sequences can be identified which are 100% complementary to the probe (homologous probing). Alternatively, stringency conditions can be adjusted to allow some mismatching in sequences so that lower degrees of similarity are detected (heterologous probing). Longer sequences hybridize specifically at higher temperatures. An extensive guide to the hybridization of nucleic acids is found in Tijssen (1993) Laboratory Techniques in Biochemistry and Molecular Biology—Hybridization with Nucleic Acid Probes, Part I, Chapter 2 “Overview of principles of hybridization and the strategy of nucleic acid probe assays”, Elsevier: New York; and Current Protocols in Molecular Biology, Chapter 2, Ausubel et al., Eds., Greene Publishing and Wiley-Interscience: New York (1995), and also Sambrook et al. (2001) Molecular Cloning: A Laboratory Manual (5^{th} Ed. Cols Spring Harbor Laboratory, Cold Spring Harbor, NY).

Specificity is typically the function of post-hybridization washes, the critical factors being the ionic strength and temperature of the final wash solution. Generally, high stringency hybridization and wash conditions are selected to be about 5° C. lower than the thermal melting point (Tₘ) for the specific sequence at a defined ionic strength and pH. The Tₘ is the temperature (under defined ionic strength and pH) at which 50% of the target sequence hybridizes to a perfectly matched probe. Typically, under high stringency conditions a probe will hybridize to its target subsequence, but to no other sequences.

An example of high stringency hybridization conditions for hybridization of complementary nucleic acids which have more than 100 complementary residues on a filter in a Southern or northern blot is 50% formamide with 1 mg of heparin at 42° C., with the hybridization being carried out overnight. An example of very high stringency wash conditions is 0.15M NaCl at 72° C. for about 15 minutes. An example of high stringency wash conditions is a 0.2×SSC wash at 65° C. for 15 minutes (see, Sambrook, infra, for a description of SSC buffer).

Exemplary hybridization conditions for the invention include hybridization in 7% SDS, 0.25 M NaPO₄ pH 7.2 at 67° C. overnight, followed by two washings in 5% SDS, 0.20 M NaPO₄ pH7.2 at 65° C. for 30 minutes each wash, and two washings in 1% SDS, 0.20 M NaPO₄ pH7.2 at 65° C. for 30 minutes each wash. An exemplary medium stringency wash for a duplex of, e.g., more than 100 nucleotides, is 1×SSC at 45° C. for 15 minutes. An exemplary low stringency wash for a duplex of, e.g., more than 100 nucleotides, is 4-6×SSC at 40° C. for 15 minutes.

For probes of about 10 to 50 nucleotides, high stringency conditions typically involve salt concentrations of less than about 1.0 M Na ion, typically about 0.01 to 1.0 M Na ion concentration (or other salts) at pH 7.0 to 8.3, and the temperature is typically at least about 30° C. High stringency conditions can also be achieved with the addition of destabilizing agents such as formamide. In general, a signal to noise ratio of 2× (or higher) than that observed for an unrelated probe in the particular hybridization assay indicates detection of a specific hybridization. Nucleic acids that do not hybridize to each other under high stringency conditions are still substantially identical if the proteins that they encode are substantially identical. This occurs, e.g., when a copy of a nucleic acid is created using the maximum codon degeneracy permitted by the genetic code.

The following are exemplary sets of hybridization/wash conditions that may be used to hybridize nucleotide sequences that are substantially identical to reference nucleotide sequences of the invention: a reference nucleotide sequence preferably hybridizes to the reference nucleotide sequence in 7% sodium dodecyl sulfate (SDS), 0.5 M NaPO₄, 1 mM EDTA at 50° C. with washing in 2×SSC, 0.1% SDS at 50° C., more desirably in 7% sodium dodecyl sulfate (SDS), 0.5 M NaPO₄, 1 mM EDTA at 50° C. with washing in 1×SSC, 0.1% SDS at 50° C., more desirably still in 7% sodium dodecyl sulfate (SDS), 0.5 M NaPO₄, 1 mM EDTA at 50° C. with washing in 0.5×SSC, 0.1% SDS at 50° C., preferably in 7% sodium dodecyl sulfate (SDS), 0.5 M NaPO₄, 1 mM EDTA at 50° C. with washing in 0.1×SSC, 0.1% SDS at 50° C., more preferably in 7% sodium dodecyl sulfate (SDS), 0.5 M NaPO₄, 1 mM EDTA at 50° C. with washing in 0.1×SSC, 0.1% SDS at 65° C. The sequences of the invention may be detected using all the above conditions. For the purposes of defining the invention, the high stringency conditions are used.

“Transformation” is a process for introducing heterologous nucleic acid into a host cell or organism. In particular, “transformation” means the stable integration of a DNA molecule into the genome of an organism of interest.

“Transformed/transgenic/recombinant” refer to a host organism such as a bacterium or a plant into which a heterologous nucleic acid molecule has been introduced. The nucleic acid molecule can be stably integrated into the genome of the host or the nucleic acid molecule can also be as an extrachromosomal molecule. Such an extrachromosomal molecule can be auto-replicating. Transformed cells, tissues, or plants are understood to encompass not only the end product of a transformation process, but also transgenic progeny thereof. A “non-transformed”, “non-transgenic”, or “non-recombinant” host refers to a wild-type organism, e.g., a bacterium or plant, which does not contain the heterologous nucleic acid molecule. As used herein, “transgenic” refers to a plant, plant cell, or multitude of structured or unstructured plant cells having integrated, via well known techniques of genetic manipulation and gene insertion, a nucleic acid representing a gene of interest into the plant genome, and typically into a chromosome of a cell nucleus, mitochondria or other organelle containing chromosomes, at a locus different to, or in a number of copies greater than, that normally present in the native plant or plant cell. Transgenic plants result from the manipulation and insertion of such nucleic acid sequences, as opposed to naturally occurring mutations, to produce a non-naturally occurring plant or a plant with a non-naturally occurring genotype. Techniques for transformation of plants and plant cells are well known in the art and may comprise for example electroporation, microinjection, Agrobacterium-mediated transformation, and ballistic transformation.

The nomenclature for DNA bases and amino acids as set forth in 37 C.F.R. § 1.822 is used herein.

### DETAILED DESCRIPTION

This invention relates to a genetically improved line of corn that produces the insect control protein, FR8a, and a phosphomannose isomerase enzyme (PMI) that allows the plant to utilize mannose as a carbon source. The invention is particularly drawn to a transgenic corn event designated event 5307 comprising a novel genotype, as well as to compositions and methods for detecting nucleic acids from this event in a biological sample. The invention is further drawn to corn plants comprising the event 5307 genotype, to transgenic seed from the corn plants, and to methods for producing a corn plant comprising the event 5307 genotype by crossing a corn inbred comprising the event 5307 genotype with itself or another corn line. Corn plants comprising the event 5307 genotype of the invention are useful in controlling coleopteran insect pests including Diabrotica virgifera virgifera, the western corn rootworm, D. virgifera zeae, the Mexican corn rootworm, and D. longicornis barberi, the northern corn rootworm. Corn plants comprising the event 5307 genotype of the invention are also able to utilize mannose as a carbon source.

In one embodiment, the invention encompasses a transgenic corn seed of an event 5307 corn plant. An example of said seed being deposited as ATCC Accession No: PTA-9561. The transgenic seed of event 5307 comprises SEQ ID NO: 1, SEQ ID NO: 2, SEQ ID NO: 3, SEQ ID NO: 4, SEQ ID NO: 5, or SEQ ID NO: 6, and complements thereof. These sequences define a point of insertion of a heterologous DNA sequence inserted into the corn plant genome of corn event 5307. In another embodiment, the invention encompasses a preferably isolated nucleic acid molecule comprising SEQ ID NO: 1, SEQ ID NO: 2, SEQ ID NO: 3 or SEQ ID NO: 4. In another embodiment, the invention encompasses a preferably isolated nucleic acid molecule, wherein the nucleic acid molecule is comprised in a corn seed deposited as ATCC Accession No. PTA-9561

In one embodiment, the invention encompasses a nucleic acid molecule, preferably isolated, comprising at least 10 or more (for example 15, 20, 25, or 50) contiguous nucleotides of a heterologous DNA sequence inserted into the corn plant genome of corn event 5307 and at least 10 or more (for example 15, 20, 25, or 50) contiguous nucleotides of a corn plant genome DNA flanking the point of insertion of a heterologous DNA sequence inserted into the corn plant genome of corn event 5307. Also included are nucleotide sequences that comprise 10 or more nucleotides of contiguous insert sequence from event 5307 and at lease one nucleotide of flanking DNA from event 5307 adjacent to the insert sequence. Such nucleotide sequences are diagnostic for event 5307. Nucleic acid amplification of genomic DNA from the 5307 event produces an amplicon comprising such diagnostic nucleotide sequences.

In another embodiment, the invention encompasses a nucleic acid molecule, preferably isolated, comprising a nucleotide sequence which comprises at least one junction sequence of event 5307 selected from the group consisting of SEQ ID NO: 1, SEQ ID NO: 2, SEQ ID NO: 3, SEQ ID NO: 4, and complements thereof, wherein a junction sequence spans the junction between a heterologous expression cassette inserted into the corn genome and DNA from the corn genome flanking the insertion site and is diagnostic for the event.

In another embodiment, the invention encompasses a preferably isolated nucleic acid linking a heterologous DNA molecule to the corn plant genome in corn event 5307 comprising a sequence of from about 11 to about 20 contiguous nucleotides selected from the group consisting of SEQ ID NO: 1, SEQ ID NO: 2, and the complements thereof.

In another embodiment, the invention encompasses an nucleic acid molecule, preferably isolated, comprising a nucleotide sequence selected from the group consisting of SEQ ID NO: 1, SEQ ID NO: 2, SEQ ID NO: 3, SEQ ID NO: 4, and complements thereof.

In one embodiment of the invention, an amplicon comprising a nucleotide sequence selected from the group consisting of SEQ ID NO: 1, SEQ ID NO: 2, SEQ ID NO: 3, SEQ ID NO: 4, and the complements thereof is provided.

In another embodiment, the invention encompasses flanking sequence primers for detecting event 5307. Such flanking sequence primers comprise an isolated nucleic acid sequence comprising at least 10-15 contiguous nucleotides from nucleotides 1-1348 of SEQ ID NO: 5 (arbitrarily designated herein as the 5′ flanking sequence), or the complements thereof, also disclosed as SEQ ID NO: 111. In one aspect of this embodiment the flanking sequence primers are selected from the group consisting of SEQ ID NO: 8 through SEQ ID NO: 14, and complements thereof. The flanking sequences can be extended to include chromosome 5 sequences, with specific emphasis on nucleotide comprised with SEQ ID NO: 103, useful in detecting sequences associated with the 5307 corn event. In the context of SEQ ID NO: 103, an “N” is defined as any base “A”, “T”, “G”, or “C”. SEQ ID NO: 110 is the reverse complement of this sequence. In the context of SEQ ID NO: 110, an “N” is defined as any base “A”, “T”, “G”, or “C”.

In another embodiment, the invention encompasses flanking sequence primers that comprise at least 10-15 contiguous nucleotides from nucleotides 1-1093 of SEQ ID NO: 6 (arbitrarily designated herein as the 3′ flanking sequence), or the complements thereof. In one aspect of this embodiment the flanking sequence primers are selected from the group consisting of SEQ ID NO: 69 through SEQ ID NO: 72, and complements thereof.

In still another embodiment, the invention encompasses a pair of polynucleotide primers comprising a first polynucleotide primer and a second polynucleotide primer which function together in the presence of a corn event 5307 DNA template in a sample to produce an amplicon diagnostic for the corn event 5307, wherein the first primer sequence is or is complementary to a corn plant genome flanking the point of insertion of a heterologous DNA sequence inserted into the corn plant genome of corn event 5307, and the second polynucleotide primer sequence is or is complementary to the heterologous DNA sequence inserted into the corn plant genome of the corn event 5307.

In one aspect of this embodiment the first polynucleotide primer comprises at least 10 contiguous nucleotides from position 1-1348 of SEQ ID NO: 5 or complements thereof. In a further aspect of this embodiment, the first polynucleotide primer comprises the nucleotide sequence set forth in SEQ ID NO: 8 through SEQ ID NO: 14, or the complements thereof. In another aspect of this embodiment the first polynucleotide primer least 10 contiguous nucleotides from position 1-1093 of SEQ ID NO: 6 or complements thereof. In another aspect of this embodiment, the first polynucleotide primer comprises the nucleotide sequence set forth in SEQ ID NO: 69 through SEQ ID NO: 72, or the complements thereof. In yet another aspect of this embodiment, the second polynucleotide primer comprises at least 10 contiguous nucleotides of SEQ ID NO: 7, or the complements thereof. In still a further aspect of this embodiment, the second polynucleotide primer comprises the nucleotide sequence set forth in SEQ ID NO: 15 to SEQ ID NO: 68, or the complements thereof.

In another aspect of this embodiment, the first polynucleotide primer, which is set forth in SEQ ID NO: 8, and the second polynucleotide primer which is set forth in SEQ ID NO: 41, function together in the presence of a corn event 5307 DNA template in a sample to produce an amplicon diagnostic for the corn event 5307 as described in Example 4. In another aspect of this embodiment, the first polynucleotide primer, which is set forth in SEQ ID NO: 69, and the second polynucleotide primer which is set forth in SEQ ID NO: 72, function together in the presence of a corn event 5307 DNA template in a sample to produce an amplicon diagnostic for the corn event 5307 as described in Example 4.

It is well within the skill in the art to obtain additional sequence further out into the genome sequence flanking either end of the inserted heterologous DNA sequences for use as a primer sequence that can be used in such primer pairs for amplifying the sequences that are diagnostic for the 5307 event. For the purposes of this disclosure, the phrase “further out into the genome sequence flanking either end of the inserted heterologous DNA sequences” refers specifically to a sequential movement away from the ends of the inserted heterologous DNA sequences, the points at which the inserted DNA sequences are adjacent to native genomic DNA sequence, and out into the genomic DNA of the particular chromosome into which the heterologous DNA sequences were inserted. Preferably, a primer sequence corresponding to or complementary to a part of the insert sequence should prime the transcriptional extension of a nascent strand of DNA or RNA toward the nearest flanking sequence junction. Consequently, a primer sequence corresponding to or complementary to a part of the genomic flanking sequence should prime the transcriptional extension of a nascent strand of DNA or RNA toward the nearest flanking sequence junction. A primer sequence can be, or can be complementary to, a heterologous DNA sequence inserted into the chromosome of the plant, or a genomic flanking sequence. One skilled in the art would readily recognize the benefit of whether a primer sequence would need to be, or would need to be complementary to, the sequence as set forth within the inserted heterologous DNA sequence or as set forth in SEQ ID NO: 3 or SEQ ID NO: 4 depending upon the nature of the product desired to be obtained through the use of the nested set of primers intended for use in amplifying a particular flanking sequence containing the junction between the genomic DNA sequence and the inserted heterologous DNA sequence. Further more, one skilled in the art would be able to design primers for a multitude of native corn genes for the purposes of designing a positive control. One such example is the corn Adh1 gene, where examples of suitable primers for producing an amplicon by nucleic acid amplification are set forth as SEQ ID NO: 79 and SEQ ID NO: 80.

In another embodiment, the invention encompasses a method of detecting the presence of DNA corresponding to the event 5307 in a biological sample, wherein the method comprises: (a) contacting the sample comprising DNA with a probe that hybridizes under high stringency conditions with genomic DNA from corn event 5307 and does not hybridize under high stringency conditions with DNA of a control corn plant; (b) subjecting the sample and probe to high stringency hybridization conditions; and (c) detecting hybridization of the probe to the DNA. In one aspect of this embodiment the amplicon comprises a nucleotide sequence selected from the group consisting of SEQ ID NO: 1, SEQ ID NO: 2, SEQ ID NO: 3, SEQ ID NO: 4, and complements thereof.

In another embodiment, the invention encompasses a method of detecting the presence of a DNA corresponding to the 5307 event in a biological sample, wherein the method comprises: (a) contacting the sample comprising DNA with a probe that hybridizes under high stringency conditions with genomic DNA from corn event 5307 and does not hybridize under high stringency conditions with DNA of a control corn plant; (b) subjecting the sample and probe to high stringency hybridization conditions; and (c) detecting hybridization of the probe to the DNA. Detection can be by any means well known in the art including but not limited to fluorescent, chemiluminescent, radiological, immunological, or otherwise. In the case in which hybridization is intended to be used as a means for amplification of a particular sequence to produce an amplicon which is diagnostic for the 5307 corn event, the production and detection by any means well known in the art of the amplicon is intended to be indicative of the intended hybridization to the target sequence where one probe or primer is utilized, or sequences where two or more probes or primers are utilized. The term “biological sample” is intended to comprise a sample that contains or is suspected of containing a nucleic acid comprising from between five and ten nucleotides either side of the point at which one or the other of the two terminal ends of the inserted heterologous DNA sequence contacts the genomic DNA sequence within the chromosome into which the heterologous DNA sequence was inserted, herein also known as the junction sequences. In addition, the junction sequence comprises as little as two nucleotides: those being the first nucleotide within the flanking genomic DNA adjacent to and covalently linked to the first nucleotide within the inserted heterologous DNA sequence.

In yet another embodiment, the invention encompasses a kit for detecting the presence of event 5307 nucleic acids in a biological sample, wherein the kit comprises at least one nucleic acid molecule of sufficient length of contiguous nucleotides homologous or complementary to a nucleotide sequence selected from the group consisting of SEQ ID NO: 1, SEQ ID NO: 2, SEQ ID NO: 3, and SEQ ID NO: 4, that functions as a DNA primer or probe specific for event 5307, and other materials necessary to enable nucleic acid hybridization or amplification. A variety of detection methods can be used including TAQMAN (Perkin Elmer), thermal amplification, ligase chain reaction, southern hybridization, ELISA methods, and colorimetric and fluorescent detection methods. In particular the invention provides for kits for detecting the presence of the target sequence, i.e., at least one of the junctions of the insert DNA with the genomic DNA of the corn plant in event 5307, in a sample containing genomic nucleic acid from event 5307. The kit is comprised of at least one polynucleotide capable of binding to the target site or substantially adjacent to the target site and at least one means for detecting the binding of the polynucleotide to the target site. The detecting means can be fluorescent, chemiluminescent, colorimetric, or isotopic and can be coupled at least with immunological methods for detecting the binding. A kit is also envisioned which can detect the presence of the target site in a sample, i.e., at least one of the junctions of the insert DNA with the genomic DNA of the corn plant in event 5307, taking advantage of two or more polynucleotide sequences which together are capable of binding to nucleotide sequences adjacent to or within about 100 base pairs, or within about 200 base pairs, or within about 500 base pairs or within about 1000 base pairs of the target sequence and which can be extended toward each other to form an amplicon which contains at least the target site

In another embodiment, the invention encompasses a method for detecting event 5307 protein in a biological sample, the method comprising: (a) extracting protein from a sample of corn event 5307 tissue; (b) assaying the extracted protein using an immunological method comprising antibody specific for the insecticidal or selectable marker protein produced by the 5307 event; and (c) detecting the binding of said antibody to the insecticidal or selectable marker protein.

Another embodiment of the invention encompasses a corn plant, or parts thereof, comprising the genotype of the transgenic event 5307, wherein the genotype comprises the nucleotide sequence set forth in SEQ ID NO: 1, SEQ ID NO: 2, SEQ ID NO: 3, SEQ ID NO: 4, or the complements thereof. In one aspect of this embodiment, the corn plant is from the inbred corn lines CG5NA58, CG5NA58A, CG3ND97, CG5NA01, CG5NF22, CG4NU15, CG00685, CG00526, CG00716, NP904, NP948, NP934, NP982, NP991, NP993, NP2010, NP2013, NP2015, NP2017, NP2029, NP2031, NP2034, NP2045, NP2052, NP2138, NP2151, NP2166, NP2161, NP2171, NP2174, NP2208, NP2213, NP2222, NP2275, NP2276, NP2316, BCTT609, AF031, H8431, 894, BUTT201, R327H, 2044BT, and 2070BT. One skilled in the art will recognize however, that the event 5307 genotype can be introgressed into any plant variety that can be bred with corn, including wild maize species, and thus the preferred inbred lines of this embodiment are not meant to be limiting.

In another embodiment, the invention encompasses a corn plant comprising at least a first and a second DNA sequence linked together to form a contiguous nucleotide sequence, wherein the first DNA sequence is within a junction sequence and comprises at least about 10-15 contiguous nucleotides selected from the group consisting of nucleotides SEQ ID NO: 5, SEQ ID NO: 6, and complements thereof, wherein the second DNA sequence is within the heterologous insert DNA sequence selected from the group consisting of SEQ ID NO: 15 through SEQ ID NO: 68, and complements thereof; and wherein the first and the second DNA sequences are useful as nucleotide primers or probes for detecting the presence of corn event 5307 nucleic acid sequences in a biological sample. In one aspect of this embodiment, the nucleotide primers are used in a DNA amplification method to amplify a target DNA sequence from template DNA extracted from the corn plant and the corn plant is identifiable from other corn plants by the production of an amplicon corresponding to a DNA sequence comprising SEQ ID NO: 1 or SEQ ID NO: 2

Corn plants of the invention can be further characterized in that digesting the plant's genomic DNA with the restriction endonucleases SmaI and PmeI results in a single hybridizing band using a full length probe under high stringency conditions. Exemplified herein is a full length probe comprising a nucleotide sequence set forth in SEQ ID NO: 7.

In one embodiment, the invention provides a corn plant, wherein the event 5307 genotype confers upon the corn plant resistance to insects or the ability to utilize mannose. In one aspect of this embodiment, the genotype conferring resistance to insects upon the corn plant comprises a FR8a gene. In another aspect of this embodiment, the genotype conferring upon the corn plant the ability to utilize mannose comprises a PMI gene.

In one embodiment, the invention provides a biological sample derived from a event 5307 corn plant, tissue, or seed, wherein the sample comprises a nucleotide sequence which is or is complementary to a sequence selected from the group consisting of SEQ ID NO: 1, SEQ ID NO: 2, SEQ ID NO: 3 and SEQ ID NO: 4, and wherein the sequence is detectable in the sample using a nucleic acid amplification or nucleic acid hybridization method. Thus, the genetic sequence functions a means of detection. In one aspect of this embodiment, the sample is selected from corn flour, corn meal, corn syrup, corn oil, corn starch, and cereals manufactured in whole or in part to contain corn products.

In another embodiment, the invention provides an extract derived from a event 5307 corn plant, tissue, or seed comprising a nucleotide sequence which is or is complementary to a nucleotide sequence selected from the group consisting of SEQ ID NO: 1, SEQ ID NO: 2, SEQ ID NO: 3 and SEQ ID NO: 4. An example of such seed is deposited at the ATCC under Accession No. PTA-9561. In one aspect of this embodiment, the sequence is detected in the extract using a nucleic acid amplification or nucleic acid hybridization method. In another aspect of this embodiment, the sample is selected from corn flour, corn syrup, corn oil, cornstarch, and cereals manufactured in whole or in part to contain corn products.

In yet another embodiment, the invention provides a method for producing a corn plant resistant to at least corn rootworm infestation comprising: (a) sexually crossing a first parent corn plant with a second parent corn plant, wherein said first or second parent corn plant comprises corn event 5307 DNA, thereby producing a plurality of first generation progeny plants; (b) selecting a first generation progeny plant that is resistant to at least corn rootworm infestation; (c) selfing the first generation progeny plant, thereby producing a plurality of second generation progeny plants; and (d) selecting from the second generation progeny plants, a plant that is at least resistant to corn rootworm infestation; wherein the second generation progeny plants comprise a nucleotide sequence selected from the group consisting of SEQ ID NO: 1, SEQ ID NO: 2, SEQ ID NO:3 and SEQ ID NO: 4.

In another embodiment, the invention provides a method of producing hybrid corn seeds comprising: (a) planting seeds of a first inbred corn line comprising a nucleotide sequence selected from the group consisting of SEQ ID NO: 1, SEQ ID NO: 2, SEQ ID NO: 3, and SEQ ID NO: 4, and seeds of a second inbred line having a different genotype; (b) cultivating corn plants resulting from said planting until time of flowering; (c) emasculating said flowers of plants of one of the corn inbred lines; (d) sexually crossing the two different inbred lines with each other; and (e) harvesting the hybrid seed produced thereby. In one aspect of this embodiment, the first inbred corn line provides the female parents. In another aspect of this embodiment, the first inbred corn line provides the male parents. The invention also encompasses the hybrid seed produced by the embodied method and hybrid plants grown from the seed.

In another embodiment, the invention provides a method of selecting markers associated with corn event 5307 comprising: (a) screening corn event 5307 chromosome 5 sequences, (b) comparing these with a non-transgenic NP2222 sequences, (c) comparing the sequences for the purpose of detecting sequence variations, (d) using these sequence variations as a means to develop markers associated with corn event 5307, (e) using the markers to screen lines, and (f) detecting marker confirming the presence of corn event 5307 sequences on chromosome 5.

One skilled in the art will recognize that the transgenic genotype of the invention can be introgressed by breeding into other corn lines comprising different transgenic genotypes. For example, a corn inbred comprising the transgenic genotype of the invention can be crossed with a corn inbred comprising the transgenic genotype of the lepidopteran resistant Bt11 event, which is known in the art, thus producing corn seed that comprises both the transgenic genotype of the invention and the Bt11 transgenic genotype. Examples of other transgenic events which can be crossed with an inbred of the invention include, the glyphosate herbicide tolerant events GA21 and NK603, the glyphosate tolerant/lepidopteran insect resistant MON802 event, the lepidopteran insect resistant event DBT418, the lepidopteran insect resistant event DAS-06275-8, the lepidopteran insect resistant event MIR162, the male sterile event MS3, the phosphinothricin tolerant event B16, the lepidopteran insect resistant event MON 80100, the phosphinothricin herbicide tolerant events T14 and T25, the lepidopteran insect resistant event 176, the coleopteran insect resistant event MIR604 and the coleopteran insect resistant event MON863, all of which are known in the art. It will be further recognized that other combinations can be made with the transgenic genotype of the invention and thus these examples should not be viewed as limiting.

One skilled in the art will also recognize that transgenic corn seed comprising the transgenic genotype of the invention can be treated with various seed-treatment chemicals, including insecticides, to augment or syngergize the insecticidal activity of the FR8a protein. For example, the transgenic corn seed of the invention can be treated with the commercial insecticide Cruiser®. Such a combination may be used to increase the spectrum of activity and to increase the efficacy of the expressed protein and chemical.

### Breeding

The transgenic genotype of the invention can be introgressed in any corn inbred or hybrid using art recognized breeding techniques. The goal of plant breeding is to combine in a single variety or hybrid various desirable traits. For field crops, these traits may include resistance to insects and diseases, tolerance to herbicides, tolerance to heat and drought, reducing the time to crop maturity, greater yield, and better agronomic quality. With mechanical harvesting of many crops, uniformity of plant characteristics such as germination and stand establishment, growth rate, maturity, and plant and ear height, is important.

Field crops are bred through techniques that take advantage of the plant's method of pollination. A plant is self-pollinated if pollen from one flower is transferred to the same or another flower of the same plant. A plant is cross-pollinated if the pollen comes from a flower on a different plant.

Plants that have been self-pollinated and selected for type for many generations become homozygous at almost all gene loci and produce a uniform population of true breeding progeny. A cross between two different homozygous lines produces a uniform population of hybrid plants that may be heterozygous for many gene loci. A cross of two plants each heterozygous at a number of gene loci will produce a population of hybrid plants that differ genetically and will not be uniform.

Corn can be bred by both self-pollination and cross-pollination techniques. Corn has separate male and female flowers on the same plant, located on the tassel and the ear, respectively. Natural pollination occurs in corn when wind blows pollen from the tassels to the silks that protrude from the tops of the ears.

A reliable method of controlling male fertility in plants offers the opportunity for improved plant breeding. This is especially true for development of corn hybrids, which relies upon some sort of male sterility system. There are several options for controlling male fertility available to breeders, such as: manual or mechanical emasculation (or detasseling), cytoplasmic male sterility, genetic male sterility, gametocides and the like.

Hybrid corn seed is typically produced by a male sterility system incorporating manual or mechanical detasseling. Alternate strips of two corn inbreds are planted in a field, and the pollen-bearing tassels are removed from one of the inbreds (female). Providing that there is sufficient isolation from sources of foreign corn pollen, the ears of the detasseled inbred will be fertilized only from the other inbred (male), and the resulting seed is therefore hybrid and will form hybrid plants.

The laborious, and occasionally unreliable, detasseling process can be avoided by using one of many methods of conferring genetic male sterility in the art, each with its own benefits and drawbacks. These methods use a variety of approaches such as delivering into the plant a gene encoding a cytotoxic substance associated with a male tissue specific promoter or an antisense system in which a gene critical to fertility is identified and an antisense to that gene is inserted in the plant (see: Fabinjanski, et al. EPO 89/3010153.8 publication no. 329,308 and PCT application PCT/CA90/00037 published as WO 90/08828).

### Development of Corn Inbred Lines

The use of male sterile inbreds is but one factor in the production of corn hybrids. Plant breeding techniques known in the art and used in a corn plant breeding program include, but are not limited to, recurrent selection, backcrossing, pedigree breeding, restriction length polymorphism enhanced selection, marker assisted selection and transformation. The development of corn hybrids in a corn plant breeding program requires, in general, the development of homozygous inbred lines, the crossing of these lines, and the evaluation of the crosses. Pedigree breeding and recurrent selection breeding methods are used to develop inbred lines from breeding populations. Corn plant breeding programs combine the genetic backgrounds from two or more inbred lines or various other germplasm sources into breeding pools from which new inbred lines are developed by selfing and selection of desired phenotypes. The new inbreds are crossed with other inbred lines and the hybrids from these crosses are evaluated to determine which of those have commercial potential. Plant breeding and hybrid development, as practiced in a corn plant-breeding program, are expensive and time-consuming processes.

Pedigree breeding starts with the crossing of two genotypes, each of which may have one or more desirable characteristics that is lacking in the other or which complements the other. If the two original parents do not provide all the desired characteristics, other sources can be included in the breeding population. In the pedigree method, superior plants are selfed and selected in successive generations. In the succeeding generations the heterozygous condition gives way to homogeneous lines as a result of self-pollination and selection. Typically in the pedigree method of breeding five or more generations of selfing and selection is practiced: F₁→F₂, F₂→F₃; F₃→F₄; F₄→F.₅; etc.

Recurrent selection breeding, backcrossing for example, can be used to improve an inbred line and a hybrid that is made using those inbreds. Backcrossing can be used to transfer a specific desirable trait from one inbred or source to an inbred that lacks that trait. This can be accomplished, for example, by first crossing a superior inbred (recurrent parent) to a donor inbred (non-recurrent parent), that carries the appropriate gene(s) for the trait in question. The progeny of this cross is then mated back to the superior recurrent parent followed by selection in the resultant progeny for the desired trait to be transferred from the non-recurrent parent. After five or more backcross generations with selection for the desired trait, the progeny will be homozygous for loci controlling the characteristic being transferred, but will be like the superior parent for essentially all other genes. The last backcross generation is then selfed to give pure breeding progeny for the gene(s) being transferred. A hybrid developed from inbreds containing the transferred gene(s) is essentially the same as a hybrid developed from the same inbreds without the transferred gene(s).

Elite inbred lines, that is, pure breeding, homozygous inbred lines, can also be used as starting materials for breeding or source populations from which to develop other inbred lines. These inbred lines derived from elite inbred lines can be developed using the pedigree breeding and recurrent selection breeding methods described earlier. As an example, when backcross breeding is used to create these derived lines in a corn plant-breeding program, elite inbreds can be used as a parental line or starting material or source population and can serve as either the donor or recurrent parent.

### Development of Corn Hybrids

A single cross corn hybrid results from the cross of two inbred lines, each of which has a genotype that complements the genotype of the other. The hybrid progeny of the first generation is designated F₁. In the development of commercial hybrids in a corn plant-breeding program, only the F₁ hybrid plants are sought. Preferred F₁ hybrids are more vigorous than their inbred parents. This hybrid vigor, or heterosis, can be manifested in many polygenic traits, including increased vegetative growth and increased yield.

The development of a corn hybrid in a corn plant breeding program involves three steps: (1) the selection of plants from various germplasm pools for initial breeding crosses; (2) the selfing of the selected plants from the breeding crosses for several generations to produce a series of inbred lines, which, although different from each other, breed true and are highly uniform; and (3) crossing the selected inbred lines with different inbred lines to produce the hybrid progeny (F₁). During the inbreeding process in corn, the vigor of the lines decreases. Vigor is restored when two different inbred lines are crossed to produce the hybrid progeny (F₁). An important consequence of the homozygosity and homogeneity of the inbred lines is that the hybrid between a defined pair of inbreds will always be the same. Once the inbreds that give a superior hybrid have been identified, the hybrid seed can be reproduced indefinitely as long as the homogeneity of the inbred parents is maintained. Much of the hybrid vigor exhibited by F₁ hybrids is lost in the next generation (F₂). Consequently, seed from hybrids is not used for planting stock.

Hybrid seed production requires elimination or inactivation of pollen produced by the female parent. Incomplete removal or inactivation of the pollen provides the potential for self-pollination. This inadvertently self-pollinated seed may be unintentionally harvested and packaged with hybrid seed.

Once the seed is planted, it is possible to identify and select these self-pollinated plants. These self-pollinated plants will be genetically equivalent to the female inbred line used to produce the hybrid.

As is readily apparent to one skilled in the art, the foregoing are only some of the various ways by which the inbred of the invention can be obtained by those looking to introgress the transgenic genotype of the invention into other corn lines. Other means are available, and the above examples are illustrative only.

### EXAMPLES

The invention will be further described by reference to the following detailed examples. These examples are provided for purposes of illustration only, and are not intended to be limiting unless otherwise specified. Standard recombinant DNA and molecular cloning techniques used here are well known in the art and are described by Ausubel (ed.), Current Protocols in Molecular Biology, John Wiley and Sons, Inc. (1994); J. Sambrook, et al., Molecular Cloning: A Laboratory Manual, 3d Ed., Cold Spring Harbor, NY: Cold Spring Harbor Laboratory Press (2001); and by T. J. Silhavy, M. L. Berman, and L. W. Enquist, Experiments with Gene Fusions, Cold Spring Harbor Laboratory, Cold Spring Harbor, NY (1984).

### Example 1. Transformation and Selection of the 5307 Event

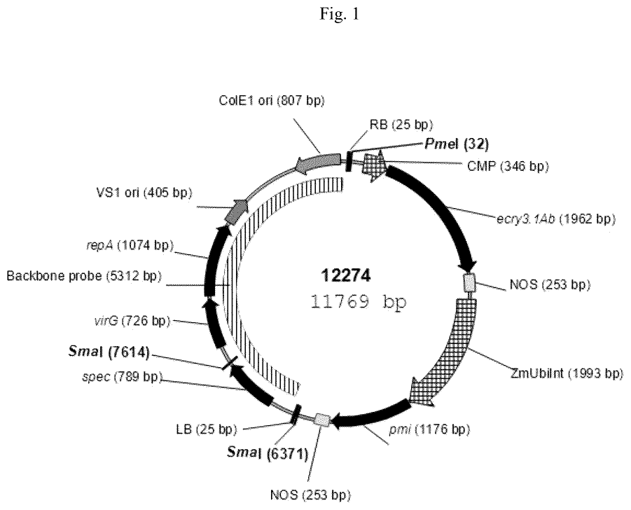

The 5307 event was produced by Agrobacterium-mediated transformation of the inbred corn (Zea mays) line NP2222. Immature embryos were transformed essentially as described in Negrotto et al. (Plant Cell Reports 19:798-803, 2000), incorporated herein by reference, using a DNA fragment from plasmid pSYN12274 (FIG. 1). pSYN12274 contains a nucleotide sequence comprising tandem expression cassettes. The first expression cassette is comprised of a CMP promoter sequence (U.S. Pat. No. 7,166,770) operably linked to a FR8a coding sequence further operably linked to a nopaline synthase 3′ end transcription termination and polyadenylation sequence. The second expression cassette is comprised of a maize ubiquitin promoter (ZmUbiInt) (Christensen et al. 1992 PMB 18:675) operably linked to a PMI coding sequence further operably linked to a nopaline synthase 3′ end transcription termination and polyadenylation sequence.

Immature embryos were excised from 8-12 day old ears and rinsed with fresh medium in preparation for transformation. Embryos were mixed with the suspension of Agrobacterium cells harboring the transformation vector pSYN12274, vortexed for 30 seconds, and allowed to incubate for an additional 5 minutes. Excess Agrobacterium solution was aspirated and embryos were then moved to plates containing a non-selective culture medium. Embryos were co-cultured with the remaining Agrobacterium at 22° C. for 2-3 days in the dark. Embryos were transferred to culture medium supplemented with ticarcillin (100 mg/ml) and silver nitrate (1.6 mg/l) and incubated in the dark for 10 days. Embryos producing embryogenic callus were transferred to cell culture medium containing mannose.

Regenerated plantlets were tested by TAQMAN® PCR analysis (see Example 2) for the presence of both the PMI and FR8a genes, as well as for the absence of the antibiotic resistance spectinomycin (spec) gene. Plants positive for both transgenes, and negative for the spec gene, were transferred to the greenhouse for further propagation. Positive events were identified and screened using insect bioassays against corn rootworm. Insecticidal events were characterized for copy number by TAQMAN analysis. Event 5307 was chosen for further analysis based on having a single copy of the transgenes, good protein expression as identified by ELISA, and better insecticidal activity against corn rootworm when compared to other events made with the same construct.

The T₀ 5307 event was backcrossed to inbred corn line NP2460, creating the T₁ population. The T₁ plants were self-pollinated to create the T₂ generation, and this process was repeated to create a T₃ generation. Progeny testing of the T₃ plants was employed to identify homozygous (converted) families. The event 5307-converted NP2460 inbred was crossed to other elite inbred lines to create hybrids used in further studies.

### Example 2. Event 5307 Detection by TAQMAN PCR

TAQMAN analysis was essentially carried out as described in Ingham et al. (Biotechniques, 31:132-140, 2001) herein incorporated by reference. Briefly, genomic DNA was isolated from leaves of transgenic and non-transgenic corn plants using the Puregene® Genomic DNA Extraction kit (Gentra Systems, Minneapolis, MN) essentially according to the manufacturer's instruction, except all steps were conducted in 1.2 ml 96-well plates. The dried DNA pellet was resuspended in TE buffer (10 Mm Tris-HCl, pH 8.0, 1 mM EDTA).

TAQMAN PCR reactions were carried out in 96-well plates. For the endogenous corn gene control, primers and probes were designed specific to the Zea mays alcohol dehydrogenase (Adh) gene (Genbank accession no. AF044295). It will be recognized by the skilled person that other corn genes can be used as endogenous controls. Reactions were multiplexed to simultaneously amplify FR8a and Adh or PMI and Adh. For each sample, a master mixture was generated by combining 20 μL extracted genomic DNA with 35 μL 2× TAQMAN Universal PCR Master Mix (Applied Biosystems) supplemented with primers to a final concentration of 900 nM each, probes to a final concentration of 100 nM each, and water to a 70 μL final volume. This mixture was distributed into three replicates of 20 μL each in 96-well amplification plates and sealed with optically clear heat seal film (Marsh Bio Products). PCR was run in the ABI Prism 7700 instrument using the following amplification parameters: 2 min at 50° C. and 10 min at 95° C., followed by 35 cycles of 15 s at 95° C. and 1 min at 60° C.

Results of the TAQMAN analysis demonstrated that event 5307 had one copy of the FR8a gene and one copy of the PMI gene.

Examples of suitable primer/probe sequence combinations which were used are:

| Primer Name | Primer Sequence | SEQ ID NO: |
|---|---|---|
| FR8a-forward | 5′-TACGAGAGCTGGGTGAACTTCA-3′ | SEQ ID NO: 73 |
| FR8a-reverse | 5′-CGATCAGGTCCAGCACGG-3′ | SEQ ID NO: 74 |
| FR8a-probe | 5′-CCGCTACCGCCGCGAGATGA-3′ | SEQ ID NO: 75 |
| | (5′ label = FAM, 3′ label = TAMRA) | |
| PMI-forward | 5′-CCGGGTGAATCAGCGTTT-3′ | SEQ ID NO: 76 |
| PMI-reverse | 5′-GCCGTGGCCTTTGACAGT-3′ | SEQ ID NO: 77 |
| PMI-probe | 5′-TGCCGCCAACGAATCACCGG-3′ | SEQ ID NO: 78 |
| | (5′ label = FAM, 3′ label = TAMRA) | |
| ZmADH-267 forward | 5′-GAACGTGTGTTGGGTTTGCAT-3′ | SEQ ID NO: 79 |
| ZmADH-337 reverse | 5′-TCCAGCAATCCTTGCACCTT-3′ | SEQ ID NO: 80 |
| ZmADH-316 probe | 5′-TGCAGCCTAACCATGCGCAGGGTA-3′ | SEQ ID NO: 81 |
| | (5′ label = TET, 3′ label = TAMRA) | |

The PM1271, MIC5307a and MIC5307b TAQMAN assays are designed as an event specific assay, which covers the 3′ junction sequence.

Examples of suitable primer/probe sequence combinations which were used are:

| Primer Name | Primer Sequence | SEQ ID NO: |
|---|---|---|
| PM1277-forward | 5′-GCCGTATCCGCAATGTGTTA-3′ | SEQ ID NO: 82 |
| PM1277-reverse | 5′-GGCCCAGGGAAGAGGGTATAT-3′ | SEQ ID NO: 83 |
| PM1277-probe | 5′-AAGTTGTCTAAGCGTCAAT-3′ | SEQ ID NO: 84 |
| | (5′ label = TET, 3′ label = TAMRA) | |
| MIC5307a-forward | 5′-TGTCTAAGCGTCAATTTGTTTACACC-3′ | SEQ ID NO: 82 |
| MIC5307a-reverse | 5′-TTTGCCAGTGGGCCCA-3′ | SEQ ID NO: 83 |
| MIC5307a-probe | 5′-ACAATATACCCTCTTCCCTGGGCCAGG-3′ | SEQ ID NO: 84 |
| | (5′ label = TET, 3′ label = TAMRA) | |
| MIC5307b-forward | 5′-GCCGTATCCGCAATGTGTTA-3′ | SEQ ID NO: 82 |
| MIC5307b-reverse | 5′-AAGTTGTCTAAGCGTCAAT-3′ | SEQ ID NO: 83 |
| MIC5307b-probe | 5′-GGCCCAGGGAAGAGGGTATAT-3′ | SEQ ID NO: 84 |
| | (5′ label = TET, 3′ label = TAMRA) | |

### Example 3. Event 5307 Detection by Southern Blot

Genomic DNA used for southern analysis was isolated from pooled leaf tissue of ten plants representing the backcross six (BC6) generation of event 5307 using essentially the method of Thomas et al. (Theor. Appl. Genet. 86:173-180, 1993), incorporated herein by reference. All plants used for DNA isolation were individually analyzed using TAQMAN PCR (as described in Example 2) to confirm the presence of a single copy of the FR8a gene and the PMI gene. For the negative segregant controls, DNA was isolated from pooled leaf tissue of five plants representing the BC4 generation of event 5307. These negative segregant plants were individually analyzed using TAQMAN PCR and the assays were negative for the presence of the FR8a gene and the PMI gene, but were, as expected, positive for the assay internal control, the endogenous maize Adh gene.

Southern analysis was carried out using conventional molecular biology techniques. Genomic DNA (7.5 μg) was doubly digested with SmaI and PmeI restriction enzymes, which have single recognition sites within the event 5307 T-DNA insert from plasmid pSYN12274 (FIG. 1). This approach allows for determination of the number of copies of the elements, corresponding to the specific probe used for each Southern, which have been incorporated into event 5307. This results in one hybridization band per copy of the element present in event 5307. This results in one hybridization band per copy of the element present in event 5307. Following agarose gel electrophoresis and alkaline transfer to a Nytran® membrane, hybridizations were carried out using element-specific full-length PCR-generated probes. The full length probe used in the Southern blots comprises the nucleotide sequences set forth in SEQ ID NO: 7. The probe was labeled with 32P via random priming using the Rediprime™ II system (Amersham Biosciences, Cat. No. RPN1633).

The following high stringency hybridization conditions were used: 1-2 million cpm/ml are added to PerfectHyb (Sigma) supplemented with 100 μg/ml Calf Thymus DNA (Invitrogen) pre-warmed to 65° C. Pre-hybridization takes place in the same solution as above, at the same temp overnight or for at least one hour. Hybridization was carried out at 65° C. for 3 hours followed by washing 2× in 2×SSC, 0.1% SDS for 20 minutes at 65° C. and 2× in 0.1×SSC, 0.1% SDS for 20 minutes at 65° C.

Included on each Southern were three control samples: (1) DNA from a negative (non-transformed) segregant used to identify any endogenous Zea mays sequences that may cross-hybridize with the element-specific probe; (2) DNA from a negative segregant into which is introduced an amount of SmaI-PmeI digested pSYN12274 that is equal to one copy number based on probe length, to demonstrate the sensitivity of the experiment in detecting a single gene copy within the Zea mays genome; and (3) SmaI-PmeI digested pSYN12274 plasmid that is equal to one copy number based on probe length, as a positive control for hybridization as well as to demonstrate the sensitivity of the experiment.

The hybridization data provide confirmatory evidence to support the TAQMAN PCR analysis that event 5307 contains a single copy of the FR8a and PMI genes, and that 5307 event does not contain any of the vector backbone sequences present in pSYN12274. As expected for both the FR8a and PMI probes, the SmaI-PmeI digest resulted in a single hybridization band of the correct size, demonstrating that a single copy of each gene is present in the 5307 event. Additionally, for the backbone probe lack of hybridization demonstrates the absence of any pSYN12274 vector backbone sequences being incorporated into event 5307 during the transformation process.

### Example 4. T-DNA Insert Sequencing

The nucleotide sequence of the entire transgene DNA insert present in event 5307 was determined to demonstrate overall integrity of the insert, contiguousness of the functional elements and to detect any individual basepair changes. The event 5307 insert was PCR amplified from DNA derived from the BC5 generation as two individual overlapping fragments. Each fragment was amplified using one polynucleotide primer homologous to plant genomic sequences flanking the event 5307 insert and one polynucleotide primer homologous to the FR8a gene. To generate the 5′ fragment, a first polynucleotide primer homologous to the 5′ flanking sequence, SEQ ID NO: 8 through SEQ ID NO: 15, was combined with a second polynucleotide primer homologous to the inserted DNA the FR8a gene, SEQ ID NO: 33 through SEQ ID NO: 41, the Ubiquitin promoter, SEQ ID NO: 42 through SEQ ID NO: 53 or the PMI gene, SEQ ID NO: 54 through SEQ ID NO: 60. To generate the 3′ fragment, a first polynucleotide primer homologous to the 3′ flanking sequence, SEQ ID NO: 69 through SEQ ID NO: 72, was combined with a second polynucleotide primer homologous to the inserted DNA within the FR8a gene, SEQ ID NO: 9 through SEQ ID NO: 17, the Ubiquitin promoter, SEQ ID NO: 18 through SEQ ID NO: 26 or the PMI gene, SEQ ID NO: 27 through SEQ ID NO: 32.

PCR amplification was carried out using the Expand High Fidelity PCR system (Roche, Cat. No. 1732650) and the following amplification parameters: 2 min at 94° C. for 1 cycle, followed by 10 cycles of 15 s at 94° C., 30 s at 55-65° C. and 5 min at 68° C., followed by 20 cycles of 15 s 94° C., 30 s at 55-65° C., and 5 min+5 s/cyc of 72° C., followed by 1 cycle of 7 min at 72° C.

The amplicon resulting from the PCR amplification using SEQ ID NO: 8 and SEQ ID NO: 41 comprised the 5′ junction sequence (SEQ ID NO: 1). The amplicon resulting from the PCR amplification using SEQ ID NO: 69 and SEQ ID NO: 72 comprised the 3′ junction sequence (SEQ ID NO: 2). Each sequencing fragment was individually cloned into the pCR®-XL-TOPO vector (Invitrogen, Cat. No. K4700-20) and three separate clones for each fragment were identified and sequenced. Sequencing was carried out using the ABI3730XL analyzer using ABI BigDye® 1.1 or Big Dye 3.1 dGTP (for GC rich templates) chemistry. The sequence analysis was done using the Phred, Phrap, and Consed package from the University of Washington and was carried out to an error rate of less than 1 in 10,000 bases (Ewing and Green, 1998). The final consensus sequence was determined by combining the sequence data from the six individual clones (three for each sequencing fragment) to generate one consensus sequence of the event 5307 insert. To further validate any individual basepair discrepancies between the event 5307 insert and the pSYN12274 plasmid, small (approximately 300-500 bp) PCR products specific to any regions where a basepair discrepancy was seen in the initial consensus sequence were amplified using the same methodology above. For all putative basepair discrepancies in the event 5307 insert, direct PCR product sequencing resulted in single clear peaks at all basepairs in question, indicating these discrepancies are likely present in the event 5307 insert. Alignment was performed using the ClustalW program with the following parameters: scoring matrix blosum55, gap opening penalty 15, gap extension penalty 6.66 (Thompson et al, 1994, Nucleic Acids Research, 22, 4673-4680).

The consensus sequence data for the event 5307 T-DNA insert demonstrates that the overall integrity of the insert and contiguousness of the functional elements within the insert as intended in pSYN12274 have been maintained.

### Example 5. Analysis of Flanking DNA Sequence

Corn genome DNA sequence flanking the heterologous DNA inserted into the corn plant genome of event 5307 was obtained using OmniPlex™ Technology essentially as described in Kamberov et al (Proceedings of SPIE, Tools for Molecular Analysis and High-Throughput Screening, 4626:1-12, 2002), incorporated herein by reference.

The 5′ and 3′ flanking sequences and junction sequences were confirmed using standard PCR procedures. The 5′ flanking and junction sequences were confirmed using a first polynucleotide primer set forth in SEQ ID NO: 8 through SEQ ID NO: 14 combined with a second polynucleotide primer set forth in SEQ ID NO: 33 through SEQ ID NO: 41. The 3′ flanking and junction sequences were confirmed using a first polynucleotide primer set forth in SEQ ID NO: 69 through SEQ ID NO: 72 combined with a second polynucleotide primer set forth in SEQ ID NO: 27 through SEQ ID NO: 32. It will be recognized by the skilled person that other primer sequences can be used to confirm the flanking and junction sequences.

The event 5307 insert was found to be flanked on the right border (5′ flanking sequence) by the corn genomic sequence shown in SEQ ID NO: 5 and flanked on the left border (3′ flanking sequence) by the corn genomic sequence shown in SEQ ID NO: 6. The 5′ junction sequence is set forth in SEQ ID NO: 1. The 3′ junction sequence is set forth in SEQ ID NO: 2. The integration site of the pSYN12274 vector insertion is comprised within SEQ ID NO: 103 or its reverse complement SEQ ID NO: 110, depending on the orientation of the nucleic acid used.

### Example 6. Detection of Event 5307 Protein via ELISA

To characterize the range of expression of FR8a (the active insecticidal principle) and phosphomannose isomerase (PMI) (the selectable marker) proteins in event 5307 plants, the concentrations of FR8a protein and PMI were determined by ELISA in several plant tissues. The hybrids were hemizygous for the transgenes in event 5307, whereas the inbred was homozygous for the transgenes.

Whole plants and individual parts (except pollen) were reduced to a fine powder by processing using either a coffee grinder, blender, Grindomix™ grinder (Brinkmann Instruments; Westbury, NY, USA), mortar with a pestle or mill, or a combination of these devices. All processing was done in the presence of either dry ice or liquid nitrogen. Samples were mixed well to ensure homogeneity. The entire plant tissue sample, or a representative sub-sample, was retained for analysis, allowing sufficient sample size for archival storage of reserve plant tissue samples. The percent dry weight of each sample was determined and the processed samples were stored at ca. −80° C. until lyophilization.

Fresh tissue (except pollen and silage) and whole-plant samples were extracted. For each sample analyzed, a 1.0 g aliquot of the powdered fresh material was weighed into a 15-ml polypropylene tube, suspended in 3 ml extraction buffer [50 mM CAPS, 0.1 M NaCl, 2 mM EDTA, 1 mM dithiothreitol, 1 mM 4-(1-aminoethyl)benzenesulfonyl fluoride HCl, 1 mM leupeptin, pH 10], and extracted using an Autogizer® homogenizer (Tomtek; Hamden, CT, USA). After centrifugation for 15 min at 10,000×g at 4° C., the supernatant was used for FR8a and PMI analysis by ELISA. After treatment with iodoacetamide as described by Hill and Straka (1988), total protein in the extracts was quantitated using the BCA™ Protein Assay Reagent (Pierce; Rockford, IL, USA).

Pollen extracts were prepared by suspending pollen 1:30 (w/v) in extraction buffer. After 30 min on ice, the pollen suspensions were disrupted by three passages through a French pressure cell at ca. 15,000 psi, followed by centrifugation at 14,000×g for 5 min at 4° C. Cry3A055 and PMI analyses by ELISA were performed on the supernatants as described below. Total protein was quantitated as described above.

Silage extracts were prepared by suspending silage 1:25 (w/v) in 2× extraction buffer. After 30 min on ice, the silage suspensions were extracted using a Brinkmann Polytron® Homogenizer (Brinkmann; Westbury, NY, USA). After centrifugation for 15 min at 10,000×g at 4° C., the supernatant was used for FR8a and PMI analysis by ELISA. Total protein was quantitated as described above.

### FR8a Quantification

The extracts prepared as described above were quantitatively analyzed for FR8a by ELISA (Tijssen, 1985) using immuno-affinity purified monoclonal, anti-mCry3A antibody and immuno-affinity purified polyclonal anti-Cry1Ab antibody. The lower limit of quantification of the double-sandwich ELISA was estimated based on the lowest concentration of pure reference protein lying on the linear portion of the standard curve, the maximum volume of a control extract that could be analyzed without background interference, and the corresponding weight of the sample that the aliquot represented.

Quantifiable levels of FR8a protein were detected in all event 5307-derived plant tissues. In most cases, results are presented as means of the five replicate tissue samples. Control sample levels were below the limit of quantification for all tissues.

Across all growth stages, mean FR8a levels measured in leaves, roots and pollen ranged from ca. 18-29 μg/g fresh wt. (77-113 μg/g dry wt.), ca. 1.8-4.1 μg/g fresh wt. (22-41 μg/g dry wt.) and ca. <LOD—0.15 μg/g fresh wt. (<LOD—0.15 μg/g dry wt.) respectively. [limit of detection (LOD)=0.08 μg/g fresh wt., 0.08 μg/g dry wt.].

The levels of FR8a were generally similar among the inbred and hybrid genotypes for each tissue type at each time point

### PMI Quantification

The extracts prepared as described above were quantitatively analyzed for PMI by ELISA (Tjissen, 1985) using Protein A-purified polyclonal rabbit and immunoaffinity-purified polyclonal goat antibodies specific for PMI. The lower limit of quantification of the double-sandwich ELISA was estimated based on the lowest concentration of pure reference protein lying on the linear portion of the standard curve, the maximum volume of a control extract that could be analyzed without background interference, and the corresponding weight of the sample that the aliquot represented.

PMI protein was detected in most of the event 5307-derived plant tissues analyzed. In most cases, results are presented as means of the five replicate tissue samples. Control sample levels were below the limit of quantification for all stages and tissues.

Across all plant stages, mean PMI levels measured in leaves, roots and pollen ranged from ca. 0.4 to ca. 0.6 μg/g fresh wt. (1.5-2.3 μg/g dry wt.), ca. 0.1-0.2 μg/g fresh wt. (0.9-1.5 μg/g dry wt.) and ca. 16.7-30.6 μg/g fresh wt. (17.1-31.1 μg/g dry wt.) respectively. [limit of detection (LOD)=0.08 μg/g fresh wt., 0.08 μg/g dry wt.].

The levels of PMI were generally similar among the inbred and hybrid genotypes for each tissue type at each time point.

### Example 7. Field Efficacy of Event 5307

### Western and Northern Corn Rootworm

Event 5307 plants were tested for efficacy against western and northern corn rootworm at 12 locations in the United States. Event 5307 was tested with and without the addition of the insecticidal seed treatment Crusier®. Control groups consisted of seed treated with two different rates of Cruiser® and an untreated check. Treatments consisted of four replications of two 17.5-20 foot rows spaced 30″ on center designed in a randomized complete block. Ten plants per treatment were chosen at random and evaluated for efficacy using a 0-3 scale wherein 0=No feeding damage (lowest rating that can be given); 1=One node (circle of roots), or the equivalent of an entire node, eaten back within approximately two inches of the stalk (soil line on the 7th node); 2=Two complete nodes eaten; 3=Three or more nodes eaten (highest rating that can be given). Damage in between complete nodes eaten was noted as the percentage of the node missing, i.e. 1.50=1½ nodes eaten, 0.25=¼ of one node eaten.

Event 5307 efficacy was compared with commercial granular insecticide standards applied in-furrow. The experimental design was as described above. Results in Table 2 demonstrate that the efficacy of event 5307 was comparable to the commercial standards in protecting plants against corn rootworm feeding damage.

TABLE 2
Comparison of efficacy of event 5307 with
commercial insecticides applied in-furrow.

| | Root Damage Rating |
|---|---|
| Treatment | (0-3 CRW Scale) |
| 5307 | 0.06 |
| Force ® 3G | 0.23 |
| MIR604 | 0.13 |
| Untreated Check | 2.05 |

### Mexican Corn Rootworm

Event 5307 plants were evaluated for resistance to the Mexican corn rootworm at two locations in Texas. Experimental design was essentially the same as described above.

A clear rate response was evident. Results shown in Table 3 demonstrate that the efficacy of event 5307 was comparable to the commercial standards in protecting plants against Mexican corn rootworm feeding damage.

TABLE 3
Efficacy of event 5307 compared with commercial insecticides
applied in-furrow against Mexican corn rootworm.

| | Root Damage Rating |
|---|---|
| Treatment | (0-3 CRW Scale) |
| Event 5307 | 0.025 |
| Force ® 3G | 0.084 |
| MIR604 with Cruiser ® | 0.104 |
| Untreated Check | 0.710 |

All publications and patent applications mentioned in this specification are indicative of the level of skill of those skilled in the art to which this invention pertains. All publications and patent applications are herein incorporated by reference to the same extent as if each individual publication or patent application was specifically and individually indicated to be incorporated by reference.

Although the foregoing invention has been described in some detail by way of illustration and example for purposes of clarity of understanding, it will be obvious that certain changes and modifications may be practiced within the scope of the invention.

### Example 8. Use of Event 5307 Insertion Site for Targeted Integration in Maize

The event 5307 flanking sequences disclosed in SEQ ID NO: 5 and SEQ ID NO: 6 were used to search maize genome databases. Identical matches to both flanking sequences where found on a BAC clone, ZMMBBc0077H14, of chromosome 5 (NCBI Accession No. AC202955). More specifically, the event 5307 insert lies between a 5′ marker, designated herein as the public molecular marker umc1475 (SEQ ID No: 104), and a 3′ marker, designated herein as the public molecular marker uaz190 (SEQ ID No: 107). Using this information, it was determined that the heterologous DNA inserted into event 5307 displaced 38 nucleotides of maize genomic DNA, which lies between the 5′ flanking sequence (upstream of the deleted sequence) and the 3′ flanking sequence (down stream of the deleted sequence). Primers useful for identifying molecular marker uaz190 are set forth as SEQ ID NO: 108 and 109. Primers useful for identifying molecular marker umc1475 are set forth as SEQ ID NO: 105 and 106. Further markers were developed for the purposes of fine mapping the insertion site. These markers are designated as SM1108C, SM0584B, SM0377D and SM0501D. Primers and probes useful for detecting these markers are as follows: SM1108C, SEQ ID NO: 91 through SEQ ID NO: 93; SM0584B, SEQ ID NO: 94 through SEQ ID: 96; SM0377D, SEQ ID NO: 97 through SEQ ID NO: 99; and SM0501D, SEQ ID NO: 100 through SEQ ID NO: 102.

Consistent agronomic performance of the transgene of event 5307 over several generations under field conditions suggests that these identified regions around the event 5307 insertion site provide good genomic locations for the targeted integration of other transgenic genes of interest. Such targeted integration overcomes the problems with so-called “positions effects,” and the risk of creating a mutation in the genome upon integration of the transgene into the host. Further advantages of such targeted integration include, but are not limited to, reducing the large number of transformation events that must be screened and tested before obtaining a transgenic plant that exhibits the desired level of transgene expression without also exhibiting abnormalities resulting from the inadvertent insertion of the transgene into an important locus in the host genome. Moreover, such targeted integration allows for stacking transgenes rendering the breeding of elite plant lines with both genes more efficient.

Using the above disclosed teaching, the skilled person is able to use methods know in the art to target transgenes to the same insertion site as that in event 5307 or to a site in close proximity to the insertion site in 5307. One such method is disclosed in US Patent Application Publication No. 20060253918, herein incorporated by reference in its entirety. Briefly, up to 20 Kb of the genomic sequence flanking 5′ to the insertion site (SEQ ID NO: 5) and up to 20 Kb of the genomic sequence flanking 3′ to the insertion site (SEQ ID NO: 6) are used to flank the gene or genes of interest that are intended to be inserted into a genomic location on Chromosome 5 via homologous recombination. These sequences can be further flanked by T-DNA border repeats such as the left border (LB) and right border (RB) repeat sequences and other booster sequences for enhancing T-DNA delivery efficiency. The gene or genes of interest can be placed exactly as in the event 5307 insertion site or can be placed anywhere within the 20 Kb regions around the event 5307 insertion sites to confer consistent level of transgene expression without detrimental effects on the plant. The DNA vectors containing the gene or genes of interest and flanking sequences can be delivered into plant cells via one of the several methods known to those skilled in the art, including but not limited to Agrobacterium-mediated transformation. The insertion of the DNA vector into the event 5307 target site can be further enhanced by one of the several methods, including but not limited to the co-expression or up-regulation of recombination enhancing genes or down-regulation of endogenous recombination suppression genes. Furthermore, it is known in the art that cleavage of specific sequences in the genome can be used to increase homologous recombination frequency, therefore insertion into the event 5307 insertion site and its flanking regions can be enhanced by expression of natural or designed sequence-specific endonucleases for cleaving these sequences.

An example of this technique is demonstrated in Shukla et al. (Nature vol. 459, 21 May 2009). This method uses zinc finger nucleases for the purposes of targeting heterologous sequences to a specific locus based upon the use of homologous sequences within the target plant. One skilled in the art could use the event 5307 insert between a 5′ marker, designated herein as the public molecular marker umc1475 (SEQ ID No: 104), and a 3′ marker, designated herein as the public molecular marker uaz190 (SEQ ID No: 107) to create a locus for targeted insertion.

### Example 9. Use of Event 5307 Insertion Site and Flanking Sequences for Stabilization of Gene Expression

The genomic sequences flanking the event 5307 insertion site may also be used to stabilize expression of other gene(s) of interest when inserted as a transgene in other genomic locations in maize and other crops. Specifically, up to 20 Kb of the genomic sequence flanking 5′ to the insertion site (SEQ ID NO: 5) and up to 20 Kb of the genomic sequence flanking 3′ to the insertion site (SEQ OD NO: 6) are used to flank the gene or genes of interest that are intended to be inserted into the genome of plants. These sequences can be further flanked by T-DNA border repeats such as the left border (LB) and right border (RB) repeat sequences and other booster sequences for enhancing T-DNA delivery efficiency. The gene or genes of interest can be placed exactly as in the event 5307 insertion site or can be placed anywhere within the 20 Kb regions around the event 5307 insertion sites to confer consistent level of transgene expression. The DNA vectors containing the gene or genes of interest and event 5307 insertion site flanking sequence can be delivered into plant cells via one of the several methods known to those skilled in the art, including but not limited to protoplast transformation, biolistic bombardment and Agrobacterium-mediated transformation. The delivered DNA can be integrated randomly into a plant genome or can also be present as part of the independently segregating genetic units such as artificial chromosome or mini-chromosome. The DNA vectors containing the gene(s) of interest and the event 5307 insertion site flanking sequences can be delivered into plant cells. Thus, by surrounding a gene or genes of interest with the genomic sequence flanking the event 5307 insertion site, the expression of such genes are stabilized in a transgenic host plant such as a dicot plant or a monocot plant like corn.

### Deposit

Applicants have made a deposit of corn seed of event 5307 disclosed above on 15 Oct. 2008 in accordance with the Budapest Treaty at the American Type Culture Collection (ATCC), 10801 University Boulevard, Manassas, VA 20110-2209 under ATCC Accession No. PTA-9561. The deposit will be maintained in the depositary for a period of 30 years, or 5 years after the last request, or the effective life of the patent, whichever is longer, and will be replaced as necessary during that period. Applicants impose no restrictions on the availability of the deposited material from the ATCC; however, applicants have no authority to waive any restrictions imposed by law on the transfer of biological material or its transportation in commerce.

All publications and published patent documents cited in this specification are incorporated herein by reference to the same extent as if each individual publication or patent document was specifically and individually indicated to be incorporated by reference.

What is claimed is:

1. A kit for detecting the presence of corn event 5307 nucleic acids in a biological sample, the kit comprising at least one nucleic acid molecule comprising a sufficient length of contiguous nucleotides that is or is complementary to SEQ ID NO: 1, SEQ ID NO: 2, SEQ ID NO: 3 SEQ ID NO: 4, SEQ ID NO: 5, SEQ ID NO: 6 or SEQ ID NO: 7 that functions as a DNA primer or probe specific for corn event 5307.

2. The kit for detecting the presence of corn event 5307 nucleic acids in a biological sample of claim 1, wherein the first DNA primer sequence is selected from any one of SEQ ID NO: 8 through SEQ ID NO:14 or SEQ ID NO:69 through SEQ ID NO: 72, or their complements; and the second DNA primer sequence is selected from any one of SEQ ID NO: 15 through SEQ ID NO:68, or their complements.

3. The kit for detecting the presence of corn event 5307 nucleic acids in a biological sample of claim 1, wherein the primer pair and probe sequences are:
(i) primer sequences SEQ ID NO: 82 and SEQ ID NO: 83 and probe sequence SEQ ID NO: 84,
(ii) primer sequences SEQ ID NO: 85 and SEQ ID NO: 86 and probe sequence SEQ ID NO: 87, or
(iii) primer sequences SEQ ID NO: 88 and SEQ ID NO: 89 and probe sequence SEQ ID NO: 90.

4. A nucleic acid molecule comprising the amplicon detected by the kit of claim 1.


SEQUENCE LISTING (111 sequences)

SEQ ID NO: 1 - DNA, 20 bp, synthetic construct
  misc_feature 1..20: note = 5' genome-insert juction
  source 1..20: mol_type = other DNA
caactcacgaactgatagtt

SEQ ID NO: 2 - DNA, 20 bp, synthetic construct
  misc_feature 1..20: note = 3' insert-genome junction
  source 1..20: mol_type = other DNA
ccacaatataccctcttccc

SEQ ID NO: 3 - DNA, 200 bp, synthetic construct
  misc_feature 1..200: note = 5' genome + insert sequence
  source 1..200: mol_type = other DNA
gtcgactcaaacggctagttctgacagctagccgttggacagatggcataccggacagtccgatacgctgtccggtgtgcctctaaaattcaactcacgaactgatagtttaaactgaaggcgggaaacgacaatctgatcatgagcggagaattaagggagtcacgttatgacccccgccgatgacgcgggacaagccg

SEQ ID NO: 4 - DNA, 200 bp, synthetic construct
  misc_feature 1..200: note = 3' insert + genome sequence
  source 1..200: mol_type = other DNA
gccctgcaggaaatttaccggtgcccgggcggccagcatggccgtatccgcaatgtgttattaagttgtctaagcgtcaatttgtttacaccacaatataccctcttccctgggccaggctgggcccactggcaaagggtgcaccggacagtccggtgccccaaagccagaaaccctagcttctgttttgtgctgttttt

SEQ ID NO: 5 - DNA, 1548 bp, synthetic construct
  misc_feature 1..1548: note = 5' genome + insert sequence
  source 1..1548: mol_type = other DNA
tacaagaatattgagacgtgagtacatagcattggcattttcattagcaagcatttcaaaagaatttaattttctcatagcaatgtgatatctctcctcacgctcaattctagttccttcatgtagagcacatatgtccatccacaaatcatgacaatttttatggtttctaactctattaaacacatctttgcaaaggcctctaaaaagggtgtttttggccttagcattccatttctcatagttcaactcttcacctacaagatttgtgggatctctaggttcggggaatctttgtgtggcggctttgtagacaccaatgtctatagcctctaaatatgcttccatacgaattttccaatatggaaaatcgtcaccataaaaaacgggagaaggtccatccccaccggacatcgttactctagcggttaagctaatctaagagcaacaaggctcttataccaattgaaaggatcacgatgcccaagagggggggttgaattgggcttttctaaaaatcaacactaactaaaatctaagcaagagcccaacttcaccccgacaactagcactaagagaataatactagaaatacaacaatgctaagataatacttcaaatacttgctaaacaaatacacaatgtaaaatacttgaattaagtgcggaatgtaaagcaaggtttagaagactcctccaatttttctagaggtatcaaagagtcggcactctcccctagtcctcgttggagcacctgcgtaagggtatcgctctcccttggtcatcgcaagaaccaagtgctcacaacgagatgatcctttgccactccggcgcggtggatccctcacgaccgcttacaaacttgagtcgggtcaccaacaagatctccacggtgatcaccgagctcccaacgccaccaagccgtctaggtgatgccgatcaccaagagtaataagccatagactttcacttgaccaagagaagcctaatgcatgcggtgtgtgctctaggtggctctcgctagcgttaatgaggtccaaatgcgggattaagattctcaagtcacctcactaggctttgtggtgcttgcaatgctctaccaatgtgtaggagtaaatgtgggcagcaagaccatcaatatggtaggtggatggggtataaatagccctcacccaccaactagccattaccaggaatctgctgcgcatgggcgcaccggacagtccggtgtgccaccggtgcgccaacggtcgactcaaacggctagttctgacagctagccgttggacagatggcataccggacagtccgatacgctgtccggtgtgcctctaaaattcaactcacgaactgatagtttaaactgaaggcgggaaacgacaatctgatcatgagcggagaattaagggagtcacgttatgacccccgccgatgacgcgggacaagccgttttacgtttggaactgacagaaccgcaacgctgcaggaattggccgcagctgccatttaaatcaattgggcgcgccgaattcgagctcggtacaagctt

SEQ ID NO: 6 - DNA, 1093 bp, Zea mays
  source 1..1093: mol_type = genomic DNA
ccctcttccctgggccaggctgggcccactggcaaagggtgcaccggacagtccggtgccccaaagccagaaaccctagcttctgttttgtgctgttttttcaatttggtttttgttctaacttgtgagtatgttctagagttacacctagcactatatgtgagtgtgaatatgcaccaacactacactagaactcttttggtcaaactacttatcgacaacccctctttatagtacggctaaaacaaaataaaagacctaactatatcacgagtgtccgcaactccttgacactcggaatacgaagaccttcactttttgtttcgtcgctttagccgttgcttcaagtttttatctccgggattgttttcaccattgtagtacatctacctgtaatgcgacctaacttaccatttgcctctgcaaaacacatgttagtcacatataaaattacgttgtcattaatcactaaaaccaaccaggggcctagatgctttctagtttaaatccccaacaagtcaaaattctttctatttttttttgcaagttccaattgacatctgaaaggttgtaaggtacacgtttggctctcattgataacgggggaaagatacagtgcaaaccaccatataatgacccacttctaatcgaatggacctgtaacgacgaaataccctgtgagaactatggttcactcatgttaattcattgaaattgttgtagtgaattgacatggttgggagcctgcttagagagtatagattgtcacttttttttggaccgcaacttatttttaaaagatattgcgatcgcttgtttagtagctgtttcaggccccaatgcagtttctatcgtgatccatttaagtcactcaacattctcatacttctcattttgcattaattcattccaatctccactactataaaatactagcttcgatggtcgtcatacgccatgcacgaagcatgtagatcaatccgcataccagtgggcatctatagataggctgtgaaaaccacccaaatccctactagtggacattttatctatagatggaccgtgagaaaccacacaagtctaacacgacagg

SEQ ID NO: 7 - DNA, 6206 bp, synthetic construct
  misc_feature 1..6206: note = Vector insert
  source 1..6206: mol_type = other DNA
ctggcagacaaagtggcagacatactgtcccacaaatgaagatggaatctgtaaaagaaaacgcgtgaaataatgcgtctgacaaaggttaggtcggctgcctttaatcaataccaaagtggtccctaccacgatggaaaaactgtgcagtcggtttggctttttctgacgaacaaataagattcgtggccgacaggtgggggtccaccatgtgaaggcatcttcagactccaataatggagcaatgacgtaagggcttacgaaataagtaagggtagtttgggaaatgtccactcacccgtcagtctataaatacttagcccctccctcattgttaagggagcaaggatccaccatgactagtaacggccgccagtgtgctggtattcgcccttatgacggccgacaacaacaccgaggcctggacagcagcaccaccaaggacgtgatccagaagggcatcagcgtggtgggcgacctgctgggcgtggtgggcttccccttcggcggcgccctggtgagcttctacaccaacttcctgaacaccatctggcccagcgaggacccctggaaggccttcatggagcaggtggaggccctgatggaccagaagatcgccgactacgccaagaacaaggcactggccgagctacagggcctccagaacaacgtggaggactatgtgagcgccctgagcagctggcagaagaaccccgctgcaccgttccgcaacccccacagccagggccgcatccgcgagctgttcagccaggccgagagccacttccgcaacagcatgcccagcttcgccatcagcggctacgaggtgctgttcctgaccacctacgcccaggccgccaacacccacctgttcctgctgaaggacgcccaaatctacggagaggagtggggctacgagaaggaggacatcgccgagttctacaagcgccagctgaagctgacccaggagtacaccgaccactgcgtgaagtggtacaacgtgggtctagacaagctccgcggcagcagctacgagagctgggtgaacttcaaccgctaccgccgcgagatgaccctgaccgtgctggacctgatcgccctgttccccctgtacgacgtgcgcctgtaccccaaggaggtgaagaccgagctgacccgcgacgtgctgaccgaccccatcgtgggcgtgaacaacctgcgcggctacggcaccaccttcagcaacatcgagaactacatccgcaagccccacctgttcgactacctgcaccgcatccagttccacacgcgtttccagcccggctactacggcaacgacagcttcaactactggagcggcaactacgtgagcacccgccccagcatcggcagcaacgacatcatcaccagccccttctacggcaacaagagcagcgagcccgtgcagaaccttgagttcaacggcgagaaggtgtaccgcgccgtggctaacaccaacctggccgtgtggccctctgcagtgtacagcggcgtgaccaaggtggagttcagccagtacaacgaccagaccgacgaggccagcacccagacctacgacagcaagcgcaacgtgggcgccgtgagctgggacagcatcgaccagctgccccccgagaccaccgacgagcccctggagaagggctacagccaccagctgaactacgtgatgtgcttcctgatgcagggcagccgcggcaccatccccgtgctgacctggacccacaagagcgtcgacttcttcaacatgatcgacagcaagaagatcacccagctgcccctgaccaagagcaccaacctgggcagcggcaccagcgtggtgaagggccccggcttcaccggcggcgacatcctgcgccgcaccagccccggccagatcagcaccctgcgcgtgaacatcaccgcccccctgagccagcgctaccgcgtccgcatccgctacgccagcaccaccaacctgcagttccacaccagcatcgacggccgccccatcaaccagggcaacttcagcgccaccatgagcagcggcagcaacctgcagagcggcagcttccgcaccgtgggcttcaccacccccttcaacttcagcaacggcagcagcgtgttcaccctgagcgcccacgtgttcaacagcggcaacgaggtgtacatcgaccgcatcgagttcgtgcccgccgaggtgaccttcgaggccgagtacgacctggagagggctcagaaggccgtgaacgagctgttcaccagcagcaaccagatcggcctgaagaccgacgtgaccgactaccacatcgatcaggtgtaggagctgagctctagatccccgaatttccccgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgggaattgggtaccagcttgcatgcctgcagtgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgtcgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcaccggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcaaggtacgccgctcgtcctccccccccccccctctctaccttctctagatcggcgttccggtccatggttagggcccggtagttctacttctgttcatgtttgtgttagatccgtgtttgtgttagatccgtgctgctagcgttcgtacacggatgcgacctgtacgtcagacacgttctgattgctaacttgccagtgtttctctttggggaatcctgggatggctctagccgttccgcagacgggatcgatttcatgattttttttgtttcgttgcatagggtttggtttgcccttttcctttatttcaatatatgccgtgcacttgtttgtcgggtcatcttttcatgcttttttttgtcttggttgtgatgatgtggtctggttgggcggtcgttctagatcggagtagaattctgtttcaaactacctggtggatttattaattttggatctgtatgtgtgtgccatacatattcatagttacgaattgaagatgatggatggaaatatcgatctaggataggtatacatgttgatgcgggttttactgatgcatatacagagatgctttttgttcgcttggttgtgatgatgtggtgtggttgggcggtcgttcattcgttctagatcggagtagaatactgtttcaaactacctggtgtatttattaattttggaactgtatgtgtgtgtcatacatcttcatagttacgagtttaagatggatggaaatatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatgcagcagctatatgtggatttttttagccctgccttcatacgctatttatttgcttggtactgtttcttttgtcgatgctcaccctgttgtttggtgttacttctgcagggatccccgatcatgcaaaaactcattaactcagtgcaaaactatgcctggggcagcaaaacggcgttgactgaactttatggtatggaaaatccgtccagccagccgatggccgagctgtggatgggcgcacatccgaaaagcagttcacgagtgcagaatgccgccggagatatcgtttcactgcgtgatgtgattgagagtgataaatcgactctgctcggagaggccgttgccaaacgctttggcgaactgcctttcctgttcaaagtattatgcgcagcacagccactctccattcaggttcatccaaacaaacacaattctgaaatcggttttgccaaagaaaatgccgcaggtatcccgatggatgccgccgagcgtaactataaagatcctaaccacaagccggagctggtttttgcgctgacgcctttccttgcgatgaacgcgtttcgtgaattttccgagattgtctccctactccagccggtcgcaggtgcacatccggcgattgctcactttttacaacagcctgatgccgaacgtttaagcgaactgttcgccagcctgttgaatatgcagggtgaagaaaaatcccgcgcgctggcgattttaaaatcggccctcgatagccagcagggtgaaccgtggcaaacgattcgtttaatttctgaattttacccggaagacagcggtctgttctccccgctattgctgaatgtggtgaaattgaaccctggcgaagcgatgttcctgttcgctgaaacaccgcacgcttacctgcaaggcgtggcgctggaagtgatggcaaactccgataacgtgctgcgtgcgggtctgacgcctaaatacattgatattccggaactggttgccaatgtgaaattcgaagccaaaccggctaaccagttgttgacccagccggtgaaacaaggtgcagaactggacttcccgattccagtggatgattttgccttctcgctgcatgaccttagtgataaagaaaccaccattagccagcagagtgccgccattttgttctgcgtcgaaggcgatgcaacgttgtggaaaggttctcagcagttacagcttaaaccgggtgaatcagcgtttattgccgccaacgaatcaccggtgactgtcaaaggccacggccgtttagcgcgtgtttacaacaagctgtaagagcttactgaaaaaattaacatctcttgctaagctgggagctcgatccgtcgacctgcagatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatctgctagccctgcaggaaatttaccggtgcccgggcggccagcatggccgtatccgcaatgtgttattaagttgtctaagcgtcaatt

SEQ ID NO: 8 - DNA, 28 bp, synthetic construct
  misc_feature 1..28: note = Primer
  source 1..28: mol_type = other DNA
cacgaccgcttacaaacttgagttgggt

SEQ ID NO: 9 - DNA, 21 bp, synthetic construct
  misc_feature 1..21: note = Primer
  source 1..21: mol_type = other DNA
ctcccaacgccaccaagccgt

SEQ ID NO: 10 - DNA, 25 bp, synthetic construct
  misc_feature 1..25: note = Primer
  source 1..25: mol_type = other DNA
cctcactaggctttgtggtgcttgc

SEQ ID NO: 11 - DNA, 24 bp, synthetic construct
  misc_feature 1..24: note = Primer
  source 1..24: mol_type = other DNA
gagtaaatgtgggcagcaagacca

SEQ ID NO: 12 - DNA, 24 bp, synthetic construct
  misc_feature 1..24: note = Primer
  source 1..24: mol_type = other DNA
cccaccaactagccattaccagga

SEQ ID NO: 13 - DNA, 23 bp, synthetic construct
  misc_feature 1..23: note = Primer
  source 1..23: mol_type = other DNA
aaacggctagttctgacagctag

SEQ ID NO: 14 - DNA, 21 bp, synthetic construct
  misc_feature 1..21: note = Primer
  source 1..21: mol_type = other DNA
atacgctgtccggtgtgcctc

SEQ ID NO: 15 - DNA, 18 bp, synthetic construct
  misc_feature 1..18: note = Primer
  source 1..18: mol_type = other DNA
ggtagtttgggaaatgtc

SEQ ID NO: 16 - DNA, 18 bp, synthetic construct
  misc_feature 1..18: note = Primer
  source 1..18: mol_type = other DNA
atacttagcccctccctc

SEQ ID NO: 17 - DNA, 17 bp, synthetic construct
  misc_feature 1..17: note = Primer
  source 1..17: mol_type = other DNA
atgactagtaacggccg

SEQ ID NO: 18 - DNA, 18 bp, synthetic construct
  misc_feature 1..18: note = Primer
  source 1..18: mol_type = other DNA
gccgacaacaacaccgag

SEQ ID NO: 19 - DNA, 17 bp, synthetic construct
  misc_feature 1..17: note = Primer
  source 1..17: mol_type = other DNA
ctacgccaagaacaagg

SEQ ID NO: 20 - DNA, 17 bp, synthetic construct
  misc_feature 1..17: note = Primer
  source 1..17: mol_type = other DNA
gagaggagtggggctac

SEQ ID NO: 21 - DNA, 17 bp, synthetic construct
  misc_feature 1..17: note = Primer
  source 1..17: mol_type = other DNA
ccaccttcagcaacatc

SEQ ID NO: 22 - DNA, 18 bp, synthetic construct
  misc_feature 1..18: note = Primer
  source 1..18: mol_type = other DNA
agttcagccagtacaacg

SEQ ID NO: 23 - DNA, 17 bp, synthetic construct
  misc_feature 1..17: note = Primer
  source 1..17: mol_type = other DNA
agaagatcacccagctg

SEQ ID NO: 24 - DNA, 17 bp, synthetic construct
  misc_feature 1..17: note = Primer
  source 1..17: mol_type = other DNA
ccttcaacttcagcaac

SEQ ID NO: 25 - DNA, 17 bp, synthetic construct
  misc_feature 1..17: note = Primer
  source 1..17: mol_type = other DNA
aggtgtaggagctgagc

SEQ ID NO: 26 - DNA, 18 bp, synthetic construct
  misc_feature 1..18: note = Primer
  source 1..18: mol_type = other DNA
tctagatccccgaatttc

SEQ ID NO: 27 - DNA, 19 bp, synthetic construct
  misc_feature 1..19: note = Primer
  source 1..19: mol_type = other DNA
cccctctctagagataatg

SEQ ID NO: 28 - DNA, 18 bp, synthetic construct
  misc_feature 1..18: note = Primer
  source 1..18: mol_type = other DNA
tttgcaaatagcttcacc

SEQ ID NO: 29 - DNA, 17 bp, synthetic construct
  misc_feature 1..17: note = Primer
  source 1..17: mol_type = other DNA
atgccagcctgttaaac

SEQ ID NO: 30 - DNA, 17 bp, synthetic construct
  misc_feature 1..17: note = Primer
  source 1..17: mol_type = other DNA
cctcctcctcctctcac

SEQ ID NO: 31 - DNA, 20 bp, synthetic construct
  misc_feature 1..20: note = Primer
  source 1..20: mol_type = other DNA
tctgttcatgtttgtgttag

SEQ ID NO: 32 - DNA, 18 bp, synthetic construct
  misc_feature 1..18: note = Primer
  source 1..18: mol_type = other DNA
gatgatgtggtctggttg

SEQ ID NO: 33 - DNA, 20 bp, synthetic construct
  misc_feature 1..20: note = Primer
  source 1..20: mol_type = other DNA
tgtttcaaactacctggtgt

SEQ ID NO: 34 - DNA, 17 bp, synthetic construct
  misc_feature 1..17: note = Primer
  source 1..17: mol_type = other DNA
tagccctgccttcatac

SEQ ID NO: 35 - DNA, 20 bp, synthetic construct
  misc_feature 1..20: note = Primer
  source 1..20: mol_type = other DNA
tcattaactcagtgcaaaac

SEQ ID NO: 36 - DNA, 18 bp, synthetic construct
  misc_feature 1..18: note = Primer
  source 1..18: mol_type = other DNA
tccgaaaagcagttcacg

SEQ ID NO: 37 - DNA, 20 bp, synthetic construct
  misc_feature 1..20: note = Primer
  source 1..20: mol_type = other DNA
aaacacaattctgaaatcgg

SEQ ID NO: 38 - DNA, 17 bp, synthetic construct
  misc_feature 1..17: note = Primer
  source 1..17: mol_type = other DNA
aatcggccctcgatagc

SEQ ID NO: 39 - DNA, 20 bp, synthetic construct
  misc_feature 1..20: note = Primer
  source 1..20: mol_type = other DNA
tggttgccaatgtgaaattc

SEQ ID NO: 40 - DNA, 19 bp, synthetic construct
  misc_feature 1..19: note = Primer
  source 1..19: mol_type = other DNA
aacgaatcaccggtgactg

SEQ ID NO: 41 - DNA, 17 bp, synthetic construct
  misc_feature 1..17: note = Primer
  source 1..17: mol_type = other DNA
gtcataagggcgaatac

SEQ ID NO: 42 - DNA, 19 bp, synthetic construct
  misc_feature 1..19: note = Primer
  source 1..19: mol_type = other DNA
acgctgatgcccttctgga

SEQ ID NO: 43 - DNA, 17 bp, synthetic construct
  misc_feature 1..17: note = Primer
  source 1..17: mol_type = other DNA
ccttgttcttggcgtag

SEQ ID NO: 44 - DNA, 17 bp, synthetic construct
  misc_feature 1..17: note = Primer
  source 1..17: mol_type = other DNA
tagaactcggcgatgtc

SEQ ID NO: 45 - DNA, 17 bp, synthetic construct
  misc_feature 1..17: note = Primer
  source 1..17: mol_type = other DNA
gatgttgctgaaggtgg

SEQ ID NO: 46 - DNA, 17 bp, synthetic construct
  misc_feature 1..17: note = Primer
  source 1..17: mol_type = other DNA
ctgtacactgcagaggg

SEQ ID NO: 47 - DNA, 17 bp, synthetic construct
  misc_feature 1..17: note = Primer
  source 1..17: mol_type = other DNA
gctgggtgatcttcttg

SEQ ID NO: 48 - DNA, 17 bp, synthetic construct
  misc_feature 1..17: note = Primer
  source 1..17: mol_type = other DNA
ttgctgaagttgaaggg

SEQ ID NO: 49 - DNA, 17 bp, synthetic construct
  misc_feature 1..17: note = Primer
  source 1..17: mol_type = other DNA
gtcacgtcggtcttcag

SEQ ID NO: 50 - DNA, 20 bp, synthetic construct
  misc_feature 1..20: note = Primer
  source 1..20: mol_type = other DNA
gccaaatgtttgaacgatcg

SEQ ID NO: 51 - DNA, 21 bp, synthetic construct
  misc_feature 1..21: note = Primer
  source 1..21: mol_type = other DNA
caatgctcattatctctagag

SEQ ID NO: 52 - DNA, 20 bp, synthetic construct
  misc_feature 1..20: note = Primer
  source 1..20: mol_type = other DNA
gtgacaaaaaaaatatgtgg

SEQ ID NO: 53 - DNA, 18 bp, synthetic construct
  misc_feature 1..18: note = Primer
  source 1..18: mol_type = other DNA
ctgcacttcaaacaagtg

SEQ ID NO: 54 - DNA, 22 bp, synthetic construct
  misc_feature 1..22: note = Primer
  source 1..22: mol_type = other DNA
tgaagtattatataggtgaagc

SEQ ID NO: 55 - DNA, 19 bp, synthetic construct
  misc_feature 1..19: note = Primer
  source 1..19: mol_type = other DNA
acaggctggcattatctac

SEQ ID NO: 56 - DNA, 17 bp, synthetic construct
  misc_feature 1..17: note = Primer
  source 1..17: mol_type = other DNA
gttagactcgtcgacgg

SEQ ID NO: 57 - DNA, 17 bp, synthetic construct
  misc_feature 1..17: note = Primer
  source 1..17: mol_type = other DNA
ctatttattacggcggg

SEQ ID NO: 58 - DNA, 17 bp, synthetic construct
  misc_feature 1..17: note = Primer
  source 1..17: mol_type = other DNA
gacgtacaggtcgcatc

SEQ ID NO: 59 - DNA, 20 bp, synthetic construct
  misc_feature 1..20: note = Primer
  source 1..20: mol_type = other DNA
ggtagtttgaaacagaattc

SEQ ID NO: 60 - DNA, 23 bp, synthetic construct
  misc_feature 1..23: note = Primer
  source 1..23: mol_type = other DNA
gtaactatgaagatgtatgacac

SEQ ID NO: 61 - DNA, 17 bp, synthetic construct
  misc_feature 1..17: note = Primer
  source 1..17: mol_type = other DNA
acaacagggtgagcatc

SEQ ID NO: 62 - DNA, 17 bp, synthetic construct
  misc_feature 1..17: note = Primer
  source 1..17: mol_type = other DNA
agtcaacgccgttttgc

SEQ ID NO: 63 - DNA, 17 bp, synthetic construct
  misc_feature 1..17: note = Primer
  source 1..17: mol_type = other DNA
aggaaaggcagttcgcc

SEQ ID NO: 64 - DNA, 17 bp, synthetic construct
  misc_feature 1..17: note = Primer
  source 1..17: mol_type = other DNA
aggctggcgaacagttc

SEQ ID NO: 65 - DNA, 20 bp, synthetic construct
  misc_feature 1..20: note = Primer
  source 1..20: mol_type = other DNA
gcaaccagttccggaatatc

SEQ ID NO: 66 - DNA, 19 bp, synthetic construct
  misc_feature 1..19: note = Primer
  source 1..19: mol_type = other DNA
agcttgttgtaaacacgcg

SEQ ID NO: 67 - DNA, 18 bp, synthetic construct
  misc_feature 1..18: note = Primer
  source 1..18: mol_type = other DNA
ccagcttagcaagagatg

SEQ ID NO: 68 - DNA, 17 bp, synthetic construct
  misc_feature 1..17: note = Primer
  source 1..17: mol_type = other DNA
taacacattgcggatac

SEQ ID NO: 69 - DNA, 21 bp, synthetic construct
  misc_feature 1..21: note = Primer
  source 1..21: mol_type = other DNA
gcctggcccagggaagagggt

SEQ ID NO: 70 - DNA, 25 bp, synthetic construct
  misc_feature 1..25: note = Primer
  source 1..25: mol_type = other DNA
cagcacaaaacagaagctagggttt

SEQ ID NO: 71 - DNA, 26 bp, synthetic construct
  misc_feature 1..26: note = Primer
  source 1..26: mol_type = other DNA
ccgagtgtcaaggagttgcggacact

SEQ ID NO: 72 - DNA, 26 bp, synthetic construct
  misc_feature 1..26: note = Primer
  source 1..26: mol_type = other DNA
cttgaagcaacggctaaagcgacgaa

SEQ ID NO: 73 - DNA, 22 bp, synthetic construct
  misc_feature 1..22: note = Primer
  source 1..22: mol_type = other DNA
tacgagagctgggtgaacttca

SEQ ID NO: 74 - DNA, 18 bp, synthetic construct
  misc_feature 1..18: note = Primer
  source 1..18: mol_type = other DNA
cgatcaggtccagcacgg

SEQ ID NO: 75 - DNA, 20 bp, synthetic construct
  misc_feature 1..20: note = Probe
  source 1..20: mol_type = other DNA
ccgctaccgccgcgagatga

SEQ ID NO: 76 - DNA, 18 bp, synthetic construct
  misc_feature 1..18: note = Primer
  source 1..18: mol_type = other DNA
ccgggtgaatcagcgttt

SEQ ID NO: 77 - DNA, 18 bp, synthetic construct
  misc_feature 1..18: note = Primer
  source 1..18: mol_type = other DNA
gccgtggcctttgacagt

SEQ ID NO: 78 - DNA, 20 bp, synthetic construct
  misc_feature 1..20: note = Probe
  source 1..20: mol_type = other DNA
tgccgccaacgaatcaccgg

SEQ ID NO: 79 - DNA, 21 bp, synthetic construct
  misc_feature 1..21: note = Primer
  source 1..21: mol_type = other DNA
gaacgtgtgttgggtttgcat

SEQ ID NO: 80 - DNA, 24 bp, synthetic construct
  misc_feature 1..24: note = Primer
  source 1..24: mol_type = other DNA
tgcagcctaaccatgcgcagggta

SEQ ID NO: 81 - DNA, 20 bp, synthetic construct
  misc_feature 1..20: note = Probe
  source 1..20: mol_type = other DNA
tccagcaatccttgcacctt

SEQ ID NO: 82 - DNA, 20 bp, synthetic construct
  misc_feature 1..20: note = Primer
  source 1..20: mol_type = other DNA
gccgtatccgcaatgtgtta

SEQ ID NO: 83 - DNA, 21 bp, synthetic construct
  misc_feature 1..21: note = Primer
  source 1..21: mol_type = other DNA
ggcccagggaagagggtatat

SEQ ID NO: 84 - DNA, 19 bp, synthetic construct
  misc_feature 1..19: note = Probe
  source 1..19: mol_type = other DNA
aagttgtctaagcgtcaat

SEQ ID NO: 85 - DNA, 26 bp, synthetic construct
  misc_feature 1..26: note = Primer
  source 1..26: mol_type = other DNA
tgtctaagcgtcaatttgtttacacc

SEQ ID NO: 86 - DNA, 16 bp, synthetic construct
  misc_feature 1..16: note = Primer
  source 1..16: mol_type = other DNA
tttgccagtgggccca

SEQ ID NO: 87 - DNA, 27 bp, synthetic construct
  misc_feature 1..27: note = Probe
  source 1..27: mol_type = other DNA
acaatataccctcttccctgggccagg

SEQ ID NO: 88 - DNA, 20 bp, synthetic construct
  misc_feature 1..20: note = Primer
  source 1..20: mol_type = other DNA
gccgtatccgcaatgtgtta

SEQ ID NO: 89 - DNA, 19 bp, synthetic construct
  misc_feature 1..19: note = Primer
  source 1..19: mol_type = other DNA
aagttgtctaagcgtcaat

SEQ ID NO: 90 - DNA, 21 bp, synthetic construct
  misc_feature 1..21: note = Probe
  source 1..21: mol_type = other DNA
ggcccagggaagagggtatat

SEQ ID NO: 91 - DNA, 25 bp, synthetic construct
  misc_feature 1..25: note = Primer
  source 1..25: mol_type = other DNA
ccccacgattaaatgtcaaactgat

SEQ ID NO: 92 - DNA, 25 bp, synthetic construct
  misc_feature 1..25: note = Primer
  source 1..25: mol_type = other DNA
gctcagccttgtttttgtacattca

SEQ ID NO: 93 - DNA, 20 bp, synthetic construct
  misc_feature 1..20: note = Probe
  source 1..20: mol_type = other DNA
aattttcatagctttttgtg

SEQ ID NO: 94 - DNA, 27 bp, synthetic construct
  misc_feature 1..27: note = Primer
  source 1..27: mol_type = other DNA
cgctcttaagtctgctgtttgtttact

SEQ ID NO: 95 - DNA, 24 bp, synthetic construct
  misc_feature 1..24: note = Primer
  source 1..24: mol_type = other DNA
cacacgccacttcttgtcttctat

SEQ ID NO: 96 - DNA, 13 bp, synthetic construct
  misc_feature 1..13: note = Probe
  source 1..13: mol_type = other DNA
cgcgagctcatgc

SEQ ID NO: 97 - DNA, 25 bp, synthetic construct
  misc_feature 1..25: note = Primer
  source 1..25: mol_type = other DNA
gctgcagctcacttgaaggtataat

SEQ ID NO: 98 - DNA, 20 bp, synthetic construct
  misc_feature 1..20: note = Primer
  source 1..20: mol_type = other DNA
ggcaccaccctgtaaaagca

SEQ ID NO: 99 - DNA, 17 bp, synthetic construct
  misc_feature 1..17: note = Probe
  source 1..17: mol_type = other DNA
aaccattagatgcttcc

SEQ ID NO: 100 - DNA, 16 bp, synthetic construct
  misc_feature 1..16: note = Primer
  source 1..16: mol_type = other DNA
ccgtcgacgaggcgaa

SEQ ID NO: 101 - DNA, 16 bp, synthetic construct
  misc_feature 1..16: note = Primer
  source 1..16: mol_type = other DNA
gcggcgagctgttcag

SEQ ID NO: 102 - DNA, 16 bp, synthetic construct
  misc_feature 1..16: note = Probe
  source 1..16: mol_type = other DNA
tctgagcttcggatac

SEQ ID NO: 103 - DNA, 161748 bp, Zea mays
  misc_feature 2151..2250: note = n is a, c, g, or t
  misc_feature 6108..6207: note = n is a, c, g, or t
  misc_feature 9770..9869: note = n is a, c, g, or t
  misc_feature 18125..18224: note = n is a, c, g, or t
  misc_feature 33520..33619: note = n is a, c, g, or t
  misc_feature 44173..44272: note = n is a, c, g, or t
  misc_feature 67063..67162: note = n is a, c, g, or t
  misc_feature 91565..91664: note = n is a, c, g, or t
  variation 136173..136272
  misc_feature 148532..148631: note = n is a, c, g, or t
  misc_feature 154026..154125: note = n is a, c, g, or t
  misc_feature 158039..158138: note = n is a, c, g, or t
  source 1..161748: mol_type = genomic DNA
cccggccgctgatgaatcagcttgattcgttctgttatcacgggtggtcactcaacgagcaggtccaaaggaaaggtactcaggaaaatagcctgagtctcctaaagtgccataagaacatcatcgtaatcataataacaacatcatatcataaatattcgcatcatgtttgttgattaaagtggagcaatagcttgaagcttaccataataacccaaaaggtaaacaaggacaagataaatacagactagtcaaaccttaggtttcaattaagtaaagggggacagtgaattatgaagtaagtaggacataataggtcagaggacacttgccttcaccaggttgttgcccaggaagatcttcggcaacacactcaggaaccatagactgcttgttgtctacgcaaagcgatcatgcattcaacacatttcgataatgataaagaaacaatacaccaaaaatatacaatcaagtgaacactaattcaaaagaaagtaacaaactcaagcgaagcctagggtctagggtggaccaatacacatataggtttgtggttctctaagtattacttatctcaatagattacataacttaatttcatttatcttaatgagacaaaagaattataccagggataggttcatatattacatattattaacccacaaagttaaacatctaactaccattatggttttccttttatccttcttattaataaataagccatcagttacactaacctatagtctaggcataaaattagcacatgcagacagtaaaaggttataatttaaacaggtagagaataaccttacaaacattttgcaatttgaatcactcaatttggagttcatatgcaaaagatatgaaataaacaagttttggaattcaaaatacaaaactaggtctaattatgtgataacctaaaagattaggggcctttctgcaaaagtacaggggcatgcgtgcgaaaaccagggacgatgggttgattctcagaaagccgagggcctttttaacaaaactaccacgcaaaggggtatcagctgatctcgactgcatgatcacagatcaacggccaggattagatttgagcgcgagcacgagcacgagctaacaggtgggccaggatagtcagcgacctaggggcgaggcggactgtctggccgggcctagctgcagggcgcgggtgaggtggcggatccgagtggccagatctccatcggacagctgggatcagatcgagtttaattgaagccaggtcgttagatctcagatggatgcctgaaatctgatggcaagctcgggcggggttgctaggctgctcatggcgccgccgcccaatttcgcggcgtggcgcggccatggtgagggtctgggcgctgggaaaaggctcaggcgagctcagggtgacacggcgggctcagccatgggcacgacaccggcgtagaggcaccagagagcacggtccgaggcaaagcagccccacggcggcgcagcttaactctggcgagcgattgcatggacaacagggcagtaaatgggaaattaagggcatgggtgggttggttacgtcgagagatgactctagagcgcttgagcaacggcgaggacaccgcgagggccctggtggacggtggcggagactcggctgcatggtgataggtccggtgagcgaaccaagggaaatagaggggctggggaaaaccagagggtgtctcgtgttgctggcgaggaggcgaagatcagtagggcaatggacgcgacaggaactcgacgacggccacggaacggacggtggactacggcagtgctccacggctgtgcgctcggtgcgagagagaggtgcgagggggtcggctgtgggacgctactgagcgaggggagtgagcgagtgagtgtgggctccaaaaaagtcaggcgcgtggggggagtggccgaaaaacacgcgacatgtgtgcatccacggcggggtgcgcgagcgggtggttagggaaaggggaggtggctgacaggtggggtccgcttgccagcgagggtgaatacgcgaacgagcggttctgcgctgacaggccgacccaccgaggcaaaaaggagcgggcgtgttgcgtgaaagaaaccggcaccgacaaaccggcctccgcgcgnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnctgcgggtaccacgttctacaaggtttgatgatagtgaggaaggaagaatttctggcactgaagcaagggcccctgtctgtcagtgagtacatggacaaattcctgcaactatctcacaatgcacccgaggatgtcaacattgatgctaagaggtactacaggtttccgagagggttggttgacccctgcactactagttgatgaaccacacattccctaccttccaacatctgattgatagggcaataatgactgagaggaagcgccaggagatggaagaccaaaagcgcaagattggtggaccctaggccaggagcagcagtcgtctcccgtttctggcaatccaccctagcagttcaagtagatccaccctcagggataccaacaccagaaccaatgttcgcaccagtagcaattccagaggcagttccctcaacagcagcatgtcacacccgggttttaggggtccaaaacccaggcgcgaaattcaccaagtgctgggatcgagtctcacacatatgatgactcatggtatagaaacaaatgtcacatctttactatataatagaagttctgcacaaaataactaaataattacatcatacgatgacgacgatccatcaacccaaagtttactgtgagacgacggcctagacctctcatgaactcatcgcgacatccttcatgctcctcatcttgcggtacctgttcttgaccagggggatttgagtacagcaagggtgagctcacatacgttcatcgctcaacaagttgtggggaataatgtgtatgaactcaccaaaggtgggagctcatgtgaagtgtaaggcttaccaaaggagatgggtaaagatgagcatgacttttaaagttggtcaaaattttattagcagttactaagtataagtagataccgacccaaataaataagagattaaattaataacaacacccacaatgcaatgcatatgacaatttaagtttagttccataatttactcatgtgagggtccgagctgctcatgaccgtgagcacggctgatataacagttttacagtctgcacaggttgcacatctttacccacaagtcatgttacctatttgccaagggatcgcgacttctcattcatctctaccgagaagacaaggtaggttaccactacgaggcctttacaaacttccactagcttccgaaaacccgctacggtttctaagaaggaaaatataggaatccctcgtccaaaaagccatcgcagcatgatcgactcgagaacctccctatacgcatgctcctctaccgcccttgcccctttcgggtaaggtagtcttccactagctttcttaattagtcagccaagggcgtcccataccacccttgtggtagcactgttttcctgggtggttgctccatgttccaattaacatagcaatcttatcatgaacaataattaaaataacaaaagaattgtaacatgatcataatgtaacattaatttcccaaaaccaggtagagcaatagcaatactacccaatagtgcttttgtttgcaaggtaggggataaacaatactaggaaaacctattgggtcccatcaaattaacctgagcatgtcacagtgattaataggaacattattaggtaaagaaaagtgatcaagggcacaacttggctgagactcaagattcctaggtaccagcttggtcttcaagattctcgtaacctcgctgctaatcatagcaatacaaacaaacatggtataggcaaaattaacatcacaccaaacataaagaacaaactgcataataatgatctacgcaccacaacgagatcctaggttcgagaaccactaaattcggagttacggttaacaagatgtggttttcggaagacctatgtgattaaatatgagactaggtctttatgttgattttataaattatgtgataaagatattaaagaaataactttaatctacatcatactagagtagacataatattttagttaccttataatcatagacaaactaactttgattagtaggaataatctactaagcatatattaaatgaatatttattttttggaaacatgctatttgctaaaataattttacagaagcgtaggcaaaattattacgaagctaacgcaacatgaatacattaaatcagagttaaaatgaaagagatatgtatttattaagttttaggatttaattctataattattaaatatttctggattggggacactattctataaaagatcagggggctccatataatatttaggacttatccgcaatgatttctacctatacccggactgcgggctgatttgcaagaagtctggggtctcttttataagttagtcacggtgaaggggtacacgtgactaattccttggatcatcagccaagcgcccagagtagaagatttgcccgccgaaccggtacgcatcctagatcgtcggatctacgataaacggcccacgcttaaaataatagagatcgatcctcatatgcaagatccagatcagacgacccggatcgattcggatgaaacgttacgtgtgatctaatcacagccgatacctcccagatccacggttcacgcgaggcccagccatgccctgatcgtgatcgctcacccatgatctaacggctgctgcatttccttccacctcacgacggaaagcagagcactggtgcgggcacgccgcggccatgccccaccacaccaccagtgatatcccgcccggctccccatttcctagtatcgagcgtgggtacgtgaatcacggagaggaggaggctccaagtatgctagggctgttcttaccaaggatcacggtgtttcaagtgttgaccccaccacgcagttgctccgtggcgccgcgggtcaccagcgaagcatgcactggtcgttgttctcgcacgaggtgccttctagaatcctgcacgcgtcccacggatgacccaacccgacgccgagaccgcaataccggcgtgcccgggaacccccgtcggtggcaattcaccccctgtgttctccttctcccttacgacgatggtgatggcgccttctctcccgatcggcagaccgagcgtagcccacgatgctgaaggagaggaaactagagctgcacccatggccgaggttggagcgtccgttatatatggccaggggtacggctagcagtgggcgggtgcaccatggcacgaaggtcgttgcacagtttacaggaggcgagcttgcagcggacgagcaggatcgccatggggaggatagacttgacggccatggcccacatgccagacgcggctgcaggcgcgagagtgggcaggagcgggctgcgccggagcagggaaatagagttgggcccgctaacgaaggaaagaaactgggccgagaagccagagatccggcccatagcgcagaaagcttcccctttttctttattctttaatgattttctgttttatcttccctttcatatttctttcccttattttaaactctaatctaaatgctcaatccaaaactccggcatgatatgcaataattacatatatctgtttagttttgtttattttatccaaatattttaagtatgcaatgcacacacatagagtaaaaattacttctttgaatgtatagtccatttaaaattatgttcataatttttaagatagaggatttttttgtgtgtatagtatttattaaggttttttaagcttaattcttttggagaatatctctaatcatgttattcaacaagggttggtttaaattatatgagggtcttttatttaatctctcattataaaagacttctatttaaatcttggaattcnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnngggggtttttctttatctcgtgcgtggttatccatctaatcacgtgggagtttgttggctatctcttaggaaaaggtccagacctcctcccctataaatataaaggggtacggccgattgagaacccccgaacacattccaatcgaaccaattaccttatttacttttcctgccctaggagtagatgtagcatagttctagttgtagtcttccacatatccacctccacccctattcaactctacgtcgtctagatccgtcttgggtggcctgccgatcccaagacgaccctaggatctcacccctcccggggggcaagatctagttgtccatccaagacttcttcctcgatttgatctcttaattcctaggcgactccacgtcgtctggggacgccccgggtgacctgtcgacccggagcaccttaagatctttccccccaggggacgagatctagattccagcaaggagtaggaagacgaccctgtcgccaggtcgcggaccgtccggcccagagctgcggaccgtccggtgtgacgcagggaagacaccgctcctgcgcccaggtcgcggaccgtccggcccaaggctgcggaccgtccggcccaaggctgcggaccgtccgcgcctgaccagagggcaccgccacggttcttgttgagtgtttggcgctccaaaaaggcgtcaacatactttttggcgactccgctggggaagaagttgcagatctacaaaatcaggcttacatggccgattctaaagatctcaacagtgcttctccaaacagcaacacaaggctgactaatttatcggccgctgagcataaaaaattagaagatgacatgaagaaaatagacgaggaggcccaccgacaaaaggatcaggtgctcaaggtggcggacaagtggtacctctcgcacttcaaggtagactgccaccagaagaccgtccaagagagggagataaacgccgagtatatgttagccgtgctgcaacagctccccacaataggtgatgccaggtcagccgatgatattccatctattaaaatttcttttgataatcggattaaaagtatcacggaggatatagagaggatgacacatgcattaggaaaaactcacatgcctaattttttatcacataaattaggcgatgaaacaattgcgccaaacacatcggcggcaaatgggtttccccagccatattctggtatgccgatggactcatatctaggacgaccgtcatcaccatctttgctaaatggtgagtcaaccctgggcacagccggaccgtccgcacacaattgcggaccgtccggccctctgtcggaccgtccggcaccctacgccggacagtctggagttacacagagcccaccacaagggtcacaggtgttgcctgacgtgaccggactgtccgaggatagtaccggaccgtccgatccacccgcagaccgtccgactgtgcaagtcggaccgtccggggcaccagaagtcacctgtgatccacctagtgcggaaggccgacataaatataatcggccacccaagccccaagaactaaaaaagtcacatgtccctgagcttgtttggcccactaaggccaaaccttctgttcgctcttacccgcactcgaaacaaaaggaaaaggttaagttcacatttaatattactaaatgtgataaaatatttgatgagttgcttaaacatggtaatattaaattgtcacatgtaattcctccggttgaacaattaaaagggcgtgtttattgcaaatggcatggctcctttctccataacaccaatgattgtgccgtcttccgtcggcaaatacaatcggctataaacgaaggccggttgaggtttcaaaaagaggtgaaaattgacaggccacctgttcctgtcaccacattagagcccatgagcaaaaaggccataattcggccttgtgcggccgataaaagtaaaaataaaaatatcgtcattggtgatcctcgcacaccaaatatgtcacgcagaatggttactctgaaggctccggacaaaagaaagaccggaggcaccggggggcaagcacgatcggacacccgatcacggtcgcctgtcatgcgtacgccggacgatccgggtactaaggccgaacagtccgagacaggcgcggacagtccggctatgatggccggacggtccgcagatggtcagaagcagcaacctcagaccatcggaccacaacgttccaacacaagtgttaggaaacaaaacactactaagacgtctggacgactcagtagagtcggccctacttttggtcagttgcttgccaaatatatgaagaaggccgttccacacaaccggccaataaaacaaacaaagtcaatagggcgatctgtgcgaaagcaaaagccgactaaacggacccaaagggtagcacaaccaatatcgccttatcatcctcctccagggatagcatggtgcgtcccattctatccatcgccgatgtgttgtcctactcatgtgtggggtggtacggcgatgaatttgtattactggcccaatccgtttgcttatttgggctggggggcaccacaagtttttgcctattgacaggttgatcagatagacatggctgaagaggatgcgatccgaaacggcctctgtgcattaaagtcccatcaagtatttatattatctgatcgcaagagccgatgacttgcatcgagctgagtccttacttcggaaaaaaaaacctcatgaggtcaattgtttccgaagttttcgctaatgcttttggttcgccatgctccaccaaaaggcaggggggcatatgttggacaccaaaatgagcggacggtccggcccatgggcccggacggtccgcgtgtcccgagattagattaactcggatgtttatccttatctcgtgcgtggttatccatctaatcacgtgggagtttgttggctatctcttaggaaaaggtccagacctcctcccctataaatataaaggggtacggccgattgagaacccccgaacacattccaatcgaaccaattaccttatttacttttcctgccctaggagtagatgtagcatagttctagttgtagtcttccacatatccacctccacccctattcaactctacgtcgtctagatccgtcttgggtggcctgccgatcccaagacgaccctaggatctcacccctccggggggcaagatctagttgtccatccaagacttcttcctcgatttgatctcttaattcctaggcgactccacgtcgtctggggacgccccgggtgacctgtcgacccggagcaccttaagatctttcccccaggggacgagatctagattccagcaaggagtaggaagacgaccctgtcgccaggtcgcggacgtccggcccagagctgcggacgtccggtgtgacgcagggaagacaccgctcctcgcccaggtcgcggaccgtccgacccaaggctcggacgtccgcccaaggctgggaccgtccgcgcctgaccagagcacgccacggtctgtgaggttgcaagatgcnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnntaatctatacagacgatctgagattcgtctcattttgagcccgtctcaagaatccctttaatgtctcttgggttagagatttttcctgtaaaaagaatacccaagtgaagcgagaataatcatccacaataactagacagtacttactcccgccgatacttatgtaagcgatcgggccgaataaatccatgtggaggagctccagtggcctgtcacttgtcattatgttcttgtgtggatgatgagtgccaacttgcttcccggcttggcatgcgctacaaatcctgtctttctcaaaatgaacatttgttaatcctaaaatgtgttctccctttagaagcttatgaagattcttcatcccaacatgggctagtcggcggtgccagagccaacccatgttagtcttggcaattaagcatgtgtcgagttcagctctatcaaaatctactaagtatagctgaccctctaacacacccttaaatgctattgaatcgtcacttcttctaaagacagtgacacctacatcagtaaaaagacagttgtagcccatttgacacaattgggaaacagaaagcaagttgtaatctaatgaatcaacaagaaaaacattggaaatagtatggtcaggtgatatagcaattttacccaatcctttgaccaaacctcgatttccatccccgaatgtgatagctcgttggggatcttggtttttctcatatgaggagaacatccttttctccccggtcatgtgggtttgtgcacccgctgtcgagtatccaacttgagcccccggatgcataaacctacaaaaacaaatttagttcttgactttaggtacccaaatggttttgggtcctttggcattagacacaataactttgggtacccaaacacaagtctttgaccccttgtgcttgcccccaacatatttggcaactactttgccggatttgtttgtaagcacataagaagcatcaaaagttttaaatgaaatagcatgatcatttgatgcaataggagttttctttctaggcaacttggcacgggttggttgcctagagctagatgtctcacccttatacataaaagcatgattagggccagagtgagacttcctagaatgaattttcctaattttgctctcgggataaccggcagggtacaaaatgtaaccctcgttatcctgaggcatgggagccttgcccttaacaaagttagacaagtttttaagaggggcattaagtttgacattgtctcccctttggaagccaatgccatccttaatgtcagggcgtctcccattataaagcatgctacgagcaaatttaaatttctcattctctaggttgtgctcggcaattttagcatctaattttgctatatgatcattttgttgtttaattaaagccatatgatcaagaatagcattaacatcaacatctctacatctagtacaaatagatacatgctcatcaatagatgtagagggtttgcaagaattaagttcaacaatcttagcatgaagaatatcattcttatctctaagatcggaaattgtaactttgcaaacatcaaaatctttagccttagcaatcaaattttcattctctaatctaaggctagcaagagaaatgtttaattcttcaatcctagcaagcaactcatcattattatctctaggattgggaattgaaacattacaaatatgagaatcaaccttagcatttaaactagcattttcatttctaaggttgtcaatcatctcacggcaagtgcttagctcactagacaatttttcacatttctcaacttctagagcataagcctttctaaccttaacatgtttcttgttttctttaattagacaatcctcttgggaatccaaaaggtcatccttttcatgaatagcactgactaattcatttaatttttccttttgagctatgttaaggttggcaaagaggatacgcaaattttcctcctcatcactagcattatcatcactagacgattcatatttagtggaggagttggatttaaccttcttctttttgccgtcctttgccatgaggcacttgtggccgacgttggggaagagaagtcccttggtgacggcgatgttggcggcatcctcgtcgtcggaggagtcgcttgagctctcgtcggagtcccatttgcgacaaacatgggcatcgccgcccttcttcttgtaatacctcttcttctcctttcttctccccttcttgtcgtcgcctcggtcactgtcactagatattggacatttagcaataaaatgaccgggcttaccacatttgtagcaaaccttcttggagcgggacttgtagtctttccccctcctttgtttgaggatttggcggaagctcttaatgacgagcgccatctcctcattgtcaagcttggaggcgtctattggttgtcgacttggtgtagactcctccttcttctcctccgttgccttgaatgcaacgggttgggcttcggatgagtcgccaagctcgttgattttcctcgagccttctatcatgcactcaaaacttacaaaatgcccgataacttcctcgggggtcattttagtatatctaggattaccacgaatcaattgaacttgagtgggattaagaaaaatgagagatcttaaaataacatttaccacttcgtgatcgtcccacttcttgctcccgaggttgcgcacttggttcaccaaagtcttgagccggttgtacatgtgttgtggctcctctcctttgtgaagccggaaccgaccgagctccccctcgatcgtttcccgcttggtgatcttggtgagctcgtctccctcgtgcgcggttttgagtacatcccaaatctccttggcgctcttcaacccttgtactttgttatactcctctctacttagagaggcgaggagtattgttgttgcttgagagttgaagtgctcgatttgggccacctcatcctcatcatagtcctcatcccctacggatggtacctgcgcgccaaactcaacaacatcccatatgcttttgtggagcgaggttagatgaaatcgcattaaatcgctccacctagcgtaatcttcaccatcaaaagttggtggtttgcctaatgggacggaaagtaaaggtgtatgtttggaaatgcgagggtagcgtagggggatcttactatacttcttgcgctcttggcgcttagaagtgacggagggcgcatcggagtcggaggtcgatgttgatgaagtgtcggtctcgtagtagaccaccttcctcatccttttgtgcttgtcgcctttccgatgcggcttgtgggaagaagatttttccttcttctctttgtggtgagaagaagatttcttctccttccctttgttggaggagctcttcttcttctccctccttttggtgcgagactcttccgatgaagtgctcccgtggcttgtagtgggcctttcgccggtctccatctccttcttggcgtgatctcccgacatcacttcgagcggttaggctctaatgaagcaccgggctccgataccaattgatagtcgcctagaggggggtgaatagggcgaaactgaaatttgcaaatataaacacaactacaagccggggttagcgttagtaataaggaatgagtccgcaagagagggcgcaaaacaaatcccaagcgaatgagcaagtgagacacggagatttgttttaccgaggttcggttcttgcaaacctactccccgttgaggaggccacaaaggccgggtctctttcaacccttccctctctcaaacgatccacggatcgagtgagcttctcttctcaaatcaaagccgggaacaaaacttccccgcaagggccaccacacaattggtgcctcttgccttgattacaatggagttttgatctcaagaacaagtgagaaagaaaagaagcaatccaagcgcaagagctcaaatgaacacgacaaatcactctcactagtcactagggctttgtgatgaattggagaggatttgatctctttgtatgtgtctagaattgaatgcctagctcttgtagtagttgggaagtggaaaacttggatgctatgaatggtggggtggttggggtatttatagccccaaccaccaaacttgaccgttggctggaggcgtctgctcgatggcgcaccggacagtccggtgcacaccggacagtccggtgcccctgccacgtcatcactgccgttggattctagccgttgaagcttccgacttgtgggcccgcctgggtgtccggtgcacaccggacatgtactgtttgatgtccggtgcaccggtatgggcgtgcctggcgtctgcgcgcgctgcgcgcgcattaaatgcaccgcagggagccgttggcgccgcagggagccgttgctccgctggcacaccggacagtccggtgcacaccggacagtccggtgaattttagcggagcggctgccgcgcgaacccgaggctagcgagttcctgaggccgacctcccttggcgcaccggacactgtccggtgtacaccggacagtccggtgaattatagccgagtcgccttagaaattcccgaaggtggcgagtttgagtctgagtcccctggtgcaccggacaggtactgttcactgtccggtggcacaccggacagtccggtgcgccagaccaggggtgccttcggttgcccctttgctcttttgttgaatccaaaacttggtctttttattggctgagtgtgaaccttttactcctgtatacactatacacttgggcaaacaagttagtccaaaagatttgtgttgggcaattcaaccaccaaaattatttaggaactaggtgtaagcctaattccctttcaatctccccctttttggtgattgatgccaacacaaaccaaagcaaatatagaagtgcataattgaactagtttgcataatgtaagtgtaaaggttgcttggaattgagccaatataactacttacaagatatgcatggaatgtttctttctttatttagcattttggaccacgtttgcaccacatgttttgtttttgcaaattcttttgtaagtccatttcaaagatcttttgcaaatagtcaaaggtgaatgaataagatttttgcaaagcattttcaagattttgaagttttctccccctgtttcaaatgcttttcctttgactaaacaaaactccccctaaattaaatcctcctcttagtgttcaagagggttttgatatatcatttttgaaatactactttctcccccttttgaacacgataggatgccaattgataaatatttcttggaaaacactaagtttttgaaattggtggtggtgcggtccttttgctttgggctcctttctccccctttttggcatgaatcgccaaaaacggaatcattagagccctcgaagtaatttcttctcctttggtcataagtaaatgagttaagattataccaaagacgaagtccttttctttgatgctcatttctcccccaaagaatagagagatggttggagtgatggcgaaggatgagttacggagtggaagcctttgtcttcgccgaagactccaattcccttccaatatacctatgacttggtttgaaatagacttgaaaacacattagtcatagcatataaaagagatatgatcaagggtattcaaatgagctatgtgtgcaagctagcaaaagaaatttctagaatcaagaatattgagctcatgcctaagtctggtaaaagattgttcatcaagtggcttggtaaagatatcggctaattgatctttagtattaatgtaagaaatctcgatatcccccttttgttggtgatccctaagaaaatgataccgaatggctatgtgcttagtgcggctatgctcgacgggattgtcggccattttgattgcactctcattatcacatagcaaagggactttggttaatttgtaaccatagtcccgcagggtttgcctcatccagagcaattgcgcgcaacaatgtcctgcggcaatgtactcggcttcggcggtggaaagagcgaccgagttttgcttctttgaagcccaagacaccaaggatcttcccaagaactggcaagtccccgatgtgctcttcctattaattttgcaccccgcccaatcggcatccgaataaccaatcaaatcaaacgtggatccccgagggtaccaaagcccaaacttaggtgtataagccaaatatctcaagattcgttttacggccgtaaggtgggattccttagggtcggattggaatcttgcacacatgcaaacggagagcataatgtccggtcgagatgcacataaataaagcaatgaaccaatcatcgaccggtataccttttgatccacggacttacctcccgtgtcgaggtcgagatgcccattggttcccatgggtgttttgatgggcttggcatccttcattccaaacttgcttaggatgtcttgagtgtactttgtttggctaatgaaagtgccctcttggagttgctttacttgaaatcttaagaaatacttcaactcccccatcatagacatctcgaatttctgtgtcataatcctactaaactcttcacatgtagactcgttagtagacccaaatataatatcatcaacataaatttggcatacaaacaagtcattttcaagagttttagtaaagagtgtaggatcggccttgccgactttgaagctattagaaataaggaaatctcttaggcattcataccatgctcttggggcttgcttgagcccataaagcgccttagagagcctatagacatggttagggtactcactgtcttcaaagccgggaggttgctcaacatagacctcttccttgattggtccattgaggaaggcacttttcacgtccatttgataaagcttaaagccatggtaagtagcatatgccaataaaatgcgaattgactcaagcctagctacgggtgcataggtttcaccgaaatccaaaccttcgacttgggagtatcccttggccacaagtcgagctttgttccttgtcaccacaccatgctcatcttgcttgttgcggaagacccatttggttcctacaacattttggttaggacgtggaaccaaatgccatacctcattccttgtgaagttgttgagctcctcttgcattgccaccacccaatccgaatcttgtagtgcttcctctaccctgtgtggctcaatagaggaaacaaacgagtaatgttcacaaaaatgtgcaatacgagatctagttgttacccccttatgaatgtcgccgaggatggtgtcgacggggtgatctcgttgtattgcttggtggactcttgggtgtggcgcccttggttcttgctcatcctccttttcttgattatttgcatctcccccttgatcattgccatcatcttgaggtggctcatttgattgatcttcttcttcatcgacttgagcttcttcctcatcttgagttggtggagatgcttgcatggaggaggatggttgatcttgtgcatttggaggctcttcggattccttaggacacacatccccaatggacatgttccttaatgcgatgcatggagcctcttcatcacctatctcatcaagatcaacttgctctacttgagagccgttagtttcatcaaacacaacgtcacatgagacttcaactagtccagtggacttgttaaagaccctatatgcccttgtgtttgagtcataaccaagtaaaaaaccttctacagttttaggagcaaatttagattttctacctcttttaacaagaataaagcatttgctaccaaaaactctaaagtatgaaatgttgggctttttaccggttaggagttcatatgatgtcttcttgaggattcggtgtagatacaatcggttgatggcgtagcaggcggtgttgaccgcctcggcccaaaaccgatccgaagttttgtactcatcgagcatggtccttgccatgtccaatagagttcgattcttcctctccactacaccattttgttgaggggtgtagggagaagagaactcatgcttgattccctcttcctcaagaaagctttcaatttgagagttcttgaactccgttccgttgtcgcttcttattttcttgacccttaagccgaactcattttgagcccgtctcaagaatccctttaatgtctcttgggtttgaggacgaattttctaagaattnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnttttcaactctgagaatcagcttgattcgttcttctggcatggcttctactggccaactgctctctaggagggagccgagttggtgaagtcctgcgaagcatgtcagtttcatgcaaagcagacacacacacacaccagctcaggctctgcaaatgattccaccctcttggccattcgccgtatggggggtggatatcctgggaccatttcctagggctgtcggcgggtaccgttttctctttgtcgccatctacaaattcataaagtggtcggaggccacccctatggtcagtatcacccaaggtgctgctgttgccttcctcaattcgattgtatgcagatttggggtcccaagccatatcattacggacaatgggacccagttcaaaagtcgactcttccaagagtattgcgagggcattggcacccagctctgctttacatctgtgtctcatcccaggagcaacgaccaggctgagagggcaaacacagaaatccttaggggactcaaggcacacacctacgactgcttaaaaaagcatggtgccaattgggccaatgagcttccgtccgtactatgggggaaccggaccacacccagccgagctaccggggagaccccgttcttcttggtctacggggccgaagcctgccttctcccggaaatcattatgggctccccatgagtccagtctttcgatgagtctatgcaggaatagctacgacgtgaggacatggacttcatcgacgaacgcagatggcaagcggtgatccgaaatgcacggtacaaccaagcgctcaggcgctaccaccaacggtttgtgcatagtagggagctcagggtcggggacctagtcctaaggcgagtactgaaccgagaagggctccacaaactctcccccagttgggaaggacccttcaaggtgacagaaatatgccgaccatggtgtgtccgccttgccacaacagaaggagtgcctcttcccaatccctggaatatagagcatctctgtaagttctatccataatagcaaaactggggggttgagttttcttcctttgtaactaggttacgcatatgtgtatgtcaattcggtgaggcccgccctcgtaagcccatctgttggtctacacccatgtatatcgagttataaggaaaggatttaccccctagatgtgattttgtgatggttttattctacttcggtttacatgcattattttttatctaacccacccatatagtttcccacccttgttggtatgatgacatccgaattgagtagacaggcttgcagttcaagacccctttactgctacagggggtccggcaaactgcggaccagttctagagaatgggcgctagcctcctggaggggtccggagttgtgtagccgcttagcatggttccgtaccctaagcctgcatgctccaccactctataacgggtgccctagtatttggaactgtgatcctatgggtccaggcatacggcttggcttcccaggctaaatcctgcaggtcctgttgcataaatcaaaggatggcagataccagacgatggatcctatggtgtgctcctaacactttaaagccgaagctgtgtacaagtccaggtcccagtccagtagtaggtagtctcaaactgtagagactacctcctaggggccggaccaccaattttatctttggtatactggtatccagcctcgacacgtcgagcctacctcccagggggccaagtaccaaggggaagttgatgacactacacataacaaggacaaataacatacaaataagtttaagttccaatgctacctcattagcggttcttataatatcttacaaaatcaaaagttattacaaccgcttcccagtggaacccttgctttgtctctataggtcgtcagcaggatcgtgctggaagcgctcggccaccagctccacggcgtcttgtacactctcccaggcggcgtcttctatggcggctaccggaccagcgatcactagcgccaaggatatggtggggtcgtgactccggaagcatgttaggacttattcaaccactgcccgacagagcttgctgccctctgcctctaggcgggccccgaggatctgatccaggcgacggaggcgatcggcggtagagtccagcaccgggagcgcatcagagatcgacgctggtagctccgacattgggatggggctcatccctagtggcactagtgccgtgcttgcctcgccggcccacgcgacaatacactggacctcgacgcggtgctccgcttggagatcttcaagggccttcctggtggcctccatcgcttggggacccggtgccgcctgcgctgcattgaactagcggatctgctcctccagcttcctctctttctcctctgcctcgagcttgtgcttggccagcaactcgcctcgccgggtgagcatttcttccctgaagctgagatccgtctcttgcctggcgaggtccgtctcccaccggtccaaggactgctccttcgccttaagattttcctcggcgagggtggcattgctagccttgccctcgagctcctgctaccacttttgcagcctctccacgaccctgacctgctgggcccgctgggcttccagagtctggtccagggcactcagcttggcctggtactctgttgtgagggtctcccgctgggtcacgacctcctccttcctggtcaccttcttctccctccgggacgcctccagctccctggcgcacaccctctggaggtccctcttgtactccttatggtcctgctcgagttgggaccgctcggagatgaattgttgggacgccgttctggtgcgctcctccagttgggtgcgccagtcacttaggcgctggtgctcagcctcaagcgcctcccactcccgcaagattgctgccccagtgtcactaaggacctagtgggcacgagacattatgcgagggagggggactggcgccgcttcttgctcggcacccgaccggagtcgccgcccaaacaccacctccatctcctccggagcaggcggggggttggagctggacatgcctactgcgtcacccccagtgtcgagagcgggcgcggatcccccagctgggacctccttcgccactgcgacgccgcctgacgctgccaccggacccccggctggggcatgagaagccgctggtgctgtcttggcagcagctgggggtgggccgccggcaccactctcagcaggttcctgctgttgagagccagacccgtcggtgggcctggtatctggtggaggaggcatgaccttgggagcggcgaaggaagaagccctagcgaacagatgatgggttaaaactggtcggcatgatgattagactcatggaaaaggggctacgcttacttggggccctagactttctagtgaccctggaagcggggcgatcaccgctcctgctgttgctatcgctgttgctgttgctgttgctgctgctggtgcccctgggggtgaggactgacgcgcctctgcgcgccgtgggcctgggagctagcttcctcggccccacctgcagccctctgacgcttctggggggggctccgaaatgagcgacccatcagcgcgacatggcctgcgttgcctctcctcctccgaccccctcggagctacctggggcggaggcactgctcgcagcccctttgcctttgtccaaggggctaggggccacggcggggttggtgctgggagccgcaccagtgggctgggaacctccaatcggtgcattagaaatctggatcccacggagggggtcccggccaccggtctagcgaaccgccatgccgctctcgtcgagggtcggcaacgtggccaagatcaccatcctcaggcctggatcgtcgcagagcgcagggatgttctgggggagtatcagggactcagggacaaaagtttccccaataatccctcccatcaggactgctagctcgtcccaggacagaacggtgcccggcctgcgttggatcctatcgatgtcgtttgggccggtgaaccaacagcacatacgcggtctcctctgcagcggcgcgatccggtgcttcaggagatcgccgaccacgtgcattgatggcaggccgcccgtagccaagcccttgattctgtccaatacaggcaggaactctagcaagagggacggcttagtcctccactgcttgcggtcgagcgctggcccatcgctcggcaggacgaggcggtcgttggcctcggcgctggcaatcacccaatcgttgcgccagttttcccacctcgcaccgccaaaggtggggatgtatacgacggctggatctggcctcgtctggaagtagtaggcaccgatgtggtccctagtcttcccgaacttgaccagcacgaagaagcagcggaagagggaagtacagggggccacacctacgaacatctcacagaggtggacgaagatggctgcctggaggacggagtggggtgtgaggtgttgaagctgaagcccaaactcctccagcagcagcaagaagaagggcgagaatcggcaacgccaacccgtagaagatgtaggaggtgaacagcacgaactccccggcggtgagatcgccatgagggacggcgccggcgcggaacttccggcgagccctggcgcgctccatccaagcaggccgcgcaccaggttgagcgcctccttagactgaaagcagtcaggatgaccaagcgaggccatggcgtgtgcggcggcgcgagcgtggaacagaggagcacgaaggcaaaggggtgcaggcgattgggagagaatgcgaaaaggtaactgctgcacgcggggtgaatcctttttcaaggaaacctgagtccttgttcagggaaacccttccgtgcgcccttgaattgccacaggaaatctcgcccgatgcgcacataggacccaggcagcccactctatgacacggtggcccgggtccacaagtcatacagattgtgtgctggatttcgagtgcggaaagagcgaatcgccatgcgaactgccgcgcacgatagcgcacctcctcggggccgctgcagaagacaaaaggttatgcagcggcaacgaggcgtcccacgcgtggcccgacgaaaccaccaggcatggggccatgggtcagtcagctgcagagacagatatggcagttgacgtgactgaaggcggattgacagcgggcgtgtctgcagacgcgctaaaacggcatgccaatcaccgatcaggtcacgttgaagcaaagtacaagctttggccccacatgcaggctcgcatcctcccctaaggtgggtccgggggccactttcggcaccctgaaacaagggtaccccttactactgtataaatacgcagtacccacgcgactatctttagtcgcgtggtaaaagagctgtatgtgggaccaaaccatgactcgccctagcctcgggcgactactctaggccagcaacagcacctgaccccaccacatgggcgggtccggggccgccatgtgtccagagaaagtgatgtactccaaggcatcaatagtgagtccggacccccataggagagtgccgaacccatgccagacccctgtatatacggtccaggcctccaagtttggtcatgcgttactctgtcagcattagttatttacataatctatttcttccattatgctcctaggcccgcatgtcgaggctcagcatccttgtatgtgcctcctgtgacaccccagtgtcacctagggtttctcttaaaaagccaaaccaaggaccattattttatgtgaaccaaagtaagcatgagcatcaaaataacttaagtaagaaagaattcaccaagtatatgcttaaaagtgtcatgatcaagacaattgagtctcttaaaggataagaatgtgcaaccctaattaagaaccctaagtgaaccccatgaacaaaattcaagaaaataagcaaaagggaatgaaaagtttaaaattttgagttgagccaattatataagttaaagtatatttgataagcaacaagatagattgagaaagcttagccaaaataattcaagaaaacccccaaatcaagcttcttttgttgggactcattgggaattctgaatttcagaattctgaaattcagaccttgagccaaagatcagggatgttcaccttgatccctaactcgaatcctaatggccccattgacaaaattgtgtctaactaacccctctgtcttgtgccagaagatggcattgggacgcgagccctagacacgacaaaacttgggatttgcctcgggtttgggcagggagacagaccagatttcctggctccatatctctgcaaccagtaggcaaaatcctatgacctccacacaagaatggtagcttgtagggaggagaagaggttttgtgcactgaccaaggcgagagcaggctcggatgagcgaccacacgcgccagagcttgggcagaacgcacgggcacacgtgttcgaccctggtcggcacgccagagctcgcccaacccgcgcgcgcgctcgccccggcgtccggtcaagtccgccgcgcgcccacgccctcggccgtgcccgcccgcgcctataaagcctccccgggcgcacctctcttcgccccgcactcaccctcaccggccagccactgttccttagctccggcgagctcatttccgcccgccattgccgccagaactacggccgccgtggccagcccactccagccaccctccagcccaaccagtgctcggctagctccgccagtagcccgtgaagcttgccaagccctcggacccgaccggaacttcaccgggaggcccgaagaatcaacctcaccggacttcggtcttccgccgccgcgcgtggaccaagctatccagtgagtctcccgcccgattcctttcgctcatgtcttctctggcatcccgtggacctccatgacctatttgattgaactatctcgccgcgaccaggccggtctcctcgccgccgacgagcatccccgcctgcgcgcgtggaccgaccgactccggccatctccgacggtgttccgcacaccgttgtgatccccgcgacctccccttcaccctcggccacttcaccggaacagtctcgccgccggtaagcccctccgcccttttcttcgccgcggctactgtttaaggtagaagaaggacctcgggttaggttctgtagaacccgaggggtttttcgtaatgtcagcgactcatgagaatagtaacctaaggactgaattgcgaggaaaacttagaaaaccgccagggaccccagtgcaaagtggatttccatttaatcaattttgttatttctttttaaaatgaccagagaacttagaaaatccataacttgatgaaatcttaatgaaaagctgtcaaaccaattttgctagctctggaattttatgacctatcatttaaaaatagtgaaccatatgctttctgttctaaattttagagtttaaaattaaaaacagaaaccccctaaaccttgtttaattaaggaaaattagtttttcttttgtgctgagcttaagaaaatttgtagatgcttataccttaattagacactgtttaaaaatagtaggagccctagcattagagattatgatgtagttattcatttaaagccattttgtccaaaacttagagaaaatcagaaaggccttagagattaatgaacagtgattagtaatatttttcctagattacttatgcagcagagaacctaggaaaaatgcagagaccattaatttggaccagtttctaattaagatgctttaattagcattatgtagactgaaaatcaattattagaattgcaaaactataaccaaagtggttaacaaaaatccagtgaacttataaccaccagagccccactacaaaaatacagagcaccccagcctaactttttaagtagggaaaataaatacagaatgataataaggcattttcccactaaatcatgagcaaccccaaataatgtgataatgggcaaccaaaattttgctaagtccatgatgagataaaccaccagagaaaaatacaaacccatgaaaaagaagtgaacccatgccttttgctagtaatttgtgaggaaggccatttagctcaaataatgcaaaccaccccttcccttaggcaaaaggaagccaaactccagaatgattgctcttgcacaaaatactagctaagaaaaataagaactctgttgtttgatgtttttcaagtatagtggtagtagaaagcacccctttggctagaaaccttaagaaaatcttagggaaagaattaaagggtattaatgactagaaatttgtatcaagtcatgttataacacctaaaagccagcaaaaataagtttttgagaattacccactattaaataatagttgtagttcaaagtaccccttctgccctaaaatttggtaattttgtccagagaaaaccattcactttctgaaccccaaattttgagacagagaaccatacaccagtaacaagccactgtaatttttgcagaatttttggaattttataaaagcaacttgtagttcaaacctactccaaaacattaaagagaataaaagaaaagagaagaagaaataaacctcatcccaataagactaacccaatttaccaagtataccactaaagggttttacataagtaaagttaactggttttaaatcaaaagatcatacatctttaaagttataaattctaaagcacatatcatatcatgcatatatcttacgcattgcattcattagattgtaatcttgccgacggagagtacgtgctcatccctgagcaaggacctatccaagaggaggaccaggagcaggcttcagaggctgctattgaggatctccccgcagccccagcaattgaaggcaagccccggttttatgcataaccatgttattatatgctactttactacacttaatgcttgtaggattgcaatgtgcacttaagtgtaggagttgcttgaaacctctagttgcatgaacttaggattcctttttgagatgaatactagtatgctaggtcgagtagctgcttgctaatcaggatctcggtagaagtcgagtgatttttctagcactcgcgcgaggtcaggaattgattgtattcatcttgataatggggtatatgttagtccgtggacttgggtccagggaggatgccatgtccatgagacgggaaaaatgaattaaggattaatgtgtggatacctgagtcaagcttttgaacgtactaagcacatgccgggaaaaatggtaaccggtaaacctagtacctgagtgaagccgggcgcggactttatccctcatgcgacctgagacagggtctcccatgctagctatggtgggtacaagtgcggccactgcatgacggcagtcggggtcagtggagcattgtatgccaaggcggtgaggcctggacgcgaacggggaatcgatggggacggttgtcatgtgtggggtcggagtaccctgacatgccgtgtgtttaggtttaccttgcaaggtttaaaaactcgattcgaatcgtctgcttctcgcagctaatgagactgcttgattccttgtactgcatcgagtaagaagtgaaatgtggattatatgagataacttgttgactgaactaattgattgttaccatgtatgcttagaaggagcaaatctagctaagttaatgatggtagaatttgaaaagctaaaagttgattttagaaacagctagtgcttttggcaaaccaaacccctcagccaaacagctgcatagtctagaggtagaggagtagactcctcacaccggttaagtctagctgagtattagtatactcagccttgcttgtggcaccatttttgcaggtaccatgcaggatgtagttgatggtgtgacttggcctaccaccctgccaccgggttggacggtcgagtgggatgttgctccggcaggagaggagcatgaggagtagtgggctaggccttgcccatttcctcattaccgacgacatcgattatccgctgcactttaatttatgaactttattcgctactcaaaaactccgatttatgtaataactcagtacttaatttgaggtttcctgttttattgtatttcttctgtgactcaccttcgagtgagattgtgggatttgatcctggttaagtggcttcatcagactagatctgagggactgacgggttattccgatttaagtgtgttacggcccctgaggcgtgacttaggcacttaagctggaataattcgggcggttctgccacagctggtatcagagcaaattccaccacagagaagggcaataaaccatgaataccaattttcaaaatctaaaacctgcctagaagctactacggatcgtcaggactagaccgctagacctaggacgaaaggccttaggcatagagggagaaataggtggctaactaattaggccctgtgggccaatacttatattttaggatgccctaaaaaggcaccctattttccttttgagaggcaacgtttctttccgcatgcatgcattataaaacataaagaggaattaaaattgagctaacccccttttcttcgaaatcatccgggctctctttttctttttccttccaccataatctttatctttgattcccttccgcagatgaattcacccacccccgccagtggaggagactctcgtttcagttctgacttcctttctcgcgatggcttcccttccattttgtgggaagtgcttaattccgccggttaccctacgccccctttgtacacggtgcagttgtatgaggagcatcgggtacctcgttgtcgggtctggctaactttggaggctcatccccttcagccgggttggcgttctcttgactctgagacgattggactcaggacggacgacaccgttgaggcagcagccatgaagactctgacgactttttgtggctaccatcccctggagatggtgatgcaccccttgggactcttccccgctgagaagaaggatgatcccatgtggtgtaaccgcgtgagccatgtgaaggatgtgtgggcaatgtatcctgacttggttgggagggtcactgttcagtgcatgagtgcgctgtaccgccttcaggcccttcagagcgatgctatgacacttcttgccaataccgctcaggcagccaagctcaccctcgacagtcgggaagattttgtggtcgacctatccacagagttggtggaaaaggatctgcaggtggagaggctgaaccagcgtattaccaccctggagcagcaagtggagatccgagataacactattgatgtcttggagaaccagcttcacgacgtgcagagggaactcgaggaagcaaatgaccacttggacatgcaccacctggagatggaggccaatgaagcaggaagcgagggagaagaggctcccgaggagctaggaccagcccctggtgccaatgggactacctccgcgatacctccttcacccgtatccagtgtcgcttccaccgctcagggttaagcagtcgctttgacatttttaggcggatagaaacctatgcgagcttagtggtatcacattttggactaggcttgtgggtaccttcccctgattaatgtaaccctgtaaacttttgatatctgtgggatccttgtcaccatgttatcttcattcgaacctaatattatgattatggcattttccttccatatgagatgatatcttgtcgttcggaaatgtgaattgggataacaatggcgacaatctctgttttcagatggcagcgaggcagcgtcgcgggcaaaatgagcaagctcccccgccacctcctccagctcccacagtgcaggagctgatggcccagcagaatgagattctgcgacagctcttgcagcgccagccccaccctcagcatcctggtggaggccagcatcagcgacctccggctatggcaacataccaggagtttctgagcacgcagccgcccttgttcaccaaggcagaggatccattggacgccgacgtgtggcttcgcgtcgtcgagtccaagtttcccctcctcacaggagactgccctgatgaggccaaggctcgcttcgccgcacagcagcttcgcggccctgctcggacttggtgggatcacttccgtgctatgctccccggtgatcgtgaagtatcttgggaggaattcaagactgccttcagagggcaccacattccagctggcattcttgatcggaagttgaacgaattcctggccctcaatcaaggaacccgcacggtactgcagtatgcgcaagccttcaacgacttatgccagtatgcagggtatcatgctgattctgatgaaaagaagagggatcgcttccgcaggggtctcaataccaagctgcgggaacgactcaacactgtccgggccgatagcttcaatgagttggtcaacatggccatctctcaggaggattgcattgttgctcaccgggcagagaagaagagaaaggcaccaatggcagcaccatccgctcaggctcagaggttccggattgtttctcacaatcagagcaggggttttcagcagcaggcaggcagatgggtgatcaggccacctcagcagcagcagcagccggcacccaaccgctatccagctcccgccccaagaaacaatcagcctccgcagcagcagcagttccgccagggcaatgggaacaagtgtttcacttgtggcaatgtgggccactatgccaagaattgtcccaggaaccagcagaggcagatgccagcaccaaatcaagacaagggaagaaagcagaaggtacaagtcaggcaagggaagctcaacttcactgctctagaggaagtgccagaaggagctcccatcatgaccggtaccttttcagtttataatcaacctgctttaattctgtttgattctggtgcatctcatagtttcattagccaaaagttcagtgctaattgcaaacttccattctctcactcaaaagggtcattcatgatagtcacacctgggggtaaaattgcaactaatcaattaaaccaaagtgtgcctattcaactgggaagccacattatcaaaaccactcttcttgtgttgggattggaaaatgtggacattattctaggagcaaattggatgaccttgcaccaagttgtgctcgacgtagccagtcgtaccgtggaagttaattctcccttctgcgggaatttcactttgattctgcctagtcagggttcttctcagtcatgtgctttctctatgacggaattacccctgaagaagatcccagtggtctgtgagtatgcagatgtctttcctgatgaattgccaagaatgccactggaccgggatattgagttcgccatcgagttgcaaccgggaacggccccaatttccaagaggccctaccgaatgccacccgctgagttggcagagttgaagaagcagttgcaagagttgctggataagggatttattcgcccaagcacttcgccttggggctgtccagcactgtttgtgaagaagaaggatgaaagcttgaggttgtgtatagattaccgccctcttaatgcggtaactatcaagaacaagtatcctttgcctcgtattgatgttctctttgaccagttggtcggggccaaggtgttttccaagatagaccttcgctctggctaccatcagatcaaaatacgagcaagtgatattccgaagacggcattctcaaccagatatgggctatatgaattcttggtgatgtcattcgggctgacgaatgcaccagcatatttcatgtatctgatgaattctgttttcatgccagaattggacaagttcgtggtggttttcatcgatgatattctggtgtactcaaggaacgaagaagaacatgccgggcatttgcatgtagtacttcaacgtctgcgagatcaccacctttatgccaagttatccaaatgtgatttttggctaaaggaaatcaaattcttgggtcacactatctctcaggctggaatagctgttgatcctgataaagtgcaagaggtgatgaactggaggccaccaacgactgttcgccagattcggagttttctgggattggctggttattaccgaagatttattccggacttctctcgaattgcgaagcctattactgagttgctgaagaaagaagtcaaatttgtgtggagtcagaagtgcgaagatgccttccatgcattaaggcagcatctgaccacagcaccagtattggcgcaacccgacagcagcaagccttttgatgtatattgtgatgcctctggcaccgggctaggttgtgtcttgatgcaagacaaccgagtcattgcttatgcctcaagagcactcaggcctcatgagcaaaattatcctactcatgaccttgagttagcagcagtggttcatgcattgaagatgtggaggcactatctaatgggaacccactgcaacatcttcactgatcataagagccttaagtacatttttactcaggctgatctcaacatgaggcagagaagatggctagagctgatcaaggattatgacctggaggtacattatcacccagggaaagctaatgtggtagcagatgccttgagtcggaagttgcagtgcaactgtattctgatggattctcgtgttaacaccttgtgtgatgagttgagcaagatgcaaattgaagtgattccttctggttctttgtctcacattgctgttgagccagccttgcaagaccagattatcatggcccagctcagtgacaagggagtgcaaattatcaagaagaatctccatcagaaggttgagaagtataattgtttccgccaggatgagaagggtgtgttatggttcaaaagcagattggtaattcctaaggaccaggatctcaagaagaaaattttggatgaggctcatctctccaaattctctatgcatccgggaagcaccaagatgtaccatgatttgaagcataacaatccccacccttttcctataagtctcacccttcgcttcaccctgggaggactctggcccgaatctcgggacgagattcctttaaggggggaaggctgtgacaccctagtgtcacctacggtttctcttaaaaatgccaaaccaagaaccattattttatgtgaaccaaagtaagcatgaggatcaaattaacttaggaataaagaattcnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnngggtgctaatcatgaaccagtccagagcaacactatccgatggccattgcgtccggtcgcacgagagacgcgtgcggaacgtcccgtaggagcggccaaccccccattttgcagctagcagccgtccagtagggacagccgccgagctccccgacatgtctccttcgggaccgggcttctatttcaagctgcgggacggtgcggtcaatccatgtggacaccatgcgagttcgcgcttcactatctgggctggggacccacctccatcaatggtctgcatgacgcaggatattccatcagccatggtgcagtggaatccgttccagaggatggcctctgcaccaaacgctcgccaaggtaacagaagcaatccaggcccatcgggcgtgatctcccatcgatccgtatcgatcctttgacatgaaaaggcaatcacgggctcacgcttttcggagtgtaattcaggctcccgggtgcagctttttgcgcgcttgcggcagggggcatctggtggacatcaaatgatatgggcttgcttggtccagggaaccggcagcacctgctgtcccgagatcagttgtgatgctatgtcatccgtcgatagtcggagcttatccagctcggatcaggtgatacgcttccctttcggagaggtttgagtttcagacctggtgctcagttatgataaaaagggtcggcagtgagagaaaccccgaaaacttgtcaatcgaaccaattaccttatttacttttcctgccctaggagtagatgtagcatagttctagttgtagtcttccacatatccacctccacccctattcgactctacgtcgtctagatccgtcttgggtggcctgccgatcccaagacgaccctaggatctcacccctcccggggggcaagatctagttgtccatccaagacttcttcctcgatttgatctcttaattcctaggcgactccacgtcgtctggggacgccccgggtgacctgtcgacccggagcaccttaagatctttccccccaggggacgagatctagattccagcaaggagtaggaagacgaccctgtcgccaggtcgcggaccgtccggcccagagctgcggaccgtccggtgtgacgcagggaagacaccgctcctgcgcccaggtcgcggaccgtccgacccaaggctgcggaccgtccggcccaaggctgcggaccgtccgcgcctgaccagagggcaccgccacggttcttgttgagtgtttggcgctccaaaaaggcgtcaacagtagccgtcacatcatctattgtgtggctatgcttaagtgtgccttgatataatttagaataagtcgagtctctagaacgcggcaatttttaaaagtaaacagaagctgaatttattgattgctgttttgggctgcacgcactgttttagttgtgctgtttgtttgataaaccaaatcatgttttctgtagaaaagtcatatagaagagttgtagatgacatgattatcttgcttgtactaaaatttgacagccataaacctgattgtttaggagttgtgcttttcacaagcccagcacctgaatctgtcaaatttctgaacatatttcagaaattgcaatggttgcttaagttaatgttgaaattagttattggtggtcacaagaaagttgtagataactttattatcgtacttgtgttaaaatttgacaggcataagtctaattgtttaggagttatgttttttacaaattcagtaactgaatctgtccactttctgtacagatttcagaagctgcattgtttgcttaagttaatgttagaatcagcccttgtagattataagaaaagttgtagaggcttttcttatcttgcttgtgttaaaatttcataactataggcctgacggtttaagagttatgaattttacaaactggttgctgtgttctgtccaccgtcagaacagatttcgaaaactgtaatatttgatttagttaaacctggaatcacttcttggtgattatgaaagttgtgtagtacttttgctaagattttcaaaaagtcttagatcactctttttggtggtctgaagattaagttacatgtgtttgaagtgtgaagactgaatctgtccagttttggacagcacagccttcatagtatattttaaccttgatacatgctaaaccagcctgggatgtttataaataatttgtagaacatttaattagctttccagaaagtctaggatcaatttgtttggatgtctgaatcttcagttatgaatttttaaaatcacaagtctgaatctgtccaaatctggacagagctgttgtgattgcactttttgaccttgctaagtgtttaatcatgctgtgatgaaaataccaaaattgtagagcactttctaaactttccagaaagttttagtttgctatttttggattaatatttgaaaagttattattaaaacaagtaactgctgtgctgctgtccaaaaaatctgcacgtgctcaaatgaatatttagttcaccattttggctaaaaacgcttagttagcacttaacggacatagacttgtgatggctaaacttaggttaacatgtgttccatgattaatgtgcttgcttgctatagttgattgtgatagaggagtccatcgacattgatgcatcggtcctttattaaacttgtgtttgtgatgcttttgtgtgatcaatagaagaactaatgaaaagccgtagcaactaaataaatgcttgtacatatgatatcgtgttgcgttggttaattgtaggtagtgatcattgtctttccagtggtagtgtttacgtgtgcccaatgacacataaataactagtgtttgcgtatagttgttgcagtgtcttactaattaatgtttagttcgccactgtgtcttggtatatcttatgttacttttattatattcatacatatgcatcttgcacctcatataggaccgagagatgatgatcgagccagtgatgtggtgccaaccacaagatgccgttgatggacgacctaaagaatggacttaaccagtggatgctcgccaagcgagtacctcccccagcaaacactacctaagtgttaaattaaaggcaagccccggttttatgcataactgttatatatatgctattttactgcacttaatgtttgtaggcttgtaccatgcacttaagtgtaggagttgaatgaaaccctagttgcatgaactcaggattccctttgagatggatactagtatgctaggttgagtagctgctttgctaattagggatctcggtagaagtcgagtgatttttctagcactcgcgcgaggtcaggaattggttgtatccactttgataacataatggtgatggtctgtggacacgggtccatggggacgcgtggtctacgagatgaaattggaataaggattaacgtgcggatacctgtgtcaagcgtttgaacgtactaaacacatgccgagaaatatggtaaatcggtaagcctagtacctgagtgaacctgcccgcagattgccctcctcaggcgacctgagacgtggtctcccattccggttatggtgggtacaagtgcggtcactgcacgacggcagtcggggtcagtgaggcattgtacgccaaggcggtgagcccctttctgttgccagggaatcgatggggacggttgatgtgtgtggggacggagtgcccctacatgtcgtgtgtttaggtttaccttgcaaggtttaaaaacttgattcgaatcgtctgcttctcgcagctaatgagacttcttgatccattgtactgcattgagtaataagtggaaatgaggtgattggcaaaagatgttgtttgataaaaattcttgatatcatgtatgattagctaggtacacatctagtcaaaaaggatcatactaaaacttgaaaagctaaaacttgattttagactcagctagtgcttttggcaaaccaaacccctcagccaaacagctgcatgtctagaggtagagaagtagactcctcacaccgggtaagtctagttgagtaatgtatactcagccttgcttgtggcataatttttgcagatattcattaggatgattggttgatggtgtgacttggcctccatccctaccaccgggatagatggtcgagtgggttactgcttccgcaagagaggaccaggaggagtagagtggccaggcttcgccatgttactcggttcttctccgttagttatttctgctgcattaaaatttatggttattatttctgaaactccgataatgtaatcactaatgatacttattaaatttgtggtattatgttttattgtatttctctgtgtctcaccttcgagtgagctagtggtattcgatcctggataagtggctttatcggactagatccgagggactgacggtttattcctatttaagtgtggtctagcctctaaggcgggacttgggcacttaagtttgaataattcgggcggttccgccacagctggtatcggagcgaataccatcacagagaagtcaataagtcatgattaccaaccttttctaaaagtaaaacttgctagaaaccaatgttggatagatgtcaggacgataaggatagacttaggacgtgaagccttaggaaatagatgggtagctaggtggctatttatataggccataaaggctactactactattaataaggatgctgtagaagcaaccgaaaaagtagttaggtctgagaagacgactagaatgagcatgcatcatgattgtcgcattataattgtcttttgtgcaccaacatgcttctctcacctttattcaaataataaaaaaaattgtgaataatgtgctgtattgctaggaactgcaaaaaaaatgtcttatcttgtgtgtcatgatagtctttactaggttatgttatgtgcttctcttgtcttgctatctaggtagtattgtaattgttcaaccctttttgcaaaacattttgttgcttgttctgttcataaaaagactcctccaaacaaccttgagtttagcaagtgaacccgcttttaaaaaaatgcttgtgttggcgttttctagcccttgtgggttttacccttgaagttacacctgcacagcttgtagattcccatagcttgactcctagatcgaccaaagcttccttgtgcactggttacgtcaaaaaaaatttgttgtttggtgtctagttgcgcaaaccctatcaaggccatgtttctttccataaattccttgcccctaaaacttcatagcattcctgttgatcatccagctgatcttgttgcctacctctcctttcgcatggatctagtgatctttttccttgtgaatcatgttgtgaccttatcatccgaatctctgatctttcatgattctgccctattatcttgttatctactataacccgttctcaagtatcgaatgttgatctacctaagtctctcaattctggtcattctcatactcgttctctgaggatcatgacgatgtttatcaactttatctctaaacagtgtatccatttggttcaagggatgttgttgtcatcttgtggttctctcatgtctctacaagttcatcaacatgatctctggagtgcttccttctcatatcaaatctcgtactaatcgctggcctgctaatccccgtgatgatcataaaataactctatgagttgaagaaaattctcatgtgatgatcttttgccaataatctctgcttcaactctgatcacattcttattttctgagccatactctcatgggctccaactatcagtgctatgtgaatttcttattggttgcgtttggtaatgatgtcatgactaacgactgatggtgccgcgacgaaaccgagagcctactatggtgcacacatggttgagctgctcggcacgcgctagtatcgcggttaatagtcgtgatccattacgagactatactgatgtgctatttttttgtggacactctcagaatgatcgctgcattttgtctcgatatgtcgcgatattctaaccaaatctgtctccagtatcttgtcagataccctctcatgaatttgcatctatcttcagtctgggagttacatgcttctccacccataaatatcctcattcgaatctcgggacgagattctttttaaggggggaaggctgtgacaccccaggtgtcagtttcgtgttacgtcgcgagatttatcctaatctcggatgctcagtaaaaatttctatttctcgctcgcgtatgtccctgattatccagattattcattcacgtttcaccgaattcggagttactcagtctcacagaaggccaattttggagcctgttaaaacttttatcgtcggcacaaatgcgaactcaaaaatcattctcgaattataaacctcatctgaagctcattaaatcaaactctcgacgactgttatttgatctgtgtccgaatccaatttctcgatgttcgatcgatgtccaactattttaatccgagtccatactcacaaacgaaataatcaatatgtcgtcctctaatcaaatcttactcgactcagcttagcatctctgtatccaatccgatttcaaaatcaacatcggcaacgatttttatatatcacgattcgctttctccgactaaaaatccaaaaccgatcaaatctcaggacgatttattttcgatttacgcgtagggaattattttcaagcgaaatctaaacagactctcggccgagttaatcgcgcaaccttccgttcgtccgaactcttttcgctctgtttctcagtagcgacgaattccgcaggaacatttttagtccggaaaatatttagcgcgacccaatttagtgttttgggccaaatccagtccagcccattcggcccataagaaaccctaccctaatttctcctctataaatatgggcttccctcccttgcattctgaaaattttccatttccaccccagccgccaacacccttctcttcctcctctaccattttccagccgtgggctccttcaagcacgtagagctggagctccttccccagcgcgcaggggcttccatggccgggcgttccttccctccagcgcgccgaagctcttcccgtggcgtcctctgcctttcttcttccctgcttcacagcagcaaggccaccagcaggctccctgctccccgcgcccccagccatggcatccttcactcccctactgtttttctcccagggcgcagcagcaaatcccatgcagcggctccatggccgagcgccctgcccggtgctccagccggcctcctctgcccctgccattttccacaggagccgagctcctacctgcagcaggcgccccctgctctttcctatccgcgaccagggagcttcagctggcgtgaaacttcacttgcgcacggcggccagcaccctctccttgggctccaacagcttggatgccgaacccctttcttccttcccctggccgagctcgagcttcccatggcgccattcctccctctctctgttgtacatagcgccaagcagcaactccattttccctgcccgcgcccaaggtcggcgaccagcctccccttccctgttcttgctgtggccgagccaccacttccccagccgtagccctctccccctccattgtttcagcgcctgaaacaaacacctggccgccatccacacttgtgctcgatgaaatgtgcagcagccccgacggctccgcgcgctgacggcttgctgttttgttgcgcagtgagcagcacgccgtgatgccgccgtgtgttcgctgtttttgcgcagccccaaacgtcgtcgtcgttcaccccggtgagaccgcgacgctccttgttcgattccgcatcgatgttattttcctatgattaattatgtatgtgtgttgctttgttttatttttgtggaggagagaaccccgtgttttgcgaggagaaagcaagtcgcttaacgctcgtcggatgtttggagcgatgcacgaatcggaatcaccgtcattcttgcaaacatcgtttgggtttgtttatggtgagccgatgcatgtcgctctcgatcgactcgattaatcattttgtatggatgtgtgtaaaatgttcgattatgcgcattggtaggatcatgtttgcgattggagaacaagaggttaattgatgtgcgcgatttgtagttgtctaattatgttttggtcgatgatgtgcatgtggttatatgtgtgtaaaagtataattttataaatggacgcgtgtagggaagaaaatgaaatacaaaagaactcgagtatttttattttgataggaaaatatgcgatgcgttgtttgatgcgaaaactaagttacaaaatgtggattttgttttggaaaatgcatcgatgtgtttatgtgaaaagtgtatttgttttaagcaatgtgatgggattcgtaattttagaggggatatatttattgatgtgacgagtagtttagagaatgctagtttgcgtagaggatgtatcgttaagacatgagtgtcggagtccatttatactagtggtcgcgccacatggattgaagtgtctcgagtgcacgccataatatggttgtatgcgagacagggttatgcgtacgatgagtttagtaaaaattccatcggtgtcagttgtgttaagttgaagtttatttgtgcgtataaagtagtaaggtatttaatgcttacgactcttaatcgatggtagaaattgtcttgacttaaatagagaggtggtgacatgccagagtagtcatcgctttctctatatttataggtcaagtcatgacgatgcgtattatgcgttcgttaaaattatgtttcgtatatagtgtatgattgtgctcacgatttcgagtagacacttcaaataagtcaagtagctttgtaatgcaagatgtgtgatgaagttagtttgttttaggatatgtgttgaaatgctccattcctgtgatagacatgtagggttatttcaaaacgggtcgatgtgtgtgatgatgatattcatgatttaagtagatgtcctgaaattatgtggcgaagcttaggttaagttgcaagcgatgtggaaatgttttcgtaaagatatatgtggaatgtgaacgagtcattcaatgtattcggtatgtcgtgtagtggtggtatgaaaaatgagttaggaatcgatcggctaaatgccaagttcggttagagttattttgatagttgggattgtggggtgaagtgatggcatgactacgtagctgttggacaccaaaatgagcggacggtccggcccatgggcccggacggtccgcgtgtcccgagattagattaactcggatgtttatccttatctcgtgcgtggttatccatctaatcacgtgggagtttgttggctatctcttaggaaaaggtccagacctcctcccctataaatataaaggggtacggccgattgagaacccccgaacacattccaatcgaaccaattaccttatttacttttcctgccctaggagtagatgtagcatagttctagttgtagtcttccacatatccacctccacccctattcgactctacgtcgtctagatccgtcttgggtggcctgccgatcccaagacgaccctaggatctcacccctcccggggggcaagatctagttgtccatccaagacttcttcctcgatttgatctcttaattcctaggcgactccacgtcgtctggggacgccccgggtgacctgtcgacccggagcaccttaagatctttccccccaggggacgagatctagattccagcaaggagtaggaagacgaccctgtcgccaggtcgcggaccgtccggcccagagctgcggaccgtccggtgtgacgcagggaagacaccactcctgcgcccaggtcgcggaccgtccggcccaaggctgcggatcgtccggcccaaggctgcagaccgtccgcgcctgaccagagggcaccgccacggttcttgttgagtgtttggcgctccaaaaaggcgtcaacatactttttggcgactccgctggggaagaagttgcagatctacaaaatcaggcttacatggccgattctaaagatctcaacagtgcttctccaaacagcaacacaaggctgactaatttatcggccgctgagcataaaaaattagaagatgacatgaagaaaatagacgaggaggcccaccgacaaaaggatcaggtgctcaaggtggcggacaagtggtacctctcgcacttcaaggtagactgccaccagaagaccgtccaagagagggagataaacgccgagtatatgttagccgtgctgcaacagctccccacaataggtgatgccaggtcagccgatgatattccatctattaaaatttcttttgataatcggattaaaagtatcacggaggatatagagaggatgacacatgcatttgttaaaactcacatgcctaattttttaaaacataaattaggcgatgagaacgattactctagatttcnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnngctgagcaatattgccaagagcggtaggaccggtcgtccaaagagaataaagtttatgactatgttcagaaataaagaaaggatcatataaacaagcgcgattaattcacgataggagtcctcatttgttgcagagcatgggggcagtagacacgatgagggacgccgagtgataagaaaaaaggagataagccgctcaaattcgccaccccaatcggtttgcatagcaatgatttttctattgagcaagcgctcaacaaggctttgaaattctttgaagaactgaaacacctcagacttatggcgaagaagatagatccaagtaaatttactataatcatcaatgaagctgacataataccttttattacaaaaagaatcaatggcgggtccccagacatcgaaaaacaccagatctaaaggagcagcagactgactggtcgacttaggataaggcaactgatgggccttagcacgaaggcaggcatcacaaacatactccgaggaatctaagcctgaacacactaaattattatttctaatgacacgagcgacaatatcacgcgatggatgacctaatctgcaatgccaacgctcataggatggctttattgcggcaaggtcgtgcttctgggtaggtgtgcaagagatgtcaatgggtagaggccacccctacatggtccgcgcaccagcacttgcctcgtggcctgatccttaatcaagaaaaagaacggatggaactcaataaaggtgttattatcaagattgaaacgatgaatggaaacaagatttttatgggtatgagggacacgaaggacatgatttaggtgcagagggcggaaggaagtgggcaaaacagaataaccaatgtgagtaatctccatacctgcaccattagccgcccgaatctgatcattgccattgtaacgatcatgctgttagcttttccagctcgtcggtgatgtgatcagtcacaccgaagtcaaggtaccagtttggatcagcagcagtggaggatgatgccatggccgcaacccgatcatcaggagtgaattcttcataaaagcggtaccaacagatattagctctgtgaccgactttaaggtagacctagcagcgtggacaagactggccaccggattgatctgtcggtggaccggaactgcgcctgaagtagttgttgttgctgtagttgccgcgcgaagacgacgaaggatagccatggccatttccgcgcgagcgtccgcgaccgtgattttggagaaccatcatgccaggagccaccacggccacgagtagccgtattggctgatccatgagcagcgtacctgccgccggactgcttcgcaagccgaagctcatagctgagcatctgcgagtataacttggcagaggagattggctcgacgcaagtgacgatggacgacacaagcgggttgtagatttcttcatcaaggtcggtgaggacataggcgacgaactcctcatcgcccagaggttggccggacgccgacatctcatcggcataactcttcatcttggattagaatccggccattgtggtcgtgcctttcttcgtggtggcgagcgcaatgcgcgtgttgacagaacgcgcacgtgtgcaagatccgtacatagccgcgagggagctccagacgtcggccgaggtcgtggctgtcgtgacacccatcaagacctcacgcatcagagaggagaggatatatcccagcagcgcttgatcgtgagtcacccagttgatgtactcgggattgggcgtctccatataggcgtcgttagtcatcacagagacagtcttaaccagcatcttttctttgccgatgagcagaccgtagagctgtgcagattggatgggcggtaggatttgggcactccataggcggtagttggttttggtgagtttttcggtgaccgggatcgagaaggaggaggggatggtggtggaatttgagaatctactcgccatgatggatgtgttgtagaggacctggctatggtaccatgtagattggaatggttgatgtggcagaaccgcccggattattccagtttaagtgcccaagtcacgccttaaaggccgcaatgcacttaaatcggaataagccatcagtccctcagatctagtctaataaagccacttatccaggatcaaataccacaagctcactcgaaggtgagtcacagaagaaatacaataaaacaggaaaacctcaaattaaagtactggagttattacataaatcagagtttttcaagtagctgagaaaagttcacaaaataaactgcagcggataatcgatgtcgtcaaaagcgaggaatagggcaaggcctggcccactacttctcctgctcctctcctgccggagcagcatcccactcgaccgtccaacccggtgacagggttgtaggccaagttacaccgtcaaccatatcctagagcgtacctgcaaaaattatgccacaagcaaggctaagtatactaatactcagctagacttacccggtgtgaggaatctactcttctacctctagaccatgtagctgtttggttgaggggtttggtttgccaaaagcactagttgtatctaaggtcaactttatcttttccatttctagtatcattattgtagctaagtttgctctttctaagcatacatggtaacaatcatttaatacaatcaacaagttatctcatgtaatcctcatttcacttcttactcaatgtagtacaaggggtcaagcagtctcattagctgcgagaagcagacgattcaaatcgagtattaaccttgcaaggtaaacctaaacacacgacatgtcagggcactccgtccccatcgattcccctttcgcggccagggctcaccgccttggcatacaatgctccactgacccgggctgccgccgtgcagtgaccgcacttgtacccaccaaagctagcataggagacccagtctcaggacgagtgaggagaaaagtccgcgcccagcttcaatcaggtactaggtttaccggttaccatatttcccgacatgtgtttagtacgttcaaacgcttgactcaggtatccacacattaatccttaattcattttcctgtctcatggacaaggcatccaccctggatccaagaccatagaccatcatagatcccattatcaagatgaatacaatcaattcctgacctcgcgcgattgctagaaaaatcactcgacttctaccgagatcctaattagtaaagcagctactcgacctagcatactagtatccatctcaaaaaggaatcctgagttcatgcaactaagggtttcaagcaactcctacacttaagtgcacattacaagcctacaaacactaagtgtagtaaagtagcatatataaattggttatgcataaaaccggggcttgcctccaaatgatggggctgcggggagatcctcgatggcagtctcgggagcttgctcctggtcttcctcgtggacagctccttgctcagggatgagcacgtactctccatcagcgaggttgcaatctaatgaatgcaatgagtaagatatatgcatggcatgatatttaatttagcaattaaaatttgatggaggatgatcaatttaatagggtagacctcattctcactactggagatttttggtggtacactcaccaacttagggtcaagttgattactgaatggttaacccatttttagtgttctactgattttcttctttatatcttatggatattttaacaagattcttagctgccatgttggggtaatacttattaatctttctaattcctcccttctttattccttttatgcttttaaggtgggtttgaactacaagatagcttaataaatttccagaaattctgcaaacattacagtagcttcttactggtgtataattttctgtctcaaaatttggggcttaaaaagtgaggggttctctctgtacaaaattagcaagtgttagggcaaaggggatgttttgaactacaactctcttttaacagtgggttattctttaagacttatttttgctggcatttagatgttataacatgattttgtacaaattttcagccactaatatttattagttattttattatgattttctaaagtttctagccaaaggggtgctttctactaccactatacttgaaaaatatcaaacaacagatttccaatttttcctatcttcttctttgcgcaagagcaatcattctaaaatttggtaacctttttcttaagggaagggtggtaggaatttcttgaattaaatggcctttttcatgaagtaggggcaatgggtattactttgtagtttgaataggttttgcattttgctctggtgatctattccattaataatctagtaaaaatttattcgcccattgttgcacactttttggcttgcttatgatttaattggaatatggctcaatatcaagttttatttgttcaacccacttaaaatgatgggctaggtatttatcatttttgtagtggtgtcctagtggttacaagtctactgaatttttcttaccaattttgaaattgttctcatatttctaataattgcccttctagctttattagtgcctaataaaacatttcaccttgaatttgctctggactagtgttccttttattttttctaggttcttcattacttaagtgggctaggaaaaatatttgcatccactgttcattattttctagtacctttcttattttcctaagttttggacaattatggcttttaatagataaccctgtttaaatcttcaatactagggtgctcaatatttttaaacagtgtctaagtggggtttgaacttctacaaattttcttaagttcagcacagaagcataactaattttcttcattttaataaggtttggtcagtttctttaattaattctaaactccaaaatttaaaacagaaagcacagggttcaatatttttatgtgatagttcataatattttgaatctagtaaaattggtttgactaaatttggttgaatatttctcaagatacaaatttcctaagtcctttactgaatttaaaaagaataaacagaaatggataaaggaaaaagggttttgcactggggtccctggcgaaaggttttaagtgtattacagacaggtccttggttcactatttatctgagtctatgactctgcagaaaacccctagggttttgcgaaatcgaacccgcgatccttcccctaatggaatagtgaccgcagtggaagaaaagggcggaggggcttaccggcggcgaggttgctccggtgaggggtcgggtgaggtccggggtctctggcgatcacgtcgaggtgcggatcgtcggcggtggtggtcggagtaggttggtccacgtgcacaggcggggagctcgtcggcggcgagggatccggcctgctcacggcgcgatagtccaattgaacaggttagggagcttcaccagaggtcaaggaagacatgcgcgcgaggaatttgagaatgaatcaccggattgctcggtctacgcgcggctgcgggtgaccgaagtccagcgaggtcgatcctgggtctctggtgaaactctgttgggtccgaggacttggaaagcttcacgggccactggcgaagctaaccgagtgactggtgcagcttggaagtggctggagggagctggccgcggtggccgaggctcgggcggtgatggcgggcgggggagagctcgcggagttggagttcttgctcgaggcgtgaggcggagtgaagggcagaccattgtgcatccagggtacttataggcgccctcaggcatggctgagtgcaggcgcgggggacagaagccgaccgtgcatggcgcgcgatcagagggcagccagtgcgcggccaagcgcttgagcacgcgatcgaacacgtggaagtgtgattctgcccgagttcaaacgcctgttggccgaccaaaacgtgcatatcttgccaaggatcctgtgtagcgtctcttcaccgtgccaaggtcttcctgtcgtgtgtgagtcccgagtgaagatatggcctaggtgagaagatatgatggcctgaagatagctctgttagcactgtccaaaccgagacaaaacttatgtcaagtcgtgtcaaacgattcgggtttgatctcaaacttctccaaagtgttcctagggtattttggcgccactttgatatttggactttgtggattcgagttttggaaaacagggaacacatctgaactttgggaaagggtttgaaattcagttttctgaatttctgaatttccccatagggcattggttcatgggctgatttgggattttggaaaattcaaatggcaaaactttcttactatattttgttggttatttagtgcactaaaactttgttatttggttcttaccaaaattttgtattttcccaagtcttttcccaaattccctttatgtgcttaaatggtccacttaggattaattagggtttgagagttcttcttaccttgaggtgcatggcatgattaaggagaatttcttaagatgaaaaagactcacttaaaccttgttcttaatttttttatgttcattcctctttttggttcacatgtgataatggttggagtcaactctaggaaaaaccctacgtgacactggggtgtcacagttgaagcgttctaccacactaggtggccaaggattgcatgtttatataggcacaaggctgggtgcaacaacttatacaataaggtaaccgaatcaatctattgttggagtttctatctatgcacagcctagaatatatcctttctatctataggagattgattcggttggctaaagattacatgcacaagaaacttctagaatatcgtaacttcatctaacagttacaactcatgaacacaatataatattctgctatagaaatcatgattgtgtaattgtttgttgcaatatgttatatttgatttatggttgatctgttttatatcagctagggggttgagctagattatggaaatgtcaccagcaggatcacaatcaacactgatcatggtctctcaagttacaacaagcaatatgcaagggactcttcaaaaaagtgatgccttaactaccagcttcagaggctagccatgcttcgagaataccaacaacaaaatgttgatgaaaatcactgaaccaacagtgacaccacaaagcaggaatgccaggaccacttctaaggtatattctaactcacatttgacagtaatttgtgaaatcactcaaacaacagaatacagttcgcatgtttgactaccaatttgattttttgtacactcatattttattcttaaatctgtggaagatgatatgaatctgcacatcatgagtgcagtttctgcaagttgctttgcgaggtcaacagaaacacagaaaactgatggtgatgcccttatacctaaggtaaatttttcttctaactgaagcctcttttcgccttggaactcattcctttagctaatactaagagatgatggaaattctctcattccaatgtcaccagcagtatgatgctaatttctgtcaaatgttcttgccatattaatcttagcatttcattgaatttacatagtacttgaaaataaaataacatgagacaccatgtctaaaatataatggtaatctatgtgcttgatcgcgggttgctacagatctttgatgctagtgtgaacctggggtggttctataaccgggacacagaagagtggtataaaaaaggtaacctttgtaacgcaaaaatctacttatttgtttccataatacatatgagatcttatcctattgttgattgcaatctactgataggacttacccacccttcccctgccaaaaaagggcaaagaaactcttccaagattgtgactttgaagatgttgatggtgatgcctctgccaaagatgaggctgagctagggtactcagcctatctatttctcaatttcatcatatttataattgtcaatgcaattggagatgataaaaatgctctattttacataaaaacactgatcttgatttggattgtttgctaaattgtctctttatttgatggtcttggctatacttgtctctggtagatttttgcatcacagggtgagcgatgcttagccaccaagaaagaaaaaaataccactacctctctggtttccttttgtattggatatttatgtctcttgtctttgtttttgctccaaagtcttatacattatcgttgactgcattttagtccttctcccaaaaattcacttgttagtggcgaggatatcataataattgttggggacttgttctcaaatgctatgagttaagaacaaggcaacacaaaatgttaaatgttaatgtccttcgtccttcgaagcattatttcccttaggagataacgatcttcggacgaaggttatgaaggacataccttcataagtatgacatgtataaacaaaggatgaagcttatgaaacataggaagacaacataaacaattatataacatcttaacataaatatttattattaaataatcataagaacataagaataatatcaaattacatttataccttgagcttgatagaaggcaaagataaaagtaagatgcgaaagcgtgaacagtacgagggtactgttcacctatttataggcacagggcgcagcctgtgtaaatttacattcatgtcctctacaaatgattacaatcataacatagattatcatgggcccaattcgtcatttcatctttaagtcggtgcatctggaaatacgctacgaagctctctgattggtagcttcggcatcattcctgttctggccttccgaaggtgttttttctcacaggaccttcggcgacgaaacagacccccaacagtagccccttcacggtgccagatcattttttgtaacgagctcgacccgtgaaaaattcttttaggcttcggaatgccgaaggtccgaaaaacaccttccctgagctcgttgtcgagaaacgatttaagtattcctagtgcgaggtggtcccaccataggacgggtacgcacgatctggtgattctccttctcgcgccatgcggtccaccgttcagtgaatgcgagcgactgttcggcgggtgcaggtggcttgatgattcaccttcccacctgtagcactatataaacagacgggtaggtgtgaagttaccacagcattcattactatcgtattgttgtgctgctgaaaaatttgaccatagccgaagcttattcttcgtattctcaattagagcatcgtcttgttctttagcttcgtcaaaagagggagcttcggcaaaatcaaaaagtaatcaactttgtcaaaaccgcgagaaattcagcatcaaatggccagggtgcgttcaactgctagagtcacacgcgacggggaggaggccgaagctgccgagaccgccccaatctccgaagtaatgagacaatcaggcttggttgtgctagagggtgtttctgacgaaggtgcacgtgctgccgaaaccgagcaggctgacattgaagaaggtgaggctgatgaagaggagatagattatttcgtcatgccatctaaacccagccacttggaatttggaaagtctaccgtctctgaggccgatatgcccatgatgacgaagctaggctacttcggggaagccgagaagaagctaattcgttttggcggagaataaatcactccgaagctagaaaatgatgaggtggtagttttcagaagtttctttaaagcaggactgaggtttcctctgcatgggatgattgtggatgttttggaaaatttcgaaatttattttcatcagctgactcctaacgctatcgttaggcttagcgtctttatctgggctcttcgaagccaaggagtggagccgcttgccgaagccttctaccgggtgcacgaacttcactatcagacgaaggctagagaagatggactgcacgagaacttcggctgctataattttgcctaccgcaaagacatgaagacaccgttggttagctaccgcaccaaatggacaaccggttggaaaactgaatggttttatgttaaggttgatgagaagaaggagaagctagtttagagcccactgggcctaaccttcgggttaactaggccccagtgtcgcatgacgctgggatcatcatgcccagatgttgtgggtgaatttagagttgtgtccgagcatatcggaactagggatttggttcaggaatacttagccaatagagtattcccaacgttaaaggaatggagtatgccgaagcttaaaggagagaagaaaaagaatgaacttgttcgactgccctatcattttaagttcaagaaacacttcaaagaaccctgccaagaatggttggatacgatcgaagttatgtgcaatgaaatattgggcaattatacgaagaaagaagatcaattgatgacggcagccttcggcacccgaccgaaacgaaggctaaaccgagtaatgaacactctgaaatttgaatacccagactatgaacggttaagtaaaggtgccgaagggccaaaacaaaaaagagctgtcagtgttatgcaaagacaagctgccagaatgataaaagaagatgaaaatttagcaaaaaagaaaaaaaatccagccctgagccgaaggtggccgtttcgaagaaaagaaaagctacagctccgaagccaaaagctgatttagaagaagttccctcaacaccttctgccactgacgcagaagaaattttaaaggtaatgaccgaatctctacctaataagctaagcccgctgggaccggaactgatgaagcttttacagaagaagaagaaggaaccttcggttgccgagaagcccgctgaaccaaaaaagcgaaggattattactatcattgaggctattgaagaaacaccatcgtcggcctcagtgctaaaaacagcagcagccaaagctgctccagccgaagcttctacttccgaagttgcagcagccgaagccacaaatttggaaaacacgcttactgacattgatgaaataattttgaatatggctgaggaagaaactgctgcagctgctgaggaaaccccggctacagtgcctgaaaaggagaaggagcttgccgaagatgcttcggaagaaagaaatatcaactttcaaaacataattggacaagagttgtctaaggctaaaaaagaagagctgagggactttgctatatcttgcgggtaccagccaggggcactgctcttcggtggtatagacgaagagagcttaggttgcctttgagaccggactggggagaaagttgtcaggactttatcgaaaagtgttggttttccgaaactcgaagccgatctcagcagataccgacgacagcatatcgtcggtagcctattttattctaactttaaggtaaaattcttcccttaactttttattgttttgatatgaagatgttttctgatgaaggttattttgtcagagcctactactaagcaaaaccttgaggatgcaacaagacctcgaggacaagaaaaacgaagttataattgagggcttagagaacaagattaaagatcatgaagctgccctagaaaagaaagacttcataattcaaacaatggaaggttcactggcagaagctcaagccgagatcgccagactgaatagtgaactttccatgaagtcaaaaagcattgagcaagagaagaaagatttcgaaacaaaactcgaagctgaagttgaaaaaagttcaaatctgcagaaatcactcaaagatcttcaagaagcatggtcttgtacttgtttggtgacttgtgcccgcttgatttctgctgagagccgaggcaagggctgagcgcttggtcacgtacccgagcccccctgacaagggggttgcccatgccgtagtggttgacacagtactgagtatggcaaaaagtccctaagtaatatgtcagctctgcagtatatggtgacgttgggcgcctttccgttgtggatattgaggctagagtcgggctcgggcgaggcagaagtccgcccgaggtcacgaccgagcccgctccagtattcgcggggagcaggtaaacgaggccgggctcaggcgaggcgaagtttgtcccgaggccgaggtcgccttcagcgaggcagagttcacgtccgagagccatcctgcactcttgtcgtattgtacgtcccatcaggggttgacagatggcatgtgggaatagtggtcgcatgcgtcatcgtagttggtgaagcttgacaggaccgcggtcttgttgctcctgttcacctgcaactctacgtggggtaggtatgcatattgaatgctcctgccccctgcagactttggttgagtcttgcattggggttgtcttccttacccgagatgtgctcgggcgaggcaaagacttttgttctgggagatggagcctcggccgggacgagaattctccctagagcacaccatgtccgagggcaggcttgagcgaagcggacctatggtgacccctgagcggggcctcgggcgaagcgcggtttatgatcctttgatctcggggaatgtgtcttgaaggtggtctaagggttaagtgtgttttaggggcataatctgggtacccctaattatgatacccgacaagtggtattgattagaaatggctcaacaaaagataatggatggttgaacaaaatgtgaatggctgacatcagttttatagtgtatgtgtgtatatatgtgtgcacacatacaatatctctcctttatataacataaacagacataagttatagtggtagaagacgctcgcttgtatcgaaagagcatggtttgaatccccacgtcctattttttgtgtggttattccacgcgcctggctggctggttcgtgactaggtcggacccatgcaactggctagcccaaatttccccaattatttcataaccaacctctcatttgttctcctttatctttatgttattaggatcaatcatttgtagttatcaaggtgaatcacttgtacttttatcaaggtcaatcattatagttactaggatcagtcgtgtatttatcagggtcattcattgtaattattagggtcattttattttttaccagggccagtcattgtattttatcaggatcagtcattgtacttcttctattagggtctacattttatcaaggtcagttattgtagttatcaggatcaatcattatattttaatcagtgtcagtcaatgtatttattaaggtcaatcattgtattattaggatcagtcattgtatttatatcagagtcactcattatagttatcaaggtcggtcattgtatttttttattagggtcagtcattgtatttagcaggatatttttatcagggttagttattgtattattaggttcaatcattgtattttatcagggtcactcattatagctatcaagataagtcattgtattttttattagagccagtcatcgtatttattaggaccaatcattgtatttattagggtcggacattgcgattaaataaaaaattgaaaaagatatagcatgagtgtctagttttgttcgaaaatctcataaacacgaatataacaaaaaaagggattttggttttttatgcctatatatgcgggttgcatgactgcatacacgcatactcgctgagcgtggtgccaaatagtatccactgcgtgccctgcgctctaaccggatgctctatccatcacacctcaataacccattgagcatccctccccccacacgcctgtgctccaatcagatgcttgtttgactaatagcaaggagattctccaatatcatgctaagaatagctaggatttccagaagaagatgtcattcgtttgatgagaaataaaaaggaatatcgagaattcgcgtggctaaagctgaagcaactactttcgaagtaacagaaagaaaagcaacgattggagtgggggagtcagagtcaaaaagagaattcctcgcttctttctctcatgcaaaaccgtgcatgagactttcatctcgcacggcttctaagtgataaaagaaagaagtccaatcgtgataaaaataattacatcaatttaatagaaaggaatgacttaaaaacatattatgagtctctggatgaataaactattggatgacttaaaatatttgtaagaaagtcttgtaacaactgttgacaatatgaaatattttaaataagtcataaaatgactaaatgacatgtgatgactagaattgtaacagaatgacttaatttaacataatatgtactgaatgacctaacgagtgaatgactgagaaaaaaatagaatgttttaaataatcatcaaaatgtcttaaatgattaagaaatacttgattatcttataaaataactagtacaacacatgtgcgctgcgacgacatacaatcatatttgataccaataaaaaaataatatcaaatatcaaagtgaacatatggtccatatatcagatactaaactgataaaaacaaatattacgcttttatcttagctaaaatatcaggaaaggtatgagttgaaagaagcctgactacttttttaaagcttgctcgatggcttgtcctcctttaggtagtgaggtggttctatgtgggagcgctgcgctgcgtttggcttccctgtcgtgttagacttgtgtggtttctcacggtccatctatagataaaatgtccactagtagggatttgggtggttttcacagcctatctatagatgcccactggtatgcggattgatctacatgcttcgtgcatggcgtatgacgaccatcgaagctagtattttatagtagtggagattggaatgaattaatgcaaaatgagaagtatgagaatgttgagtgacttaaatggatcacgatagaaactgcattggggcctgaaacagctactaaacaagcgatcgcaatatcttttaaaaataagttgcggtccaaaaaaaagtgacaatctatactctctaagcaggctcccaaccatgtcaattcactacaacaatttcaatgaattaacatgagtgaaccatagttctcacagggtatttcgtcgttacaggtccattcgattagaagtgggtcattatatggtggtttgcactgtatctttcccccgttatcaatgagagccaaacgtgtaccttacaacctttcagatgtcaattggaacttgcaaaaaaaaatagaaagaattttgacttgttggggatttaaactagaaagcatctaggcccctggttggttttagtgattaatgacaacgtaattttatatgtgactaacatgtgttttgcagaggcaaatggtaagttaggtcgcattacaggtagatgtactacaatggtgaaaacaatcccggagataaaaacttgaagcaacggctaaagcgacgaaacaaaaagtgaaggtcttcgtattccgagtgtcaaggagttgcggacactcgtgatatagttaggtcttttattttgttttagccgtactataaagaggggttgtcgataagtagtttgaccaaaagagttctagtgtagtgttggtgcatattcacactcacatatagtgctaggtgtaactctagaacatactcacaagttagaacaaaaaccaaattgaaaaaacagcacaaaacagaagctagggtttctggctttggggcaccggactgtccggtgcaccctttgccagtgggcccagcctggcccagggaagagggttccctgcgcgcagaaacccgagagcgcgctgttcgtgagttgaattttagaggcacaccggacagcgtatcggactgtccggtatgccatctgtccaacggctagctgtcagaactagccgtttgagtcgaccgttggcgcaccggtggcacaccggactgtccggtgcgcccatgcgcagcagattcctggtaatggctagttggtgggtgagggctatttataccccatccacctaccatattgatggtcttgctgcccacatttactcctacacattggtagagcattgcaagcaccacaaagcctagtgaggtgacttgagaatcttaatcccgcatttggacctcattaacgctagcgagagccacctagagcacacaccgcatgcattaggcttctcttggtcaagtgaaagtctatggcttattactcttggtgatcggcatcacctagacggcttggtggcgttgggagctcggtgatcaccgtggagatcttgttggtgacccgactcaagtttgtaagcggtcgtgagggatccaccgcgccggagtggcaaaggatcatctcgttgtgagcacttggttcttgcgatgaccaagggagagcgatacccttacgcaggtgctccaacgaggactaggggagagtgccgactctttgatacctctagaaaaattggaggagtcttctaaaccttgctttacattccgcacttaattcaagtattttacattgtgtatttgtttagcaagtatttgaagtattatcttagcattgttgtatttctagtattattctcttagtgctagttgtcggggtgaagttgggctcttgcttagattttagttagtgttgatttttagaaaagcccaattcaaccccccctcttgggcatcgtgatcctttcaattggtataagagccttgttgctcttagattagcttaaccgctagagtaacgatgtccggtggggatggaccttctcccgttttttatggtgacgattttccatattggaaaattcgtatggaagcatatttagaggctatagacattggtgtctacaaagccgccacacaaagattccccgaacctagagatcccacaaatcttgtaggtgaagagttgaactatgagaaatggaatgctaaggccaaaaacaccctttttagaggcctttgcaaagatgtgtttaatagagttagaaaccataaaaattgtcatgatttgtggatggacatatgtgctctacatgaaggaactagaattgagcgtgaggagagatatcacattgctatgagaaaattaaattcttttgaaatgcttgctaatgaaaatgccaatgctatgtactcacgtctcaatattcttgtagaggaagtaaatggcttggggcttacacaaatttcacaaccggatgttgtgaggaagattctcagtgtcctcccaattgataaatatggacacattgtcactgtgctgcatcagatggatctttcagttgtcactcctacacaaattttgggaaagatcaatgcacatgagatgtacatgcacatcaatgacaaggatgagtcatcttacaagagaaaggatttggctctcaaagaaaatcaagaaagagaaggaaaagctaaagtacaagttgaggaggaatcctcaagtgacgatgatcttaatgctaacattgccttgatggtgaggaagaccaccaagatattaaagaagctcaacagagaaggcatcaaatttgactcaagaaagaagaaattcttttccagcaaaagaaagcccatttcttaaatggattgctacaactgtggagagcttggtcatcttgctcatcaatgtaacaagtccaagaagaacaagttcaagggcaagaaagaagatgacagtgatgatgagaaaaatgaaaagagattcttcaagaggaaggatggaaagcataagaggttccacaaaaagaaaaatgtaaaggcatacattgttggtgattggctcactgacattgagtcgtcaagtggatcttcttcaagtgaagaagaaaatgatgaaaaagttaccgccatcgctggggacttctcttcaccaccaccatctccatcatcgacttctcacctatgcctcatggctagaggtgaacgaaaagtacaaaatgataatgatattattgatgatagtgatagtgatagtgatgaagaatttgcttcaccttcctatgatgaactagttgacttgcttaatgaatacactcaactcattaggaagtcaaaagctaaatgtgataagttgaaagatgaaaatgaatttttaaatgctaaatatgacatagttatgaaagctagtaatgaaatgaaagaagaaaacaaaactatgtcatccactgtaaatgagcttacatcctccctaaaagatgctaaggataaatgtgacaagttaaatgaagctaatagggagttgaaagatagactagtaaaaaataaggaagactatactaagattaaatttgatcatgataatcttcttgttgaaaatgaacttttatcttgcaatacacatgaggctattaaccctgttgttaatattgatgtagcaacctcatgtgatgatttgagtcaaggtgatcaaactagtctacatgatgaattgactgaaaaagttgaagtcttgacattagacaaccaaaaattgaagagatacttgactgatgcaactactagaggaaaggttgccattgagaacaatgacttcaacaatgagttggcagtggataaagaaaggcttaaaatgaggtcaagaaacttaagcgtgaaaatgaacatcttgcaacaagtgtgcaaaagttcaacaagggccaatacctctaaaatgaattgctcatgaacactgtcatgaaaaacaacaagagtggtattggatataactcctttgtgcaaaagaaagctacaactcaatacaagccaaatcagactcataagcatatcaaatgctttgagtgtggaaaagaaggtcatttttcccacaactgcaaagccaaaccaccaactcccctgccaaagcactcaagaccatttgccttcaatgctcattatgttttaagaagtagcaaatggaaaagtcgaagttacattcctaggtccaccaagcaagagtagacctagacaaatttgggttgcaaagtccttgattgagaaagtcactggtcctatgcaatatagggccctcaaaacttaggcttgatttgtctgtggatgtaggtgaactacaagaccggtgggagccattgggttattgatagtggatgcacataacatatgataggcaacccacggatgttcacctcacttgatgataatgttgatggacaagacaaaatcacatttggggacaattcaaagggaaaagttcaaggacttggcaaggtggcaatttcaaatgatctatcaatttcaaatgttctcttggttgcacctttaagattcaacttattatcagtgggtcaactctgtgttcttggacttcaatgcttattcactccaacagaggttattgtatcaaaaatggatgatgaataaatggtgctcaaaggatttagatacaacaatctctacttagtggatttcacctctgaagatgcagacttaagaacttgcctctttaccaaagcatctcttggatgactatggcatagaaggcttgcacatgttggaatgagcacactgaagaaagtattaaagaaggacatggttagaggactaaaggatgttatatttgaaaaggacaagccttgtagtgcttatcaagctggaaagcaagttgctaacacacatcctacaaaagctttcatgtcaacatcaaggccactggaactacttcacatggatctatttggaccaacaacttatgcaagtgctggtggcaacctctactgtctggtgatagttgatgatttctcaagatacacttgggtgtttttctccatgataaatctgaagttgcatctatattcaagaagtttgccaagaaagctcaaaatgaatttgattacaagatcaagaagattagaagtgataatggaaaagaatttgacaacaccaacattcatgaatactgtgatgagattgggatcaagcatgaagtatcagcaacatatacacctcaacaaaatggagttgttgaaaggaaaaataggaccttgatcacacttgcaaggacaatgattgatgagtataacacaccggagaggttttgggccgaagctatcaacactgcatgttatgcatcaaacaggctatttcctcactggctacttgcgaagactctctatgaactgctaaatgggaaaaagccagacgtctcattcttttgggtgtttggatgcaaatgctacatttacaagaaacgccatcacctagggaagtttcaaagacgttgtgatattggttttcttctgggttattcattaaagtccaaagcatatcgagtattcaatcatgccactggcgtggtagaataaacatatgatgtggagtttgatgagactaatggctcccaaggagcacttgaaaatcttgatgatgtaggtgatgagccacttaaggaagccatgaagaacatgccaattggagctatcaaaccaaaagaagatgaagaagaggtgcaaaacattaataggccttcttcatcaagtgtaccacaagatgatgaaaaagatgagaggcatgcaaatgaagatacatttgtctctcatgaacaagcaaggatacaagccgaagatgttgatgctccaggatcttcttcctaagtggttgataggagaaactcatcactgcttcaagcacacccacaagatcaaatcattggaagtccttcacaaggggttattactcgatcacataaacatgcttcttttattgaacatcactcctttgtttcttgtgttgagcctactgtatagatgaggcgctacaggatccggactgggtgaatgccatgcatgaacaactaaacaacttcacccgtaaccaagtttggaccctggagaagcctccacaagatgcaaggatcattggaacaaagtggttattcagaaacaaacaagatgatcaaggcgtgattgtgaggaacaaggcaagacttgttgcaaagggcttctctcaagttgaaggtttagattttggagagacctttgcaccggttgctcgacttgaagccatctgtatcctactcgcatatgcatcatgctatgataaaaagctttatcaaatggatgtaaaaagtgcatttttaaatggcttcataaatgaacttgtatatgttgagcaaccacccgggtttgaagaccctagatatcctaaccatgcttataggttgtccaaggcgctatatgggttaaagcaagctccaagggcttggtatgagcgtcttcgcgacttcctcatcaaaaagggcttcaagatcaagaccgtcgacacaactctattcacaaagaaacataacggtgatattttcatttgtcaagtatatgttgatgacataatctttggctcgataaatcgctatcattgcaaggaatttggtgagttgatgtcgaaggagttcgagatgtcaatgattggtgagctgatgtatttcctcggctttcaagtgaagcaaatgaaagatggtaacttcctctcacaagagaagtataccaaagacttgttgaaaaggttcaacatggagatcacttgttgaaaagatggtaactctctaccgttctatgattggtagtttattgtatcttattgcatctaggcccgatatcatgtttagtgtatgcatgtgtgctagatttcaatcaaatcctaagaaagctcatatttgcgctcttaaaagaattcttaggtatctcaagcacaccccaagtgttggcctttggtatcccaaaggagctacttttgatttaattggctattccgattcggattatgccggttgcaaaattgatagaaaaagtacttctaggggtgtaatttgcttgggagatcactactattatggacatccaaaaagaaaaatagtgttgccttgtcaaccgccgaagcggaatacattgccgctggtgcttgttgcacacagattttatatatgaaacaaactcttctagactatggtgtagttctagaaaaggtacctttgttgtgtgacaatgagagtgctgttaaaattgctaataatcttgtacaacactctcgcaccaagcacattgatattcgtcatcacttccttagagatcatattgctaaaggagacattattttagaagaagtgaggtcggaagatcaattagaggatattttcactaagcctcttgataaaacccgcttttgcatgttgagaaatgaattaaacatacttgatctcagaaattttatttaaagatctcaaaatagtgttgtcaagcctgcattgcatatttaaatttcttgtattgcatctagggcttgtctaacctagttaagataaccgccaacaaagcgagtgaaaaaagcttaactcgggctcaaacttgacaagtcttagctttaagcttttagtacttaaattcttatttactatgccattgttggttcttgagatatgcatgtagtactacacttagggggggagtattcaaaactcaaattattcatgaaaacccctagttcaaagctaaaatgcaaatctcaccatttgactattttctctaaaaattgactagcctatggcaaaatatttttgaaaattatgggaaaatatatgagggggccaatacctatcccaataggtgttcttttgtatgattataagttgggatttggtttggttaaaatttagatcgaaaaatttgaaaattttcaaaatcacctctgcctaggctcaccggaaagtccggtgcactgtgcactgtnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnagctactcgacctagcatactagtatccatctcaaagggaatcctgagttcatgcaactagggtttcattcaactcctacacttaagtgcacggtacaagcctacaaacattaagtgcagtaaaatagcatatatataatggttatgcataaaaccggggcttgcctttaatttaacacttaggtagtgtttgctgggggaggtactcgcttggtgagcatccactggttaagtccattcttcaggtcgtccatcaacggcatcttgtggttggcaccacatcactggctcgatcatcatctctcggtcctatatgaggtgcaagatgcatatgtatgaatataataaaagtaacataagatataccaagacacagtggcgaactaaacattaattagtaagacactgcaacaactatacgcaaacactagttatttatgtgtcattgggcacacgtaaacactaccactggaaagacaatgatcactacctacaattaaccaacgcaacacgatatcatatgtacaagcatttatttagttgctacggcttttcattaattcttatattgatcacacaaaaacatcacaaacacaagtttaataaaaggaccgatgcatcaatgtcgatggactcctctatcacaatcaactacagcaagcaaacacattaattatggaacacatgttaacctaagtttagccatcacaagtctatgtccgttaagtgcttactaaagcgtttttagccaaaatggtgaactaaatattcatttgagcacgtgcagatttttaggacagcagcacagcagctacttgttttaatcataacttttaaaatattaatccaaaaatagcaaactaaaactttctggaaagtttagaaagtgctctacaattttggtattttcatcacagcatgattaaacacttagcaaggtcaaaaagtgcaatcacagcagctctgtccagatttggacagattcagacttgtgattttaaaaattcataactgaagattcagacatccaaacaaattgatcctagactttctggaaagctaattaaatgttctacaaattatttataaacatcccaggctggtttagcatgtatcaaggttaaaatatactatgaaggctgtgctgtccaaaactggacagattcagtcttcacacttcaaacacatgtaacttaatcttcagaccaccaaaaagagtgatctaagactttttgaaaagcttagcaaaagtactacacaacttttataatcaccaagaagtgattccaggtttaactaaatcaaatattacagttttcgaaatctgttctgacggtggacagaacacagcaaccagtttgtaaaattcataactcttaaaccgtcaggcctatagttatgaaattttaacacaagcaagacaagaaaagcctctacaactttcttataattgacaagggctgattctaacattaacttaagcaaacaatgcagcttttgaaatctgtacagaaagtggacagattcagttactgaatttgtaaaaaacataactcctaaacaatcagacttatgcctgtcaaattttaacacaagtacgataataaagttatctacaacttttttgtgaccaccaataactaatttcaacattaacttaagcaatcattgcaatttctgaaatatgttcagaaatttgacagattcaggtgctgggcttgtgaaaagcacaactcctaaacaatcaggtttatggctgtcaaattttagtacaagcaagataatcatgtcatctacaactcttctatatgacttttctatagaaaacatgatttggtttatcaaacaaacagcacaactaaaacagtgcgtgcagcccaaaacagcaatcaataaattcagcttctatttacttttaaaaattgccgcgttctagagactcgacttattctaaattatatcaaggcacgcttaagcatagccacgcaatagatgacgtgacggctacgtagtcatgccatcacttcaccccacaatcccaactatcaaaataactgtcggagaccataattaggggtaccctcaagactcctaattctcagctggtaacccccaccagcataaagctgcaaaggcctgataggtgcgattaagtcagggatcagtccattcgagcgactcgatcacgcctcgcccgagcctagcctcggacaagggcagccgaccccagaggatttccgtctcgcccgaggcccccctctaacggcggacacatcttcggctcgcccgaggccctgccttcgctaagaagcaaccctgactaaatcgccgcaccgaccgaccaagtcgcaggagcatttaacgcaaaggtggcctgacacctttatcctgacgcgcgccctccggcagagccgaagtgaccgccgtcacttcgccgctccactgaccggtctgacagaaggacagcgccgcctgcgccacttcgactgcagtgccacttgacagagtgatattgacaggaagccaggccctgccaaaggcgccataggaagctccgcccgacccagggctcggactcgggctcagccccggaagacggcgaactccgctccgcccgacccagggctcggactcgggctcagccccggaagacggcgaactccgctccgcccgacccagggctcggactcgggctaagacccggaagacggcgaactccgctctgcccgacccagggctcggactcgggctaagacccggaagacggcgaactccgctccgcccgacccagggctcggactcgggctaagacccggaagacgacgaactccgcttcgcccgaccccagggctcgggctcgagctcagccccagaagacgacgaattccgcttcacccgagcccagggctcggacaccgccctggacttttgccgacgaccttccgccttggcccgacccagtgggcttcggactcgaccctcggccatggaagatccactccacctcggcttcggaggagcctccacgtacccccagactagggcgcaggccagccacgtcaacaggaagcgccatcattaccctaccccgagctgactcggaccgtagagaacaagaccggtgtcccatctggctgtctccaccagataggcaatgatggcgccccgcatgccctgtgacgacggcagctctcagctctcttacggaagcaggaggacgtcggcaaggacacaaccgctccgacagctgtccctccgccaggctccgccgctcctccgacggccacgacatcacactagctgggttccaagatctctccggctgccacattggcatgtactcagggcactagctctccctcgctagacacgtagcactctgctacacccccattgtacacatggatcctctccttgcgtctataaaaggaaggaccagggccctcttagagagggttggccgcgcgggacgaggacgggacaggcgctctcttggggccgctcgcttccctcacccgcgtggacacttgtaaccccctactgcaagcgcacccgacctgggcgcgggacgaacacgaaggccgcgtgattcccacctctctcacgccggtctccggccgcctcgctcctttccccccttcacgcttgcccacgcgctcgacccatctgggctggggcacgcggcactcactcgtcggcctgagggaccccccggtctcgaaacgcctacagttggcgcgccaggtaggggcctgctgcgtgttgacgaacagcttcccgtcgagctccagatgggcagtctccaacaacctctccaacccgggacggtgctccgtttcgggagtcttgagttcatgtccctcgacggcagctacgacatgatactccttccaccgccgcgcgacaacgacgatggcggccgacagcccgcccgccggcggcggaatcgacgacgtcttccccgcgtggcggaagaacaacattcgagctcgccccgtcctctcccccgccaacggaggaggaggcggggcaacaaaggccaagcaggaggccgcgcctcgtcggctgtcgagcgagtcgacgtccctagcaccccaacggggggcgcgttgggcgtcgacctcgcgtttgagacaaaggcgagcgccgtctccccgcgacacgccaatcccgagcaagtggacgacgccagcgcgcttgcgaaaagcttgcaggacatcgccctcgtacctgaggcgacgatgcggtcagtcctcgacgtgacttcatcgccgctcgacgaccaaaaggtaccaaccgattcccatcctacgtcatttgtactcagcctcaacccgtctagcaatcttgctttggcgggcgcccttgtagaggcgagtacaaaccctctggggtttcgcttgcggtcgccttgggaccggctgacggacgtctcgacctacgggccctctgggtccgaggaagatgacgaccccaacatctgttgggatttctctggatttggcaaccctagtgccagcggaacttcatgaccgcatgtgactactgcctctccgactgttccgacggtagccgcagcctcgacgacgaggactgcggcccaagccgcgaatgtttccacgtcgatctagggggtccctccgaaggcaatcatctcggcatgccggaggacggtgctccccctgggccggtgcctcgcgctgacatcccgcgggagctagttgtggtccctgttccggcggggggttacgacccacagctcgagcaagtccgcggggcgcaggccaggatcgacgagggagcaggagcgcttgagccgatccgccgggacgtcgggcaggcatgggcgggccaacccccggccggagaaatacgtcacctgccccagggtctccagcaccgcgtcgccgatgtcgtcagggtcaggccaccacctgcatccagtggggtcggtcagaacctggtcgcagcagcgatgctcctccgcgcgatgccggagccatccaccaccgagggtcggcgaatctagggagagctcaaaaatctcctggaaggcgccacggtccgacgggccgagagcactgcctcccgaaggcaaggatacccctcggaacctcatgccgcgacttcccgattcatgcgggaagcctcggtctacaccgggcgcacgcgcaacaccgcgcctgcggccccgggccacctcggcaacgagcgccatcactgcgaccgtcgagcccacctcgacgagagggtgcgctgaggctatcaccccaggcgtgggggacgctacgacagcggggaggatcggagtccctcgcccgaaccacccggtccgcaggccttcagccgggccatccggcgggcaccgttcccgacccggttccgacccccgactactatcacaaagtactcgggggaaacgaggccggatttgtggctcgcggactaccgcctggcctgccaactgggtggaacagacgacgacaacctcatcatccgcaacctccccctgttcctctccgacaccgctcgcgcctggttggagcacctgcctccggggcagatctccaactgggatgacctggtccaagccttcgccggaaatttccagggcacgtatgtgcgccctgggaattcctgggacctccgaagctgctgacagcagccgggagagtctcttcgggactacatccggcgattctcgaagcagcgcaccgagctgcccaacatcaccgactcagatgtcatcggcgcgttccttgccggcaccacctgccgcgacctggtgagcaagttgggtcgcaagacccccaccagggcgagcgagctgatggacatcgccaccaagttcgcctctggccaggaggcggtcgaggctatcttccgaaaggacaagcagccccagggccgcccgtcggaagatgctcccgaggcgtctactccgtgcggcgccaagaagaaaggcaagaagaagtcgtaagcgaaacgcgacgccgccgacgcggaccttgtcgccgccgccgagtacaagaaccctcgaaagccccccggaggtgccaacctctttgacaagatgctcaaggagccgtgcccctatcatcaggggcccgtcaagcacacccttgaggagtgcgtcatgcttcggcgccacttccacagggccgggccacccgcggagggtggcagggcccgcgacgacgacaagaaggaagatcaccaagtaggagagttccacgaggtccgcgactgcttcatgatctacggcgggcatgtggcgaatgcctcggctcagcatcgcaagcaagagcgccgggaggtctgctcggtgaaggtggcggcgccagcctacctagactggtccgacaagcccatcaccttcgaccaagctgatcaccccgaccacgtgccgagcccggggaaatacccactcgtcgtcgaccctgtcatcggtgacgtcaggctcaccaaggtccttatggacgggggcagcagcctcaacatcatcaacgccgagaccctcgggctcctgcgcgtcgatctgtcctccgtccgagcaggcgctgcgcccttccacgggatcattcccgggaagcgcgtccagcccctcggacgactcgacctccctgtctgtttcggaacaccctccaacttcggaagggagactctgacgttcgaggtggtcgggttccgaggaacctaccacgcggtgctggggaggccatgctacgcgaagttcatggccgtccccaactacacctacctgaagctcaagatgccgggccccaacggggtcatcaccgtcggccccacgtacaaacacgcgttcgaatgcgacgtggagtgcgtggagtacgccgaggccctcgccgagtccgaggccctcatcgccgacctggagagcctctccaaagaggtgccagacgtgaagcgtcatgccggcaacttcgagccagtggagacggctaaggccgtccccctcgaccccagtggcgacgcctccaagcagatccggatcggttccgggctcgagcccaaataggaagcagtgctcgtcgactttctccgcgcgaacgccgacgtcttcgcgtggagtccctcagacatgcctagcataccgagggatgtcgccgagcactcgctggatattcgggccggagcccgaccggtcaagcagcctctgcgccgattcgacgaggagaagcgcagagcgataggcgaggagatccacaagctaatggcagccgggttcatcaaagaggtattccatcccgaatggctcgccaaccctgtgcttgtgagaaagaaaggggggaaatggcggatgtgtgtagactacactggtctcaacaaagcatgtccgaaggttccttaccctctgcctcgcatcgatcaaatcgtggattccactgctgggtgcgaaaccctgtctttcctcgatgcctactcagggtatcatcaaatcaggatgaaagagtccgaccagctcgcgacttctttcatcacgcccttcggcatgtactgctatgtcaccatgccgttcggtttgaggaatgcgggtgcgacgtaccagcggtgcatgaaccatgtgttcggcgaacacatcggtcgcacggtcgaggcctacgtcgatgacatcatagtcaagacaaggaaagcttccgacctcctctccgaccttgaagtgacattccggtgtctcaaggcaaaaggcgtcaagctcaatcccgagaagtgtgtcttcggggtgccccggggcatgctcttggggttcatcgtctccgagcggggcatcgaagccaacctggagaagatcgcagccatcaccagcatggggcccatcaaggacttaaaaggtgtacagagggtcatgggatgtctcgcggccctgagccgcttcatctcacgcctcggcgaaagaggcctgcctctgtaccgcctcttaaggaaggccgagtgcttcacttggacccctgaggccgaggaagctctcgtagacctgaaggcgctcctcaccaaggtgcctatcttggtgcccccagctgatggagaaaaagccctcttggtctacgtcgccgcgaccactcaggtggttagcgccgcgattgtggtcgagaggcaagaagaggggcatgcattgcccattcagaggctagtttacttcgtcagtgaggtactgtccgaaaccaagatccgctacccacaagttcagaagctgctgtatgcagtgatcctgacgaggcggaagttgcgacactactttgagtctcacccggtaactgtggtgtcatccttccccctgggggagatcatccagtgccgagaggcctcgggcaggattgcgaagtgggcggtggaaatcatgggcgagaccatctcgttcgcgcctcggaaggccatcaagtcccaggtcttggcggacttcgtagccgaatgggtcgacacccagctaccgacggctccgatccaaccggagctctggaccatgtttttcgacgggtcattgatgaagacaggagccggcgcgggcctactcttcgtctcacccctcgggaaacacctacgctatgtgctacgcctccatttcccggcgtcgaacaatgtggctgagtacgaagctctgaccaacggattgcgaatcgccatcgagctaggggtccgacgcctcgacgctcgcggcgactcgcagctcgtcatcgaccaagtcatgaagaactcccactatcgcgactcgaagatggaggcctattgcgatgaggttcggcgcctggaagacaagttctacgggctcgagcttaatcacatcgctcggcgctacaacgagactgcagacgagctggcaaaaatagcctcggggcgaacaacggttccccggacgtcttctcccgggatctgcattagccctccgtcaagatcgatgaccctcccgagcccgaggcgccctcggaccagcccgaggtacgctcggcacggcccgaggcaccctcagctcaacccgaggtaccctcggtctccgagggcgaggcatcgcgcatcgaggaggagcgaagtggggccatgcctgatcgaaattggcagaccccgtacctgcaatatctccgccaaggagagctacccctcgaccgagccgaggctcgacggatagcgcgacgcgccaagtcgttcgtcttgctgggcgatgagcaggagctctaccaccgcaatccctcgggcatcctccagcgatgcatctccatcgccgaaggtcaggaactcctgcaagagatacactcgggggcttgcggccatcacgcagcgcctcgagccctcgttgggaatgctttccggcaaggcttctactggccaacggcggtggctgacgccactagaattgtccgcacctgcgaagggtgtcaattctatgcaaagtagacccacctgcccgctcaggctctgcagacaatacccatcacctggcccttcgctgtgtggggtctggacctcgtcggccctttgcagaaggcgcccgggggctacacgcacctgctggtcgccatcgacaaattctccaagtgggtcgaggtccgacctctgaacagcatcaggtccgagcaggcggtgacgttcttcaccaacatcatccatcgcttcggggtcctgaactccatcatcaccgacaacggcacccagttcaccggcagaaaattcttggacttctgcgaggatcaccacatccgggtggactgggccgccgtagctcatcccatgtcgaatgggcaagtagagtgtgccagcggcatgattctacaagggctcaagcctcggatttacaacgacctcaacaagttcggcaagcgatggatgaaggaactcccctcggtggtctggagcctgaggacgacgccgagccgggccacgggttttcacgccgttcttcctggtctacggggctgaggccgtcttgcccactgacctagaatacggctccccgaggacgagggcctacgacgatcaaagcaaccaagctagccgagaagactcgctggaccagctggaagaggctcgggacaaggccttactacactcggcgcggtatcagcagtccctgcggcgctaccacgcccgaggggtccgaccccgagacctccaggtgggcgacctggtgcttcggctgcggcaagacgcccgagggaggcacaagctcacgcccccctgggaggggccattcgtcatcgccaaagttctgaagcccggaacgtacaagctggccaacagtcaaggcgaggtctacggcaacgcttggaacatccaacagctacgtcgcttctacccttaagatgttttcaggtcgttcatatacctcgcacccacgcaaagtttagtcatcaaggaagggtcggcctcgcctcggcaaagcccgaccctccctcgggggctaaaagggggggaaccccctctgcgtcgaaattttcctcgaaaaaaggtctcttctgccagaatatctttcgtgctttttgactacttcgaaaagtggatcctgaaaacgacggagtacacgtaagcagtcaaggcggaccgagccgagggactcctacgcctccgggatacggatacctcactcatcaccttctgcgataagtaactcgcgttcggataaagtgattccgcggaccgaacaagtcttcatgttcggaagttcttctgccgaagcaatccttcgagccttctcgactgagtcggtggcagggcctcatggacgagtgaaagtacgtgtaagcggcaaggccgaccgagccgagggacttccacgcctccgggatacggatacctcactcatcaccttccgcgagaagcaactcccactcacacaaacatccctgttaccgacaaaaaagtcaagatactcgaaacaagaggaaaggagacgcagctttacaacacagcgagggcgtgtattctggcctcggcggctgcagaaggcacacgctacaagacaatctgaccctacaggctcgggtcttgacgctggaagggggcagcaacaccctcggcatcgatgacaccttcagcgaggcccgacctagcctcggacggcgacgcggtccgaggatctccgctccgaaggacgatgtcatcaccacgcccgggcaatcgctgccagggacttctccgggaatccggcccgagcaggcggctcggccggttacccctggggcctcggccgaccatctcccaagggcgccagcccgacctgaggcctcggctgatcagccccgacgtcggtcccgccaacggacaacccggctaggctccgaccaaccaggtttcattttcgagccaactccgcctctgttcacactgatatcgctacccctggcctcggctcgtcgaagagcggccgaggggtccctttaactaagctagaggagcctcggacagcaaggccgaccgagccgagggactcctacgcctccgggatacggatacctcactcgtcaccttgacacggggcgactcatgcttggtgaagcggttcagataatcaacagacgagtcttagcgctcaaaaatgaggaaaaacacggctccgtgccggaattacatacatgttcaggccccgaaagccgcaatgaacaaaaacaccggcattcgaagtgccattacaaacggaactccggttcccccctccgcaggtacgaacagccccactcgataggggtgggcctacggagcaacagaagactgacgagcggctcgccgccgcccgctctgactacgacgacatgcaagcaactgcaccgccacttgcgccaccaccgcgcctcctcgattgcggaaccaataccgcgactcgaggcgacccagcgtgcgacccagcagcgccagcctgacgcggcggtcaacacggccaaaagtgggccggcagtaatgacggtggcaggcgcgtgggagcagcggtcacgtcgtcagccaagctcacgtcccatccgggggcagcaagagaaccccctctcacggcgtgaagacaacgcgcccgtgatccgttcctcgaacggctcgcgcacgcgcaacggctgccccgccaactactcgcctcgtcgcattaactccgcggctggacaggcggcgcttctggcaggagcagcgggcgacacttcgccttcgccgaaataaccgcgccaaaaaaggtacgccgcgtcgttcggtttcgtatccttttccctttttcctctttctctatctcttgcgacagggaccgggaaagggggataccccgaaagggatccttccccgtgaaggaaccaggctccgagcctccttactgatcagaggttcgaaggctggccccccgaagggttcaacagccgcctcagatcgcgtgggccctacacccactactggtcagaggttcgaaggccggccccccgaagggttccacggtcgcctcaggctactcgggctccgtgcccattactgatcaggggttcgaaggctggcccccgaagggttcacagccgcctcagacgccgagcgagggatgaccaggggtacgttcgatacataaccaaggctcgggctgcgctcctgaggtaccctaggacatttccgagaccagcgggagcgatcttgtaatggaatcccatcggagggaggcatcgagccctcggaccccgtcgccaggggaccgggtccggcagatcacccgcaggtacttttgggcgtgcctctgggcccctagccgacccctaacgaacggggcacggacgtccactcggattacctgcttgcagctcaccggagacaccatgttcggcgcccatcgagggtaacatggcgccctccccctagtcctccttgcggaaaggcgacgcaggggcatatgtaaaaaagccgagtctgtccctgatcgccctcttgccctgtgcagaggctcaggggctgctctcgcaaacccggctccggccaaaccgttgacagcgtcaacataccagcccgagaacttgggccccgaccgtacacccgggctacggccagctcgcatgagggaacaaccagaccagccgaagcattacgcaaggcattaagacctcgaaggagtgaaaccactcctccgaggcctcgggggctacacccggcgggtgcgctcgcgcgcacccaccggaacaaaatgcaaccgagaaaggctggtcccttgcaaaaaagtgcgacgaaagcctccaagcgagtgctaacactcctttcgaggctcgggggctactgtcggggaccataattaggggtaccctcaagactcctaattctcagctggtaacccccatcagcataaagctgcaaaggcctgatgggtgcgattaagtcagggatcagtccattcgagcgactcgatcacgcctcgcccgagcctagcctcggacaagggcagccgaccccggaggatttccgtctcgcctgaggcccccctctaacggcggacacatcttcggctcgcccgaggccctgccttcgctaagaagcaaccctgactaaatcgccgcaccgaccgaccaagtcgcaggagcatttaacgcaaacgtgacctgacacctttatcctgacgcgcgccctccggcagagccgaagtgaccgccgtcacttcgccgctccactgaccggtctgacagaaggacagcgccgcctgcgccacttcgactgcagtgccacttgacagagagatactgacaggaagccaggccctgccaaaggcgccataggaagctccgcccgacccagggctcggactcgggctcagccccggaagacggcgaactccgctccgcccgacccagggctcggactcgggctcagccccggaagacggcgaactccgctccgcccgacccagggctcggactcgggctaagacccggaagacggcgaactccgctccgtccgacccagggctcggactcgggctaagacccggaagacggcgaactccgctccaaccgacccagggctcggactcgggctaagacccggaagacgacgaactccgcttcgcccgaccccagggctcgggctcgggctcagccccagaagacgacgaactccgcttcgcccgaccccagggctcggacaccgccctggcctctgccgacgacctccgcctcgcccgacccaggggctcggactcgtcctcggccatggaagacagactcgacctcggcttcggaggagcctccacgtcgcccaacctagggcgcaggccagccacgtcaacaggaagcgccatcatcaccctaccccgagctgactcgggccgtagagaacaagaccggtgtcccatctggctgtctccaccagataggcaatgatggcgccccgcatgccctgtgacgacggcagctctcagctctcttacggaagcaggaggacgtcagcaaggacacaaccgctccgacagctgtccctccgccaggctccgccgctcctccgacggccacgacatcacactagctgggttccaagatctcttcggctgccacattggcatgtactcagggcactagctctccctcgctagacacgtagcactctgctacacccccattgtacacctggatcctctccttgcgtctataaaaggaaggaccagggtcctcttagagagggttggccgcgcgggacgaggacgggacaggcgctctcttggggccgctcgcttccctcacccgtgtggacgcttgtaaccccctactgcaagcgcacccgacctgggcgcgggacgaacacgaaggccgcgggattcccacctctctcacgccggtctccggccgcctcgctcctttccccccttcgcgctcgcccacgcgctcgacccatctgggctggcgcacgcggcactcactcgtcgacctgagggaccccccggtctcgaaacgccgacaataactctaaccgaacttggcatttagccgatcgattcctaacccatttttcataccaccactacatgacataccgaatacattgaatgactcgttcacattccacatatatctttacgaaaacatttccacatcgcttgcaacttaacctaagcttcgccacataatttcaggacatctacttaaatcatgaatatcatcatcacacacatcgacccgttttgaaataaccctacatgtctatcacaggaatggagcatttcaacacatatcctaaaacaaactaacttcatcacacatcttgcattacaaagctacttgacttatttgaagtgtctactcgaaatcgtgagcacaatcatacactatatacgaaacataattttaacgaacgcataatacgcatcgtcatgacttgacctataaatatagagaaagcgatgactactctggcatgtcaccacctctctatttaagtcaagacaatttctaccatcgattaagagtcgtaagcattaaataccttactactttatacgcacaaataaacttcaacttaacacaactgacaccgatggaatttttactaaactcatcgtacgcataaccctgtctcgcatacaaccatattatggcgtgcactcgagacacttcaatccatgtggcgcgaccactagtataaatggactctgacactcatgtcttaacgatacatcctctacgcaaactagcattctctaaactactcgtcacatcaataaatatatcccctctaaaattatgaatcccatcacattgcttaaaacaaatacacttttcacataaacacatcgatgcatttcccaaaacaaaatccacattttgtaacttagttttcgcatcaaacaacgcatcgcatattttcctatcaaaataaaaatactcgagttcttttctatttcaatttcttccctacacgcgtccatttataaaattatacagttacacacatataaccacatgcacatcatcgaccaaaacataattagacaactacaaatcgtgcacatcaattaacctcttgttctccaatcgcaaacgtgatcctaccaatgcgcataatcgaacattttacacacatccatacaaaatgattaatcgagtcgatcgagagcgacatgcatcggctcaccataaacaaacccaaatgatgtttgcaagaatgacggtgattccgattcgtgcatcgctccaaacatccaacgagcgttaagcgacttgctttctcctcgcaaaacacggggttctctcctccacaaaaataaaacaaagcaacacacatacataattaatcataggaaaataacatcgatgcggaatcaaacaaggagcgtcgcggtctcaccggggtgaacgacgacgacgtttggggctgcgcaaaaacagcgaacacacggcggcatcacggcgtgctgctcactacgcaacaaaacagcaagccggcagcacgcggagccgtcggggctgctgcacatttcatcgagcacaagtgtggatggcggccaggtgtttgtttcaggcgctgaaacaatggagggggagagggctacggctggggaagtggtggctcggccacggcaagaacagggaaggggaggctggtcaccgaccttgggcgcggccagggaaaatggagttgctgcttggcactatgtacaacagagagagggaggaatggctccatgggaagctcgagctcggccaggggaaggaagaaaggggttcggcatccaagctgttggagcccaaggagagggtgctggccgccgtgcgcaagtgaagtttcacgccagctgaagctccctggtcgcggacaggaaagagcagggggcgcctgctgcaggtaggagctcgactcctatggaaaatggcaggggcagaggaggccggctggagcaccgggcagggtgctcggccatggagccgctgcatggatttgctgctgcgccctgggagaaaaacagtaggggagtgaaggatgccatggctgggggcgcggggagcagggagcctgctggtggccttgctgccgtgaagcggggaagaagaaaggcagaggacgctacgagaagagcttcgacgcgctggagggaaggaacgcccggccatggaagcccctgcgcgctggggaaggagctccagctctacgtgcttgaaggagcccatggctggaaaatggtagaggaggaagagaagggtgttggcggctggggtggaaatggaaaattttcagaatgcaaggtagggaagcccatatttatagaggagaaattagggtagggtttcttatgggccaaacgggctggactggatttggcccaaaacactaaattgggtcgcgctaaataatttccggactaaaaatgttcctgcggaattcgtcgctactgagaaacagagcgaaaagagttcggacgaacggaaggttgcgcgattaactcagccgagagtctgtttagattttgcttgaaaataattccctacgcgtaaatcgaaaataaaccgtcctgagatttgatcggttttggatttttagtcggagaaagcgaatcgtgatatataaaaatcgttgccgatgttgattttgaaatcggattggatacagagatgctaagctgagtcgagtaagatttgatcagaggacgacatattgattatttcgtttgtgagtatggactcggattaaaatagttggacatcgatcgaacatcgagaaattggattcggacacagatcaaataacagccgtcgagagtttgatttattgagcttcagatgaggtttataattcgagaatgatttttgagttcgcatttgtgccaaggataaaagttttaacaggctccaaaattggccttctatgagactgagtaactccgaattcggtgaaacatgaatgaataatctggataatcagggacatacgcgagcgagaaatagaaatttttactgagcatccgagattaggataaatctcgcgacgtaacacgaaactgacacctggggtgtcacaactccagcactgccaccctgctggcaggcggatccgtcgaagaaaagcatccagtggggctcagtgaagaccgaagcccttggctccgcaggtgtggtgtccgaatcgggatctggacccccaggagcgctcggggaaggggtccactccacgatgaagtcagccaggacctggctcttgacagcgtggcggggctggaactccagttggaactcagcaagctccgtggcccacttggcgatgttgcctgtggcgtttgagttgtggagaatggcccttaacgggaaggaggtcaccaccacaactctgtgtgcctaaaaatagtggcgcaatttcctggacacaacaagtatagcatagataagcttgtgcgtctcaaggtacctggcttttgcctcatggaggacctcgctgacgtagtagaccggcttctggatggttcggacccctgcattcagtcccgagtcctcaaactcctggccttctgtcaacatcgtggtggtcagaccaccaccttctcctaggggaactttatgactcccctagggatgttgtgtcgtactttcgacgaccagcaccatgctcaccgcctctgtagccgctgcaatgtactagtataatggctctcctggctctggagctaccagtattgatagggacacatggtgctgcttcaactcttgaaaggcttgttctgtctctttggtccaagagaatgggtcggacttccgcaatagcttgaagaagggtagtgccctctcaaccagtcttgagatgaagcgactaagggcggccagtgaccccgtaagcttctggacgtctttgattcaggccggaggcctcattgtctctattgctttgatcttctctgggtttgcttcaatgccccggtgtgaaaccaggaatcctagcaacttccctgcagagacaccaaagacgcacttgtccgggttcagcttcatgcgtgttgcctgcagcttgtcaaagactagggttaagtcttccactagggtcgaccctcccttagtcttgactacgatgtcatcgacgtatacctctaccctgtccctaatcaagtcaccaaaagtattactcatcgcccgtacaaatgttggcaaggcgtttttcagactgtaaggcattacaacataacagtaaagtccatccacagttacaaaagcggtatgcttcctatcttgcctagacatctcgatctgatggaaactagagtaagcatccaggaaggataggaggttgcacccagaggtagaatccacgatttgatctattcgtggaagtggatatgggtccttgggacaggccttattgaggctggtgtagtcgatgcacatccaaagcttcccgttagccttggggacgatgactagattggccagccatactgggtgatggacctcttcgatgaaaccagcgtccagcagcttccggacctccttacggatgaaatcctgccgctcgatggactgtctttgaggcttctgactcaccggtttggcgtcagggtggatcttcagatgttgctcgatcacctccctagggatcccaggcatctgcgatagttcccatgcgaatacattggcatttgcctggaggaaggcgatgagcgcgatttcctatttctcctccagatcgcccgtgatgcgagtggtctgggaggaatccccgttgagccggatggtcttgacagggacgccgtctgccccagatggttgcaccttaggcaccttagcaggcatcttggtacaggaagtcgaggggtccctcccctcgtcatccgggcgagcagcttctgccgctagggcatgcaacttctcgatagctgcaagcgcagcgggacggtcgccccgcatggtgaggaccccagcaggggatggcatcttgaggaccaagtacctgtaatgggcaatggacatgaaccggtacagggccggcctgccaatgatggcattgaaagggaggttaacctccgcaacatcgaactagacattcttagtgtggaagttatcctcagtcccgaatgtaaccaggagtgtgatgctcccaaggggatacaccggtttagggcccactccagagaacgtgcgagagggtcctagtcgggatcctgggatctgcagctgcttgaacgcagcgtggctgatgacgttgagcccaaccccaccatcaatcagcacatgatgcaacttcatgttggcgatgacaggggcagtgatgagtggtagtataccagcccctgccatgttttcggggcagtcgggtgccccgaaggagatagtggtgctccgccaccgctgatgtggggctgccttcgggacccctggggtcgccaaaaggacctcgcggcgcagggacttcacgttcctacgggaggtgagctcccagcttccaccatacattacgtacagcttcttgcggcggtcgttgtcatcaccggagtcggagtctccagtgaggatatccttgaggacttgctcgggggcctaattctcgaggtcccattctcccgtggccaggtcaccttcgtcgaccttctccttgccaggccggcgccgaggcggcgagccatccctggaggcatgctcgcgccgctcactgatgcgcttcacgagcttcaggatctctcgtcattctgaggcactgtggcgactgttggggtggacagggcatgacccaatgtcacttccctgttgccgtggatgcttgccgcgctcgtcccggtccccagccgtagctacagcaactggagcaccagactacggcctatcgtgacacggtgcttcttctttttcttgccaccaccctgggtggcagcacctgagccacccatttgggtgactctggtttgcagcgtcgagtgccatgcacggccctcagtagctctggcacatttgtcggccagagtgaagagcgtagtgacggtttccacgtcatgcgtcgccaatttctccaacatcttcttatcacgcacccccctgttggaaagcagtgataatggaggcatcggagatgcgaggtatagtcccctgtaccttggtgaagcgggagatgaaagcccggagagtctcctcgggttcctgcctcactgcatggagatgagcctccacgccatgctactgataagcactggcgaagttcattgtgaaccgtgcacagagctcttcccaggagtagatcgaccccggggtgaggttcatgagccaggtctgtgccggcacattcaaggctacatggaaatagcttaccattacagcagtgttcccaccagctgccgtaatggcggtgacatagacctacaggaatttcgacaggttcgatgtcccatcgtacttctccggcaggtgtggccggaacatgggtggccaagtcgccgcgcggagatgatctgctagtgcggcgcagcccacgccgaccaatgggacacccatctggattcgggcgtcccttgcagtgaagtcttggtcgaggttgcgaccctcgaagttttgccggcgctcacgcgccctctccagagagattcgagcatcctcgcctgcacgcctgtggttgagttctgctcgcaggtcgttagtctgtgccccctcactgagggtgaatgcacagacgttgacgcctcgcgctgatgccggaatgatcgaggcctggacctggccgagctaggatgggccatgccgaggagacggtcgacatcttcacgccactgcctcatggcccccggggaggccgtggaacttggtgggttacgcagcaactccctggctgcagacaatggcccaccataggtagccctcgacggagtcctagaggtctgtgcgggcgtgtgttgctgcgcagcgtgcatagcagcagcaccaggcacagcgcggttggagcctcgtggcatggaagataatgccccttcctccatcaagaagtcctcgggagacaagccacggtgctcgacgatctgaaccatcgtgtcgagcaagaaaacaggcaaaaacctaaagccaaagccccctacctggagcaccaaatgtcgaagggaaaatcctccggccgggtggcggaatgcacccgccctaatcctaagatgaggagggggcctaagcggttgcctgtttggtgaattcgggatgaacacaagaggacacgagggattatagtggttcaggccgccggagcgtaatacactacctccactgtgtgtatgttgtattgagtgtgtacagcgtgtcccttgtaacgttgtgtgccttcccttttatagtttaagggaggcacatacaaggatgctgagccccgacatgtgggcccaggagcataatggaagaaatacattatgtgaataactaatgctgacagagtaacacatgagtaatcagcgggagtcatgatggctgcagtccatgcagcattgatagacagtaacccttttcttggaaacatacgagtaatggtgagtcattgccctcgatatggtaacgtgtgagtaactgcatggcccacgtatcgtggactgagcatgccgcctgtcagtggaatggacaggcgcacatcttctccgtaatgaatgcgaaggcacgcgtagcccagaggcatcatgccaggttccacccgttggtttatgccgcgcgcagtatgccacgtggcagcatcgggtctccgcctgagcagggagaaggagtgtatgcggataggtccggatcccaccagaccaggtctagacacgtgtcggctccggacccccacctgggtcctaatcaaggcccgggtatgttctgtcctagaaccctgggaccccactatgggtggcccagacccatacggggggttcggatcccatcctaggggtccggtttgtacacgtggaggtcctggaccaaacttggaggcctggaccgtatatacaggggtctggcactggtccggcactctcccatggggtccggactcactgttgatgccttggagtacatcactttctctggacacatggcggccccgaacccgcccatgtggtggggtcaggtgctgttgctggcccagagtagtcgcccgaggctagggcgagtcatggtctggtcccacatacagcttatttaccacgcgactaaagatagtcgtgtgggtactgcgtctttatacagtagtaaggggtaccctagttttagggtgccgacacacatcttcctctagaacaccatgaagaaacgcgttctgcacatccagttggcagaggctccaaccctgagagacagcaagagacaaaataaggcggacagtagcaaatttaactactaggctaaaagtgtcatcatagtcaatgtcgtagcgctgtttaaaacctttagccaccaatcgagctttatgatggtcaatagactcatcagcttttctcttgagtttataaacccacttgcaatcaatcaaatttctgtcaggtgcgggaggaaccaagtgccatgttttattccgcataagggcagaaaattctaggtccatggcagcttttccagtttgggtcaaacaatgcaacagacaagctggagggttcttcacaaattgccaaatttccatacctgatcgtgccatctgtaaactttctgggcttcacaataccactctgtagccgagtgcgcctagcaggaagcggaataggacacgaggctgatggcgagggcagatggctgtcagtgagagagggaccagccgcgccagagtcggcacgaggcaatcccgaagtggctgctgctattgatgcggccgtggtggtgggaagcaccgcgttgctgggcgcacctgtagccgcgtcggaggggtgtggcgtggagcctagcaacagatcagcaccgggattgaggccaccagccgggacagaatttgcagcagggatcattggtggctgcaaaagctggttaggccacaaaatcggagcaagcatgctggattcagcaggagaattagtcacaagatcatctgagttggcccgagaattattaggatcgggtagaagaagcacgtcagaggtatatcgagcaccgactgtgggatggagagcagcaaagggaaaagcgtctcatcaaaaacaacatcatgtgaaatataaacacggcccgttgagatgtcaagacacttgtaacccttgtgaaggttgctatagactagaaaagcacaccaaatggaccgaaactagagtttatgggtgttgtatggccgcaaatttggctaacatgcatagccaaagacgcgtagattagagtaatctggggtagcacctaagagacggtggagcgatgtgtcataatcaagaagcttagtaggagttctattgataagatgtgttgaggtgaggaacgcttggtcccaaaacttgagcggtattgtccattagcgagtaaagagaggcccatctcaacaatgtgcaacgaatcaagctgatacataagannnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnncaattctagaagatttcgtcgatcttgatggtgtcgttggccttgatgagcgggtcggggtagcggatggtgcggtcgtcgtaggtgtttaggcaggggatgcctttctggccaaactgaacagaccttaccttgcagagcatgaactgcacacaaaccaatagaaaagcagtgagaatttcacaggcgtactatgaaagggcatgggaatttccagcgatgtaaatggatagatagacagagcaacatctattaatagtcctaacgattgtagcacatgacattttcaatgcaagactttcatgcacacaacatatatggacagtatagcaaggataaggtacatagatctacagaaaaaaaagaacaacctgaagcattagacaaatggggaagtacagaagattgtaggtaccaaagctagaaaatattgttttgtcggcgtttcgaccccggggggtccctggaccgacgagtaaattgtcgctgcgtgtcccagcccagatgggtcgacgcgagacagaacacaaaggggggaaaacagcaaaggggaacccgcggccttcgtgttgtcctgcgcccagggcggatgcgcttgcagtagggggttacaagcgttcgtgtgggagagagagagagccttgtgcgtcagcccgttctcccgcgcggccaaccctctcgtacgagagccctggaccttccttttatagacgtaaggagagggcccaggtgtacaatggggggtgtagcaatgtgctaacatgtctagcagagaggagacagagccctaagtacatgccgtcgtggctgtcggagaggttttggcgccctgttcatgtgatgtcgtggccgtcggaggagcgtttgagccctgtggaagtacaactatcggggctgtcggatccttgctgacgtctccttgcttccgtaaggggctgagagccgccgtcgtcacggagcacgcggggtgccatcattacttgtttaccggggcgagccagatgggacgccggtcttgttccccatagcctgagctagctaggggtagggtaatgatggctccccctgcgacgtgtcggtccgagcctgaggtcgggcgaggcggaggctcctccgaggtcgaggttgagcccgagccctaggatcgggcgaggcagagtccgtcttccgaggtcgaggctaagtccaagccctggggtcgggcgaggcggagtccatcgtccgaggtcgaggctgagtccgagccctggggtcgggcgagccggagtccgtctttcgaggtcgaggttgagtccgagccctggggtcgggcgaggcggagtccgtcgtccgacgtccaggttgagcccgagctctggggtcgggcgaggcggagcttcccatggcgcccgaggctggacttagctgctgtcagcctcactctgtcgagtggcatagcagtcggagcagggcaggcgatgctattttcccgtcaggtcggtcagtggagcggcgatgtgactgcagtcacttcggccctgtcgactgaggagcacgcgtcaggataaggtgtcagtcgatccttgcattaaatgctcctgcgatacggttggttggcgtggcgatctggccaaggttccttctccgcgaagcttgggcctcgggcgagccgaaggtgcgtccgttgcttgaggggaccctcgggcaagacgtgaatcctcctgggtcggctgcctttgcctgaggctaggctcgggcgaggcgggatcgtgtcccttgagtggacagagccttgacctgaattgcgcccatcaggcctttgcagctttgtgctgatgggggttaccagctgagattaggagtcttgggggtacccctaattatggtccccgacagtagcccccgagcctcgaagggagtgttggtactcacttggaggcttttgtcgcacttttttgcaaggggaccggcctttctcggttgcgtttcgttccggtgggtgcgcgcgagtgcacccgccgggtgtagcccctgaggcctcggaggagtggtttgactccttcgaggtcttagcacgtttcgtgatgcttcggccggtctggttgttccctcatgcgaactggccgtagcccgggtgcatagtcaggttccaagttctcgggctggtttggttgttccctcatgcgagagcagcccccgagcctccgcacagagcgagaggacggccaaggactgactcggcttttttcatacgcccctgcgtcgcctttccgcaaggaggaggggggggaaagcgccatgttgccctcagagggcgtcgaacatggtgtctccagtgagttgctaacggttgatccgagtggacgcccgtgccccgttcgataagggtcggctagtggcccagaggcgcgctccaaaagtacctacaggtgatttgccggacccggtcccgtttgatagggtccgagggctcgatgcctccctctgatgggattccgttacagaatcgctcctgttggtctcggaaatgtcctagggtacctcgggagcgtagcccgagcctcggccatgtatcggacgtacccagagtcatccctcgctctgcgtgctctgaggcggctggcgaatccttcgggggccagcctacaaacccctgatcagtagtgggcgcagagctcgagtggcttgaggcggctgtcgaacccctccgaggggctagccttcgaacctctgaccagtagtgggcacggaacccgagtgctctgaggcggctgtcgaacccttccgaggggccagccttcgaacctctgatcagtaggagggcgcggagcccgagtgctctaaggcgactgtcgaacccttccgaggggccagccttcgaacctctgattagtaggagggctcggggcccgcttccttcgcggagaaggatccctttcggagtatcctctttcccggtccctatagcaagagagagaaagaggaagggtaaaaggatacgaaatcaaacgacgtggcgcaccttttttgacgcggtcattaaggcggaggtgaagcgtcacctgcttcgcctgccaaaggtgccgcctgtcctgccgcagagttaatgcgacgggatgagtggttcgcggggcagccgttgtgcgtgcgctagccgttcgaggaacggaacacgggcgtgtcgtcttcacgccgtgggagggggctctctcgctgtcccaggaggggacgtgagcctacagacgacttgaccgctgcttccgcccgcctgccgccgccattactgccggcccacttttggccatatcaaccatcgcgccttctcccgcggctgactgacccgtgatcgatgtgctcggttggcactgttgggccatgcgcagggttgcctcgagtcgcggcaccggttccgcagtcgagaaggcgcggtactagcacaagtggcggtgcagtttctcgcgcgtagtaaccggcgcgccggttacatgacgtgtgggcctgggcccccgtgctggacgcgtcggagtcgaaagggtgcacccccttggtgcggttgcatgccgcctgcatggcggtccgccctttcacccgccggtctgggcgaaagtggaggagtgcttgtaaccgctgggcagttacgcactctgcgcgcgacggtttggcttcttctgccctgggccagcttgcatgacgcgtgggacccagcccccatgtcgtagggggaggaccttggagcgtgttggtgaagactcagtccgcgacggctgaggacgcaagtggggagagtcgcctttaaaaggagggcgacccccttggatggcaaccatgtcttcacactcccttcatgcatcgcgcccttccaacttccgagcccccggatggggagcgcccgcgttgctttcgtcttgtcgtcgttggaggaacgcaacttcgcggaagttggtacctttcagccatcgctcggcttcaaggattttcatcaggcggcccggctgcatcccctcgctggtggtcacccaagacggtgaccaccagtttgatggtggggacgtgggcgagggccttgtcgcagcagcgtctgcactgaggtcatcgctgctgctgtttggctgtccggagcggaggtcgttgtcgctgctgccagagcgggcctcggcgagctgtctagggttttgttgctgaaagttccctttgacccgggaacaggatctggatgtcgcctagagggggggtgaataggcgaataaaacttttcactttaaaacttaaattcttactctactcgaagacttagtatgcagtggagtgagaagactcttcaagtaggttgcagcggaatagaagatcctgtctcaaaatgtcctgcacttcaaataaagcttataccacagataagtattgaagtgcagatataaaggcgagtagaaagagagtcaggatacaatacagaacagagcacacagacgcaaggatttatcctgaggttcggccaagcctgaaatgcttgcctagtcctcgttggagttagccacacctgggcttggagtctatttcaactccttcctccgtttgctcagatctgtcagtatgacagatagagcctttcactattgagtggggttacaacagaaccgcggctgcttacagacttcttggcagcaccccggtagagtaacgatatgctcaagaccttgctctagctcttagcagcactactcctctctctaaggcttatagttgtgccttctacacaaactatagagttacacacaagagggagagtgagaattgattccagtggagtctacacttgttggctgcacttctattttgctggaggcacctaggggtcccttttatagacacaaggggcctaggagccgttggaagcaatccaggaaggcaaatcttgccttctgtcgggtggcgcaccggacagtccggtgcacaccggacactgtccggtgcccgatttctttccttctacgtcgaagccgaccgttggcagtcttggagccgttcgcgcaccggacatgtccggtgcacaccggacagtccggtgcctccatctagccgttggctcggccacgtgtcccgcgcagatcgcgcggccaaccgttggcccggccgaccgttggctcaccggatagtccggtgcacaccggacagtccggtgaattatagccgtacgtcgccggtgaattcccgagagtggccagttcgccagagttcagcctggcgcaccggacactgtccggtgcaccaccggacagtctggtgtgccagactgaactaagtcttggctgtacacagccaagcctttcacacctcttcccttttcttcttctttctgtttctaacacttagacaagtatattagtccccaaaaccaatgtactaagtctagaaacataccttctattaatcattacatctatagcatttcacaagcttgagctttgatgttggactcataaattatcaagtcagcttgacttgatctagattgacatcgcttggctccaacatcctgtaaaggtcacatagaacatctccaaacataggaacaacccaaactaaagatcaaagtgaacttagctcttttgggctgcttccagttctggtttcgacacttgttctccttctagtgaccttgatctcctccttagagcttgatcttgagccttatgacttacaccacataactatagctgttacctcattggctgtaagtcacgtccttatgtagtgatccttgatgtgccgtagctgttctcaactcgatcacccttgactttgcaagccttcttcttcacccttggctttgggttcctcagcctccttgaccttctcccatgcatttggtacctcgaagcttttcttgcctccgtccttggcttgatcagttgtcttcgagctacgcacccgagtctcactttgtgcaatgtccatcttacttgtgatgtccattatgtatccataatccagttcttggaccatcacatttgttcacttgtgttgaaccctgtaggctttaccttaagcacatgttcaacacttagtatacttgttagtcctttaattgagttgtcatccaaacaccaaaactcacaagagagctttcaatctccccctttttggtgattggtggcaacacaattaaagcttacataagaataagatttgaagcacaaatttgaattctaagattatagaatgctccccctaaataagtgcttacttcaaaaacctaattttgaccacaaacgtcaatttgcacatacttaggaaaattgaaacatttctacaccttagcactttttaggatgcattatgtcaagaatcaaaccatgatgctataacacacaaatgcacataatcagagttaaacaccattcaaattagtggatatatcacaggaatatcaacctaccactattcaccattaagataccaacttaaactaagatatcaatttaaagcaatcttaaagcaccattaaccacatgactatctatttcactatagaagccaaataattcatcgcagcggaaacactggtctagtccatatgatcaacacgtataatactgcaagaaacatatgaatataaaacactagtctagtccatatgatcaacacgtataatactgcaagaaacatatgaatatcacacttggcaaagctcaaactaacacatcacccattaggataagctttcctctcaggttgagataagctttaatgcacaacttctccccctttgacatcaaacaccaaaaaccatactcaagcaagaacatatgatgatgtcaagggacagcagggtgttaaggggaaaaacgactatcaaaactcccccttatttattgaacatatgtcctatcaacatttaggtaagatacatatatgcaaaaagattaatacttccttttgtacctttaccatgatgtagtgtacttcccatcttgaaagtagttaatctctcgagagcttctccacacttgtgcctgattctctctcctaactttttcttgttgctaagacaccaaacttagaacaagttatagtattgggcacaagaagaaacttctattctcatgattatcaaaagatgtcaattgaagcgaactattacggctaccaattgaaagataccaattgcaaagttcatttattatcatggctccatgatatttaagaataagcatctattatcaccagatattatagagcatgagcaatctaaaaatatgcacttactcacaacttgagataccaattttcttgacttacagaggtacccaagtcctgattgctccatttcttgcttatcttctcttttccacctagagactatacaagattgctcaagaaacagttagtctcaaaagacacaagttatgtgtgctccccctcaagttgtgcatcaagtatttgaatgacttgcactttgcacattctagcttccttagaattagaggggatcacaacataccttggtcaaggcatactctaccactttcatcacccaaagatgccaatttgaatatcaaatgaaacgccacataacaccaattgaaggctaaatgaaaggttgactaaatacaacaatgcacgcctcagtggcacctaagccaattgaatactcacaggaagtctaacatttacgcaacttgtacatgcttcatatttaactatcattgtatataccaattaaagataaacacaatcgaaatatctaagcatgttataattaagaaggtttcttaggtgcacaaaagaaacaacattttaaaggcataaattacctaagccaagatattaccaattgaaaggcaagaacatagctatgatcacaatgaatggaatttcaagaatatttaatgaaattgcatagctccattttccatacctttgcctttatgagagcccttgttatcgccaatttagggctccttttgcttacgcacctcatagctcaaaagggcacgacatggatttgaaattcacacagtaccaaactagggtaatcatgtgaacatggactaaacaaaatgtcataattgcacatagcatgacttacaaaagttacaggtttatccatatacatcaagagagttatcgttgtggatataacaaatgaaatagctacccatgaatgattcaaaagatatatcctttatagcaccagtcatgattaagcaaccatcattatgatcaatttaacacaggcaatcataaagcataactactctaaggacaggtagcacaacaagccaacttaagagcaatactaaattgcaattatgtacttaaaatacacgggtaccgtcctttggagagcaggttgtagattctcatcaagatcctttacttgattcaccaataatgattcaggacctatacaccttatttctcttgagatgaacatgggattagtgtttcacaataattcaaccttgggtcaataaacactaaaacaattaacagcttaagcatagagttttagataaccgtcttaatctttcccatggtctccagtccatctcgaggcacctgcatggtctagttggcacagtttggtatccatctcgggatgggtacataatgatcatgtaaatgtgcctttggtacccaaattgcctttgtgctagttctaggtgatctcgttatagatctagcacaagtgtatgatttgggtctcctatgcgaataagattgacacaaattcacttgtttaggaatcttacctttgtaacataccttggatagatgaccttgctcaccacacttgtagcagaagcgtcgctcaacttgacatgacgcttgttttgtgtcattttccttggtgagggtcactttggaggatgcatatccttgattcttcatgaccggacatgaagtgatcatgtggtccttattgttgcatccaaaacaactccttgttctttcatccttgtctttgtacggacaagacgcgatgaggtggcctgtctccttgcatttgaagcacctcctttttcctcttccctttttgtgcttgaatgacatggagagatgatcagtgcaaacaacatgactaattgaatttttacctttttccttgttcatgttgatttcttcatttatagctttgggaacattcttcttattgagcttaacacttgctgcagtttttcctttctcaagcttcttcaccacgcgcccgtggatatcttgagagagttgagcaatgtgtcttctccttagttgtctttgcttcttgttcccacagaatttcttttgtgaccctaaaacttgttgctcaatcaatgattggctttcttttgagcaacaggggttagcacatggtgatatacacttcaaatgcgcacacgtgcgagaatgaggttcacatgaatttaagtttgcaattataacctcatgagcaacattaagcatgatatggtcatcaactaatttattatgagaatttgacaacatatcatactttttacctagagcacgtttttctaagttcattgtttctacttgactcttaagcatagaattttccgttttaagttgagcaatattagataatgcatcatgactatttctttgctcaattaaaacatattcatacctttggaccaaatcatcatgagagcgccttagcttctcatgttctttggtcatcttctccaggctgttgttggttttgatgagggactcctctagcctgagaagcgtctcgccttgttccttgttccttctcaacagcttaaccaagagtgccttgtcctctttgttgagatggatgtagaaacggtgaatctcctcttcctccacatcatcggtctcattttccctgtcattaatgtcagtggaagcaatataggaaaatgtaccttgtgatgatgaagattcatcctcatatttctccttatcatggctttcgtctccaccgtcattgttagcaataaaacatttatcactagtggagaacaaacctgtcgacgaggtggattcatcgtttggatgccatcgttcttgttcttctccctttgaatggttagtatcacaagtaatatagggagtaggagcatcacagtttgccacaaaatatttttctttaatcctattccataaatcatgagcatcaacaaatagatcactatcactactcatgatggcaaaataggcacctctagatagagaatcaactaagatgttgcaagcatggtgatttagagttagacatcttagttcttcattggatgggtttttactaatattggagggaaaaatactactactaaagacctgtctcaaatcaggatcaacactcatgaaagcactataaatagagacagaccaagacttgtaattagagccatcgtctaaaagaagttccacagttacctcttgtgacgacatcgtcatctccggacggctaagcccacactggagaggcctagctctgataccaattgaaagttccctttgacccgggaacaggatctggatgtcgcctagaggggggggggtgaataggcgaataaaacttttcactttaaaacttaaattcttactctactcgaagacttagtatgcagtggagtgagaagactcttcaagtaggttgcagccgaatagaagatcctgtctcaaaatgtcctgcacttcaaataaagcttataccacagataagtattgaagtgcagatataaaggcgagtagaaagagagtcaggatacaatacagaacagagcacacagacgcaaggatttatcccgaggttcggccaagcctgaaatgcttgcctagtcctcgttggagttagccacacctgggcttggagtctatttcaactccttcctccgtttgctcagatctgtcagtatgacagatagagcctttcactattgagtggggttacaacagaaccgcggctgcttacagacttcttggcagcaccccggtagagtaacgatatgctcaagaccttgctctagctcttagcagcactactcctctctctaaggcttatagctgtgccttctacacaaactatagagttacacacaagagggagagtgagaattgattccagtggagtctacacttgttggctgcacttctattttgctggaggcgcctaggggtcccttttatagacacaaggggcctagaagccgttggaagcaatccaggaaggcaaatcttgccttctgtcgggtggcgcaccggacagtccggtgcacaccggacactgtccggtgcacaccggacactgtccggtgcccgatttctttccttctacgtcgaagccgaccgttggcagtcttggagccgttggcgcaccggacatgtccggtgcacaccggacaatccggtgcctccatctagccgttggctcggccacgtgtcccgcgcagatcgcgcggccaaccgttggcccggccgaccgttggctcaccggacagtccggtgcacaccggacagtccggtgaattatagccatacatcgccggtgaattcccgagagcggccagttcgccagagttcagcctggcgcaccggacactgtccggtgcaccaccggacagtccggtgtgccagactgaactaagtcttggctgtacacagccaagcctttcgcacctcttcccttttcttcttctttctgtttctaacacttagacaagtatattagtccccaaaaccaatgtactaagtctagaaacataccttctattaatcattacatctatagcatttcacatgcttgagctttgatgttggactcataaattatcaagtcagcttgacttgatctagattgacatcgcttggctccaacatcctgtaaaggtcacatagaacatctccaaacataggaacaacccaaactaaagatcaaagtgaacttagctcttttgggctgcttccagttctggtttcgacacttgttctccttctagtgaccttgatctcctccttagagcttgatcttgagccttatgacttacaccacataactatagctgttacctcattggctgtaagtcacgtccttatgtagtgatccttgatgtgccgtagctgttctcaactcgatcacccttgactttgcaagccttcttcttcacccttggctttgggttcctcagcctccttgaccttctcccgtgcatttggtacctcgaagcttttcttgcctccgtccttggcttgatcagttgtctccgagctacgcacccgagtctcactttgtgcaatgtccatcttacttgtgatgtccattatgtatccataatccagttcttggaccatcacatttgttcacttgtgttgaaccctgtaggctttaccttaagcacctgttcaacacttagtacacttgttagtcctttaattgagttgtcatccaaacaccaaaactcacaagagagctttcagttgccccgcaggccctccaatgtggggggtcgttcgtacctgtgggggcggaaccagagttctgtttgtaatggcaccttgagtgccggtgtctgttcattgcggctgtcggggcctgaagatgtgtattttggctaaagccgtattttttcctcatttcgagcactaggactcgcctgtcggctagctgaaccgcttaaccaagtgtgagttgcctcgtgcggaaggtgacgagtgaggtatccgtatcccggaggcgtaggagtccctcggatcggtcggccttgccgcccgaggcttctcttgcttagttaaagaaaccctcggccgctctgcgatgagccggagctagaggcagcggtgtcagcggtgtcagcgtggacagaggcggagttggctcaaaaagaagcttcatcggccggagcctggtcgggccgtccactggtgggaccgacgccggagtcgggttgccgaggccatgagccgggctgatgtcctcgggggacagctggctgaggctacagagcggtcggtcgagtcgtctactcgggccgggttcctggaggacacctcggcgatggcccaggcgcggtgctgacaggttccttcgagatggagatcctccgaccgtgtcgccgtccgaggctgggtcggactccgccgaaggtggagtcgacgccgagggtgctgctgctcccccactgatgtctgatcctgcaggaacaatttatctgtagtgtgcgtatgttttttgcggccgccgaggcccaaacataccgtcgtcgtgttgtaaagcggcgtttcttttccccttgtttcgagtatcgggacttgttcgtcagtaacagaattgcttatccgagcaagagttacttttcacggaaggtgatgagtgaggtatccgtatcccgaaggtgtaggagtccctcggctcggtcggccttgccgcttacgtgtactcttactcgtccgttggattctgttatcgatatagtcgagaaggcacaaaaaatcgtttcggcagaaaagctttcgaacgttaagacttgttcggccagcgggatcgcttatccgagcgtgagttacttatcgcagaaggtgatgagtgaggtatccgtatcccggaggcgtaggagtccctcggctcggtcgtccttgcctgcttacgtgtactccgtcgttttcaggatcccactttcgaagtagtcgaaaagcacgaaagatgttctggcagaaagacttttttcgaggaaaattttgacgtagagggggtgccccccttctagcccccgagggagggtcgggctttgccgaggcaaggctgacccttccttgatggttagactttgttggcgtatgtaaacgaggtgtatgaacgacttgaaaacatcttaagggtagaagcgacgtagctgtcggatgttccaagcgttgatgtagacctcgccttgactgttggccagcttgtatgttccgggcttcttagggaggcgtgagcttgtgacaccctcgggcgtcttgacgtagccgaagcaccaagtcgcccacctggaggtctcgggaccgaaccccttgggcgtggtagcgtcgcagggactgctgataccgcgccgagtgtagtaaggccatgtcccgagcctcttccagctggtccagtgagtcttctcggttggttcgattgcttcggtcgtcgtacgccctcgtccccatagactagaaaaaacagcgtgaagatggcccagtgagtctgtgggcaagatggcctcggccccatagactagaaaaaacggcgtgaagcccgtggctcagcttggtgtcgttctcagactccagaccaccgaggggagttccttcatccatcgcctgctgaacttgttgaggtcgttgtagatccgtggcttgagtccttgtagaatcatgtcgttggcacgctctagctgcccattcgtcatggggtgagctacggcggcctagtccacccggatgtggtaatcctcgcagtaggaactttctaccggtgaactgggtgccgttgtcggtgatgatggagttcgggaccccaaagcgatggatgatgttggtgaagaacgccaccgcctgttcggacctgatgctgtttaggggtctgacctcgatccacttggagaatttgtcgatggcgaccagcaggtgcgtgtagcccccgggtgccttctgcaaggggctgacaaggtccagaccccacacagcaaacggccaggtgatgggtattgtttgcagagcctgagcgggcaggtgggtctgctttgcatagaattgacacccttggcaggtgcgtacaatcctagtggcgtcggccaccgcggttggccagtagaaaccctgtcggaaggcatttccaacgagggctcgaggtgctgcgtggtgaccgcaagcccccgagtgtatttcttgtaataactcctgaccttcggcgatggatatgcaacgttgtaggacgcctgaggggctgcggtggtagagctccttcccgtcacccagcaagacgaacgacttggcgccccacgctagttgccgagcttcggctctgtcgaggggtagctctcctcggtggagatattgcaggtacagggtctgccagtttcgattaggcgtgaccccataccgctcttcctcgacgcgcagtgcctcaccctcgggggccgagggtgcctcgggcagggccaaggctttctcgggctcgggcgtgtcgctggtcttgactgagggttgatgtaggtctcgggagaagacgtccgggggaaccgttgtccgcgccgaggctatcttagccagctcatccgtagtctcgttgtatcgtcgggcgatgtggttgagctcgagcccatagaacttgtcctccaggcgccgaacctcatcgcagtaggcttccatcttcgggtcgcgacagtgggagttcttcatgacttgtcgatgacaagttgcgagtcgccgcgagcgtcgaggcgtcggacccctagctcggtggcaattcgcaacccgttaaccgagcctcgtactcggccacgttgttggacgccgggaaatggaggtgcaacacgtagcggaggtgcttcccgaggggcgagatgaagagcaggcccgcgcccgctcctgttttcatcaacgacccgtcgaaaaacatggtccagagttccagttggatcggagctgctggaagctgggtgtcgacccattcagccacaaagtccgccaagacttgggacttgatggccttccgaggggcgaatgagattgtctcgcccataatctccactgcccactttgcaatcctacccgaggcctctcggcactggatgatctctcccagggggaaggatgacaccacagtcaccggatgagactcgaagtagtgtcgcaactttcgccgcgtcagaattaccgcgtaaagtagcttctggaatttgcgggtagcggattttggtctcagacagtacttcactgatgaagtagaccggcctctggacgggcaatgcgtgcccctcttctcgtctctcgaccatgatcgcggcgctgaccacctgagtggtagcggcgacgtagaccaagagggcttctccggcaacagggggcaccaagatgggcgcgcttgtgaggagcacctttaggttcccgagggcttcctcggcctcgggggtccaagtgaagcgctcggtcttcctcaagaggcggtacagaggtaggcctctttcgccgaggcgtgagatgaaacggctcagagccgcaaggcatcccatgaccctctgtactcctttcaagtccttgatggggcccatgttggtgatggccgcgattttctccgggttggcctcgatgccccgctcggagacgatgaaccccaagagcatgcctcgggggactccgaagacacacttctcgggattgagttttacgcctttcgccttgagacacttgaatgtcgtttcaaggtcggagaggaggtcggaggctttcctcgtcttgactatgatgtcatcgacgtaagcctcaaccgttcgaccaatgtgctctccgaacacgtggttcatgcatctttggtatgtcgcacccgcattcctcaaaccgaatggcatagtaacgtagcagtacatgccaaagggtgtgatgaaagaagtcgcgagctggtcggactctttcatcctgatttggtgataccctgagtaggcatcgaggaaagacagggtttcgcacccagcagtggaatccatgatttgatcgatgcgaggcagagggagggaactttcggacatgctttgtttagaccagtgtagtctacacacatccgccatttccctcctttatttctcacaagcacagggttgacaagccattcgggatggaatacctctttaatgaaccctgcagccatcagcttgtggatctcctcgcctatggctctgcgcttttcttcgtcgaatcgatgtagaggctgcttcacgggtcgggctccagctcggatatccagcgagtgctcggcgacatccctcggtatgctaggcatgtccgagggactccatgcaaaaacctcggcgttcgcgcggagaaagtcgacgagcactgcttcctatttggggtcgagctcggagccgatccggatctgcttggaggcgtcgttgctggggccgagagggacggacttaatcgtctcagctggctcgaagttgccggcgtggcgcttcgcatctggcgcctccttggagaggctccccaggtcggcgatgagggcctcggattcggcgagggcctcggcgtactccacgcactccacgtcgcattcgtacgtgtgtcggtacgtggagccgatggtgatgaccccgttggggcccgacatcttgagcttgaggtaggtgtagttggggacggccatgaacttggcgtagcatggtctccccagcactgcgtggtaggttcctcggaacccgaccacctcgaacgtgagggtttcctttcggaagttggagggagtcccgaagcagactgacagattgagttgcccaaggggttggacgcgtttcccggggatgatcccgtgaaaaggcgtcgcaccggcccggatcgaggacagatcgatctgcaggagcccgagggtcgcggcgtagatgatgttgaggctgctgcctccgtccatgaggaccttggtaagcctgacgttgccgatgacggggtcgacaatgagagggtactttcctaggctcggcacgcggtcggggtggtcgccctggtcgaaggtgatgggcttgtcggaccagtctaggtagactggcgctgccacctttactgagcagacctcccgacgctcttgcttgcggtgccgagtcgaggcgttcgccacttgcccaccatagatcatgaagcagtcgtggacctcggggaactcctctgccttgtgatcctccttcttgtcgttgttgtgggctctgccacctttcgccggtggcccggccttgtggaagtagcgtcgaagcatgacgcattcctcaagggtgtgcttgatgggaccctgatgataggggcacgactccttgaccatcctatcgaacaggttggcgcctccgggaggcttccgagggtttctgtgctcggcggcggcgacaatgtctgtgtcggcgacgtcgcgttttgcttgtgacttcttcttgcccttcttcctcgcgccgcgctgagcggacgccttggggacgtcttccggctgacgcccctgaggctgcttgtccttccggaagatggcctcgaccgcctcctgaccagaggcgaacttggtggcgatgtccatcagctcgctcgccctagtgggagtcttgcgacccagcttgctcaccaggtcgcgacaagtggtaccggtgaggaacgcgccgatgacatccgagttggtgatgttgggcagctcggtgccctgcttcgaaaatcgccggatgtagtcccagagggattctctcggctgctggcggcaccttcggagatcccaggagttcccagggcgcacgtatgtgccctggaagttgccgacgaaagctttgaccaggtcgtcccagttggagatctgcacaggagatagatgctccagccaggctcgggcggcgtcggagaggaacaggggaaggatgcagatgatgaggttgtcatcgtccgtcccactcagctggcaggccagccggtagtccgcgagccacagttccggcttcgactcccccgagtacttggtgatggtagtcggggttcagaaccaggtcgggaacggtgcccgttgtatggcccggctgaaagcttgcggactgggtggttcgggcgaggggctccgatcctccacgctgtcgtagcgtcccccacgcctggggtggtagcctcgacgcaccttctcgtcgaggtgggcttgacggtcgcggcggtgctcgttgccgaggcgtcttggggccgcaggcgctgtgtcccgcgtgcgcccggtgtggaccgaggcttcccgcatgaatcgggaagtcgcagcgcgatgctccgggggtacccctgccttcgggaggcagagctctcggcccgtcggaccgcgacatcctctaggagatttttgagctctccttggatacgccacccctcggtggtggatggtttcggcatcgctcggagtagtatcgctgctgcagccaggttctggccgaccccactggaagccgggggcagcctcgccctggcatcgtcggtgatgcggtgctggacgtcctgggccagatgacgcgcttctccagccggtgctcggcctgcccactcctgcccgatattttgccgaagctgcacaagttgtcctgcttcctcgtcgagcctggcctgtacctcgcggatttgctcaagccgtgcgtcttgaccccccgcagggactgggaccacagctagctcccgaaggatgtcaacgcgaggcgcaggcctagggggatcaccatcctccggcataccaagatggttgccttcgtcaagaccccctagatcgacgtggaagcattcgcaccttgggccacagtcctcgtcgccgaggctgtggctgctatcggagcaatcggagaggcagtagtcacatgcggtcatgaagtcccgcatgacactggggttatcgagcccggagaaatcccaaccagagtcaggctcgtcatcttcctcggaacccgggggcccataggtcgagacggccgtcagtcggtcccaggttgaccgcatatgataccccggagggtttggacatgcctttatgaaagcgtccaccgaagcgggatcgcttggtgggtcacaactgaatctaaaaggcatgggatgggaaacggacggtacctcttgatcgacgggtggtgacgaagtcgcgtcagggacggactgcaccgttgtctcaggtacgaggttaacgcccaggaagtccttcgcgagcgtgctggcgtcatccgtctgcttggggttggcgtgttgcggggaaacgacgcttgtcttcgtctcagacgcgaggtcaacgcccgacgtgtcccccgttggggcgtcggcgccgtcgactcgctcgacagccgacgaggtgccgcctcctgattggccatgcctaccccgcctcctcctccgtcagcggggaaggtgacgggacagacccggatatcgctcttccgccacgtggggaagacgtcgtcgattccgccgccgacgggcgggctgacggccgccattgtcgttgtcgcgcggcggaggaaggagtgtcatgtcgtagctgccgtcgagggacatgaactcaagactcctgaaatggagcaccgtcccgggttggagtggttgctggagactacccatctggaacttgacgggaagctgttcgtcaccatgcagtaggcccctacctggcgtgccaactgtcagcgtttcgaccccggggggtccctggaccgacgagtaaactgtcgctgcgtgtcccattccagatgggtcggcacgagacgaaacacaaaggggggaaaacagcaaaggggaacccgtggccttcgtgttgtcctgtgcccagggcggatgcgcttgcagtagggggttacaagcgttcgtgtgggagagagagagagagagccttgtgcgtcagcccgttctcccgcgcggccaaccctctcgtacgagagccctagaccttccttttatagacgtaaggagagggcccaggtgtacaatggggggtgtagcaatgtgctaacgtgtctagcagagaggagccagagccctaagtacatgctgtcgtggctgtcggagaggttttggcgccctgttcatgtgatgtcgtggccgtcggaggagcgtttgagccctgtggaagtacagctgtcggggctgtcggatccttgctgacgtctccttgcttccataaggggctgagagccgccgtcgtcacggagcacatggggtgccatcattacttgtttaccggggcgagccagatgggacgtcggtcttgttccccgtagcctgagctagctaggggtagggtaatgatggctccccctgcgacgtggtcggtccgagcccgaggtcgggcgaggcggaggctcctccgaggtcgaggttgagcccgagccctgggatcgggcgaggcggagtccgtcttccgaggtcgaggctgagtccgagccctggggtcgggcgaggcggagtccgtcgtccggcgtcgaggttgagcccgagctctggggtcgggcgaggcggagcttctcatggcgcccgaggctggacttagctgctgtcagcctcactctgtcgagtggcacagcagtcggagcagggcaggcggcgctattttcccgtcaggtcggtcagtggagcggcgaagtgactgcggtcacttcggccctatcgactgaggagcgcgcgttaggataaggtgtcagtcgatccttgcattaaatgctcctgcgatacggttggttggcgtggcgatctgtccaaggttgcttctccgcgaagcctgggcctcgggcgagccgaaggtgcgtccgttgcttgaggggaccctcgggcgagacgtgaatcctcctgggtcggctgcctttgcccgaggctgggctcgggcgaggcgggatcgtgtcccttgagtggacggagccttgacctgaatcgcgcccatcaggcctttgcagctttgtgctgatgggggttaccagctgagattaggagtcttgggggtacccctaattatggtccccgacatgtttacttacaaaagctccaccaagcttgtcgagcatccaatgcttgggcgcattgagcctcttcaagtgcttcttcaatcccctagcctggattgcaaaataataatgatcaacaaaagcgcaacagattccagtatggcattcataggtgactcatccagattgcattagctgttaaaagtaacagcaactacacactacttgaaaacaaaagacccttttcatacatgtctatctctattacttatatatgagcagtgccatcgtcagcacctcctgtatgtatacctaggacgacatcagctggcgaggggcacggggacgcacgggcgtcttggacgggctcaccctaaaaacacactagaacgactctgttatccaaccgcccagaagagctccttcctcaatgcaaagcgtaagaagatcagttagagttttaccttattggcaaggatcccagtaccacaccgctacagtgagagcggcagtagcactttctgccttgaaaaaaaattgaggcccagtcttaaaacaactcgcagaataataaggcatttgaacagcagaccaaacaactagcagaataaaaaagaagctacgcaaatttgaaggcgaaggtatgcttagctgaccatcacgaatcccagtttcagcccatggagcgggatttgttgctcatgtctgcctttctgtccttttagatagctaatgccaatagttcatgcaaaactattatcaactgttccattgtacatgtataatacttggaaataaacacagccagtagccaccaatacccattccttatgccaaatttgtgacatgagatggaaatagtacatcaataaccaaacgaggggtgagcatagaaatttaacatccaacatcaaaacttgcaaaacttggatgtttgagtccacctctcgagcctaacggacgtgaaatcgccatgacctggcagcctttgcatcaaaaaataactccagttctatagtaaatgtaaccatgtgtgcatacgtaccttgcagttctgtgcggcctagtacttggtcacctgcacaaggtacttgtaacacccctggtgttactgcaactaaaacttgagcatagcatcataaacattggcattgcatatgtttgacacacctagagtgcattcactaggtaaaaatttcaaacaagttgtattgttttagtgttttgcaaatagaaccctagatagggaatttaaccctaaatagggattaaaggggtaagatataacccaaattgagaaaacctaaaagctctagggaaatagtcatcaaatattctcaagaataaagttgaaccacatttatacccctcggataccaaaaaccctaattggaaccctagaaaaccctaaatccaaaccctaggggcttatgtgcaaaattcgaccacttttggactaaagtgcaaaaaccaagttaaataagtatcttaagtcatttgggtcactcatatgtgaatttacaagccaaaccctaagttttggcctcatttgcaaaaaggaccctatttgaggttttatactaagtctgaaaacagtgttatgggctcaacttttgagccttgtaacttttaaatcatagggtttttgccctaggtcaccacattaaaattatagcccaatcataggagaacaactttgcttaagagtgtgagcatagttttaagaaaatattggagataattgagcctgaagttggactgtcagactgcttgaaatctgaaattcagattaacagtgggatgacatgaacttagggcttaattttaagcaagattcagtgactttttgtgggagcacattgtagcaaagttatagctggattgtagctctacaactttgctgtaggtcactggatgagttgttatttgaaattgagagaaaactgggctccaaacttgactgtcaggctgtctgaatataaatctccatggtacagtgctaccagggagatcagaccaccagcgcggcagtctctcaccgccgatgactgatcttcgctgagattcacgccgccgccgttgcgattcacgtcgccggtgaccagataagatcgctcggtaaaggcatgcgctggacggcactccggtgaacccccagtacttcccctctaccgtgcggcttgagcagataagcccgctggggatccccgtcgctcggccttacgccacgtatccgggcacctctgtcgcatcgccgtgactccccactgttgtctcatcattgccggtgagcccgccacggcggtggacacgaaatcgcgaagccgatgatcttccttatctccggccgcccacactgtccactcaaattaagcgccaccgcccctgggatctataaattgaccctgcagagagcttcacaacatcatcacccacccagccaccacgtattgctagcaattgttcgcccaagctcgcgaattttgaattcgccccaaatcaattctccgccacccgaaacccaacctcactgcggccagccttattctggtcagttcgtctccttctctccctcatttaagctttcccttaagtctacgatgcttgccgacccacacaatcgagctaggagccctttggtcgccgggaacgcgactgtcttgccgcgatgttcacggccaccgtggccagagcaagccattgggccatagatggaattaggttaggggaaatgctcgggctaggtccaatttgatgtccgccgctcgggaaccctagccgttgccccgttcggccggtgcaggcactcgccggagttcggctgggcgtgaacgccgtcgaggacctccctctgcgaagagttagaactacaggggcttctctgcaatctgtcagcgacacagtgtaatagtgatagaagccagttctgattagccaaaccccgaggacctctgtgcaaagtcgccagggcgcgagcgcgcgcgcgcgttttcccctagtactgggccggctgggctagaatcagcccaacactattcaatctttttccttttctttttttgtagagctttggaaatttgttaaaaattgtagaaaaatcctaaaattgtgaaaccaattttcctaggcttcttattttccatagaatttaataaaaatagttgtatgaattttaggttaactaaggaattttaaggtatttaaagtagtttaaggtagtggttctggatttttagaaaataaatggaatttccaaaaatgtccaaactttttacataagttctatacattatttagaggccttgggtagaatttgggttgatttggaccttgtttgatacttagaacctaaaacccccctgccctttgaactcctttactgactccggaaaccctaagttctcggagttccgtgaaggaaagttgtattcaagacttagataataaatctttattatcttcgcactctcatgagcattacatggcattcattcttatatatatacctatatggttatatttagaaaatgaagaagagattgaagtgaccaaagagaagacaccaccacctacggattctcaggccggcaattgtttctacttcgatatctgcgggactgagcctgactcacctactaacgaaggcaagccccggtgcatttaccacctccttgatgcttttaaaatctttctcacttgattgctgcattaggtgataggagttgaatgcttaaacaattcctgcactaccttccttgaatttgattaccttccttgatcacccgttttacaaaaggattttgatgctttgccttgctctagaaaaacaaaaggatttgttttacaaaagatgtttggcaaaagtgggagggttatttttgaaaataaaacttgatggtgaatctgtcaaaggccttgatggattcaacatcggaaaagatgtacctctgccaggtaccaaactttgggtttgaaatgattaagccgagaccgggcgggtgacttgcacgagaaaggagtctcggtgtagtgtctccgtctgagtcgattaaggaccgtctcgatgtaggcctgctgaccggggaccctttaactggtcacatgcctcgtcatgggtaagccttgcctcgggcagactaaggccagaataagataacacgaaatgggcgtggagcggtggcgggagtagcgtgtaccctccgtggcaagaggctggacggtggtgtatctgtgctctcggtttgtgtgaacctgatctggtcttaaaaaccccagtggcgggttgacatatgcaagggttaagtgctacatatgtcgtgtgattggagatcctcagctgagtataatcgattcggatcgccgtaccttcgcggttatgaagacttggtcactgacttacacgtagcattccactaaagatgatggttttgttaagaaattggctagtgcaggacaagtgatttgaactagggtagaaagaactctagttacaggtaattctacttaatttgacaaataaaactggatttttaaggatccactttagtaagcatttctgcaaaacagagtctttgattattgaaaagccttaccttgactcccttaaccagcatacccttgagagtcttttctttagtcgggtaagacttgctgagtaattccatactcagggtttatccctccgttgtttttaggtgaggaagcgacaaatttttattgcttctgctccaaggtggttcccaaggaagaaaaacaagagtgaagccgcgggaggacttggtcctccatataggacttttgtttaaaaactatcgggaggagtttttgcctcccttggtattgtaataatattactctgcactcctaggataactctggtctgtaataagtaacttgatcttactttttaaataaatgtaagttatgtaatcgcttctgcatttctatatcttcgatgttctgtaatgtctgcaagacgggtgaaacgttcctggaaaggtaagaaagaagataccgaacttgtgaagtaatttaggaacatctatagggtgtctgatgtctgttggacaaggacaactgtaggtgggcttaattacttgggaggttccgtcacagctggtatcggagcgtagcccttctttgcagatattatgaggcatcttcaaaaagattttctaaaagtcttacctagaaactctcttcctttcttacctaagtattctgaagagtctatcttaaagaccaggtagtaagagtgcaacatatagaaggtgtgaatcaactaaggttgattctgtaattatacatgcatcatgctaagaaccatactaatcaaattttcccccttagaaaatgccgccgcgcacaaggagaacaacgcgcaaacatactggaccgattggtgtgccgagtcaccagctgaccccaaggcatgataatagtagtagcggaagcaatgatcctataggggatcttgaagctgaagtaagtcgactccaagcgaaactccgccgcagaacgactatctgggtcatagatggcgaccgcataaatgagttgagaagagatatctgccatctgcgagatcagctcgcggaccgggatttggcacttgactgggttgttcaatcccgttcgcttgcatgggacaaggagcaaaaagctcaagctcgagtagccgagctcaacttggctgttgatgaactgcagacatattgcaataccttacatgaagagattcatgtattatattcgcaactgcatcccagtgagcctacgaatcctggtgagtcggaagccggaccctcgcatgttgcgggacacgcgcttggtggtgagttagacctttttcagccccctccttctatgaggctagtcgacgaatggtctcccacacccgacgacgaggccgccaaaagcaacggaaagcaggaataatggggtagtagaagtagaagtagtgtattgtataacaggttgctctaatgtataatattttgtactattgcataataggttgtgctattgtataataggtaatgtatcctgttgtaaaaattcgagtctgtacattactctttttggtaatgtaaaatggatggtttttccttggcatatcatattgttttccaaatgttgttgccacagatgccttccaagactcgagcacaggacggagctagtacctcctgtgggagggagtctaccccaaatccacctcctgttcctcccacactggccgaggcgattgtggccttggtaaatgcaaccgcggataatacccgttttcttagagagatggcgggtcaacaattgcaacaacaaggtgggcggggttatcaacagggcccccgtgaaacctcttacttggacttctcagagacgcgaccaccgctgtttgtcaaagccgaagacccgttagaagcagatgaatggcttcgtgtgattgagcaaaagtttggactgctgcgatgttcagaaacccagaagcctttattcgcagcccagcaactgcgcggacctgccagcacttggtggggtaattttgtggccgttcaaccggccaatcattagataacttgggaagaattcaaggtggccttccgcgagcactatataccagaaggtgttcttcacatgaagcaagaagagtttatgaagctgaaacaaggaggggatactgttaaccagtatctcaataagttcaatcatttgtcacaatatgcaatcgatcaagtgaacactgatttgaagaagaagaattgctttatgagaggattaaatgatcgactgcaaaggaagatggcaacctgcatagatcttacttatggaagagctgtcagtacagcactggcagtagaagcgaagtatgcaggcgctggtaaatccaagggttttggaggtgacaggtctagtcagggcccggtgaacaggcaacggttcgtcatccggccttctaaccagaatcgttctttcgctcgtccaccctcctttccttttaagcagccagtctttattcgtcccaataatgcccctactacatcaagtcagccgggtgccccaggcactcgattccctgctttacccagctcgtcgactggatgtttcaattgtggcaaatctgggcattttatcaaggattgcccttatccaaagcagaaccagtcaaataatcagcaaggatctgggaattcatctcaagccaaggaaaataatatgggcaaaaatacaaagaagacgggacgcatatattatacgcaagtggccactacaccggacggtgagccggtaatgatgggtacgtttcttgtggccaatcatcccgcagttattctctttgattctggtgcttcgcatacattcatcagcaagaaatttgtggagcaacattgcatctcatgccatgaatcaaaagaggggtttaaaaattcactcaccagggggacaaatatttactagagaagtggcctatcaagtgcccgtaaccttggccggatgggactttcctactaatatgatcattctgaaaggccaagatatatatgtcattttgggtatgaattggttagccagacataaagcaactctcaacactgatcagagaattatcaggttgagtcataaccaggaagaaattcttttgcctatccccattccaaccaaagctactggcagagcttatgaagccattataccggaaatcaaggatattccggtggtatgcgagtttcccaatgtctttcccgaggatttgcccggactgccacctgaacgggaggtagagtttgtaattgagttgaaacccggtacggctccagtatctagaagatcgtaccgaatgcctcctaatgagttggcagaactgaagatccaattacaagatctacttgagaaaggatttatccggccaagctcatcgccgtggggttgtccagccatattcgtcaaaaagaaggatcaaactttacaaatgtgtgtggattatcgacccctgaatgaggtcaccatcaaaaacaagtaccctcttccaaggattgacattttatttgatcaactgactggagcaagggtattttccaagattgatctcagatcgggctatcaccagatccgtattcggcccgaagatataccaaagaccgccttcactacgcggtatggattatttgaatacctggtaatgtctttcggattgacaaatgctcctgcccacttcacgtatttgatgaactcggtatttatgcccgagttggacaagtttgtggtagtcttcattgacgatattttgatatattccaagaatgaagaggagcacgcccaacatttacggatcgtgttaacgcgcttgagagaacatcagttatatgccaagtttagcaaatgcgtgttttggctggacgaaattcagtttctgggacatgtattgtctgccagggggattgcggtagatcccagcaaagtcaaggacattttggagtggaaacccccgaccactgttcatcaggtccgaagtttccttggactggctggatattaccgccgattcataccagatttttctaagcttgtgaagccaatcacaagtttattgaagaatgatattaagttcaattggtcttcaaagtgtgatgaagcttttgaacaattgaagacattagtaaccactactccggtattggctcaaccggacatcaccaagccctttgatgtatattgtgatgcatcaggcagtggactcggttgtgtgctaatgcaagaaggccgagtaattgcatatgcttcaaggcagttgcgccgacatgaggaacattatcctactcatgatctggagttagctgtggtggttcatgccctaaagatctggcgtcattatttgctgggtaatgtctgtcatatttatacagaccataaaagcttgaaatacatcttcacccagtcagaattgaatatgagacagaggcgatggctcgagctaatcaaggattatgaattagaaatccattatcacccaggaaaagcaaatgtagtggcagatgcgctcaattgcaaggcttcctgccattgtttaacagtgaggacttctgacattacattatgccaggagatggagaaattaaacctgggaatgattcaacatgggacttcaaatcatttgaagctggagtcaatcatcatacgaagaataattgacgcacaaaaagatgatgagggtatgaagcacatacgtgagaagataatggctggaacagccaaatgtttcaaagaagatgatcaaggtgtgatatggttcaataaccgcatagtggtgccgaagaatgaagaactccgccagcaaatcttagatgaagcacatcttagtcgctattctattcatctgggaagcactaagatgtatcatgatctaaagcagcactactggtggacgaagatgaaaattgaaattgcacgctatgtggctaagtgtgacacttgcagacttgtcaaggccatacacatgaagatagctggtccattacaacctttgccgatcccaacatagaaatgggaagatattagtatggacttcattgtgggattacccaggactacaaaagggtatgattctatctgggttataattgatcggcttacgaaaattgctcactttctaccggtcaagacagatcacccggttactgtctatgcccatttgtacattgctcgtattcttagtctgcatggtgttccgaagacccatagtgtcggatcgtggacctcaatttgtagccaagttttgggaagcacttcacaaatccttgggtactaagttgctccatagttcggcctaccatcctcaaaccagtggacagactgagagagtaaaccaaatacttgaagatatgctgcgggcatgtgttctggaatttccacaaaaatgggatgaatgtttgccgttagcggaattttcatataataatagctatcaagaaagcatcaagatggcaccctttgaagctttatatggacgacgatgtcgtactccgctaaattggtctgaacctggtgaaaggtacttcttcaggcctgatatggtgaaagagactgaagaaagagttcaaaggataattcataatttgaagaaagctcaagctcgtcaaaagagttacgtagacaaacggcgaatgcccttatatttccttgaaggatactatgtctacttaaaggtttcaccaatgaagggagtatcgcgtttcggagttaaaggaaagcttgcaccataatatattggtccttttcttatcctggaaagatatgggccagtggcataccgacttcagttacccgaaaccttgtttgctgtgcataatgtgtttcacgtgtcccaattgaagaagtgtcttcgggttcctgatcgaaccgttgaagtgacagatgttgtccttgaaccggacttgacatattctgagcaccctattcgagtcttggatcaaaaggacagggttacccggagaaaactctcaagttttataagatacagtggaaccaacattccgaagatgaggctacatgggaaactcaagactttttagataagaatttcccaggctttttagcttcttgtaaattgtaaagcctgtatagctgttgtaataaaggagtgattccaaaaccacccctgccttgtaccagaaataaggaaataaaagtatgtcgtgtttccttttccattacttaccctaggacttttaatctcgggacgagattcttttatggggggaaggatgtaacacccctggtgttactgcaactaaaacttgagcatagcatcataaacattggcattgcatatgtttgacacacctagagtgcattcactaggtaaaaatttcaaacaagttgtattgttttagtgttttgcaaatagaacctagatagggaatttaaccctaaatagggattaaaggggtaagatataacccaaattgagaaaacctaaaagctctagggaaatagtcatgaaatattcccaagaataaagttgaaccacatttatacctctgggataccaaaaaccctaatcggaaccctagaaaaccctaaatccaaaccctaggggcttatgtgcaaaattagtccacttttggactaaagtgcaaaaaccaagttaaataagtatcttaagtcatttgggtcactcatatgtgaatttacaagccaaaccctaagttttggcctcatttgcaaaaaggaccctatttgagattttatactaagtctgaaaaatagtgttatgggctcaacttttgagccttgtaacttttaaatcatagggttttttccctaggtcaccacattaaaattatagcccaatcataggagaacaacttttcttaagagtgtgagcatagttgttaagaaaatactggagataattgagcctaaagttggactgtcagactgcttgaaatctgaaattcagattaacagtgggatgacatgaacttagggcttaattttaagcaagatccagtgactttttgtgggagcacattgtagcaaagttatagctggattgtagctctacaactttgctgcaggtcactggatgagttgttatttgaaattgagagaaaattgggctccaaacttgactgtcaggctgtctaaatataactctccatggtacagtgctaccagggagatcagacagccagcgcggtagtctctcaccgccgacgactgatcttcgctgagattcacgtcgccgccgttgtgattcacgtcgccggtgaccagataagatcgctcggtaaaggcatgcgctggacggcactccggtgaacccccagtacttcccctctgccgtgcggcttgagcagataagcccgccggggatcaccgtcgctcggccttacaccatgtatccgagcacctctgtcgcatcgccgtgactccccactgttgtctcatcattgccggtgagcccgccacggcggtggacacgaaatcgcgaagccgatgatcttccttatctccggccgcccacactgtcggctcaaattaagcgccaccgcccctgggatctataaattgaccccgcagagagcttcacaacatcatcacccacccagccaccacgtattgctagcaattgttcgcccgagctcacgaattttgaattcgccccaaatcaattctccgccacccgaaaccgaacctcacctcggccagccttattccggtcagttcgtctccttctctccctcgtttaagctttcccttaagtctatgatgcttgccgacccacacaatcgagctaggagccctttggtcgccgggaacgcgactgtcttgccgcgatgttcacggccaccgtggccagagcaagccattgggccatagatggaattaggttaggggaaatgctcgggctaggtccaatttgatgtccgccgctcgggaaccctagtcgttgccccgttcggccggtgcaggcactcgccggagttcggctgggcgtgaacgccgtcgaggacctccctctgcgaagagttagaactgcaggggcttctctgcaatctgtcagcgacacagtgtaatagtgatagaagccagttctaattagccaaaccccgaggacctctgtgcaaagtcgccagggcgagggcgcgcgcgcgcgttttcccctggtactgggccggctgggctagaatcagcccaacactattcaatctttttccttttctttttctatagagctttggaaattttttaaaaattgtagaaaaatcctaaaattgtgaaaccaattttcctaggcttcttattttccatagaatttaataaaaatagttatatgaattttaggttaactaaggaattttaaggtatttaaagtagtttaaggtagtggttttggatttttagaaaataaatggaatttccaaaaatgtccaaactttttacataagttctatgcattatttagaggccttgggtagaatttgggttgatttggaccttgtttgatacttagaacctaaaacccccctgccctttgaactcctttactgactccggaaaccctaagttctcggagttccgtgaaggaaagttgtattcaagacttagataataaatctttattatcttcgcactctcatgagcattacatggcattcattcttatatatatatatacctatatggttatatttagaaaacgaagaagagattgaagtgaccgaagagaagacacccccaccttcggattctcaggccggcaattgtttctacttcgatatctgcgggaccgagcctaactcacctactaacgaaggcaagccccggtgcatttgccacctccttgatgcttttaaaatctttctcacttgattgctgcattaggtgataggagttgaatgcttaaacaattcctgcattaccttccttgaatttgattaccatccttgatcacccgttttacaaaaggattttgatgcttagccttgctctagaaaaacaaaaggatttgttttacaaaagatgtttggcaaaagtgggagggttgttttcaaaaataaaacttgatggtgaatctgtcaaaggccttgatggattcaacatcggaaaagatgtacctctgccaggtaccaaactttgggtttgaaatgattaagccgagaccgggcgggtgacttgcacgagaaaggagtctcggtgtagtgtctccgtctgagtcgattaaggaccgtctcgatgtaggcctgctgatcggggaccctttaactggtcacatgcctcgtcatgggtaagccttgcctcgggcagactaaggccagaataagataacacaaaatgggcgtggagcggtggcgggagtagcgtgtaccctccgtggcaagaggctggacggtggtgtatctgtgctctcggtttgcgtgaacctgatctggtcttaagaaccccggtggcgggttgacatatgcaagggttaagtgctacatatgtcgtgtgattggagatcctcagctgagtataatcgattcggatcgccgtaccttcgcggttatgaagacttggtcactgacttacacgtagcattccactaaagatgatggttttgttaagaaattggctagtgcaggacaagtgattgaactagggtagaaagaactctagttacaggtaattctacttaatttgacaaataaaactggatttttaaggatccactttagtaagcatttctgcaaaacagagtctttgattattgaaaagccttaccttgactcccttaaccagcatacccttgagagtcttttctttagtcgggtaagacttgctgagtaattccatactcatggtttattcctccgttgtttttaggtgaggaagcgacaaatttttgttgcttctgctccaaggtggttcccaaggaagaaaaacaagagtgaagccgcgggaagacttggtcctccatatagaacttttgtttaaaaaccatcgggaggagtttttgcctcccttggtattgtaataatattactctgcacttctaggataactctggtctgtaataagtaacttgatcttactttttaaataaatgtaagttatgtaatcgcttctgcatttctatatctccgatgttctgtaatgtctgcaagatgggtgaaacgttcctggaaaggtaagaaagaagataccgaacttgtgaagtgatttaggaacatctatagggtgtctgatgtctgttggacaaggacaactataggtgggcctaattacttgggaggttccgtcacagtactgatggtactccggtggcgccatttacatctcaagcaatttttctcaaagttggattcttgatccctgcatatcgctggtcgtgacccgtgggcacggcgctcggatccggcagcagcagatcgaggcgaggccgcgagggaggagaagagccatgatggggggcatcagatcatcgctcaacgacagcagtatgggcgtcctcttcctgctggtgctcctgctggatgcgggcgtcgtcctcctagccgtgctcctagcagtagaggctccagtagcaggagaagaggcaggatgcgggcgtcgtcctcctggccgtgctcctactgggcggcgtgtcgtgctcctgctggtgctcgacgactggagcctgctgcttggtggtgctcggcggatgagcaggggatccgatcgggtaggggatgaggatgagatgactgatcggatcagatgggcaggggatgaggatgagtggatgaccgaccggatgagttggtttgctcggaagctgccggctgggggatggggattagatcattagtgtttgtcggtttgggtgtttgccactttgggtctttggcggaatgatgccttagtgggcaatgggctggcgcttggcgcctgggcacaatggacaatggtgggctggcgatttgttcattggtgtccatgtgtggatcgacagtaatggactaatggttaatttcggatatccaacgaattacccgcgggtgaggtttaatatccaaatccatgtctgctttatctcggatcgggtacgggtctaacccgcaggtcaaaaaacatatccatatcctgatccgtcgggtcgaatatccgacggatatcactatccacgcattaaattgccatccctagatgtgagacttaaggcatgtttgttcgctacctaagttatcacactttgcctaactttttcgtctaaggttagttattcaattcggacgactaaacttaggcaaagtgtggcacatttagccacaaaccaaacatgcctttaaccctctggtttagatcccgtttcgtttgagctgaatatacttattaaatgtctaaagcatagcctagagcctgtcatgtcatgaatcatgaaatgacaataaaacataaacaaaagcatagcctgggagtttggagcaccgcgctgggggcactgaagacgacggatcttgcctctcagcctcggcgatgggcgtcggacgcaggagatggcattaaccaccgctatattaataaaacgtattgtatatatgtgcaatacgtatataaagagaaatattcgtggcattaaccaccgcttatcaggttgcttataccgtacaaagagacgatattataactataaacatactgttgatgagaaaataaaaaataatcatatttcaaacgtataattttatttgaagaagattcttatttaagcaagattttttacctatatgatatatagaaaccgtacgaacatacagtcagctaactagttcattttaaattccaaaaaatgtttagttcaatctaatcagaatttactattgactatgttttttcacaatatgtcctatcaaaaatatcgtacgagacggttttatgtttacaagtttctagtatactcactaacatctaagacaattttgtatagtctagatgactctaataatatctttatttgagatggtttcatatacagaagtgtctaatatactaaccaaaataaaagacacttcttgtaaacttaatgcctcaaaaggtatatttatttgagacggttttcaacatcaaactgtattaaatcaatataagacatttccaaccatatatctgcctcaaaaaccttcttcattaaagacggatatccaacaaaccgtcttaccgtactcagcaccatatgataaaagacgcttctataaaatgcactgatatttgtcttaagatgtatgtcttaaataagcatatttctagtagtggatgtccaagacatccacagagtcattaacttaggtcataatcaaaattttgaacgaaacgcagtacgataaggccttcacaggcagctaactgagggtttgccactaatctagtctagaactcgtcgaagtcctgaaactcctgaaagtcctccacgttgccttcatcttctcctgagcactagttgcaatggggacaacctggggtttggtgtttttaagcaatggtgagtacacctcaacgtactcaacaaatgtcctgtttggctaaagtggactagctgtatgtggggttaagcttaaagcagttgcttttagttggttaggtatttattaccagtagagagccatgttttagcaataaccccaagttataaacccaaacattactccctccaagaggaaataccaagaattcataatcataatcaccatcattaagcatcatcataaaagtatccagagtaactctaatcaaaggagctcccaaggctgctcataactgtgagcatggctgatatactagcttctaacactctacagaggttgcacactttacccacaagtcgtgatccctttttgcctcaggtcgatcaaaccctcaaacactaccaaggtgagtcggcaaggtttcactacgtagctgtaacaccctgaattttggggtataaaaatttccttgctctatactcaaaatctaggtgttaccctttcctttattcacttttcttttccctttatcaaaacagtagagagttattttggttctatattggtgtgagctctagaagtgtcatgattgttgcattcatgctgctacatagtgtttccaagtgatgatccgaggtgaggacgagctgaccagtcgggcccagcgctagggcacagatgactgacaagtggggcccaggggcaagggcacccacgtgaagcgatatccagcgatctagaccgctagatcaaggctaaacggctaggattaggcgtcaggggggttaacagcactgcggccggcgctgctccatccgcagcggtgaagtcgccaaagacgagacaagcgcggaccccagggggtctggggtcgctggagttggccagaccggtgagggggacccgacgaactcgatggcagggttctggccatgagaacgggactggaggtgagtgaatggcggagggggcgctctgggcgggacacttattgtgatatcctggcccctgggatgggatgtcctggcccaaggcttaatagaattaatagtgtaatcataccaacaaggtgcatcttctttttcggaagcctatctcgaaagaacctccaagttaagcgtgcttggcttggagcaatttgggatgggtgaccgaccgggaagttttctcgggtgcgcatgagtgaggacaaagtgcgcacaaaagactcgtgttggtctgtggggacaatatatgatcctagacagctgccaggagtaagtaccgccggtccagggattagacggggtgttacaagtggtatcagagccgacactcgcggtttcacgggcgtgtgtgggctagggggttcgggtatatggcgcatggcacatgtgggcccggagtggtcacatggcatggcatatgacggcactagacacacagacgtggccaagaggggaggttcctggattggggttgaccgacgaggacgtcggtcttctaaggggggtggattgtgatatcctggcccctgggatgggatgtcctggcccaaggcttaatagaattaatagtgtaatcataccaacaaggtgcatcttctttttcggaagcctatctcgaaagaacctccaagttaagcgtgcttggcttggagcaatttgggatgggtgaccgaccgggaagttttctcgggtgcgcatgagtgaggacaaagtgcgcacaaaagactcgtgttggtctgtggggacaatatatgatcctagacagctgccaggagtaagtaccgccggtccagggattggacggggtgtgtaacaccccaggtgtttattttccgctcaacaacgagttcggatttaagcacgcaatatcagtggataaaacgaattttaaattttaatcattgtcgcttatcgctattttaatatcgcatcggtgtcgtttgtcgcgagtgcgacatcgtttttatttttttatctgtccgggctcttcctaaattttcgtaatgttcggaacctagctgttccgaaaatcggtgcgtccgatgagtatttaaaatccatcgctcgcgcgaacacaaattcggaagcccgaactcactcgaatgatcttatttcgagcaaattaatttgaacttgacgactaaaatgttcagggtaaaataatctgaatcgcgcattgtctgagaaagatcgtgcgcggggatatgatctaatttgttctttagcccgcaatgtaggataaccaaatcaactgtgttttggtgacggataagtttttatctgatttcaattaaatgtaacaccgattaaaacattgtaactaaaatcatttttaattttagtcctcttacatctttccaaattctagtcccaatctccagctgataattgtatttttattcaaatttttgagtaaaagaaaacgaaggaagaaaatatctgcaaccgctcttctctctgattttatccaccgcttttcccttccatatctgaagtcactagcctggatattttctccacgtagttctcctcttcctcacgtctccttctctcttatccattggacgctagctcgctggaaaatctcacgcacgtctctcctccagccttacccagcgaccagcatttcttccatccatcagcatccaaaggcagccggctgccggctgtgctcgtcggaccctccgagcacctctgtgcccgacgacctgaccaagctcgtctccagcttgcgtccatcctgtgctcagtttccatccactagcaccgtgtctctggtcctgctcgtcgtggacatcgtcggctctagttccttgctcgagctcgccctttgcgcagaccgcgtctcccctcaccttgccgcggtcgggctggccgtcgtcgtcagcttgtgtccatgccgacgaatttgtcgaactgctcactgcatctctttaatctcgtcgcctgatttttctgtaccgcgccgcgcaacccctagaaataaaaatcacgccgccgagcgctcctatccttatcccgccaccgcccttggtctcctacaaatctccagcgcgcaggtttcttctccacgcacgcccggcagcaagccgcagccgagcagctccttcccatctcccctctgctcgctggctgaatccccagccgctcggctctgcttttctcccatggcgcggggttccctgcaggctgctcgcggtatccatctcctctgctcctgctcgtccgtccctgagctcctgtgccgcggcacctctgttcggccacgcctgatcggatttcttgtgccgtggcttcccctccgagctcgcccagctctattgccgcgcccatggccggcgctccctgcttggttccgtctgtcgcgccgtcgtcttactgctcgcctttgcgtcgcgcgcatagcgttctgttgttcttgcacgcgcgaagctctttgctcgtcaacgcttcagcctggatttcgctttgtcgcccagctcggctctacatgactacatctcccatgactgtctactctagctcgccgtagttcctgcgcgcgtcgagttttctctactctagctcgccgtagttcctgcgcgcgtcgagttttcgtgtggagctctctgctcacgcgtagctcgctctttctttgttgccgcgcgcacgaattttatctgctcgtcacagcgtgtcgagttctcacaccatcatcgcttctgtcgcaagctcgttggtcacagttgtcttgaccgcgttaactcgcgactgtggtcgtgttcatcgaattcgccaactctttgttgccgatttgactgtcgtcgcttcgcgtgttgtcgagccgtcgttttttcctgtcttgtgctcgcacggtttcctgctcgccagcgtgccctctcggctcgctcggctttaatttccaatcacgtcgtcgatctcgtcgtttgccgtcgagttgtcaaacacgtcatctccggctcgatccccacctcaccagcttaccccagacttcaatcgaaggtcatcgtcgctcgtgcgtccccaagaaaacccaagaatcgggtgaagacgaagttagcagcgcgatattccctaagcgctcgacaaattgcgtggatcgaaaaatcactgccgatctcatggattcgtgtcaactgttgaaacggtaagctgatgaattgtttagaatagttcgatcgttgaataagttaatgtgttagtgcgaggctcattagggtgctcgataaattgcgtaagtcacgaaactctcgtcgacttcgcagttcttgcgattatcgagccaggttcagttatagcgagttatttcgctattccggtcacttagctgaattagtggaccgagtagaattttagtaggcatatgtgttgataaaatattttaatcacttataaagatgtagtataatttataaggcaagggattagttcagaatttaattaattaactgataagttgtgattaggctaattatatttcttgtgtatagtttgttgttcgtgatgtttgcgttaggttcgagaagcgtaatcattgcgcgtagtcgcatattaataactagtgtttccgtacaaaattgtacaacgcctcgccactaggtgtttaatacgctatcgtatagcactatttagatttgtgctattcttgtttatatgcattcatgtgcatcgtgcatctcaattaggtacgataattgatcgcgtgatgcggaagacaagccaagtcgaccccaagcgcgggctaatccgcaggatgatgctgatggacaaacctgaaaatggtcgccaagtggacgtcgtctaacaacactaacctagtgttacccaggcaagccccggtgcatttgccacctcccttgatgtttttaaaatctttctcacttgattgctgcattaggtgacaggagttgattgattaaacaattcctgcattaccttccttgatcttgattaccctccttgaaaacctgtttttacaaaaaggttttactatgcttagtattgcttagaaaaacaaaaggatttgttttagaaaagatgtttggcaaagtgggagggttgttttcaaaaataaaacttgatggtgaatccatcatggctatgatggattcaacatcggaaaagatgtacctctgctaggtaccaagtttttggttaaaagattaagctaaggccgggcgggtgacttgcacgggaaaggagtctcggtgtagtgtctccgtctgagtcgattaaggaccttgtcgatgtaggcttgatgatcgaggaccctttaactggtcacatgcctcgtcatgggtaagccttgcctcgggcagactaaggccagaataagataacacgaaatgggcgtggagcagtggcgagagtagcgtgtaccctccgtggcaagaggctggacggtggtgtaactgtgctctcggtttgcgtgaacctgatctggtcttaagaaccccggtggcgggttgacatatgcaagggttaagtgctacatatgtcgtgtgattggagatcctcagctgagtataatcgattcggatcgccgtaccttcgtggttatgaagacttggtcactgccctacacgtagcattccactaaagatgatgggtttttgttaagaaattggctagtgcaggaccagtgattgaactagggtagaaagaactctagttacaggtaattctacttaacttgacaaataaaactggattttaaggatccacattagtaagcatttctgcaaaacagagtctttgattattgaaaagccttaccttgactcccatatacccagcatacccttgagagtcttttctttagtcgggtaagacttgctgagtaattccatactcagggttttatcctaacgaatcaagctgatcatcaacnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnggtcagcccagattgcttctgcgagcgcaccggctattgggtcttcctgtgttctgctagccgctggtgcagactctgagatgcatctcacatatttgctgggacttctcactcttctgactaccagcggcagatatgttgaggagtgggtccgtgtgttcaatgcgtcagtatggatcgaccccgatcaccagtggatgaggttccgctttgagcgagaggatgttacacttcatgctagctagattcgccagctgtttggattcaatgagtcatcgacttgtcttcatagcttgtgctatggtacctctgatcctcctcgtcgccctcacgacggagttgctccagctacagctcacatcgcggctttgttccgaccgcccttctcagatgggtcgcgacgttctccggcagatttcactacagtagccaagtacttatatcagctcatgagacggacgcttctgtcgtggatgggttatagagaggctaccactcatattcagctttggctcctcggtgccctgatctttcattcagagtttgatgttgttgacttccttatttgtgagatcgaggacacggtattggatggtcttcgtgctcggcgacagctgccaaatgctcattatctctgccacatcttcgcacagctgatccgaccaccatagttccagggcacccttgaggcctcacgcctcctatttggctcctaccatccagcccctgaggatccagtaccagtacctgatccagtgacagacattcaggcagaggatacaagtttccatcagtttgagacttagggcgcagcagttcctgacgatgatgatgatgatgatgatgatgattttgggattccgcctctgcctcctgtgcctccacgctcacatgaccatgaggcccggagttctcgtgctgcccctgctgttcctcctgccattgaccctgctctggctgcgatcctccagactcttactcagcagcaggctcatctggcagcggtgcaacagcagatgtccgagagaatgctatcgatgttttagactattcaggacagacaggacactctgcagcagcagcttttggcagacaaggctgagaaccgggccttcatgactcacatacttcagcataccggtgctcagattcctcctgttcagtctgcaccccctctagatcttcaggccgctgttgtgctagcccttcaggcaggaccccctctaccttcatttggtccttcttcctctccgctcctgccggtcaccctggttttctcgtcgccggtcatcagctccatcagcgctcagccgccagtgccaccagctcctgctgttaccactgctgttgtggcggtgtctgtgacctcttcagcttcggtagctcctgcagcacagcctccatccgagtcagtactagctccagcttctacggtagatcctggatccgaggctgactctgaccctcagctggcgtttgctcttctgccacgatcgtgatcggatgcgccccagccacctccttcctcttctggtctgtaggttcaggtttccttttggtgtttgacgccaaagggggagagatatgagagttgtgagagctagggggagttagggagttagtatagagtcattttgatgtaatatatgtgcttgatactctctgtactagatccacttttgtatgacgattttggctcacaaactctattatatgctctcgatgcttatgttgactgtgtgtgtattgtgttttcaccttatatgttatcaccagtctctagttcttgttcatcgatttgatttcacttttatatgaacaagaaacttacaatgtgtatgcactcactcttattattatgttacacactctttctgtcaaaaatttttgagtataactaaccatcttctctattgacagaaatttcaaaacaaactactctcacaatcttgtaggttgtcatcaatcaccaaaaagggggagattgaaagcatctaggcccctggttggttttagtgattaatgacaatgtaattttatatgtgactaacatgtgttttgcagaggcaaatggtaagttaggtcgcattacatgtagatgtactacaacggtgaaaacaatctcggagataagaacttgaagcgacggctaaagcgacaaaacaaaaagtgaaggtcttcgtattccgagtgtcaaggagttgcggacactcgtgatatagttaggtcttttattttgttttagtcgtactataaagaggggttgtcgatgagtagtttgaccaagagagttctagtgtagtgttggtgcatattcacactcacatatagtgctaggtgccactctagaacatactcacaagttagaacgaaaaccgaattgaaaaaacagcacaaaacagaaactagggtttctggctttggggcaccggactgtccggtgtgcaccggactgtccggtgcaccctctgccagtggggccagcctggcccaaggaagagggttccctgcgcacagaaacctgagagcgcgttgttcgcgagttgaattttagtggactgtccggtgtgccatctgcccaacggctagctgtcagaactagccattggagtcgaccgttggcgcaccgttggcgcaccggactgtccggtgcgcccatgtgcagcagattcctggtaatggctagttggtgggtgagggctatttataccccctccacccactatattgatggtcttgctacccacatttactcctacacattggtagagcattgcaagcaccacaaagcctagtgaggttatttgagaatcttaatcccgcatttggaccttattagcgctagcgagagccacctagagcatacaccgcatgcattaggcttctcttggtcaagtgaaagtctatggcttgttactcttggtgatcgtcatcacctagacggcttggtggcgttgggagctcggtgatcaccgtggagatcttgttggtgacccgactcaagtttgtaagcggtcgtgagggatccactgcgctggagtggcaaaggatcatctcgttgtgagcacttggttcttgcgaggaccaagggggagtgatacccttgcgagggtgctccaacgaggactagaggagagtgccgactcttcgatacctcgagaaaaattggagtcttctaaaccttgctttacattccgcacttaattaaaacattttacattgtgtatttgtttagcaagtatttgaaatattgtcttaacattgttgtatttctattattattctcttagtgatagttatcggggtgaagttggactcttgcttagattttaattagtgttgatttttagaaaagtccaattcaccctcctcttgggcatcgtgatcctttcaaaactcactcaattccgtctaatccacgtggattcaaaataaaacgaacagaccctaatacatgcgatccgacgctacaccggaactatcagtggtcagcttctaggcttcagcattatacgtactatgaaaatatgaatgcacttcaggtcatcatcaacaaccaaaatggatatagcaaatattcaggctcattatacttgaaaacaatagaattacattaaaaaaggccgaaaccgtgaggctggattaacaagagaaacggtaatggtacagtaattcatgaagtgaaggattttacatcaccaccagctggtgctgaaccttcccgttggatccagctaactgcccttggcaggagcatctacaaccaatacccaaagtgggttatcttacttatctagagccctggtatcgcaagcccaatatgcctcagggtcagggcaggaccaagaaatgtggtgaagttcacattcccaaggcaaccctacgtctcaatgccacctcgaagtatcatctagtaaaagcaaagttcaacagaaatgctgtgccagcaagttgtcttggaaccgacgtggtaaaatgagcatcgtttgatcactttgtttttcttctcgatgcaatctccgctgcccatgcttttcccaagtctgtctgaaatttgcctgcatgggaattaggtgcggggatatggttttgttacacaatgactctaatgctaatagcctaggctaagtttaccatccccatattcaaattccactctgcgaatagtgcaatctaagtgcaaaacagtgttttgggtgggtgaactgctggacacggtctaatacaatgtaaaaatgagatcaaacataagcacgtgataaaagaaaaccataaaaggcataggcatgtatcagttcatggtaaagaaaaccattataggtggtagtgtccagttttcaattagcaataatcattcaggcactaatatgttctgaattgctgatgaatgtttatattatctcaggaaaacatttttaagtgtaagaccaaaaaaatggcaacatccttctcagcttaaatgaactgttcaaatttatgtacaggatgctcatgaaaattgagaagagcaagatttatgtactggattgtcatgaaaattgagaagagcaagatttatgtactggatactcatgaaaattgagaagagcataacagaaagagaaaaatcacacctgctgttgattggaagaattcttcaaggtcccgtccttgctctgaaaattttaaaatacataggcgtaagtgtgatactgttaaccccatctatcaacaaggagttcaccaggtgttaagtgatagtacattgatcatatgtatcacttctcacacccagaaggccgtggagcaaattaaataatggtgtaagcacagatgggcagatctagggcggaggctgccacatgagtggggtcttgagatgggataaatcgagacaagcctcccctgcaaatgcagagaggctgtttcgaactggcaacatagtgacttagtgagactgccctcaccactacaccaggcctacccaatataagcacaaatgatgcaaagaaaaagatgtgctgtatttgaaatgtgaaatgtgagctgattttactatatacatttatttggttattacaacaagaatatttgatgaatgcatttaaatagttgtggtttgtactttatagctactgtgcatgggaaatgttagttcaaatattcaagcaccagtatgaactcacccttttcatactccagagcttgaagtatcatctcaacctggaaatataacagtgcaacaaaggattacagcatgcaaaggaaaaggaagaagtggagccatatgggttagggccataaatcataatgattgcctacattagttaaatatcctgccagttatatgcattgcctattgaatgatcacaagaactaccatctgatagcttcagacagacgttgcaatcatgccaccaacttgatggattgaaatatgaaactgtaccttgtcaaaatctttgacaaccttcgcttccaaagacgcattctcctcatactccatccaaagttcacgaatttcttgtgctgcaagacaacagcatgcagataaaggcaagtatttattatatataccatgtcaaagatcacatgaactctttagtctcgcctgtacagagaacatccttttatcctgcatgaaaaactgtttccaaaaggctgctaagatactttatttagttctaaaaggttcacttcacatgtaagggatgctggatctctccaatattttttaacgattaatgatatgaataatgagaacacaaccagaatactagaattctatgttgtgaaactcttagggaaaaaatgttggatgctatgatagccatttgagcataaataatttacgatccataatgcttcaaggtagaaaatcattagagatggaataatattatcaccatcaattacaatatcatgttcaaattccaaaactcatagtcatcaacatttgctgaatataaactcttcggttttggcttctacaaaaacatcccttatcttttcaacctccatttcaaaatgtagggcgtaaggattcaaaaaagtcaatgaaactagtcaaaatatttgtatatttattgcacaaagataaatctatagattcatatttcacatgcattttagtgagacattgcttttgtagtaattgataatatattgagttcatatattgcaagggaaattattggataaagcatatctttgaatgaaattctcaaacactaatacaccttataaaaagaaaaagagaagtataaataacagtttctctggaaataatctgagtgattttaagttaccaagagtttccttgacacctaactaagggatgtgaatactctaagaattatccaatacttatttaaactatgtatcaaaaaataagaacaaaagctgcccgctggatttctacaaaataattgccaggttatgatctgcttccctgatggaagtgaaaagtatcggatggaaaaatgaccatctaagaaataataataacagatgaatagcttttcaagggtaaaataaaatatgtatatgacctgcaagtactatagtattgtattcacaaaattcattggcatccacatattgttcttttttccttgaaactatggtactatgcacacataatgggatcattaagtctagactattgagtaatctagaaagatgatgccagtgtgcaatagcaccacattcatttcatatataactaaatcatgaaaagacaatttgaggcataagatgcctaattaactacagcataaaatgctaatgtatcacaattgcaagtttcagtattcacctcttgaaccaccaccaagcagctcgcacatatggtccaatgcttctttctccctgcggttcttctcttccttgggtacattatcagaaggggtgatgtcaccaacaattgctggagtaccaaaagaaaaaacaattgaaatgagtcaactgaacccacatcctcataggcagttagttccagaaacaggcaagctggcttaggaacagcagcaagagtccatatgagcggagggcaaaatcatgtgttcatttctaagctgagcatgcttctgaatgaaaataggaaaatgtgcacatagtttaaagttttacactttggctagcagaggtcaaagaaccaactaattggcacaagtacttgaacacacatcctacattcctactacaggtctccagtccagtggtctagttaccatctaccaacatctcaggtagtaataggctcgcatattcacaaaattgcatccctcatctcacacaaagccccaaaacttcagtgaagccgtctagacggaagtcttttgagaccataccttctgcaatgtcgtgcacaatcgccatcttgacacacctgtaattgaagggataaataaacagtgtatgaaaacggaaccgtaagaaggctaaatactgccgagctagacttgagagcgaaactgtcaggatcacctgtcgcggttgacgccgggtagatcggccgcgacgagcgccatgacgcccatccggtacatgtggtcggccaccgactcgggcgcctgcaccccgcgcttcacccaccccgccctcttggtcgtctgcaattacatccacaatctcatccatcgcgtcacatttccatccatctcaaccaagccggcccgtggaaatgcgaagcgactaaacaggggcgctcagtcgctcaccttgaggcggtagcagagcgtgaggaagtcgatggcgttggacgccgaaggggccggggcaccggcgtccaccgatgcggcgggggtcggggaggaagaggaggacatggcggcggcgaggcggtgggggagcgcgcggtgagccggggcgaaggggacggggtgctgtgggggcttggcggcggcgagggtggtggcgcagagggaggagagggaaagggctcggctcccaccacccatcgttattagctgaggccggagtaggcggaggagcggtgggcagcgcagggcaggctccgcggatggcggggtggtcgctcgcggaaccggcgcatgcccgcccgcgagcccgtggcccagcttgcgcggcgggcggaccgtggatcacgtggggtactgaggttctcctaatttgggccccagcgcacggggatcgatcgcgctagagggtcgatcctttcctttttcattttcggctgccgggcccattcggccaatccggattccggagtctgcaatgttgcggatagcccatggttggccaagaatgcggcccggcccgtgaggggtccacccccacgtggaaataacaccagcccatcaatttatatgtctttgagtctgaattttaacccagctaaatctgtcgagaacttacagcaagggaagagattaagcgctgtttggatcaaaatattagactcacttatccaataaaataggtaacacagaattttagatgatattatttacagagttgcgtttaatataggaataaaatagaggatacaataggggatcagttggagatggccttatactatcaaaaaatcttatgtgggctaatatcaaacgagaagctctagtcgtctatataacaaggaaatagttttttgtgcttctgcctcgacaaaaagagaataagccctccattgctgaggagagggttcaaggtctgaatttggaaattgcaccacagcaagtcctcccgccttgcctaattgtcttacatgataggcttcgtttccgttcgctgaataaagaagcacggtatgtcgtttttgaccgctctagacaattgtttagtagattttgttcaaactagattgttttctcgcggtcagatacatattgtagagtgatttcttactgtcagatacatattgtagattgatttatgtatacactagcatgttaaatcctgatgatttgacctgcttaatatatccaatctattacttttacttaaaaagccatcgatgtcctactaaccgcgggtcgtacgaatcaccccgatggcgaggctcgtgcgccagtcgcgtgcactacacacccaccccaccggtggcccacacgttgcgttcatgaatagatcggtcatgccggcttctagtcgtacactatgtcggcgcccccaactctgcgccttgatgtcacactgacccacgcacccatgccctgctgctggtcacgccatctcgagctgagatggttcacgctgcgtcagcccacggcgccaccccgcactgggtcgcgcttgctcggccagctggggcgcagctcgtcggcatatgcttcagccacgcctcgtcagcacgccctggaccggctcccgtgggtcatgcaatttatctatttaaatttctattattgataattagcacgcctaattaacctaaagttaattttgtgtgacggactatggttgaagacaacagaattgattcgtggagcttgtcctcaatggcaagaactaaccgacctagactaacgactgcaagtttcacctagaggcgatatagctaggaaaggagatcttctggtagggcccgaatgacacttgcctgaaacttcatgagaaagcaaaaattacgatcttcgtcgggcaccacatccatccaggcctgaagatggagtatccagaggtgaaagaccatatgatattgtggacagagctatgtgagtgtttcagtgtggagaagcatgtgatgctcccgcgggcgcaacatgaatgggccactctcgacttcaatgcagttgaggcttacaacactgtcatccatcgcattgtcgctcagctacatttctgtggccagatagccatagacttagagatgatcgagaaaactctccaaaccttctacccctccaatatggtgctccaacagcagtactgtagcaacaagtacacaaataatgtgacctcgtcaacatgttgcttggtgctaaggctcagaatgagcttctgatgcagaactactagaagcatccattcggcacgcggtcatgcataaagcacacgccaacttctagtcttaaaggaagaaaggtccctccagagaaaggggtcatgggcactgtaataatcaggggatgagagggggaatttttacgaagccaccacaaaatggcagtagagtagcaatggctatggcaaaggcaaaggcaaaggcaaaacctcagaagggctatgcaagctcctcaaagcatgccagtgaaggttgtttcaaagaaacacttgattggcatgtatcaggagtggaagaaacgcatagctcataggctcaccttatttattcatgcatctatacacgctatgattatagagcctatgtaacaccctgaatttgggggtataaaatttcttctctaatatctaccaaattcaggtgttaccacttttctcatctccgtagatttcctattttcttcctttctaatagagttttggttatatatttgggagatgtattttttttctttactatattcaaacctaggggagacatgaattgttgcatcatgctgagcttaaactttgtttttggttgatgcacatgtttgaaatattcaaatttgaatttgtggtttcgttggatttgaattcaatagagaaaataaaaataaaaggaactagaaattcagaataaaaagaaaatagaaaagcagcccagcctacgcacctgccctctctctccatctgccaggtgggcccgacctattggtgccgctcaccctcgcgcgcacgcccccgctctccctctgtgcagtgggcccagcccatcagcgctgaatcatttcctcctcacacgtgctcgtgcctctactctgtgggcccgccttgtcagtctcatcttccccgcaaccgctgctgacccgcacacgcactcacgccgaggaagccgaccacgttgcctacccacgcccccagctcccttttgagccccgcctacacccgctctccctccccttcctaatttcacccactctcaacctctctcgcgcttagccgccgccgctcaagctcgccggagaagcgcgccaccgcgtcgtctgcccggagctcctagcatcgtgtcaagcatccccgagcacactcctaaggtaaggaaccatccccgtgcccttcctgccccgattcttttccctctacggtgaatttgtgttcgctggagctctatcgcgctggtttgccgcgcccgctcggtgtccgaccgattcagccccgccccgtgcccgtgccttggccctaggcgtccctcacccctcaccgaagcttgtgctggcctcggtgcaccggattccgcctcctcacggtcgggattgctcaccggagtaaccccgacctgtggcagaacctcccaagttattaggcccacatgcacctatccttgtcccaaagacctcagaccccaaaaaacgtgcaccagataacttaacaggatctgtaagatctaccaaaggacatcggataaaccacttacaaccagaaccgcgagaaaacgaatcccaaatcacacacaccaatattgttgcagcgaacatcttactaccaaattttacaggttacaaaaattttacattagtttatcggagtgattacaaaagtataagtttgaaatatatatgctagctcaagggatcatcctcaataagaagtatagaagggttacttagactcataagaaggccgagcccaccggcacttaacaccatcaacaacagcacaaagttagaacctgaaaaacaacaaggaataaaaccctgagtatggaattactcagcaagtcttacccgactaaagaaaagactctcaagggtatgctggttatatgggagtcaaggtaaggcttttcaataatcaaagactctgttttgcagaaatgcttactaaagtggatccttaaaatccagttttatttgtcaagttaagtagaattacctgtaactagagttctttctaccctagttcaatcacttgtcctgcactagccaatttcttaacaaaaacccatcatctttagtggaatgctacgtgtagggcagtgaccaagtcttcataaccacgaaggtacggcgatccgaatcgattatacttagctgaggatctccaatcacacgacatatgtagcacttaacccttgcatatgtcaacccgccaccggggttcttaagaccagatcaggttcacgcaaaccgagagcacagttacaccaccgtccagcctcttgccacggaggtacacgctactctcgccaccgctccacgcccatttcgtgnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnttcagggattaaacaatgtcattttgagaaagactggatttgtagagcataccagtcggaagcaagtggcactcatcatccacacacgaacaaaaagacaacgaccgcccagtgaagatcctcccccaaagcaacagtcaagcatccctgacagaactcttaacgtaagtaagtaccttcaggcccttcctgccccgattcttttccctctacggtgaatttgtgttcgctggagctctatcgcggtggtttgccgcgcccgctcggtgtccgaccgattcagccccgccccgtgcccgtgccttggccctaggcgtccctcacccctcaccgaagcttgtgctggcctcggtgcaccggattccgcctcctcacggtcgggattgctcaccggagtaaccccgacctgtggcagaacctcccaagttattaggcccacatgcacctatccttgtcccaaagacctcagacggctgtgcatgtgcaccagataacttaacaggatgtgtccgattgccccaaggacatcggataaaccaatttcaaccagaaccgcgagattaagtcttgaaactcacacacggatacaaagtggtagcggaaatattattgacaaatttgacaggttacacaaatttttcatacctctatcggagggaatacaaaattctaagtctgaaatataaatgctagctcaagggatcatcctcaataagaagtatagaagggttacttagactcataagaaggccgagcccaccggcacttaacaccatcaacaacagcacaaagttagaacctgaaaaacaacaaggaataaaaccctgagtatggaattactcagcaagtcttacccgactaaagaaaagactctcaagggtatgctggttatatgggagtcaaggtaaggcttttcaataatcaaagactctgttttgcagaaatgcttactaaagtggatccttaaaatccagttttatttgtcaagttaagtagaattacctgtaactagagttctttctaccctagttcaatcacttgtcctgcactagccaatttcttaacaaaaacccatcatctttagtggaatgctacgtgtagggcagtgaccaagtcttcataaccacgaaggtacggcgatccgaatcgattatacttagctgaggatctccaatcacacgacatatgtagcacttaacccttgcatatgtcaacccgccaccggggttcttaagaccagatcaggttcacgcaaaccgagagcacagttacaccaccgtccagcctcttgccacggagggtacacgctactctcgccaccgctccacgcccatttcgtgttatcttattctggccttagtctgcccgaggcaaggcttacccatgacgaggcatgtgaccagttaaagggtcctcgatcatcaagcctacatcgacaaggtccttaatcgactcagacggagacactacaccgagactcctttcccgtgcaagtcacccgcccggtcttagcttaatcttttaacccaaaaacttggtacctggcagaggtacatcttttccgatgttgaatccatcatagccatgatggattcaccatcaagttttatttttgaaaacaaccctcccactttgccaaacatcttttctaaaacaaatccttttgtttttctaagcaatactaagcatagtaaaacctttttgtaaaaacgggttttcaaggagggtaatcaagatcaaggaaggtaatgcaggaattgtttaatcaatcaactcctgtcacctaatgcagcaatcaagtgagaaagattttaaaaacatcaagggaggtggcaaatgcaccggggcttgcctgggtaacactaggttagtgttgttagacgatgtccacttggcgaccattttcaggtttgtccatcagcatcatcctgcggattagcccgcgcttggggtcgacttggcttgtcttccgcatcacgcgatcaattatcgtacctaattgagatgcacgatgcacatgaatgcatataaacaagaatagcacaaatctaaatagtgctatacgatagcgtattaaacacctagtggcgaggcgttgtacaattttgtacagaaacactagttattaatatgcgactacgcacaatgattacgcttctcgaacctaacgcaaacatcacgaacaacaaactatacacaagaaatataattagcctaatcacaacttatcagttaattaattaaattctgaactaatcccttgccttataaattatactacatctttataagtgattaaaatattttatcaacacatatgcctactaaaattctactcggtccactaattcagctaagtgaccgaaatagcgaaataactcgctataactgaacctggctcgataatcgcaagaactgcgaagtcgacgagagtttcgtgacttacgcaatttatcgagcaccctaatgagcctcgcactaacacattaacttattcaacgatcgaactattctaaacaattcattagcttaccgaactattctaaacaattcatcagcttaccgtttcaacagctgacacgaatccgtgagatcggcagtgatttttcgatccacgcaatttgtcgagcgcttagggaatatcgcgctgctaacttcgtcttcacccgattcttgggttttcttggggacgcacgagcgacgatgaccttcgattgaagtctggggtaagctggtgaggtggggatcgagccggagatgacgtgtttgacaactcgacggcaaacgacgagatcgacgacgtgattggaaattaaagccgagcgagccgagagggcacgctggcgagcaggaaaccgtgcgagcacaagacaggaaaaacgacggctcgacaacacgcgaagcgacgacagtcaaatcggcaacaaagcgttggcgaattcgatgaacacgaccacagtcgcgagttaacgcggtcaagacaactgtgaccaacgagcttgcgacagaagtgatgatggtgtgggaactcgacacgctgtgacgagcagataaaattcgtgcgcgcggcaacaaagaaagagcgagctgcgcgtgagcagagagctccacacgaaaactcgacgcgcgcaggaactacggcgagctagagtagagaaaactcgacgcgcgcaggaactacggcgagctagagtagacagtcatgggagatgtagtcatgtagagccgagctgggcgacaaagcgaaatccaggctgaagcgttgacgagcaaagagcttcgcgcgtgcaagaacaacagaacgctatgcgcgcgacgcaaaggcgagcagtaagacgacggcgcgacagacggaaccaagcagggagcgccggccatgggcgcggcaatagagctgggcgagctcggaggggaagccacggcacaagaaatccgatcaggcgcggccgaacagaggtgccgcggcacaggagctcagggacggacgagcaggagcagaggagatggataccgcgagcagcctgcagggaaccccgcgccatgggagaaaagcagagccgagcggctggggattcagccagcgagcagaggggagatgggaaggagctgctcggctgcggcttgctgccgggcgtgcgtggagaagaaacctgcgcgctggagatttgtaggagaccaagggcggtggcgggataaggataggagcgctcggcggcgtgatttttatttctaggggttgcgcggcgcggtacagaaaaatcaggcgacgagattaaagagatgcagtgagcagttcgacaaattcgtcggcatggacacaagctgacgacgacggccagcccgaccgcggcaaggtgaggggagacgcggtctgcgcaaagggcgagctcgagcaaggaactagagccgacgatgtccacgacgagcaggaccagagacacggtgctagtggatggaaactgagcacaggatggatgcaagctggagacgagcttggtcaggtcgtcgggcacagaggtgctcggagggtccgacgagcacagccggctgccggctgcctttggatgctgatggatggaagaaatgctggtcgctgggtaaggctggaggagagacgtgcgtgagattttccagcgagctagcgtccaatggataagagagaaggagacgtgaggaagaggagaactacgtggagaaaatatccaggctagtgacttcagatatggaaggggaaagcggtggataaaatcagagagaagagcggttgcagatattttcttccttcgttttcttttactcgaaaatttgaataaaaatacaattatcagctggagattgggactagaatttggaaagatgtaagaggactaaaattaaaaatgattttagttacaatgttttaatcggtgttacatttaattgaaatcagataaaaacttatccgtcaccaaaacacagttgatttggttatcctacattgcgggctaaagaacaaattagatcatattgaaagggaattaggcttacacctagttcctaaataattttggtggttgaattgcccaacacaaatcttttggactaacttgtttgcccaagtgtatagtgtatacaggagtaaaaggttcacactcagccaataaaaagaccaagttttggattcaacaaaagagcaaaggggcaaccgaaggcacccctggtctggcgcaccggactgtccggtgtgccaccggacagtgaacagtacctgtccggtgcaccaggggactcagactcaaactcgccaccttcgggaatttctaaggcgactcggctataattcaccggactgtccggtgtacaccggacagtgtccggtgcgccaagggaggtcggcctcaggaactcgctagcctcgggttcgcgcggcagccgctccgctaaaattcaccggactgtccggtgtgcaccggactgtccggtgtgccagcggagcaacggctccctgcggcgccaacggctccctgcggtgcatttaatgcgcgcgcagcgcgcgcagacgccaggcacgcccataccggtgcaccggacatcaaattccagatgtccgcagtccgctacacactggtattgtgaagcccataaaatttaccgatggctcgatcccgtatggaaatttgacaatttgtgaagaaccctccagcttgtctgttgcattgtttgacccaaactggaaaagctgccatggacctagaattttctgcccttatgcggaataaaacatggcacttggttcctcccgcacctgacagaaatttgattgattgcaagtgggtttataaactcaagagaaaagctgatgagtctattgaccatcataaagctcgatgggtggctaaatgttttaaacagcttnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnntgaaactagagattcgtcctcagctggtttaggcgtgagcagaaggattgtcccctcatataaggaccggtttgtcatcttcactacctgtactctttaatagtacaaccactcgagactgtgtgggcagtcactcaatctgaactcgtacggtccaaccccagggttatgaaggctggggagcaccgggaggataaggagggggaaagttttgtccggtttggacatggtggtggcctgactccttcaggataaccattaaggttaggacatgcggggaaagaaagagagtcggattcgggtctcattgatcatgggatcgcagagctggactagtgggtaaagtgtacacctctgcgcagagtttgaaaacctattcgaatagtctgtgtccacaggaatggacgagtctggtatggtatggcaattaatgttttgttttccaaaaaaaagagatgcttttgaaaagtggtttttaaaaggtccggcggttgagccgtgagctatggtggacgggaagtccagtagctgtttttgaaaatgaaaaccagtgggaaactgctgagatacctggatggtttagtccaggggattttgttataatactgaaaaacttcctgctccttttggagaggatgcactttgcaaaatacaaaatgtttttcaaaacaaccctgcataaaatattgctgtttctgcaaatatcctgagctctacatattccatgcattatatctgatttccccattccgcgggtgaaggtgggctgctgagtacgtttgtactcacccttgcttatttgttgtttttcagaaaaaagagatcgggtaagagttacgactgttcccaaccttgcctgtggctgttggaccgctgaattgcttcactgcgtatatcgggctgcttcagccccactctgatgatatgtcccgagttgtggaccaactcttaaagttgatcgccacctttataggtttgtctcgtttaagcagatctgaatcatctgatgtataaatgtgtttactagcctcctgggactagtaattgtatcacatttgagtcccagaggattggggacgcttcaagctgtggcagaacctcccaagttattgggcccacatgcacctgtccttgtcccaaagacctcagacggctgtgcatgtgcaccagataacttaacaggatctgtccgattgccccaaggacatcggataaaccacttacaaccagaaccgcaggattaagtaacacaaatcacacacaccaatattgttgcagcggaaatcttactaccaaattttacaggttacaaaaattttacattagtttatcggagtgattacaaaagtataagtttgaaatatatatgctagctcaagggatcatcctcaataagaagtatagaagggttacttagacttataagaaggccgagcccaccggcacttaacaccatcaacaacagcacaaagttagaacctgaaaaacaacagggaataaaaccctgagtatggaattactcagcaagtcttacccgactaaagaaaagactctcaagggtatgctggttatatgggagtcaaggtaaggcttttcaataatcaaagactctgttttgcagaaatgcttactaaagtggatccttaaaatccagttttatttgtcaagttaagtagaattacctgtaactagagttctttctaccctagttcaatcactggtcctgcactagccaatttcttaacaaaaacccatcatctttagtggaatgctacgtgtagggcaatgaccaagtcttcataaccgcgaaggtacggcgatccgaatcgattatactcagctgaggatctccaatcacacgacatatgtagcacttaacccttgcatatgtcaacccgccaccggggttcttaagaccagatcaggttcacgcaaaccgagagcacagttacaccaccgtccagcctcttgccacggagggtacacgctactctcgccaccgctccacgcccatttcgtgttatcttattctggccttagtctgcccgaggcaaggcttacccatgacgaggcatgtgaccagttaaagggtcctcgatcatcaagcctacatcgacaaggtccttaatcgactcagacggagacactacactgagactcctttcccgtgcaagtcacccgcccggtcttagcttaatcttttaacccaaaaacttggtacctggcagaggtacatcttttccgatgttgaatccatcatatccatgatggattcaccatcaagttttatttttgaaaacaaccctcccactttgccaaacatcttttctaaaacaaatccttttgtttttctaagcaatactaagcatagtaaaacctttttgtaaaaacgggttttcaaggagggtaatcaagatcaaggaaggtaatgcaggaattgtttaatcaatcaactcctgtcacctaatgcagcaatcaagtgagaaagattttaaaaacatcaagggaggtggcaaatgcaccggggcttgcctgggtaacactaggttagtgttgttagacgacgtccacttggcgaccattttcaggtttgtccatcagcatcatcctgcggattagcccgcgcttggggtcgacttggcttgtcttccgcatcacgcgatcaattatcgtacctaattgagatgcacgatgcacatgaatgcatataaacaagaatagcacaaatctaaatagtgctatacgatagcgtattaaacacctagtggcgaggcgttgtacaattttgtacggaaacactagttattaatatgcgactacgcgctatgattacgcttctcgaacctaacgcaaacatcacgaacaacaaactatacacaagaaatataattagcctaatcacaacttatcagttaattaattaaattctgaactaatcccttgccttataaattatactacatctttataagtgattaaaatattttatcaacacatatgcctactaaaattctactcggtccactaattcagctaagtgaccggaatagcgaaataactcgctataactgaacctggctcgataatcgcaagaactgcgaagtcgacgagagtttcgtgacttacgcaatttatcgagcaccctaatgagcctcgcactaacacattaacttattcaacgatcgaactattctaaacaattcatcagcttactaaactattctaaacaattcatcagcttaccgtttcaacagctgacacgaatccgtgagatcggcagtgatttttcgatccacgcaatttgtcgagcgcttagggaatattgcgctgctaacttcgtcttcacccgattcttgggttttcttggggacgcacgagcgacgatgaccttcgattgaagtctggggtaagctggtgaggtggggatcgagccggagatgacgtgtttgacaactcgacggcaaacgacgagatcgacgacgtgattggaaattaaagccgagcgagccgagagggcacgctggcgagcaggaaaccgtgcgagcacaagacaggaaaaacgacgactcgacaacacgcgaagcgacgacagtcaaatcggcaacaaagcgttggcgaattcgatgaacacgaccacagtcgcgagttaacgcggtcaagacaactgtgaccaacgagcttgcgacagaagcgatgatggcgtgggaactcgacacgctgtgacgagcagataaattcgtgtgcgcggcacaagatagagcgagtgctcgtgagcagagagctccacacgaaactcgacgcgcgctgactacgcgagctagagtagagaaactcgacgcgcgcagactacgtgagctaagtagacagtcatggagatgtagtcatgtaaagcgagctggcgacaacgaatcagnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnatttattctaaccatttcatcagctttataaactattctaaacaattcatcagcttaccgtttcaacagctgacacgaatccgtgagatcgggcagtgatttttcgatccacgcatttgtcgagcgcttagggaatattgcgctgctaacttcgtcttcacccgattcttgggttttcttggggaacgcacgagcgacgatgaccttcgattgaagtctggggtaagctggtgaggtggggatcgagccggagatgacgtgtttgacaactcgacggcaaacgacgagatcgacgacgtgattggaaattaaagccgagcgagccgagagggcacgctggcgagcaggaaaccgtgcgagcacaagacaggaaaaacgacgactcgacaacacgcgaagcgacgacagtcaaatcggcaacaaagcgttggcgaattcgatgaacacgaccacagtcgcgagttaacgcggtcaagacaactgtgaccaacgagcttgcgacagaagcgatgatggcgtgggaactcgacacgctgtgacgagcagataaaattcgtgtgcgcggcaacaaagaaagagcgagttgcgcgtgagcagagagctccacacgaaaactcgacgcgcgcaggaactacggcgagctagagtagagaaaactcgacgcgcgcaggaacttcggtgagctagagtagacagtcatgggagatgtagtcatgtagagccgagctgggcgacaaagcgaaatccaggctgaagcgttgacgagcaaagagcttcgcgcgtgcaagaacaacagaacgctatgcgcgcgacgcaaaggcgagcagtaagacgacggcgcgacagacggaaccaagcagggagcgccggccatgggagaaaagcagagccgagcggctggggattcagccagcgagcagaggggagatgggaaggagctgctcggctgcggcttgctgccgggcgtgcgtggagaagaaacctgcgcgctggagatttgtaggagaccaagggcggtggcgggataaggataggagcgctcggcggcgtgatttttatttctaggggttgcgcggcgcggtacagaaaaatcaggcgacgagattaaagagatgcagtgagcagttcgacaaattcgtcggcatggacacaagctgacgacgacggccagcccgaccgcggcaaggtgaggggagacgcggtctgcgcaaagggcgagctcgagcaaggaactagagccgacgatgtccacgacgagcaggaccagagacacggtgctagtggatggaaactgagcacaggatggacgcaagctggagacgagcttggtcaggtcgtcgggcacagaggtgctcggagggtccgacgagcacagccggctgccggctgcctttggatgctgatggatggaagaaatgctggtcgctgggtaaggctggaggagagacgtgcgtgagattttccagcgagctagcgtccaatggataagagagaaggagacgtgaggaagaggagaactacgtggagaaaatatccaggctagtgacttcagatatggaaggggaaagcgatggataaaattagagagaagagcggttgcagatattttcttccttcgttttcttttactcgaaaatttgaataaaaatacaattatcagctggagattgggactagaatttggaaagatgtaagaggactaaaattaaaaatgattttagttacaatgttttaatcggtgttacatttaattgaaatcagataaaaacttatccgtcaccaaaacacagttgatttggttatcctacattgcgggctaaagaacaaattagatcatatccccgcgcacgatctttctcagacaatgcgcgattcggattattttaccctgaacattttagtcgtcaagttcaaattattttgctcggaataagatcattcgagtgagttcgggcttccgaattcgtgttcgcgcgagcgatggattttaaatactcatcggacgcaccgattttcggaacagctaggttccgaacattacgaaaatttaggaagagcccggacagataaaaaaataaaaacgatgtcgcactcgcgacaaacgacaccgatgcgatattaaaatcgcgataagcgacgatgattaaaatttaaaatccgttttatccactgatattgcgtgcttaaatccgaactcgttgttgagcggaaaataaacacctggggtgttacagccctccccccttaaaagaatctcgtcccgagattcaaaacgaaagacttctaagagtagagaagcatgtaacccatgtccatatcagcgataatcatgagacaattccaaacaaagtcgagtgtctcaaaatgtcgttcctctagtggacataacatgtgtcgccttaggctaatttagaaatgtccaccaatagagacgatgtctgccagaagtacacataaggttccatgtgtgcagtttactttttctgatgacactgtaatatctgagtctgttgagcgagtggtagatatgcaactttacacaaacagaatcagatgcaacctcttgggtaaaacacacagaaagagatttaccaacaagtggtcacggtaagttcatagcacacgagacgagtgtggatgtcgaataacatcacagttaactcgtgttagccagagaatccaagtccaagaaaaatgataaagacttgaaaaaaattaccagcagagggatctgtaaatgctgccttcgcaaccaatccattttatcaagcactaatcatggatctacttgatcacacatgctggaaaagcacacgtgagacgatcgaggcatgactagagcgatgtttaggtggttactggccgacttaatctcgattcttgaaagtacttccttaggatggtttggaccatagcgagtttagataactcgatgaaacgatctctaaactcgaccttcgttcacaaagcagttacaagttagtaaaaccaacttgttaaactacttttgacattgagcaagtcctctcagtaccattggtaatccaagggttgagagttcacatttgctaacaggaaatcatgcacttgggtagaaatccatttggtcacgttgttcatccgtttcttctatacaagatgaaccgacttggttagggaatacatggattaaataagagagcgaatgaacaaattcttgcatttcagcagcaggggaaacaaatctccattttgggaactaattggttgtcttgcaacactaaaaagctccaaggcttcacctttacacaaaggatgtaaagggaacttgtatgtgtgaagtcaccatcaaagtcaagagataagagatcacacatgaaagtggtatgcccttttgatccacagagatgatagatgttgcttgatcacttgacaaacaacatagaaattgtttcaagggaggactccacggaagatcacacatcagtgtacttccacaatggatcatgaccacagaccttgataccagcatccgatgagtggcacagtcctatgtgcgcattcacaggaggctctcagttttcgttgcggcaccataagtcattaatcatgaccaccactaccgaagctg

SEQ ID NO: 104 - DNA, 634 bp, Zea mays
  source 1..634: mol_type = genomic DNA
caatccagggccaggccaggccaggccaaccaaaccctaggcactgcgccacgcctagcgcgcgtggtatccatgggctgaccgcgtcccggtggggagcccggatccggagctagggttccgtcctaggcggcaccaccatggagtgggacagcgagtccgacggcgccggcagcgtcgacgccggctatgaggagcaggaggaggaggaggaggagcggggaggcgagggtggaggtggcgacgccgggggcggcggtgggatgttcacgttcgcgattgaaggcatgctgcgctcctccgggccctgcgggctagtcgtcaccgacgcgctcgagcccgattgccccatcatctacgtcaaccgcggcttcgaggaggccacgggctaccgcgccgaggaggtcctcggcaggaactgccgatttctgcagtgcagagggccattcgctcgaaggaggcaccccctagttgatgctgcactggtttcagagattcgaagatgcatagacaatggcattgagttccgtggtgatttactaaatttcagaaaagatggatctccagtgatgaacagattgcatctgacccctatttatggagatgatgaaaccataacccattatatgggcat

SEQ ID NO: 105 - DNA, 20 bp, synthetic construct
  misc_feature 1..20: note = Primer
  source 1..20: mol_type = other DNA
accaccatggagtgggacag

SEQ ID NO: 106 - DNA, 21 bp, synthetic construct
  misc_feature 1..21: note = Primer
  source 1..21: mol_type = other DNA
ttcaatcgcgaacgtgaacat

SEQ ID NO: 107 - DNA, 388 bp, Zea mays
  misc_feature 188: note = n is a, c, g, or t
  misc_feature 219: note = n is a, c, g, or t
  source 1..388: mol_type = genomic DNA
ctgaacaagatcgaccaaacagttcattcaccagctagaaaatgtgttcaaataggagtggcagaaaaataacacggtttaccagattatactgtcacaaactgttaccgaacacttaaaacaaagactagatgttccccaaaactgatgacaaagcacagctcctcagtacttgataggggcaagantctccaactgagaccccaacttctcctcggntgccttctcggccttgacacgcagcttggccaattgcttcttcctctcgtaggcaaccttgggccttctccttgctctttctcctcaagttccctgatggtgtcatggtagttccacccggcctccttagagagctcgccgaggaggcagtacttgtgtccaggctgta

SEQ ID NO: 108 - DNA, 21 bp, synthetic construct
  misc_feature 1..21: note = Primer
  source 1..21: mol_type = other DNA
cgaccaaacagttcattcacc

SEQ ID NO: 109 - DNA, 19 bp, synthetic construct
  misc_feature 1..19: note = Primer
  source 1..19: mol_type = other DNA
ctcctcggcgagctctcta

SEQ ID NO: 110 - DNA, 161748 bp, Zea mays
  misc_feature 3611..3710: note = n is a, c, g, or t
  misc_feature 7624..7723: note = n is a, c, g, or t
  misc_feature 13118..13217: note = n is a, c, g, or t
  misc_feature 25477..25576: note = n is a, c, g, or t
  misc_feature 70085..70184: note = n is a, c, g, or t
  misc_feature 94587..94686: note = n is a, c, g, or t
  misc_feature 117477..117576: note = n is a, c, g, or t
  misc_feature 128130..128229: note = n is a, c, g, or t
  misc_feature 143525..143624: note = n is a, c, g, or t
  misc_feature 151880..151979: note = n is a, c, g, or t
  misc_feature 155542..155641: note = n is a, c, g, or t
  misc_feature 159499..159598: note = n is a, c, g, or t
  source 1..161748: mol_type = genomic DNA
cagcttcggtagtggtggtcatgattaatgacttatggtgccgcaacgaaaactgagagcctcctgtgaatgcgcacataggactgtgccactcatcggatgctggtatcaaggtctgtggtcatgatccattgtggaagtacactgatgtgtgatcttccgtggagtcctcccttgaaacaatttctatgttgtttgtcaagtgatcaagcaacatctatcatctctgtggatcaaaagggcataccactttcatgtgtgatctcttatctcttgactttgatggtgacttcacacatacaagttccctttacatcctttgtgtaaaggtgaagccttggagctttttagtgttgcaagacaaccaattagttcccaaaatggagatttgtttcccctgctgctgaaatgcaagaatttgttcattcgctctcttatttaatccatgtattccctaaccaagtcggttcatcttgtatagaagaaacggatgaacaacgtgaccaaatggatttctacccaagtgcatgatttcctgttagcaaatgtgaactctcaacccttggattaccaatggtactgagaggacttgctcaatgtcaaaagtagtttaacaagttggttttactaacttgtaactgctttgtgaacgaaggtcgagtttagagatcgtttcatcgagttatctaaactcgctatggtccaaaccatcctaaggaagtactttcaagaatcgagattaagtcggccagtaaccacctaaacatcgctctagtcatgcctcgatcgtctcacgtgtgcttttccagcatgtgtgatcaagtagatccatgattagtgcttgataaaatggattggttgcgaaggcagcatttacagatccctctgctggtaatttttttcaagtctttatcatttttcttggacttggattctctggctaacacgagttaactgtgatgttattcgacatccacactcgtctcgtgtgctatgaacttaccgtgaccacttgttggtaaatctctttctgtgtgttttacccaagaggttgcatctgattctgtttgtgtaaagttgcatatctaccactcgctcaacagactcagatattacagtgtcatcagaaaaagtaaactgcacacatggaaccttatgtgtacttctggcagacatcgtctctattggtggacatttctaaattagcctaaggcgacacatgttatgtccactagaggaacgacattttgagacactcgactttgtttggaattgtctcatgattatcgctgatatggacatgggttacatgcttctctactcttagaagtctttcgttttgaatctcgggacgagattcttttaaggggggagggctgtaacaccccaggtgtttattttccgctcaacaacgagttcggatttaagcacgcaatatcagtggataaaacggattttaaattttaatcatcgtcgcttatcgcgattttaatatcgcatcggtgtcgtttgtcgcgagtgcgacatcgtttttatttttttatctgtccgggctcttcctaaattttcgtaatgttcggaacctagctgttccgaaaatcggtgcgtccgatgagtatttaaaatccatcgctcgcgcgaacacgaattcggaagcccgaactcactcgaatgatcttattccgagcaaaataatttgaacttgacgactaaaatgttcagggtaaaataatccgaatcgcgcattgtctgagaaagatcgtgcgcggggatatgatctaatttgttctttagcccgcaatgtaggataaccaaatcaactgtgttttggtgacggataagtttttatctgatttcaattaaatgtaacaccgattaaaacattgtaactaaaatcatttttaattttagtcctcttacatctttccaaattctagtcccaatctccagctgataattgtatttttattcaaattttcgagtaaaagaaaacgaaggaagaaaatatctgcaaccgctcttctctctaattttatccatcgctttccccttccatatctgaagtcactagcctggatattttctccacgtagttctcctcttcctcacgtctccttctctcttatccattggacgctagctcgctggaaaatctcacgcacgtctctcctccagccttacccagcgaccagcatttcttccatccatcagcatccaaaggcagccggcagccggctgtgctcgtcggaccctccgagcacctctgtgcccgacgacctgaccaagctcgtctccagcttgcgtccatcctgtgctcagtttccatccactagcaccgtgtctctggtcctgctcgtcgtggacatcgtcggctctagttccttgctcgagctcgccctttgcgcagaccgcgtctcccctcaccttgccgcggtcgggctggccgtcgtcgtcagcttgtgtccatgccgacgaatttgtcgaactgctcactgcatctctttaatctcgtcgcctgatttttctgtaccgcgccgcgcaacccctagaaataaaaatcacgccgccgagcgctcctatccttatcccgccaccgcccttggtctcctacaaatctccagcgcgcaggtttcttctccacgcacgcccggcagcaagccgcagccgagcagctccttcccatctcccctctgctcgctggctgaatccccagccgctcggctctgcttttctcccatggccggcgctccctgcttggttccgtctgtcgcgccgtcgtcttactgctcgcctttgcgtcgcgcgcatagcgttctgttgttcttgcacgcgcgaagctctttgctcgtcaacgcttcagcctggatttcgctttgtcgcccagctcggctctacatgactacatctcccatgactgtctactctagctcaccgaagttcctgcgcgcgtcgagttttctctactctagctcgccgtagttcctgcgcgcgtcgagttttcgtgtggagctctctgctcacgcgcaactcgctctttctttgttgccgcgcacacgaattttatctgctcgtcacagcgtgtcgagttcccacgccatcatcgcttctgtcgcaagctcgttggtcacagttgtcttgaccgcgttaactcgcgactgtggtcgtgttcatcgaattcgccaacgctttgttgccgatttgactgtcgtcgcttcgcgtgttgtcgagtcgtcgtttttcctgtcttgtgctcgcacggtttcctgctcgccagcgtgccctctcggctcgctcggctttaatttccaatcacgtcgtcgatctcgtcgtttgccgtcgagttgtcaaacacgtcatctccggctcgatccccacctcaccagcttaccccagacttcaatcgaaggtcatcgtcgctcgtgcgttccccaagaaaacccaagaatcgggtgaagacgaagttagcagcgcaatattccctaagcgctcgacaaatgcgtggatcgaaaaatcactgcccgatctcacggattcgtgtcagctgttgaaacggtaagctgatgaattgtttagaatagtttataaagctgatgaaatggttagaataaatnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnctgattcgttgtcgccagctcgctttacatgactacatctccatgactgtctacttagctcacgtagtctgcgcgcgtcgagtttctctactctagctcgcgtagtcagcgcgcgtcgagtttcgtgtggagctctctgctcacgagcactcgctctatcttgtgccgcgcacacgaatttatctgctcgtcacagcgtgtcgagttcccacgccatcatcgcttctgtcgcaagctcgttggtcacagttgtcttgaccgcgttaactcgcgactgtggtcgtgttcatcgaattcgccaacgctttgttgccgatttgactgtcgtcgcttcgcgtgttgtcgagtcgtcgtttttcctgtcttgtgctcgcacggtttcctgctcgccagcgtgccctctcggctcgctcggctttaatttccaatcacgtcgtcgatctcgtcgtttgccgtcgagttgtcaaacacgtcatctccggctcgatccccacctcaccagcttaccccagacttcaatcgaaggtcatcgtcgctcgtgcgtccccaagaaaacccaagaatcgggtgaagacgaagttagcagcgcaatattccctaagcgctcgacaaattgcgtggatcgaaaaatcactgccgatctcacggattcgtgtcagctgttgaaacggtaagctgatgaattgtttagaatagtttagtaagctgatgaattgtttagaatagttcgatcgttgaataagttaatgtgttagtgcgaggctcattagggtgctcgataaattgcgtaagtcacgaaactctcgtcgacttcgcagttcttgcgattatcgagccaggttcagttatagcgagttatttcgctattccggtcacttagctgaattagtggaccgagtagaattttagtaggcatatgtgttgataaaatattttaatcacttataaagatgtagtataatttataaggcaagggattagttcagaatttaattaattaactgataagttgtgattaggctaattatatttcttgtgtatagtttgttgttcgtgatgtttgcgttaggttcgagaagcgtaatcatagcgcgtagtcgcatattaataactagtgtttccgtacaaaattgtacaacgcctcgccactaggtgtttaatacgctatcgtatagcactatttagatttgtgctattcttgtttatatgcattcatgtgcatcgtgcatctcaattaggtacgataattgatcgcgtgatgcggaagacaagccaagtcgaccccaagcgcgggctaatccgcaggatgatgctgatggacaaacctgaaaatggtcgccaagtggacgtcgtctaacaacactaacctagtgttacccaggcaagccccggtgcatttgccacctcccttgatgtttttaaaatctttctcacttgattgctgcattaggtgacaggagttgattgattaaacaattcctgcattaccttccttgatcttgattaccctccttgaaaacccgtttttacaaaaaggttttactatgcttagtattgcttagaaaaacaaaaggatttgttttagaaaagatgtttggcaaagtgggagggttgttttcaaaaataaaacttgatggtgaatccatcatggatatgatggattcaacatcggaaaagatgtacctctgccaggtaccaagtttttgggttaaaagattaagctaagaccgggcgggtgacttgcacgggaaaggagtctcagtgtagtgtctccgtctgagtcgattaaggaccttgtcgatgtaggcttgatgatcgaggaccctttaactggtcacatgcctcgtcatgggtaagccttgcctcgggcagactaaggccagaataagataacacgaaatgggcgtggagcggtggcgagagtagcgtgtaccctccgtggcaagaggctggacggtggtgtaactgtgctctcggtttgcgtgaacctgatctggtcttaagaaccccggtggcgggttgacatatgcaagggttaagtgctacatatgtcgtgtgattggagatcctcagctgagtataatcgattcggatcgccgtaccttcgcggttatgaagacttggtcattgccctacacgtagcattccactaaagatgatgggtttttgttaagaaattggctagtgcaggaccagtgattgaactagggtagaaagaactctagttacaggtaattctacttaacttgacaaataaaactggattttaaggatccactttagtaagcatttctgcaaaacagagtctttgattattgaaaagccttaccttgactcccatataaccagcatacccttgagagtcttttctttagtcgggtaagacttgctgagtaattccatactcagggttttattccctgttgtttttcaggttctaactttgtgctgttgttgatggtgttaagtgccggtgggctcggccttcttataagtctaagtaacccttctatacttcttattgaggatgatcccttgagctagcatatatatttcaaacttatacttttgtaatcactccgataaactaatgtaaaatttttgtaacctgtaaaatttggtagtaagatttccgctgcaacaatattggtgtgtgtgatttgtgttacttaatcctgcggttctggttgtaagtggtttatccgatgtccttggggcaatcggacagatcctgttaagttatctggtgcacatgcacagccgtctgaggtctttgggacaaggacaggtgcatgtgggcccaataacttgggaggttctgccacagcttgaagcgtccccaatcctctgggactcaaatgtgatacaattactagtcccaggaggctagtaaacacatttatacatcagatgattcagatctgcttaaacgagacaaacctataaaggtggcgatcaactttaagagttggtccacaactcgggacatatcatcagagtggggctgaagcagcccgatatacgcagtgaagcaattcagcggtccaacagccacaggcaaggttgggaacagtcgtaactcttacccgatctcttttttctgaaaaacaacaaataagcaagggtgagtacaaacgtactcagcagcccaccttcacccgcggaatggggaaatcagatataatgcatggaatatgtagagctcaggatatttgcagaaacagcaatattttatgcagggttgttttgaaaaacattttgtattttgcaaagtgcatcctctccaaaaggagcaggaagtttttcagtattataacaaaatcccctggactaaaccatccaggtatctcagcagtttcccactggttttcattttcaaaaacagctactggacttcccgtccaccatagctcacggctcaaccgccggaccttttaaaaaccacttttcaaaagcatctcttttttttggaaaacaaaacattaattgccataccataccagactcgtccattcctgtggacacagactattcgaataggttttcaaactctgcgcagaggtgtacactttacccactagtccagctctgcgatcccatgatcaatgagacccgaatccgactctctttctttccccgcatgtcctaaccttaatggttatcctgaaggagtcaggccaccaccatgtccaaaccggacaaaactttccccctccttatcctcccggtgctccccagccttcataaccctggggttggaccgtacgagttcagattgagtgactgcccacacagtctcgagtggttgtactattaaagagtacaggtagtgaagatgacaaaccggtccttatatgaggggacaatccttctgctcacgcctaaaccagctgaggacgaatctctagtttcannnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnaagctgtttaaaacatttagccacccatcgagctttatgatggtcaatagactcatcagcttttctcttgagtttataaacccacttgcaatcaatcaaatttctgtcaggtgcgggaggaaccaagtgccatgttttattccgcataagggcagaaaattctaggtccatggcagcttttccagtttgggtcaaacaatgcaacagacaagctggagggttcttcacaaattgtcaaatttccatacgggatcgagccatcggtaaattttatgggcttcacaataccagtgtgtagcggactgcggacatctggaatttgatgtccggtgcaccggtatgggcgtgcctggcgtctgcgcgcgctgcgcgcgcattaaatgcaccgcagggagccgttggcgccgcagggagccgttgctccgctggcacaccggacagtccggtgcacaccggacagtccggtgaattttagcggagcggctgccgcgcgaacccgaggctagcgagttcctgaggccgacctcccttggcgcaccggacactgtccggtgtacaccggacagtccggtgaattatagccgagtcgccttagaaattcccgaaggtggcgagtttgagtctgagtcccctggtgcaccggacaggtactgttcactgtccggtggcacaccggacagtccggtgcgccagaccaggggtgccttcggttgcccctttgctcttttgttgaatccaaaacttggtctttttattggctgagtgtgaaccttttactcctgtatacactatacacttgggcaaacaagttagtccaaaagatttgtgttgggcaattcaaccaccaaaattatttaggaactaggtgtaagcctaattccctttcaatatgatctaatttgttctttagcccgcaatgtaggataaccaaatcaactgtgttttggtgacggataagtttttatctgatttcaattaaatgtaacaccgattaaaacattgtaactaaaatcatttttaattttagtcctcttacatctttccaaattctagtcccaatctccagctgataattgtatttttattcaaattttcgagtaaaagaaaacgaaggaagaaaatatctgcaaccgctcttctctctgattttatccaccgctttccccttccatatctgaagtcactagcctggatattttctccacgtagttctcctcttcctcacgtctccttctctcttatccattggacgctagctcgctggaaaatctcacgcacgtctctcctccagccttacccagcgaccagcatttcttccatccatcagcatccaaaggcagccggcagccggctgtgctcgtcggaccctccgagcacctctgtgcccgacgacctgaccaagctcgtctccagcttgcatccatcctgtgctcagtttccatccactagcaccgtgtctctggtcctgctcgtcgtggacatcgtcggctctagttccttgctcgagctcgccctttgcgcagaccgcgtctcccctcaccttgccgcggtcgggctggccgtcgtcgtcagcttgtgtccatgccgacgaatttgtcgaactgctcactgcatctctttaatctcgtcgcctgatttttctgtaccgcgccgcgcaacccctagaaataaaaatcacgccgccgagcgctcctatccttatcccgccaccgcccttggtctcctacaaatctccagcgcgcaggtttcttctccacgcacgcccggcagcaagccgcagccgagcagctccttcccatctcccctctgctcgctggctgaatccccagccgctcggctctgcttttctcccatggcgcggggttccctgcaggctgctcgcggtatccatctcctctgctcctgctcgtccgtccctgagctcctgtgccgcggcacctctgttcggccgcgcctgatcggatttcttgtgccgtggcttcccctccgagctcgcccagctctattgccgcgcccatggccggcgctccctgcttggttccgtctgtcgcgccgtcgtcttactgctcgcctttgcgtcgcgcgcatagcgttctgttgttcttgcacgcgcgaagctctttgctcgtcaacgcttcagcctggatttcgctttgtcgcccagctcggctctacatgactacatctcccatgactgtctactctagctcgccgtagttcctgcgcgcgtcgagttttctctactctagctcgccgtagttcctgcgcgcgtcgagttttcgtgtggagctctctgctcacgcgcagctcgctctttctttgttgccgcgcgcacgaattttatctgctcgtcacagcgtgtcgagttcccacaccatcatcacttctgtcgcaagctcgttggtcacagttgtcttgaccgcgttaactcgcgactgtggtcgtgttcatcgaattcgccaacgctttgttgccgatttgactgtcgtcgcttcgcgtgttgtcgagccgtcgtttttcctgtcttgtgctcgcacggtttcctgctcgccagcgtgccctctcggctcgctcggctttaatttccaatcacgtcgtcgatctcgtcgtttgccgtcgagttgtcaaacacgtcatctccggctcgatccccacctcaccagcttaccccagacttcaatcgaaggtcatcgtcgctcgtgcgtccccaagaaaacccaagaatcgggtgaagacgaagttagcagcgcgatattccctaagcgctcgacaaattgcgtggatcgaaaaatcactgccgatctcacggattcgtgtcagctgttgaaacggtaagctgatgaattgtttagaatagttcggtaagctaatgaattgtttagaatagttcgatcgttgaataagttaatgtgttagtgcgaggctcattagggtgctcgataaattgcgtaagtcacgaaactctcgtcgacttcgcagttcttgcgattatcgagccaggttcagttatagcgagttatttcgctatttcggtcacttagctgaattagtggaccgagtagaattttagtaggcatatgtgttgataaaatattttaatcacttataaagatgtagtataatttataaggcaagggattagttcagaatttaattaattaactgataagttgtgattaggctaattatatttcttgtgtatagtttgttgttcgtgatgtttgcgttaggttcgagaagcgtaatcattgtgcgtagtcgcatattaataactagtgtttctgtacaaaattgtacaacgcctcgccactaggtgtttaatacgctatcgtatagcactatttagatttgtgctattcttgtttatatgcattcatgtgcatcgtgcatctcaattaggtacgataattgatcgcgtgatgcggaagacaagccaagtcgaccccaagcgcgggctaatccgcaggatgatgctgatggacaaacctgaaaatggtcgccaagtggacatcgtctaacaacactaacctagtgttacccaggcaagccccggtgcatttgccacctcccttgatgtttttaaaatctttctcacttgattgctgcattaggtgacaggagttgattgattaaacaattcctgcattaccttccttgatcttgattaccctccttgaaaacccgtttttacaaaaaggttttactatgcttagtattgcttagaaaaacaaaaggatttgttttagaaaagatgtttggcaaagtgggagggttgttttcaaaaataaaacttgatggtgaatccatcatggctatgatggattcaacatcggaaaagatgtacctctgccaggtaccaagtttttgggttaaaagattaagctaagaccgggcgggtgacttgcacgggaaaggagtctcggtgtagtgtctccgtctgagtcgattaaggaccttgtcgatgtaggcttgatgatcgaggaccctttaactggtcacatgcctcgtcatgggtaagccttgcctcgggcagactaaggccagaataagataacacgaaatgggcgtggagcggtggcgagagtagcgtgtaccctccgtggcaagaggctggacggtggtgtaactgtgctctcggtttgcgtgaacctgatctggtcttaagaaccccggtggcgggttgacatatgcaagggttaagtgctacatatgtcgtgtgattggagatcctcagctaagtataatcgattcggatcgccgtaccttcgtggttatgaagacttggtcactgccctacacgtagcattccactaaagatgatgggtttttgttaagaaattggctagtgcaggacaagtgattgaactagggtagaaagaactctagttacaggtaattctacttaacttgacaaataaaactggattttaaggatccactttagtaagcatttctgcaaaacagagtctttgattattgaaaagccttaccttgactcccatataaccagcatacccttgagagtcttttctttagtcgggtaagacttgctgagtaattccatactcagggttttattccttgttgtttttcaggttctaactttgtgctgttgttgatggtgttaagtgccggtgggctcggccttcttatgagtctaagtaacccttctatacttcttattgaggatgatcccttgagctagcatttatatttcagacttagaattttgtattccctccgatagaggtatgaaaaatttgtgtaacctgtcaaatttgtcaataatatttccgctaccactttgtatccgtgtgtgagtttcaagacttaatctcgcggttctggttgaaattggtttatccgatgtccttggggcaatcggacacatcctgttaagttatctggtgcacatgcacagccgtctgaggtctttgggacaaggataggtgcatgtgggcctaataacttgggaggttctgccacaggtcggggttactccggtgagcaatcccgaccgtgaggaggcggaatccggtgcaccgaggccagcacaagcttcggtgaggggtgagggacgcctagggccaaggcacgggcacggggcggggctgaatcggtcggacaccgagcgggcgcggcaaaccaccgcgatagagctccagcgaacacaaattcaccgtagagggaaaagaatcggggcaggaagggcctgaaggtacttacttacgttaagagttctgtcagggatgcttgactgttgctttgggggaggatcttcactgggcggtcgttgtctttttgttcgtgtgtggatgatgagtgccacttgcttccgactggtatgctctacaaatccagtctttctcaaaatgacattgtttaatccctgaannnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnncacgaaatgggcgtggagcggtggcgagagtagcgtgtacctccgtggcaagaggctggacggtggtgtaactgtgctctcggtttgcgtgaacctgatctggtcttaagaaccccggtggcgggttgacatatgcaagggttaagtgctacatatgtcgtgtgattggagatcctcagctaagtataatcgattcggatcgccgtaccttcgtggttatgaagacttggtcactgccctacacgtagcattccactaaagatgatgggtttttgttaagaaattggctagtgcaggacaagtgattgaactagggtagaaagaactctagttacaggtaattctacttaacttgacaaataaaactggattttaaggatccactttagtaagcatttctgcaaaacagagtctttgattattgaaaagccttaccttgactcccatataaccagcatacccttgagagtcttttctttagtcgggtaagacttgctgagtaattccatactcagggttttattccttgttgtttttcaggttctaactttgtgctgttgttgatggtgttaagtgccggtgggctcggccttcttatgagtctaagtaacccttctatacttcttattgaggatgatcccttgagctagcatatatatttcaaacttatacttttgtaatcactccgataaactaatgtaaaatttttgtaacctgtaaaatttggtagtaagatgttcgctgcaacaatattggtgtgtgtgatttgggattcgttttctcgcggttctggttgtaagtggtttatccgatgtcctttggtagatcttacagatcctgttaagttatctggtgcacgttttttggggtctgaggtctttgggacaaggataggtgcatgtgggcctaataacttgggaggttctgccacaggtcggggttactccggtgagcaatcccgaccgtgaggaggcggaatccggtgcaccgaggccagcacaagcttcggtgaggggtgagggacgcctagggccaaggcacgggcacggggcggggctgaatcggtcggacaccgagcgggcgcggcaaaccagcgcgatagagctccagcgaacacaaattcaccgtagagggaaaagaatcggggcaggaagggcacggggatggttccttaccttaggagtgtgctcggggatgcttgacacgatgctaggagctccgggcagacgacgcggtggcgcgcttctccggcgagcttgagcggcggcggctaagcgcgagagaggttgagagtgggtgaaattaggaaggggagggagagcgggtgtaggcggggctcaaaagggagctgggggcgtgggtaggcaacgtggtcggcttcctcggcgtgagtgcgtgtgcgggtcagcagcggttgcggggaagatgagactgacaaggcgggcccacagagtagaggcacgagcacgtgtgaggaggaaatgattcagcgctgatgggctgggcccactgcacagagggagagcgggggcgtgcgcgcgagggtgagcggcaccaataggtcgggcccacctggcagatggagagagagggcaggtgcgtaggctgggctgcttttctattttctttttattctgaatttctagttccttttatttttattttctctattgaattcaaatccaacgaaaccacaaattcaaatttgaatatttcaaacatgtgcatcaaccaaaaacaaagtttaagctcagcatgatgcaacaattcatgtctcccctaggtttgaatatagtaaagaaaaaaaatacatctcccaaatatataaccaaaactctattagaaaggaagaaaataggaaatctacggagatgagaaaagtggtaacacctgaatttggtagatattagagaagaaattttatacccccaaattcagggtgttacataggctctataatcatagcgtgtatagatgcatgaataaataaggtgagcctatgagctatgcgtttcttccactcctgatacatgccaatcaagtgtttctttgaaacaaccttcactggcatgctttgaggagcttgcatagcccttctgaggttttgcctttgcctttgcctttgccatagccattgctactctactgccattttgtggtggcttcgtaaaaattccccctctcatcccctgattattacagtgcccatgacccctttctctggagggacctttcttcctttaagactagaagttggcgtgtgctttatgcatgaccgcgtgccgaatggatgcttctagtagttctgcatcagaagctcattctgagccttagcaccaagcaacatgttgacgaggtcacattatttgtgtacttgttgctacagtactgctgttggagcaccatattggaggggtagaaggtttggagagttttctcgatcatctctaagtctatggctatctggccacagaaatgtagctgagcgacaatgcgatggatgacagtgttgtaagcctcaactgcattgaagtcgagagtggcccattcatgttgcgcccgcgggagcatcacatgcttctccacactgaaacactcacatagctctgtccacaatatcatatggtctttcacctctggatactccatcttcaggcctggatggatgtggtgcccgacgaagatcgtaatttttgctttctcatgaagtttcaggcaagtgtcattcgggccctaccagaagatctcctttcctagctatatcgcctctaggtgaaacttgcagtcgttagtctaggtcggttagttcttgccattgaggacaagctccacgaatcaattctgttgtcttcaaccatagtccgtcacacaaaattaactttaggttaattaggcgtgctaattatcaataatagaaatttaaatagataaattgcatgacccacgggagccggtccagggcgtgctgacgaggcgtggctgaagcatatgccgacgagctgcgccccagctggccgagcaagcgcgacccagtgcggggtggcgccgtgggctgacgcagcgtgaaccatctcagctcgagatggcgtgaccagcagcagggcatgggtgcgtgggtcagtgtgacatcaaggcgcagagttgggggcgccgacatagtgtacgactagaagccggcatgaccgatctattcatgaacgcaacgtgtgggccaccggtggggtgggtgtgtagtgcacgcgactggcgcacgagcctcgccatcggggtgattcgtacgacccgcggttagtaggacatcgatggctttttaagtaaaagtaatagattggatatattaagcaggtcaaatcatcaggatttaacatgctagtgtatacataaatcaatctacaatatgtatctgacagtaagaaatcactctacaatatgtatctgaccgcgagaaaacaatctagtttgaacaaaatctactaaacaattgtctagagcggtcaaaaacgacataccgtgcttctttattcagcgaacggaaacgaagcctatcatgtaagacaattaggcaaggcgggaggacttgctgtggtgcaatttccaaattcagaccttgaaccctctcctcagcaatggagggcttattctctttttgtcgaggcagaagcacaaaaaactatttccttgttatatagacgactagagcttctcgtttgatattagcccacataagattttttgatagtataaggccatctccaactgatcccctattgtatcctctattttattcctatattaaacgcaactctgtaaataatatcatctaaaattctgtgttacctattttattggataagtgagtctaatattttgatccaaacagcgcttaatctcttcccttgctgtaagttctcgacagatttagctgggttaaaattcagactcaaagacatataaattgatgggctggtgttatttccacgtgggggtggacccctcacgggccgggccgcattcttggccaaccatgggctatccgcaacattgcagactccggaatccggattggccgaatgggcccggcagccgaaaatgaaaaaggaaaggatcgaccctctagcgcgatcgatccccgtgcgctggggcccaaattaggagaacctcagtaccccacgtgatccacggtccgcccgccgcgcaagctgggccacgggctcgcgggcgggcatgcgccggttccgcgagcgaccaccccgccatccgcggagcctgccctgcgctgcccaccgctcctccgcctactccggcctcagctaataacgatgggtggtgggagccgagccctttccctctcctccctctgcgccaccaccctcgccgccgccaagcccccacagcaccccgtccccttcgccccggctcaccgcgcgctcccccaccgcctcgccgccgccatgtcctcctcttcctccccgacccccgccgcatcggtggacgccggtgccccggccccttcggcgtccaacgccatcgacttcctcacgctctgctaccgcctcaaggtgagcgactgagcgcccctgtttagtcgcttcgcatttccacgggccggcttggttgagatggatggaaatgtgacgcgatggatgagattgtggatgtaattgcagacgaccaagagggcggggtgggtgaagcgcggggtgcaggcgcccgagtcggtggccgaccacatgtaccggatgggcgtcatggcgctcgtcgcggccgatctacccggcgtcaaccgcgacaggtgatcctgacagtttcgctctcaagtctagctcggcagtatttagccttcttacggttccgttttcatacactgtttatttatcccttcaattacaggtgtgtcaagatggcgattgtgcacgacattgcagaaggtatggtctcaaaagacttccgtctagacggcttcactgaagttttggggctttgtgtgagatgagggatgcaattttgtgaatatgcgagcctattactacctgagatgttggtagatggtaactagaccactggactggagacctgtagtaggaatgtaggatgtgtgttcaagtacttgtgccaattagttggttctttgacctctgctagccaaagtgtaaaactttaaactatgtgcacattttcctattttcattcagaagcatgctcagcttagaaatgaacacatgattttgccctccgctcatatggactcttgctgctgttcctaagccagcttgcctgtttctggaactaactgcctatgaggatgtgggttcagttgactcatttcaattgttttttcttttggtactccagcaattgttggtgacatcaccccttctgataatgtacccaaggaagagaagaaccgcagggagaaagaagcattggaccatatgtgcgagctgcttggtggtggttcaagaggtgaatactgaaacttgcaattgtgatacattagcattttatgctgtagttaattaggcatcttatgcctcaaattgtcttttcatgatttagttatatatgaaatgaatgtggtgctattgcacactggcatcatctttctagattactcaatagtctagacttaatgatcccattatgtgtgcatagtaccatagtttcaaggaaaaaagaacaatatgtggatgccaatgaattttgtgaatacaatactatagtacttgcaggtcatatacatattttattttacccttgaaaagctattcatctgttattattatttcttagatggtcatttttccatccgatacttttcacttccatcagggaagcagatcataacctggcaattattttgtagaaatccagcgggcagcttttgttcttattttttgatacatagtttaaataagtattggataattcttagagtattcacatcccttagttaggtgtcaaggaaactcttggtaacttaaaatcactcagattatttccagagaaactgttatttatacttctctttttctttttataaggtgtattagtgtttgagaatttcattcaaagatatgctttatccaataatttcccttgcaatatatgaactcaatatattatcaattactacaaaagcaatgtctcactaaaatgcatgtgaaatatgaatctatagatttatctttgtgcaataaatatacaaatattttgactagtttcattgacttttttgaatccttacgccctacattttgaaatggaggttgaaaagataagggatgtttttgtagaagccaaaaccgaagagtttatattcagcaaatgttgatgactatgagttttggaatttgaacatgatattgtaattgatggtgataatattattccatctctaatgattttctaccttgaagcattatggatcgtaaattatttatgctcaaatggctatcatagcatccaacattttttccctaagagtttcacaacatagaattctagtattctggttgtgttctcattattcatatcattaatcgttaaaaaatattggagagatccagcatcccttacatgtgaagtgaaccttttagaactaaataaagtatcttagcagccttttggaaacagtttttcatgcaggataaaaggatgttctctgtacaggcgagactaaagagttcatgtgatctttgacatggtatatataataaatacttgcctttatctgcatgctgttgtcttgcagcacaagaaattcgtgaactttggatggagtatgaggagaatgcgtctttggaagcgaaggttgtcaaagattttgacaaggtacagtttcatatttcaatccatcaagttggtggcatgattgcaacgtctgtctgaagctatcagatggtagttcttgtgatcattcaataggcaatgcatataactggcaggatatttaactaatgtaggcaatcattatgatttatggccctaacccatatggctccacttcttccttttcctttgcatgctgtaatcctttgttgcactgttatatttccaggttgagatgatacttcaagctctggagtatgaaaagggtgagttcatactggtgcttgaatatttgaactaacatttcccatgcacagtagctataaagtacaaaccacaactatttaaatgcattcatcaaatattcttgttgtaataaccaaataaatgtatatagtaaaatcagctcacatttcacatttcaaatacagcacatctttttctttgcatcatttgtgcttatattgggtaggcctggtgtagtggtgagggcagtctcactaagtcactatgttgccagttcgaaacagcctctctgcatttgcaggggaggcttgtctcgatttatcccatctcaagaccccactcatgtggcagcctccgccctagatctgcccatctgtgcttacaccattatttaatttgctccacggccttctgggtgtgagaagtgatacatatgatcaatgtactatcacttaacacctggtgaactccttgttgatagatggggttaacagtatcacacttacgcctatgtattttaaaattttcagagcaaggacgggaccttgaagaattcttccaatcaacagcaggtgtgatttttctctttctgttatgctcttctcaattttcatgagtatccagtacataaatcttgctcttctcaattttcatgacaatccagtacataaatcttgctcttctcaattttcatgagcatcctgtacataaatttgaacagttcatttaagctgagaaggatgttgccatttttttggtcttacacttaaaaatgttttcctgagataatataaacattcatcagcaattcagaacatattagtgcctgaatgattattgctaattgaaaactggacactaccacctataatggttttctttaccatgaactgatacatgcctatgccttttatggttttcttttatcacgtgcttatgtttgatctcatttttacattgtattagaccgtgtccagcagttcacccacccaaaacactgttttgcacttagattgcactattcgcagagtggaatttgaatatggggatggtaaacttagcctaggctattagcattagagtcattgtgtaacaaaaccatatccccgcacctaattcccatgcaggcaaatttcagacagacttgggaaaagcatgggcagcggagattgcatcgagaagaaaaacaaagtgatcaaacgatgctcattttaccacgtcggttccaagacaacttgctggcacagcatttctgttgaactttgcttttactagatgatacttcgaggtggcattgagacgtagggttgccttgggaatgtgaacttcaccacatttcttggtcctgccctgaccctgaggcatattgggcttgcgataccagggctctagataagtaagataacccactttgggtattggttgtagatgctcctgccaagggcagttagctggatccaacgggaaggttcagcaccagctggtggtgatgtaaaatccttcacttcatgaattactgtaccattaccgtttctcttgttaatccagcctcacggtttcggccttttttaatgtaattctattgttttcaagtataatgagcctgaatatttgctatatccattttggttgttgatgatgacctgaagtgcattcatattttcatagtacgtataatgctgaagcctagaagctgaccactgatagttccggtgtagcgtcggatcgcatgtattagggtctgttcgttttattttgaatccacgtggattagacggaattgagtgagttttgaaaggatcacgatgcccaagaggagggtgaattggacttttctaaaaatcaacactaattaaaatctaagcaagagtccaacttcaccccgataactatcactaagagaataataatagaaatacaacaatgttaagacaatatttcaaatacttgctaaacaaatacacaatgtaaaatgttttaattaagtgcggaatgtaaagcaaggtttagaagactccaatttttctcgaggtatcgaagagtcggcactctcctctagtcctcgttggagcaccctcgcaagggtatcactcccccttggtcctcgcaagaaccaagtgctcacaacgagatgatcctttgccactccagcgcagtggatccctcacgaccgcttacaaacttgagtcgggtcaccaacaagatctccacggtgatcaccgagctcccaacgccaccaagccgtctaggtgatgacgatcaccaagagtaacaagccatagactttcacttgaccaagagaagcctaatgcatgcggtgtatgctctaggtggctctcgctagcgctaataaggtccaaatgcgggattaagattctcaaataacctcactaggctttgtggtgcttgcaatgctctaccaatgtgtaggagtaaatgtgggtagcaagaccatcaatatagtgggtggagggggtataaatagccctcacccaccaactagccattaccaggaatctgctgcacatgggcgcaccggacagtccggtgcgccaacggtgcgccaacggtcgactccaatggctagttctgacagctagccgttgggcagatggcacaccggacagtccactaaaattcaactcgcgaacaacgcgctctcaggtttctgtgcgcagggaaccctcttccttgggccaggctggccccactggcagagggtgcaccggacagtccggtgcacaccggacagtccggtgccccaaagccagaaaccctagtttctgttttgtgctgttttttcaattcggttttcgttctaacttgtgagtatgttctagagtggcacctagcactatatgtgagtgtgaatatgcaccaacactacactagaactctcttggtcaaactactcatcgacaacccctctttatagtacgactaaaacaaaataaaagacctaactatatcacgagtgtccgcaactccttgacactcggaatacgaagaccttcactttttgttttgtcgctttagccgtcgcttcaagttcttatctccgagattgttttcaccgttgtagtacatctacatgtaatgcgacctaacttaccatttgcctctgcaaaacacatgttagtcacatataaaattacattgtcattaatcactaaaaccaaccaggggcctagatgctttcaatctccccctttttggtgattgatgacaacctacaagattgtgagagtagtttgttttgaaatttctgtcaatagagaagatggttagttatactcaaaaatttttgacagaaagagtgtgtaacataataataagagtgagtgcatacacattgtaagtttcttgttcatataaaagtgaaatcaaatcgatgaacaagaactagagactggtgataacatataaggtgaaaacacaatacacacacagtcaacataagcatcgagagcatataatagagtttgtgagccaaaatcgtcatacaaaagtggatctagtacagagagtatcaagcacatatattacatcaaaatgactctatactaactccctaactccccctagctctcacaactctcatatctctccccctttggcgtcaaacaccaaaaggaaacctgaacctacagaccagaagaggaaggaggtggctggggcgcatccgatcacgatcgtggcagaagagcaaacgccagctgagggtcagagtcagcctcggatccaggatctaccgtagaagctggagctagtactgactcggatggaggctgtgctgcaggagctaccgaagctgaagaggtcacagacaccgccacaacagcagtggtaacagcaggagctggtggcactggcggctgagcgctgatggagctgatgaccggcgacgagaaaaccagggtgaccggcaggagcggagaggaagaaggaccaaatgaaggtagagggggtcctgcctgaagggctagcacaacagcggcctgaagatctagagggggtgcagactgaacaggaggaatctgagcaccggtatgctgaagtatgtgagtcatgaaggcccggttctcagccttgtctgccaaaagctgctgctgcagagtgtcctgtctgtcctgaatagtctaaaacatcgatagcattctctcggacatctgctgttgcaccgctgccagatgagcctgctgctgagtaagagtctggaggatcgcagccagagcagggtcaatggcaggaggaacagcaggggcagcacgagaactccgggcctcatggtcatgtgagcgtggaggcacaggaggcagaggcggaatcccaaaatcatcatcatcatcatcatcatcatcgtcaggaactgctgcgccctaagtctcaaactgatggaaacttgtatcctctgcctgaatgtctgtcactggatcaggtactggtactggatcctcaggggctggatggtaggagccaaataggaggcgtgaggcctcaagggtgccctggaactatggtggtcggatcagctgtgcgaagatgtggcagagataatgagcatttggcagctgtcgccgagcacgaagaccatccaataccgtgtcctcgatctcacaaataaggaagtcaacaacatcaaactctgaatgaaagatcagggcaccgaggagccaaagctgaatatgagtggtagcctctctataacccatccacgacagaagcgtccgtctcatgagctgatataagtacttggctactgtagtgaaatctgccggagaacgtcgcgacccatctgagaagggcggtcggaacaaagccgcgatgtgagctgtagctggagcaactccgtcgtgagggcgacgaggaggatcagaggtaccatagcacaagctatgaagacaagtcgatgactcattgaatccaaacagctggcgaatctagctagcatgaagtgtaacatcctctcgctcaaagcggaacctcatccactggtgatcggggtcgatccatactgacgcattgaacacacggacccactcctcaacatatctgccgctggtagtcagaagagtgagaagtcccagcaaatatgtgagatgcatctcagagtctgcaccagcggctagcagaacacaggaagacccaatagccggtgcgctcgcagaagcaatctgggctgaccnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnngttgatgatcagcttgattcgttaggataaaaccctgagtatggaattactcagcaagtcttacccgactaaagaaaagactctcaagggtatgctgggtatatgggagtcaaggtaaggcttttcaataatcaaagactctgttttgcagaaatgcttactaatgtggatccttaaaatccagttttatttgtcaagttaagtagaattacctgtaactagagttctttctaccctagttcaatcactggtcctgcactagccaatttcttaacaaaaacccatcatctttagtggaatgctacgtgtagggcagtgaccaagtcttcataaccacgaaggtacggcgatccgaatcgattatactcagctgaggatctccaatcacacgacatatgtagcacttaacccttgcatatgtcaacccgccaccggggttcttaagaccagatcaggttcacgcaaaccgagagcacagttacaccaccgtccagcctcttgccacggagggtacacgctactctcgccactgctccacgcccatttcgtgttatcttattctggccttagtctgcccgaggcaaggcttacccatgacgaggcatgtgaccagttaaagggtcctcgatcatcaagcctacatcgacaaggtccttaatcgactcagacggagacactacaccgagactcctttcccgtgcaagtcacccgcccggccttagcttaatcttttaaccaaaaacttggtacctagcagaggtacatcttttccgatgttgaatccatcatagccatgatggattcaccatcaagttttatttttgaaaacaaccctcccactttgccaaacatcttttctaaaacaaatccttttgtttttctaagcaatactaagcatagtaaaacctttttgtaaaaacaggttttcaaggagggtaatcaagatcaaggaaggtaatgcaggaattgtttaatcaatcaactcctgtcacctaatgcagcaatcaagtgagaaagattttaaaaacatcaagggaggtggcaaatgcaccggggcttgcctgggtaacactaggttagtgttgttagacgacgtccacttggcgaccattttcaggtttgtccatcagcatcatcctgcggattagcccgcgcttggggtcgacttggcttgtcttccgcatcacgcgatcaattatcgtacctaattgagatgcacgatgcacatgaatgcatataaacaagaatagcacaaatctaaatagtgctatacgatagcgtattaaacacctagtggcgaggcgttgtacaattttgtacggaaacactagttattaatatgcgactacgcgcaatgattacgcttctcgaacctaacgcaaacatcacgaacaacaaactatacacaagaaatataattagcctaatcacaacttatcagttaattaattaaattctgaactaatcccttgccttataaattatactacatctttataagtgattaaaatattttatcaacacatatgcctactaaaattctactcggtccactaattcagctaagtgaccggaatagcgaaataactcgctataactgaacctggctcgataatcgcaagaactgcgaagtcgacgagagtttcgtgacttacgcaatttatcgagcaccctaatgagcctcgcactaacacattaacttattcaacgatcgaactattctaaacaattcatcagcttaccgtttcaacagttgacacgaatccatgagatcggcagtgatttttcgatccacgcaatttgtcgagcgcttagggaatatcgcgctgctaacttcgtcttcacccgattcttgggttttcttggggacgcacgagcgacgatgaccttcgattgaagtctggggtaagctggtgaggtggggatcgagccggagatgacgtgtttgacaactcgacggcaaacgacgagatcgacgacgtgattggaaattaaagccgagcgagccgagagggcacgctggcgagcaggaaaccgtgcgagcacaagacaggaaaaaacgacggctcgacaacacgcgaagcgacgacagtcaaatcggcaacaaagagttggcgaattcgatgaacacgaccacagtcgcgagttaacgcggtcaagacaactgtgaccaacgagcttgcgacagaagcgatgatggtgtgagaactcgacacgctgtgacgagcagataaaattcgtgcgcgcggcaacaaagaaagagcgagctacgcgtgagcagagagctccacacgaaaactcgacgcgcgcaggaactacggcgagctagagtagagaaaactcgacgcgcgcaggaactacggcgagctagagtagacagtcatgggagatgtagtcatgtagagccgagctgggcgacaaagcgaaatccaggctgaagcgttgacgagcaaagagcttcgcgcgtgcaagaacaacagaacgctatgcgcgcgacgcaaaggcgagcagtaagacgacggcgcgacagacggaaccaagcagggagcgccggccatgggcgcggcaatagagctgggcgagctcggaggggaagccacggcacaagaaatccgatcaggcgtggccgaacagaggtgccgcggcacaggagctcagggacggacgagcaggagcagaggagatggataccgcgagcagcctgcagggaaccccgcgccatgggagaaaagcagagccgagcggctggggattcagccagcgagcagaggggagatgggaaggagctgctcggctgcggcttgctgccgggcgtgcgtggagaagaaacctgcgcgctggagatttgtaggagaccaagggcggtggcgggataaggataggagcgctcggcggcgtgatttttatttctaggggttgcgcggcgcggtacagaaaaatcaggcgacgagattaaagagatgcagtgagcagttcgacaaattcgtcggcatggacacaagctgacgacgacggccagcccgaccgcggcaaggtgaggggagacgcggtctgcgcaaagggcgagctcgagcaaggaactagagccgacgatgtccacgacgagcaggaccagagacacggtgctagtggatggaaactgagcacaggatggacgcaagctggagacgagcttggtcaggtcgtcgggcacagaggtgctcggagggtccgacgagcacagccggcagccggctgcctttggatgctgatggatggaagaaatgctggtcgctgggtaaggctggaggagagacgtgcgtgagattttccagcgagctagcgtccaatggataagagagaaggagacgtgaggaagaggagaactacgtggagaaaatatccaggctagtgacttcagatatggaagggaaaagcggtggataaaatcagagagaagagcggttgcagatattttcttccttcgttttcttttactcaaaaatttgaataaaaatacaattatcagctggagattgggactagaatttggaaagatgtaagaggactaaaattaaaaatgattttagttacaatgttttaatcggtgttacatttaattgaaatcagataaaaacttatccgtcaccaaaacacagttgatttggttatcctacattgcgggctaaagaacaaattagatcatatccccgcgcacgatctttctcagacaatgcgcgattcagattattttaccctgaacattttagtcgtcaagttcaaattaatttgctcgaaataagatcattcgagtgagttcgggcttccgaatttgtgttcgcgcgagcgatggattttaaatactcatcggacgcaccgattttcggaacagctaggttccgaacattacgaaaatttaggaagagcccggacagataaaaaaataaaaacgatgtcgcactcgcgacaaacgacaccgatgcgatattaaaatagcgataagcgacaatgattaaaatttaaaattcgttttatccactgatattgcgtgcttaaatccgaactcgttgttgagcggaaaataaacacctggggtgttacacaccccgtccaatccctggaccggcggtacttactcctggcagctgtctaggatcatatattgtccccacagaccaacacgagtcttttgtgcgcactttgtcctcactcatgcgcacccgagaaaacttcccggtcggtcacccatcccaaattgctccaagccaagcacgcttaacttggaggttctttcgagataggcttccgaaaaagaagatgcaccttgttggtatgattacactattaattctattaagccttgggccaggacatcccatcccaggggccaggatatcacaatccaccccccttagaagaccgacgtcctcgtcggtcaaccccaatccaggaacctcccctcttggccacgtctgtgtgtctagtgccgtcatatgccatgccatgtgaccactccgggcccacatgtgccatgcgccatatacccgaaccccctagcccacacacgcccgtgaaaccgcgagtgtcggctctgataccacttgtaacaccccgtctaatccctggaccggcggtacttactcctggcagctgtctaggatcatatattgtccccacagaccaacacgagtcttttgtgcgcactttgtcctcactcatgcgcacccgagaaaacttcccggtcggtcacccatcccaaattgctccaagccaagcacgcttaacttggaggttctttcgagataggcttccgaaaaagaagatgcaccttgttggtatgattacactattaattctattaagccttgggccaggacatcccatcccaggggccaggatatcacaataagtgtcccgcccagagcgccccctccgccattcactcacctccagtcccgttctcatggccagaaccctgccatcgagttcgtcgggtccccctcaccggtctggccaactccagcgaccccagaccccctggggtccgcgcttgtctcgtctttggcgacttcaccgctgcggatggagcagcgccggccgcagtgctgttaacccccctgacgcctaatcctagccgtttagccttgatctagcggtctagatcgctggatatcgcttcacgtgggtgcccttgcccctgggccccacttgtcagtcatctgtgccctagcgctgggcccgactggtcagctcgtcctcacctcggatcatcacttggaaacactatgtagcagcatgaatgcaacaatcatgacacttctagagctcacaccaatatagaaccaaaataactctctactgttttgataaagggaaaagaaaagtgaataaaggaaagggtaacacctagattttgagtatagagcaaggaaatttttataccccaaaattcagggtgttacagctacgtagtgaaaccttgccgactcaccttggtagtgtttgagggtttgatcgacctgaggcaaaaagggatcacgacttgtgggtaaagtgtgcaacctctgtagagtgttagaagctagtatatcagccatgctcacagttatgagcagccttgggagctcctttgattagagttactctggatacttttatgatgatgcttaatgatggtgattatgattatgaattcttggtatttcctcttggagggagtaatgtttgggtttataacttggggttattgctaaaacatggctctctactggtaataaatacctaaccaactaaaagcaactgctttaagcttaaccccacatacagctagtccactttagccaaacaggacatttgttgagtacgttgaggtgtactcaccattgcttaaaaacaccaaaccccaggttgtccccattgcaactagtgctcaggagaagatgaaggcaacgtggaggactttcaggagtttcaggacttcgacgagttctagactagattagtggcaaaccctcagttagctgcctgtgaaggccttatcgtactgcgtttcgttcaaaattttgattatgacctaagttaatgactctgtggatgtcttggacatccactactagaaatatgcttatttaagacatacatcttaagacaaatatcagtgcattttatagaagcgtcttttatcatatggtgctgagtacggtaagacggtttgttggatatccgtctttaatgaagaaggtttttgaggcagatatatggttggaaatgtcttatattgatttaatacagtttgatgttgaaaaccgtctcaaataaatataccttttgaggcattaagtttacaagaagtgtcttttattttggttagtatattagacacttctgtatatgaaaccatctcaaataaagatattattagagtcatctagactatacaaaattgtcttagatgttagtgagtatactagaaacttgtaaacataaaaccgtctcgtacgatatttttgataggacatattgtgaaaaaacatagtcaatagtaaattctgattagattgaactaaacattttttggaatttaaaatgaactagttagctgactgtatgttcgtacggtttctatatatcatataggtaaaaaatcttgcttaaataagaatcttcttcaaataaaattatacgtttgaaatatgattattttttattttctcatcaacagtatgtttatagttataatatcgtctctttgtacggtataagcaacctgataagcggtggttaatgccacgaatatttctctttatatacgtattgcacatatatacaatacgttttattaatatagcggtggttaatgccatctcctgcgtccgacgcccatcgccgaggctgagaggcaagatccgtcgtcttcagtgcccccagcgcggtgctccaaactcccaggctatgcttttgtttatgttttattgtcatttcatgattcatgacatgacaggctctaggctatgctttagacatttaataagtatattcagctcaaacgaaacgggatctaaaccagagggttaaaggcatgtttggtttgtggctaaatgtgccacactttgcctaagtttagtcgtccgaattgaataactaaccttagacgaaaaagttaggcaaagtgtgataacttaggtagcgaacaaacatgccttaagtctcacatctagggatggcaatttaatgcgtggatagtgatatccgtcggatattcgacccgacggatcaggatatggatatgttttttgacctgcgggttagacccgtacccgatccgagataaagcagacatggatttggatattaaacctcacccgcgggtaattcgttggatatccgaaattaaccattagtccattactgtcgatccacacatggacaccaatgaacaaatcgccagcccaccattgtccattgtgcccaggcgccaagcgccagcccattgcccactaaggcatcattccgccaaagacccaaagtggcaaacacccaaaccgacaaacactaatgatctaatccccatcccccagccggcagcttccgagcaaaccaactcatccggtcggtcatccactcatcctcatcccctgcccatctgatccgatcagtcatctcatcctcatcccctacccgatcggatcccctgctcatccgccgagcaccaccaagcagcaggctccagtcgtcgagcaccagcaggagcacgacacgccgcccagtaggagcacggccaggaggacgacgcccgcatcctgcctcttctcctgctactggagcctctactgctaggagcacggctaggaggacgacgcccgcatccagcaggagcaccagcaggaagaggacgcccatactgctgtcgttgagcgatgatctgatgccccccatcatggctcttctcctccctcgcggcctcgcctcgatctgctgctgccggatccgagcgccgtgcccacgggtcacgaccagcgatatgcagggatcaagaatccaactttgagaaaaattgcttgagatgtaaatggcgccaccggagtaccatcagtactgtgacggaacctcccaagtaattaggcccacctatagttgtccttgtccaacagacatcagacaccctatagatgttcctaaatcacttcacaagttcggtatcttctttcttacctttccaggaacgtttcacccatcttgcagacattacagaacatcggagatatagaaatgcagaagcgattacataacttacatttatttaaaaagtaagatcaagttacttattacagaccagagttatcctagaagtgcagagtaatattattacaataccaagggaggcaaaaactcctcccgatggtttttaaacaaaagttctatatggaggaccaagtcttcccgcggcttcactcttgtttttcttccttgggaaccaccttggagcagaagcaacaaaaatttgtcgcttcctcacctaaaaacaacggaggaataaaccatgagtatggaattactcagcaagtcttacccgactaaagaaaagactctcaagggtatgctggttaagggagtcaaggtaaggcttttcaataatcaaagactctgttttgcagaaatgcttactaaagtggatccttaaaaatccagttttatttgtcaaattaagtagaattacctgtaactagagttctttctaccctagttcaatcacttgtcctgcactagccaatttcttaacaaaaccatcatctttagtggaatgctacgtgtaagtcagtgaccaagtcttcataaccgcgaaggtacggcgatccgaatcgattatactcagctgaggatctccaatcacacgacatatgtagcacttaacccttgcatatgtcaacccgccaccggggttcttaagaccagatcaggttcacgcaaaccgagagcacagatacaccaccgtccagcctcttgccacggagggtacacgctactcccgccaccgctccacgcccattttgtgttatcttattctggccttagtctgcccgaggcaaggcttacccatgacgaggcatgtgaccagttaaagggtccccgatcagcaggcctacatcgagacggtccttaatcgactcagacggagacactacaccgagactcctttctcgtgcaagtcacccgcccggtctcggcttaatcatttcaaacccaaagtttggtacctggcagaggtacatcttttccgatgttgaatccatcaaggcctttgacagattcaccatcaagttttatttttgaaaacaaccctcccacttttgccaaacatcttttgtaaaacaaatccttttgtttttctagagcaaggctaagcatcaaaatccttttgtaaaacgggtgatcaaggatggtaatcaaattcaaggaaggtaatgcaggaattgtttaagcattcaactcctatcacctaatgcagcaatcaagtgagaaagattttaaaagcatcaaggaggtggcaaatgcaccggggcttgccttcgttagtaggtgagttaggctcggtcccgcagatatcgaagtagaaacaattgccggcctgagaatccgaaggtgggggtgtcttctcttcggtcacttcaatctcttcttcgttttctaaatataaccatataggtatatatatatataagaatgaatgccatgtaatgctcatgagagtgcgaagataataaagatttattatctaagtcttgaatacaactttccttcacggaactccgagaacttagggtttccggagtcagtaaaggagttcaaagggcaggggggttttaggttctaagtatcaaacaaggtccaaatcaacccaaattctacccaaggcctctaaataatgcatagaacttatgtaaaaagtttggacatttttggaaattccatttattttctaaaaatccaaaaccactaccttaaactactttaaataccttaaaattccttagttaacctaaaattcatataactatttttattaaattctatggaaaataagaagcctaggaaaattggtttcacaattttaggatttttctacaatttttaaaaaatttccaaagctctatagaaaaagaaaaggaaaaagattgaatagtgttgggctgattctagcccagccggcccagtaccaggggaaaacgcgcgcgcgcgccctcgccctggcgactttgcacagaggtcctcggggtttggctaattagaactggcttctatcactattacactgtgtcgctgacagattgcagagaagcccctgcagttctaactcttcgcagagggaggtcctcgacggcgttcacgcccagccgaactccggcgagtgcctgcaccggccgaacggggcaacgactagggttcccgagcggcggacatcaaattggacctagcccgagcatttcccctaacctaattccatctatggcccaatggcttgctctggccacggtggccgtgaacatcgcggcaagacagtcgcgttcccggcgaccaaagggctcctagctcgattgtgtgggtcggcaagcatcatagacttaagggaaagcttaaacgagggagagaaggagacgaactgaccggaataaggctggccgaggtgaggttcggtttcgggtggcggagaattgatttggggcgaattcaaaattcgtgagctcgggcgaacaattgctagcaatacgtggtggctgggtgggtgatgatgttgtgaagctctctgcggggtcaatttatagatcccaggggcggtggcgcttaatttgagccgacagtgtgggcggccggagataaggaagatcatcggcttcgcgatttcgtgtccaccgccgtggcgggctcaccggcaatgatgagacaacagtggggagtcacggcgatgcgacagaggtgctcggatacatggtgtaaggccgagcgacggtgatccccggcgggcttatctgctcaagccgcacggcagaggggaagtactgggggttcaccggagtgccgtccagcgcatgcctttaccgagcgatcttatctggtcaccggcgacgtgaatcacaacggcggcgacgtgaatctcagcgaagatcagtcgtcggcggtgagagactaccgcgctggctgtctgatctccctggtagcactgtaccatggagagttatatttagacagcctgacagtcaagtttggagcccaattttctctcaatttcaaataacaactcatccagtgacctgcagcaaagttgtagagctacaatccagctataactttgctacaatgtgctcccacaaaaagtcactggatcttgcttaaaattaagccctaagttcatgtcatcccactgttaatctgaatttcagatttcaagcagtctgacagtccaactttaggctcaattatctccagtattttcttaacaactatgctcacactcttaagaaaagttgttctcctatgattgggctataattttaatgtggtgacctagggaaaaaaccctatgatttaaaagttacaaggctcaaaagttgagcccataacactatttttcagacttagtataaaatctcaaatagggtcctttttgcaaatgaggccaaaacttagggtttggcttgtaaattcacatatgagtgacccaaatgacttaagatacttatttaacttggtttttgcactttagtccaaaagtggactaattttgcacataagcccctagggtttggatttagggttttctagggttccgattagggtttttggtatcccagaggtataaatgtggttcaactttattcttgggaatatttcatgactatttccctagagcttttaggttttctcaatttgggttatatcttacccctttaatccctatttagggttaaattccctatctaggttctatttgcaaaacactaaaacaatacaacttgtttgaaatttttacctagtgaatgcactctaggtgtgtcaaacatatgcaatgccaatgtttatgatgctatgctcaagttttagttgcagtaacaccaggggtgttacatccttccccccataaaagaatctcgtcccgagattaaaagtcctagggtaagtaatggaaaaggaaacacgacatacttttatttccttatttctggtacaaggcaggggtggttttggaatcactcctttattacaacagctatacaggctttacaatttacaagaagctaaaaagcctgggaaattcttatctaaaaagtcttgagtttcccatgtagcctcatcttcggaatgttggttccactgtatcttataaaacttgagagttttctccgggtaaccctgtccttttgatccaagactcgaatagggtgctcagaatatgtcaagtccggttcaaggacaacatctgtcacttcaacggttcgatcaggaacccgaagacacttcttcaattgggacacgtgaaacacattatgcacagcaaacaaggtttcgggtaactgaagtcggtatgccactggcccatatctttccaggataagaaaaggaccaatatattatggtgcaagctttcctttaactccgaaacgcgatactcccttcattggtgaaacctttaagtagacatagtatccttcaaggaaatataagggcattcgccgtttgtctacgtaactcttttgacgagcttgagctttcttcaaattatgaattatcctttgaactctttcttcagtctctttcaccatatcaggcctgaagaagtacctttcaccaggttcagaccaatttagcggagtacgacatcgtcgtccatataaagcttcaaagggtgccatcttgatgctttcttgatagctattattatatgaaaattccgctaacggcaaacattcatcccatttttgtggaaattccagaacacatgcccgcagcatatcttcaagtatttggtttactctctcagtctgtccactggtttgaggatggtaggccgaactatggagcaacttagtacccaaggatttgtgaagtgcttcccaaaacttggctacaaattgaggtccacgatccgacactatgggtcttcggaacaccatgcagactaagaatacgagcaatgtacaaatgggcatagacagtaaccgggtgatctgtcttgaccggtagaaagtgagcaattttcgtaagccgatcaattataacccagatagaatcatacccttttgtagtcctgggtaatcccacaatgaagtccatactaatatcttcccatttctatgttgggatcggcaaaggttgtaatggaccagctatcttcatgtgtatggccttgacaagtctgcaagtgtcacacttagccacatagcgtgcaatttcaattttcatcttcgtccaccagtagtgctgctttagatcatgatacatcttagtgcttcccagatgaatagaatagcgactaagatgtgcttcatctaagatttgctggcggagttcttcattcttcggcaccactatgcggttattgaaccatatcacaccttgatcatcttctttgaaacatttggctgttccagccattatcttctcacgtatgtgcttcataccctcatcatctttttgtgcgtcaattattcttcgtatgatgattgactccagcttcaaatgatttgaagtcccatgttgaatcattcccaggtttaatttctccatctcctggcataatgtaatgtcagaagtcctcactgttaaacaatggcaggaagccttgcaattgagcgcatctgccactacatttgcttttcctgggtgataatggatttctaattcataatccttgattagctcgagccatcgcctctgtctcatattcaattctgactgggtgaagatgtatttcaagcttttatggtctgtataaatatgacagacattacccagcaaataatgacgccagatctttagggcatgaaccaccacagctaactccagatcatgagtaggataatgttcctcatgtcggcgcaactgccttgaagcatatgcaattactcggccttcttgcattagcacacaaccgagtccactgcctgatgcatcacaatatacatcaaagggcttggtgatgtccggttgagccaataccggagtagtggttactaatgtcttcaattgttcaaaagcttcatcacactttgaagaccaattgaacttaatatcattcttcaataaacttgtgattggcttcacaagcttagaaaaatctggtatgaatcggcggtaatatccagccagtccaaggaaacttcggacctgatgaacagtggtcgggggtttccactccaaaatgtccttgactttgctgggatctaccgcaatccccctggcagacaatacatgtcccagaaactgaatttcgtccagccaaaacacgcatttgctaaacttggcatataactgatgttctctcaagcgcgttaacacgatccgtaaatgttgggcgtgctcctcttcattcttggaatatatcaaaatatcgtcaatgaagactaccacaaacttgtccaactcgggcataaataccgagttcatcaaatacgtgaagtgggcaggagcatttgtcaatccgaaagacattaccaggtattcaaataatccataccgcgtagtgaaggcggtctttggtatatcttcgggccgaatacggatctggtgatagcccgatctgagatcaatcttggaaaatacccttgctccagtcagttgatcaaataaaatgtcaatccttggaagagggtacttgtttttgatggtgacctcattcaggggtcgataatccacacacatttgtaaagtttgatccttctttttgacgaatatggctggacaaccccacggcgatgagcttggccggataaatcctttctcaagtagatcttgtaattggatcttcagttctgccaactcattaggaggcattcggtacgatcttctagatactggagccgtaccgggtttcaactcaattacaaactctacctcccgttcaggtggcagtccgggcaaatcctcgggaaagacattgggaaactcgcataccaccggaatatccttgatttccggtataatggcttcataagctctgccagtagctttggttggaatggggataggcaaaagaatttcttcctggttatgactcaacctgataattctctgatcagtgttgagagttgctttatgtctggctaaccaattcatacccaaaatgacatatatatcttggcctttcagaatgatcatattagtaggaaagtcccatccggccaaggttacgggcacttgataggccacttctctagtaaatatttgtccccctggtgagtgaatttttaaacccctcttttgattcatggcatgagatgcaatgttgctccacaaatttcttgctgatgaatgtatgcgaagcaccagaatcaaagagaataactgcgggatgattggccacaagaaacgtacccatcattaccggctcaccgtccggtgtagtggccacttgcgtataatatatgcgtcccgtcttctttgtatttttgcccatattattttccttggcttgagatgaattcccagatccttgctgattatttgactggttctgctttggataagggcaatccttgataaaatgcccagatttgccacaattgaaacatccagtcgacgagctgggtaaagcagggaatcgagtgcctggggcacccggctgacttgatgtagtaggggcattattgggacgaataaagactggctgcttaaaaggaaaggagggtggacgagcgaaagaacgattctggttagaaggccggatgacgaaccgttgcctgttcaccgggccctgactagacctgtcacctccaaaacccttggatttaccagcgcctgcatacttcgcttctactgccagtgctgtactgacagctcttccataagtaagatctatgcaggttgccatcttcctttgcagtcgatcatttaatcctctcataaagcaattcttcttcttcaaatcagtgttcacttgatcgattgcatattgtgacaaatgattgaacttattgagatactggttaacagtatcccctccttgtttcagcttcataaactcttcttgcttcatgtgaagaacaccttctggtatatagtgctcgcggaaggccaccttgaattcttcccaagttatctaatgattggccggttgaacggccacaaaattaccccaccaagtgctggcaggtccgcgcagttgctgggctgcgaataaaggcttctgggtttctgaacatcgcagcagtccaaacttttgctcaatcacacgaagccattcatctgcttctaacgggtcttcggctttgacaaacagcggtggtcgcgtctctgagaagtccaagtaagaggtttcacgggggccctgttgataaccccgcccaccttgttgttgcaattgttgacccgccatctctctaagaaaacgggtattatccgcggttgcatttaccaaggccacaatcgcctcggccagtgtgggaggaacaggaggtggatttggggtagactccctcccacaggaggtactagctccgtcctgtgctcgagtcttggaaggcatctgtggcaacaacatttggaaaacaatatgatatgccaaggaaaaaccatccattttacattaccaaaaagagtaatgtacagactcgaatttttacaacaggatacattacctattatacaatagcacaacctattatgcaatagtacaaaatattatacattagagcaacctgttatacaatacactacttctacttctactaccccattattcctgctttccgttgcttttggcggcctcgtcgtcgggtgtgggagaccattcgtcgactagcctcatagaaggagggggctgaaaaaggtctaactcaccaccaagcgcgtgtcccgcaacatgcgagggtccggcttccgactcaccaggattcgtaggctcactgggatgcagttgcgaatataatacatgaatctcttcatgtaaggtattgcaatatgtctgcagttcatcaacagccaagttgagctcggctactcgagcttgagctttttgctccttgtcccatgcaagcgaacgggattgaacaacccagtcaagtgccaaatcccggtccgcgagctgatctcgcagatggcagatatctcttctcaactcatttatgcggtcgccatctatgacccagatagtcgttctgcggcggagtttcgcttggagtcgacttacttcagcttcaagatcccctataggatcattgcttccgctactactattatcatgccttggggtcagctggtgactcggcacaccaatcggtccagtatgtttgcgcgttgttctccttgtgcgcggcggcattttctaagggggaaaatttgattagtatggttcttagcatgatgcatgtataattacagaatcaaccttagttgattcacaccttctatatgttgcactcttactacctggtctttaagatagactcttcagaatacttaggtaagaaaggaagagagtttctaggtaagacttttagaaaatctttttgaagatgcctcataatatctgcaaagaagggctacgctccgataccagctgtgacggaacctcccaagtaattaagcccacctacagttgtccttgtccaacagacatcagacaccctatagatgttcctaaattacttcacaagttcggtatcttctttcttacctttccaggaacgtttcacccgtcttgcagacattacagaacatcgaagatatagaaatgcagaagcgattacataacttacatttatttaaaaagtaagatcaagttacttattacagaccagagttatcctaggagtgcagagtaatattattacaataccaagggaggcaaaaactcctcccgatagtttttaaacaaaagtcctatatggaggaccaagtcctcccgcggcttcactcttgtttttcttccttgggaaccaccttggagcagaagcaataaaaatttgtcgcttcctcacctaaaaacaacggagggataaaccctgagtatggaattactcagcaagtcttacccgactaaagaaaagactctcaagggtatgctggttaagggagtcaaggtaaggcttttcaataatcaaagactctgttttgcagaaatgcttactaaagtggatccttaaaaatccagttttatttgtcaaattaagtagaattacctgtaactagagttctttctaccctagttcaaatcacttgtcctgcactagccaatttcttaacaaaaccatcatctttagtggaatgctacgtgtaagtcagtgaccaagtcttcataaccgcgaaggtacggcgatccgaatcgattatactcagctgaggatctccaatcacacgacatatgtagcacttaacccttgcatatgtcaacccgccactggggtttttaagaccagatcaggttcacacaaaccgagagcacagatacaccaccgtccagcctcttgccacggagggtacacgctactcccgccaccgctccacgcccatttcgtgttatcttattctggccttagtctgcccgaggcaaggcttacccatgacgaggcatgtgaccagttaaagggtccccggtcagcaggcctacatcgagacggtccttaatcgactcagacggagacactacaccgagactcctttctcgtgcaagtcacccgcccggtctcggcttaatcatttcaaacccaaagtttggtacctggcagaggtacatcttttccgatgttgaatccatcaaggcctttgacagattcaccatcaagttttattttcaaaaataaccctcccacttttgccaaacatcttttgtaaaacaaatccttttgtttttctagagcaaggcaaagcatcaaaatccttttgtaaaacgggtgatcaaggaaggtaatcaaattcaaggaaggtagtgcaggaattgtttaagcattcaactcctatcacctaatgcagcaatcaagtgagaaagattttaaaagcatcaaggaggtggtaaatgcaccggggcttgccttcgttagtaggtgagtcaggctcagtcccgcagatatcgaagtagaaacaattgccggcctgagaatccgtaggtggtggtgtcttctctttggtcacttcaatctcttcttcattttctaaatataaccatataggtatatatataagaatgaatgccatgtaatgctcatgagagtgcgaagataataaagatttattatctaagtcttgaatacaactttccttcacggaactccgagaacttagggtttccggagtcagtaaaggagttcaaagggcaggggggttttaggttctaagtatcaaacaaggtccaaatcaacccaaattctacccaaggcctctaaataatgtatagaacttatgtaaaaagtttggacatttttggaaattccatttattttctaaaaatccagaaccactaccttaaactactttaaataccttaaaattccttagttaacctaaaattcatacaactatttttattaaattctatggaaaataagaagcctaggaaaattggtttcacaattttaggatttttctacaatttttaacaaatttccaaagctctacaaaaaaagaaaaggaaaaagattgaatagtgttgggctgattctagcccagccggcccagtactaggggaaaacgcgcgcgcgcgctcgcgccctggcgactttgcacagaggtcctcggggtttggctaatcagaactggcttctatcactattacactgtgtcgctgacagattgcagagaagcccctgtagttctaactcttcgcagagggaggtcctcgacggcgttcacgcccagccgaactccggcgagtgcctgcaccggccgaacggggcaacggctagggttcccgagcggcggacatcaaattggacctagcccgagcatttcccctaacctaattccatctatggcccaatggcttgctctggccacggtggccgtgaacatcgcggcaagacagtcgcgttcccggcgaccaaagggctcctagctcgattgtgtgggtcggcaagcatcgtagacttaagggaaagcttaaatgagggagagaaggagacgaactgaccagaataaggctggccgcagtgaggttgggtttcgggtggcggagaattgatttggggcgaattcaaaattcgcgagcttgggcgaacaattgctagcaatacgtggtggctgggtgggtgatgatgttgtgaagctctctgcagggtcaatttatagatcccaggggcggtggcgcttaatttgagtggacagtgtgggcggccggagataaggaagatcatcggcttcgcgatttcgtgtccaccgccgtggcgggctcaccggcaatgatgagacaacagtggggagtcacggcgatgcgacagaggtgcccggatacgtggcgtaaggccgagcgacggggatccccagcgggcttatctgctcaagccgcacggtagaggggaagtactgggggttcaccggagtgccgtccagcgcatgcctttaccgagcgatcttatctggtcaccggcgacgtgaatcgcaacggcggcggcgtgaatctcagcgaagatcagtcatcggcggtgagagactgccgcgctggtggtctgatctccctggtagcactgtaccatggagatttatattcagacagcctgacagtcaagtttggagcccagttttctctcaatttcaaataacaactcatccagtgacctacagcaaagttgtagagctacaatccagctataactttgctacaatgtgctcccacaaaaagtcactgaatcttgcttaaaattaagccctaagttcatgtcatcccactgttaatctgaatttcagatttcaagcagtctgacagtccaacttcaggctcaattatctccaatattttcttaaaactatgctcacactcttaagcaaagttgttctcctatgattgggctataattttaatgtggtgacctagggcaaaaaccctatgatttaaaagttacaaggctcaaaagttgagcccataacactgttttcagacttagtataaaacctcaaatagggtcctttttgcaaatgaggccaaaacttagggtttggcttgtaaattcacatatgagtgacccaaatgacttaagatacttatttaacttggtttttgcactttagtccaaaagtggtcgaattttgcacataagcccctagggtttggatttagggttttctagggttccaattagggtttttggtatccgaggggtataaatgtggttcaactttattcttgagaatatttgatgactatttccctagagcttttaggttttctcaatttgggttatatcttacccctttaatccctatttagggttaaattccctatctagggttctatttgcaaaacactaaaacaatacaacttgtttgaaatttttacctagtgaatgcactctaggtgtgtcaaacatatgcaatgccaatgtttatgatgctatgctcaagttttagttgcagtaacaccaggggtgttacaagtaccttgtgcaggtgaccaagtactaggccgcacagaactgcaaggtacgtatgcacacatggttacatttactatagaactggagttattttttgatgcaaaggctgccaggtcatggcgatttcacgtccgttaggctcgagaggtggactcaaacatccaagttttgcaagttttgatgttggatgttaaatttctatgctcacccctcgtttggttattgatgtactatttccatctcatgtcacaaatttggcataaggaatgggtattggtggctactggctgtgtttatttccaagtattatacatgtacaatggaacagttgataatagttttgcatgaactattggcattagctatctaaaaggacagaaaggcagacatgagcaacaaatcccgctccatgggctgaaactgggattcgtgatggtcagctaagcataccttcgccttcaaatttgcgtagcttcttttttattctgctagttgtttggtctgctgttcaaatgccttattattctgcgagttgttttaagactgggcctcaattttttttcaaggcagaaagtgctactgccgctctcactgtagcggtgtggtactgggatccttgccaataaggtaaaactctaactgatcttcttacgctttgcattgaggaaggagctcttctgggcggttggataacagagtcgttctagtgtgtttttagggtgagcccgtccaagacgcccgtgcgtccccgtgcccctcgccagctgatgtcgtcctaggtatacatacaggaggtgctgacgatggcactgctcatatataagtaatagagatagacatgtatgaaaagggtcttttgttttcaagtagtgtgtagttgctgttacttttaacagctaatgcaatctggatgagtcacctatgaatgccatactggaatctgttgcgcttttgttgatcattattattttgcaatccaggctaggggattgaagaagcacttgaagaggctcaatgcgcccaagcattggatgctcgacaagcttggtggagcttttgtaagtaaacatgtcggggaccataattaggggtacccccaagactcctaatctcagctggtaacccccatcagcacaaagctgcaaaggcctgatgggcgcgattcaggtcaaggctccgtccactcaagggacacgatcccgcctcgcccgagcccagcctcgggcaaaggcagccgacccaggaggattcacgtctcgcccgagggtcccctcaagcaacggacgcaccttcggctcgcccgaggcccaggcttcgcggagaagcaaccttggacagatcgccacgccaaccaaccgtatcgcaggagcatttaatgcaaggatcgactgacaccttatcctaacgcgcgctcctcagtcgatagggccgaagtgaccgcagtcacttcgccgctccactgaccgacctgacgggaaaatagcgccgcctgccctgctccgactgctgtgccactcgacagagtgaggctgacagcagctaagtccagcctcgggcgccatgagaagctccgcctcgcccgaccccagagctcgggctcaacctcgacgccggacgacggactccgcctcgcccgaccccagggctcggactcagcctcgacctcggaagacggactccgcctcgcccgatcccagggctcgggctcaacctcgacctcggaggagcctccgcctcgcccgacctcgggctcggaccgaccacgtcgcagggggagccatcattaccctacccctagctagctcaggctacggggaacaagaccgacgtcccatctggctcgccccggtaaacaagtaatgatggcaccccatgtgctccgtgacgacggcggctctcagccccttatggaagcaaggagacgtcagcaaggatccgacagccccgacagctgtacttccacagggctcaaacgctcctccgacggccacgacatcacatgaacagggcgccaaaacctctccgacagccacgacagcatgtacttagggctctggctcctctctgctagacacgttagcacattgctacaccccccattgtacacctgggccctctccttacgtctataaaaggaaggtctagggctctcgtacgagagggttggccgcgcgggagaacgggctgacgcacaaggctctctctctctctctcccacacgaacgcttgtaaccccctactgcaagcgcatccgccctgggcacaggacaacacgaaggccacgggttcccctttgctgttttcccccctttgtgtttcgtctcgtgccgacccatctggaatgggacacgcagcgacagtttactcgtcggtccagggaccccccggggtcgaaacgctgacagttggcacgccaggtaggggcctactgcatggtgacgaacagcttcccgtcaagttccagatgggtagtctccagcaaccactccaacccgggacggtgctccatttcaggagtcttgagttcatgtccctcgacggcagctacgacatgacactccttcctccgccgcgcgacaacgacaatggcggccgtcagcccgcccgtcggcggcggaatcgacgacgtcttccccacgtggcggaagagcgatatccgggtctgtcccgtcaccttccccgctgacggaggaggaggcggggtaggcatggccaatcaggaggcggcacctcgtcggctgtcgagcgagtcgacggcgccgacgccccaacgggggacacgtcgggcgttgacctcgcgtctgagacgaagacaagcgtcgtttccccgcaacacgccaaccccaagcagacggatgacgccagcacgctcgcgaaggacttcctgggcgttaacctcgtacctgagacaacggtgcagtccgtccctgacgcgacttcgtcaccacccgtcgatcaagaggtaccgtccgtttcccatcccatgccttttagattcagttgtgacccaccaagcgatcccgcttcggtggacgctttcataaaggcatgtccaaaccctccggggtatcatatgcggtcaacctgggaccgactgacggccgtctcgacctatgggcccccgggttccgaggaagatgacgagcctgactctggttgggatttctccgggctcgataaccccagtgtcatgcgggacttcatgaccgcatgtgactactgcctctccgattgctccgatagcagccacagcctcggcgacgaggactgtggcccaaggtgcgaatgcttccacgtcgatctagggggtcttgacgaaggcaaccatcttggtatgccggaggatggtgatccccctaggcctgcgcctcgcgttgacatccttcgggagctagctgtggtcccagtccctgcggggggtcaagacgcacggcttgagcaaatccgcgaggtacaggccaggctcgacgaggaagcaggacaacttgtgcagcttcggcaaaatatcgggcaggagtgggcaggccgagcaccggctggagaagcgcgtcatctggcccaggacgtccagcaccgcatcaccgacgatgccagggcgaggctgcccccggcttccagtggggtcggccagaacctggctgcagcagcgatactactccgagcgatgccgaaaccatccaccaccgaggggtggcgtatccaaggagagctcaaaaatctcctagaggatgtcgcggtccgacgggccgagagctctgcctcccgaaggcaggggtacccccggagcatcgcgctgcgacttcccgattcatgcgggaagcctcggtccacaccgggcgcacgcgggacacagcgcctgcggccccaagacgcctcggcaacgagcaccgccgcgaccgtcaagcccacctcgacgagaaggtgcgtcgaggctaccaccccaggcgtgggggacgctacgacagcgtggaggatcggagcccctcgcccgaaccacccagtccgcaagctttcagccgggccatacaacgggcaccgttcccgacctggttctgaaccccgactaccatcaccaagtactcgggggagtcgaagccggaactgtggctcgcggactaccggctggcctgccagctgagtgggacggacgatgacaacctcatcatctgcatccttcccctgttcctctccgacgccgcccgagcctggctggagcatctatctcctgtgcagatctccaactgggacgacctggtcaaagctttcgtcggcaacttccagggcacatacgtgcgccctgggaactcctgggatctccgaaggtgccgccagcagccgagagaatccctctgggactacatccggcgattttcgaagcagggcaccgagctgcccaacatcaccaactcggatgtcatcggcgcgttcctcaccggtaccacttgtcgcgacctggtgagcaagctgggtcgcaagactcccactagggcgagcgagctgatggacatcgccaccaagttcgcctctggtcaggaggcggtcgaggccatcttccggaaggacaagcagcctcaggggcgtcagccggaagacgtccccaaggcgtccgctcagcgcggcgcgaggaagaagggcaagaagaagtcacaagcaaaacgcgacgtcgccgacacagacattgtcgccgccgccgagcacagaaaccctcggaagcctcccggaggcgccaacctgttcgataggatggtcaaggagtcgtgcccctatcatcagggtcccatcaagcacacccttgaggaatgcgtcatgcttcgacgctacttccacaaggccgggccaccggcgaaaggtggcagagcccacaacaacgacaagaaggaggatcacaaggcagaggagttccccgaggtccacgactgcttcatgatctatggtgggcaagtggcgaacgcctcgactcggcaccgcaagcaagagcgtcgggaggtctgctcagtaaaggtggcagcgccagtctacctagactggtccgacaagcccatcaccttcgaccagggcgaccaccccgaccgcgtgccgagcctaggaaagtaccctctcattgtcgaccccgtcatcggcaacgtcaggcttaccaaggtcctcatggacggaggcagcagcctcaacatcatctacgccgcgaccctcgggctcctgcagatcgatctgtcctcgatccgggccggtgcgacgccttttcacgggatcatccccgggaaacgcgtccaaccccttgggcaactcaatctgtcagtctgcttcgggactccctccaacttccgaaaggaaaccctcacgttcgaggtggtcgggttccgaggaacctaccacgcagtgctggggagaccatgctacgccaagttcatggccgtccccaactacacctacctcaagctcaagatgtcgggccccaacggggtcatcaccatcggctccacgtaccgacacacgtacgaatgcgacgtggagtgcgtggagtacgccgaggccctcgccgaatccgaggccctcatcgccgacctggggagcctctccaaggaggcgccagatgcgaagcgccacgccggcaacttcgagccagctgagacgattaagtccgtccctctcggccccagcaacgacgcctccaagcagatccggatcggctccgagctcgaccccaaataggaagcagtgctcgtcgactttctccgcgcgaacgccgaggtttttgcatggagtccctcggacatgcctagcataccgagggatgtcgccgagcactcgctggatatccgagctggagcccgacccgtgaagcagcctctacatcgattcgacgaagaaaagcgcagagccataggcgaggagatccacaagctgatggctgcagggttcattaaagaggtattccatcccgaatggcttgtcaaccctgtgcttgtgagaaataaaggagggaaatggcggatgtgtgtagactacactggtctaaacaaagcatgtccgaaagttccctccctctgcctcgcatcgatcaaatcatggattccactgctgggtgcgaaaccctgtctttcctcgatgcctactcagggtatcaccaaatcaggatgaaagagtccgaccagctcgcgacttctttcatcacaccctttggcatgtactgctacgttactatgccattcggtttgaggaatgcgggtgcgacataccaaagatgcatgaaccacgtgttcggagagcacattggtcgaacggttgaggcttacgtcgatgacatcatagtcaagacgaggaaagcctccgacctcctctccgaccttgaaacgacattcaagtgtctcaaggcgaaaggcgtaaaactcaatcccgagaagtgtgtcttcggagtcccccgaggcatgctcttggggttcatcgtctccgagcggggcatcgaggccaacccggagaaaatcgcggccatcaccaacatgggccccatcaaggacttgaaaggagtacagagggtcatgggatgccttgcggctctgagccgtttcatctcacgcctcggcgaaagaggcctacctctgtaccgcctcttgaggaagaccgagcgcttcacttggacccccgaggccgaggaagccctcgggaacctaaaggtgctcctcacaagcgcgcccatcttggtgccccctgttgccggagaagccctcttggtctacgtcgccgctaccactcaggtggtcagcgccgcgatcatggtcgagagacgagaagaggggcacgcattgcccgtccagaggccggtctacttcatcagtgaagtactgtctgagaccaaaatccgctacccgcaaattccagaagctactttacgcggtaattctgacgcggcgaaagttgcgacactacttcgagtctcatccggtgactgtggtgtcatccttccccctgggagagatcatccagtgccgagaggcctcgggtaggattgcaaagtgggcagtggagattatgggcgagacaatctcattcgcccctcggaaggccatcaagtcccaagtcttggcggactttgtggctgaatgggtcgacacccagcttccagcagctccgatccaactggaactctggaccatgtttttcgacgggtcgttgatgaaaacaggagcgggcgcgggcctgctcttcatctcgcccctcgggaagcacctccgctacgtgttgcacctccatttcccggcgtccaacaacgtggccgagtacgaggctcggttaacgggttgcgaattgccaccgagctaggggtccgacgcctcgacgctcgcggcgactcgcaacttgtcatcgacaagtcatgaagaactcccactgtcgcgacccgaagatggaagcctactgcgatgaggttcggcgcctggaggacaagttctatgggctcgagctcaaccacatcgcccgacgatacaacgagactacggatgagctggctaagatagcctcggcgcggacaacggttcccccggacgtcttctcccgagacctacatcaaccctcagtcaagaccagcgacacgcccgagcccgagaaagccttggccctgcccgaggcaccctcggcccccgagggtgaggcactgcgcgtcgaggaagagcggtatggggtcacgcctaatcgaaactggcagaccctgtacctgcaatatctccaccgaggagagctacccctcgacagagccgaagctcggcaactagcgtggggcgccaagtcgttcgtcttgctgggtgacgggaaggagctctaccaccgcagcccctcaggcgtcctacaacgttgcatatccatcgccgaaggtcaggagttattacaagaaatacactcgggggcttgcggtcaccacgcagcacctcgagccctcgttggaaatgccttccgacagggtttctactggccaaccgcggtggccgacgccactaggattgtacgcacctgccaagggtgtcaattctatgcaaagcagacccacctgcccgctcaggctctgcaaacaatacccatcacctggccgtttgctgtgtggggtctggaccttgtcagccccttgcagaaggcacccgggggctacacgcacctgctggtcgccatcgacaaattctccaagtggatcgaggtcagacccctaaacagcatcaggtccgaacaggcggtggcgttcttcaccaacatcatccatcgctttggggtcccgaactccatcatcaccgacaacggcacccagttcaccggtagaaagttcctactgcgaggattaccacatccgggtggactaggccgccgtagctcaccccatgacgaatgggcagctagagcgtgccaacgacatgattctacaaggactcaagccacggatctacaacgacctcaacaagttcagcaggcgatggatgaaggaactcccctcggtggtctggagtctgagaacgacaccaagctgagccacgggcttcacgccgttttttctagtctatggggccgaggccatcttgcccacagactcactgggccatcttcacgctgttttttctagtctatggggacgagggcgtacgacgaccgaagcaatcgaaccaaccgagaagactcactggaccagctggaagaggctcgggacatggccttactacactcggcgcggtatcagcagtccctgcgacgctaccacgcccaaggggttcggtcccgagacctccaggtgggcgacttggtgcttcggctacgtcaagacgcccgagggtgtcacaagctcacgcctccctaagaagcccggaacatacaagctggccaacagtcaaggcgaggtctacatcaacgcttggaacatccgacagctacgtcgcttctacccttaagatgttttcaagtcgttcatacacctcgtttacatacgccaacaaagtctaaccatcaaggaagggtcagccttgcctcggcaaagcccgaccctccctcgggggctagaaggggggcaccccctctacgtcaaaattttcctcgaaaaaagtctttctgccagaacatctttcgtgcttttcgactacttcgaaagtgggatcctgaaaacgacggagtacacgtaagcaggcaaggacgaccgagccgagggactcctacgcctccgggatacggatacctcactcatcaccttctgcgataagtaactcacgctcggataagcgatcccgctggccgaacaagtcttaacgttcgaaagcttttctgccgaaacgattttttgtgccttctcgactatatcgataacagaatccaacggacgagtaagagtacacgtaagcggcaaggccgaccgagccgagggactcctacaccttcgggatacggatacctcactcatcaccttccgtgaaaagtaactcttgctcggataagcaattctgttactgacgaacaagtcccgatactcgaaacaaggggaaaagaaacgccgctttacaacacgacgacggtatgtttgggcctcggcggccgcaaaaaacatacgcacactacagataaattgttcctgcaggatcagacatcagtgggggagcagcagcaccctcggcgtcgactccaccttcggcggagtccgacccagcctcggacggcgacacggtcggaggatctccatctcgaaggaacctgtcagcaccgcgcctgggccatcgccgaggtgtcctccaggaacccggcccgagtagacgactcgaccgaccgctctgtagcctcagccagctgtcccccgaggacatcagcccggctcatggcctcggcaacccgactccggcgtcggtcccaccagtggacggcccgaccaggctccggccgatgaagcttctttttgagccaactccgcctctgtccacgctgacaccgctgacaccgctgcctctagctccggctcatcgcagagcggccgagggtttctttaactaagcaagagaagcctcgggcggcaaggccgaccgatccgagggactcctacgcctccgggatacggatacctcactcgtcaccttccgcacgaggcaactcacacttggttaagcggttcagctagccgacaggcgagtcctagtgctcgaaatgaggaaaaaatacggctttagccaaaatacacatcttcaggccccgacagccgcaatgaacagacaccggcactcaaggtgccattacaaacagaactctggttccgcccccacaggtacgaacgaccccccacattggagggcctgcggggcaactgaaagctctcttgtgagttttggtgtttggatgacaactcaattaaaggactaacaagtgtactaagtgttgaacaggtgcttaaggtaaagcctacagggttcaacacaagtgaacaaatgtgatggtccaagaactggattatggatacataatggacatcacaagtaagatggacattgcacaaagtgagactcgggtgcgtagctcggagacaactgatcaagccaaggacggaggcaagaaaagcttcgaggtaccaaatgcacgggagaaggtcaaggaggctgaggaacccaaagccaagggtgaagaagaaggcttgcaaagtcaagggtgatcgagttgagaacagctacggcacatcaaggatcactacataaggacgtgacttacagccaatgaggtaacagctatagttatgtggtgtaagtcataaggctcaagatcaagctctaaggaggagatcaaggtcactagaaggagaacaagtgtcgaaaccagaactggaagcagcccaaaagagctaagttcactttgatctttagtttgggttgttcctatgtttggagatgttctatgtgacctttacaggatgttggagccaagcgatgtcaatctagatcaagtcaagctgacttgataatttatgagtccaacatcaaagctcaagcatgtgaaatgctatagatgtaatgattaatagaaggtatgtttctagacttagtacattggttttggggactaatatacttgtctaagtgttagaaacagaaagaagaagaaaagggaagaggtgcgaaaggcttggctgtgtacagccaagacttagttcagtctggcacaccggactgtccggtggtgcaccggacagtgtccggtgcgccaggctgaactctggcgaactggccgctctcgggaattcaccggcgatgtatggctataattcaccggactgtccggtgtgcaccggactgtccggtgagccaacggtcggccgggccaacggttggccgcgcgatctgcgcgggacacgtggccgagccaacggctagatggaggcaccggattgtccggtgtgcaccggacatgtccggtgcgccaacggctccaagactgccaacggtcggcttcgacgtagaaggaaagaaatcgggcaccggacagtgtccggtgtgcaccggacagtgtccggtgtgcaccggactgtccggtgcgccacccgacagaaggcaagatttgccttcctggattgcttccaacggcttctaggccccttgtgtctataaaagggacccctaggcgcctccagcaaaatagaagtgcagccaacaagtgtagactccactggaatcaattctcactctccctcttgtgtgtaactctatagtttgtgtagaaggcacagctataagccttagagagaggagtagtgctgctaagagctagagcaaggtcttgagcatatcgttactctaccggggtgctgccaagaagtctgtaagcagccgcggttctgttgtaaccccactcaatagtgaaaggctctatctgtcatactgacagatctgagcaaacggaggaaggagttgaaatagactccaagcccaggtgtggctaactccaacgaggactaggcaagcatttcaggcttggccgaacctcgggataaatccttgcgtctgtgtgctctgttctgtattgtatcctgactctctttctactcgcctttatatctgcacttcaatacttatctgtggtataagctttatttgaagtgcaggacattttgagacaggatcttctattcggctgcaacctacttgaagagtcttctcactccactgcatactaagtcttcgagtagagtaagaatttaagttttaaagtgaaaagttttattcgcctattcacccccccccctctaggcgacatccagatcctgttcccgggtcaaagggaactttcaattggtatcagagctaggcctctccagtgtgggcttagccgtccggagatgacgatgtcgtcacaagaggtaactgtggaacttcttttagacgatggctctaattacaagtcttggtctgtctctatttatagtgctttcatgagtgttgatcctgatttgagacaggtctttagtagtagtatttttccctccaatattagtaaaaacccatccaatgaagaactaagatgtctaactctaaatcaccatgcttgcaacatcttagttgattctctatctagaggtgcctattttgccatcatgagtagtgatagtgatctatttgttgatgctcatgatttatggaataggattaaagaaaaatattttgtggcaaactgtgatgctcctactccctatattacttgtgatactaaccattcaaagggagaagaacaagaacgatggcatccaaacgatgaatccacctcgtcgacaggtttgttctccactagtgataaatgttttattgctaacaatgacggtggagacgaaagccatgataaggagaaatatgaggatgaatcttcatcatcacaaggtacattttcctatattgcttccactgacattaatgacagggaaaatgagaccgatgatgtggaggaagaggagattcaccgtttctacatccatctcaacaaagaggacaaggcactcttggttaagctgttgagaaggaacaaggaacaaggcgagacgcttctcaggctagaggagtccctcatcaaaaccaacaacagcctggagaagatgaccaaagaacatgagaagctaaggcgctctcatgatgatttggtccaaaggtatgaatatgttttaattgagcaaagaaatagtcatgatgcattatctaatattgctcaacttaaaacggaaaattctatgcttaagagtcaagtagaaacaatgaacttagaaaaacgtgctctaggtaaaaagtatgatatgttgtcaaattctcataataaattagttgatgaccatatcatgcttaatgttgctcatgaggttataattgcaaacttaaattcatgtgaacctcattctcgcacgtgtgcgcatttgaagtgtatatcaccatgtgctaacccctgttgctcaaaagaaagccaatcattgattgagcaacaagttttagggtcacaaaagaaattctgtgggaacaagaagcaaagacaactaaggagaagacacattgctcaactctctcaagatatccacgggcgcgtggtgaagaagcttgagaaaggaaaaactgcagcaagtgttaagctcaataagaagaatgttcccaaagctataaatgaagaaatcaacatgaacaaggaaaaaggtaaaaattcaattagtcatgttgtttgcactgatcatctctccatgtcattcaagcacaaaaagggaagaggaaaaaggaggtgcttcaaatgcaaggagacaggccacctcatcgcgtcttgtccgtacaaagacaaggatgaaagaacaaggagttgttttggatgcaacaataaggaccacatgatcacttcatgtccggtcatgaagaatcaaggatatgcatcctccaaagtgaccctcaccaaggaaaatgacacaaaacaagcgtcatgtcaagttgagcgacgcttctgctacaagtgtggtgagcaaggtcatctatccaaggtatgttacaaaggtaagattcctaaacaagtgaatttgtgtcaatcttattcgcataggagacccaaatcatacacttgtgctagatctataacgagatcacctagaactagcacaaaggcaatttgggtaccaaaggcacatttacatgatcattatgtacccatcccgagatggataccaaactgtgccaactagaccatgcaggtgcctcgagatggactggagaccatgggaaagattaagacggttatctaaaactctatgcttaagctgttaattgttttagtgtttattgacccaaggttgaattattgtgaaacactaatcccatgttcatctcaagagaaataaggtgtataggtcctgaatcattattggtgaatcaagtaaaggatcttgatgagaatctacaacctgctctccaaaggacggtacccgtgtattttaagtacataattgcaatttagtattgctcttaagttggcttgttgtgctacctgtccttagagtagttatgctttatgattgcctgtgttaaattgatcataatgatggttgcttaatcatgactggtgctataaaggatatatcttttgaatcattcatgggtagctatttcatttgttatatccacaacgataactctcttgatgtatatggataaacctgtaacttttgtaagtcatgctatgtgcaattatgacattttgtttagtccatgttcacatgattaccctagtttggtactgtgtgaatttcaaatccatgtcgtgcccttttgagctatgaggtgcgtaagcaaaaggagccctaaattggcgataacaagggctctcataaaggcaaaggtatggaaaatggagctatgcaatttcattaaatattcttgaaattccattcattgtgatcatagctatgttcttgcctttcaattggtaatatcttggcttaggtaatttatgcctttaaaatgttgtttcttttgtgcacctaagaaaccttcttaattataacatgcttagatatttcgattgtgtttatctttaattggtatatacaatgatagttaaatatgaagcatgtacaagttgcgtaaatgttagacttcctgtgagtattcaattggcttaggtgccactgaggcgtgcattgttgtatttagtcaacctttcatttagccttcaattggtgttatgtggcgtttcatttgatattcaaattggcatctttgggtgatgaaagtggtagagtatgccttgaccaaggtatgttgtgatcccctctaattctaaggaagctagaatgtgcaaagtgcaagtcattcaaatacttgatgcacaacttgagggggagcacacataacttgtgtcttttgagactaactgtttcttgagcaatcttgtatagtctctaggtggaaaagagaagataagcaagaaatggagcaatcaggacttgggtacctctgtaagtcaagaaaattggtatctcaagttgtgagtaagtgcatatttttagattgctcatgctctataatatctggtgataatagatgcttattcttaaatatcatggagccatgataataaatgaactttgcaattggtatctttcaattggtagccgtaatagttcgcttcaattgacatcttttgataatcatgagaatagaagtttcttcttgtgcccaatactataacttgttctaagtttggtgtcttagcaacaagaaaaagttaggagagagaatcaggcacaagtgtggagaagctctcgagagattaactactttcaagatgggaagtacactacatcatggtaaaggtacaaaaggaagtattaatctttttgcatatatgtatcttacctaaatgttgataggacatatgttcaataaataagggggagttttgatagtcgtttttccccttaacaccctgctgtcccttgacatcatcatatgttcttgcttgagtatggtttttggtgtttgatgtcaaagggggagaagttgtgcattaaagcttatctcaacctgagaggaaagcttatcctaatgggtgatgtgttagtttgagctttgccaagtgtgatattcatatgtttcttgcagtattatacgtgttgatcatatggactagactagtgttttatattcatatgtttcttgcagtattatacgtgttgatcatatggactagaccagtgtttccgctgcgatgaattatttggcttctatagtgaaatagatagtcatgtggttaatggtgctttaagattgctttaaattgatatcttagtttaagttggtatcttaatggtgaatagtggtaggttgatattcctgtgatatatccactaatttgaatggtgtttaactctgattatgtgcatttgtgtgttatagcatcatggtttgattcttgacataatgcatcctaaaaagtgctaaggtgtagaaatgtttcaattttcctaagtatgtgcaaattgacgtttgtggtcaaaattaggtttttgaagtaagcacttatttagggggagcattctataatcttagaattcaaatttgtgcttcaaatcttattcttatgtaagctttaattgtgttgccaccaatcaccaaaaagggggagattgaaagctctcttgtgagttttggtgtttggatgacaactcaattaaaggactaacaagtatactaagtgttgaacatgtgcttaaggtaaagcctacagggttcaacacaagtgaacaaatgtgatggtccaagaactggattatggatacataatggacatcacaagtaagatggacattgcacaaagtgagactcgggtgcgtagctcgaagacaactgatcaagccaaggacggaggcaagaaaagcttcgaggtaccaaatgcatgggagaaggtcaaggaggctgaggaacccaaagccaagggtgaagaagaaggcttgcaaagtcaagggtgatcgagttgagaacagctacggcacatcaaggatcactacataaggacgtgacttacagccaatgaggtaacagctatagttatgtggtgtaagtcataaggctcaagatcaagctctaaggaggagatcaaggtcactagaaggagaacaagtgtcgaaaccagaactggaagcagcccaaaagagctaagttcactttgatctttagtttgggttgttcctatgtttggagatgttctatgtgacctttacaggatgttggagccaagcgatgtcaatctagatcaagtcaagctgacttgataatttatgagtccaacatcaaagctcaagcttgtgaaatgctatagatgtaatgattaatagaaggtatgtttctagacttagtacattggttttggggactaatatacttgtctaagtgttagaaacagaaagaagaagaaaagggaagaggtgtgaaaggcttggctgtgtacagccaagacttagttcagtctggcacaccagactgtccggtggtgcaccggacagtgtccggtgcgccaggctgaactctggcgaactggccactctcgggaattcaccggcgacgtacggctataattcaccggactgtccggtgtgcaccggactatccggtgagccaacggtcggccgggccaacggttggccgcgcgatctgcgcgggacacgtggccgagccaacggctagatggaggcaccggactgtccggtgtgcaccggacatgtccggtgcgcgaacggctccaagactgccaacggtcggcttcgacgtagaaggaaagaaatcgggcaccggacagtgtccggtgtgcaccggactgtccggtgcgccacccgacagaaggcaagatttgccttcctggattgcttccaacggctcctaggccccttgtgtctataaaagggacccctaggtgcctccagcaaaatagaagtgcagccaacaagtgtagactccactggaatcaattctcactctccctcttgtgtgtaactctatagtttgtgtagaaggcacaactataagccttagagagaggagtagtgctgctaagagctagagcaaggtcttgagcatatcgttactctaccggggtgctgccaagaagtctgtaagcagccgcggttctgttgtaaccccactcaatagtgaaaggctctatctgtcatactgacagatctgagcaaacggaggaaggagttgaaatagactccaagcccaggtgtggctaactccaacgaggactaggcaagcatttcaggcttggccgaacctcaggataaatccttgcgtctgtgtgctctgttctgtattgtatcctgactctctttctactcgcctttatatctgcacttcaatacttatctgtggtataagctttatttgaagtgcaggacattttgagacaggatcttctattccgctgcaacctacttgaagagtcttctcactccactgcatactaagtcttcgagtagagtaagaatttaagttttaaagtgaaaagttttattcgcctattcaccccccctctaggcgacatccagatcctgttcccgggtcaaagggaactttcagcaacaaaaccctagacagctcgccgaggcccgctctggcagcagcgacaacgacctccgctccggacagccaaacagcagcagcgatgacctcagtgcagacgctgctgcgacaaggccctcgcccacgtccccaccatcaaactggtggtcaccgtcttgggtgaccaccagcgaggggatgcagccgggccgcctgatgaaaatccttgaagccgagcgatggctgaaaggtaccaacttccgcgaagttgcgttcctccaacgacgacaagacgaaagcaacgcgggcgctccccatccgggggctcggaagttggaagggcgcgatgcatgaagggagtgtgaagacatggttgccatccaagggggtcgccctccttttaaaggcgactctccccacttgcgtcctcagccgtcgcggactgagtcttcaccaacacgctccaaggtcctccccctacgacatgggggctgggtcccacgcgtcatgcaagctggcccagggcagaagaagccaaaccgtcgcgcgcagagtgcgtaactgcccagcggttacaagcactcctccactttcgcccagaccggcgggtgaaagggcggaccgccatgcaggcggcatgcaaccgcaccaagggggtgcaccctttcgactccgacgcgtccagcacgggggcccaggcccacacgtcatgtaaccggcgcgccggttactacgcgcgagaaactgcaccgccacttgtgctagtaccgcgccttctcgactgcggaaccggtgccgcgactcgaggcaaccctgcgcatggcccaacagtgccaaccgagcacatcgatcacgggtcagtcagccgcgggagaaggcgcgatggttgatatggccaaaagtgggccggcagtaatggcggcggcaggcgggcggaagcagcggtcaagtcgtctgtaggctcacgtcccctcctgggacagcgagagagccccctcccacggcgtgaagacgacacgcccgtgttccgttcctcgaacggctagcgcacgcacaacggctgccccgcgaaccactcatcccgtcgcattaactctgcggcaggacaggcggcacctttggcaggcgaagcaggtgacgcttcacctccgccttaatgaccgcgtcaaaaaaggtgcgccacgtcgtttgatttcgtatccttttacccttcctctttctctctcttgctatagggaccgggaaagaggatactccgaaagggatccttctccgcgaaggaagcgggccccgagccctcctactaatcagaggttcgaaggctggcccctcggaagggttcgacagtcgccttagagcactcgggctccgcgccctcctactgatcagaggttcgaaggctggcccctcggaagggttcgacagccgcctcagagcactcgggttccgtgcccactactggtcagaggttcgaaggctagcccctcggaggggttcgacagccgcctcaagccactcgagctctgcgcccactactgatcaggggtttgtaggctggcccccgaaggattcgccagccgcctcagagcacgcagagcgagggatgactctgggtacgtccgatacatggccgaggctcgggctacgctcccgaggtaccctaggacatttccgagaccaacaggagcgattctgtaacggaatcccatcagagggaggcatcgagccctcggaccctatcaaacgggaccgggtccggcaaatcacctgtaggtacttttggagcgcgcctctgggccactagccgacccttatcgaacggggcacgggcgtccactcggatcaaccgttagcaactcactggagacaccatgttcgacgccctctgagggcaacatggcgctttcccccccctcctccttgcggaaaggcgacgcaggggcgtatgaaaaaagccgagtcagtccttggccgtcctctcgctctgtgcggaggctcgggggctgctctcgcatgagggaacaaccaaaccagcccgagaacttggaacctgactatgcacccgggctacggccagttcgcatgagggaacaaccagaccggccgaagcatcacgaaacgtgctaagacctcgaaggagtcaaaccactcctccgaggcctcaggggctacacccggcgggtgcactcgcgcgcacccaccggaacgaaacgcaaccgagaaaggccggtccccttgcaaaaaagtgcgacaaaagcctccaagtgagtaccaacactcccttcgaggctcgggggctactgtcggggaccataattaggggtacccccaagactcctaatctcagctggtaacccccatcagcacaaagctgcaaaggcctgatgggcgcaattcaggtcaaggctctgtccactcaagggacacgatcccgcctcgcccgagcctagcctcaggcaaaggcagccgacccaggaggattcacgtcttgcccgagggtcccctcaagcaacggacgcaccttcggctcgcccgaggcccaagcttcgcggagaaggaaccttggccagatcgccacgccaaccaaccgtatcgcaggagcatttaatgcaaggatcgactgacaccttatcctgacgcgtgctcctcagtcgacagggccgaagtgactgcagtcacatcgccgctccactgaccgacctgacgggaaaatagcatcgcctgccctgctccgactgctatgccactcgacagagtgaggctgacagcagctaagtccagcctcgggcgccatgggaagctccgcctcgcccgaccccagagctcgggctcaacctggacgtcggacgacggactccgcctcgcccgaccccagggctcggactcaacctcgacctcgaaagacggactccggctcgcccgaccccagggctcggactcagcctcgacctcggacgatggactccgcctcgcccgaccccagggcttggacttagcctcgacctcggaagacggactctgcctcgcccgatcctagggctcgggctcaacctcgacctcggaggagcctccgcctcgcccgacctcaggctcggaccgacacgtcgcagggggagccatcattaccctacccctagctagctcaggctatggggaacaagaccggcgtcccatctggctcgccccggtaaacaagtaatgatggcaccccgcgtgctccgtgacgacggcggctctcagccccttacggaagcaaggagacgtcagcaaggatccgacagccccgatagttgtacttccacagggctcaaacgctcctccgacggccacgacatcacatgaacagggcgccaaaacctctccgacagccacgacggcatgtacttagggctctgtctcctctctgctagacatgttagcacattgctacaccccccattgtacacctgggccctctccttacgtctataaaaggaaggtccagggctctcgtacgagagggttggccgcgcgggagaacgggctgacgcacaaggctctctctctctcccacacgaacgcttgtaaccccctactgcaagcgcatccgccctgggcgcaggacaacacgaaggccgcgggttcccctttgctgttttcccccctttgtgttctgtctcgcgtcgacccatctgggctgggacacgcagcgacaatttactcgtcggtccagggaccccccggggtcgaaacgccgacaaaacaatattttctagctttggtacctacaatcttctgtacttccccatttgtctaatgcttcaggttgttcttttttttctgtagatctatgtaccttatccttgctatactgtccatatatgttgtgtgcatgaaagtcttgcattgaaaatgtcatgtgctacaatcgttaggactattaatagatgttgctctgtctatctatccatttacatcgctggaaattcccatgccctttcatagtacgcctgtgaaattctcactgcttttctattggtttgtgtgcagttcatgctctgcaaggtaaggtctgttcagtttggccagaaaggcatcccctgcctaaacacctacgacgaccgcaccatccgctaccccgacccgctcatcaaggccaacgacaccatcaagatcgacgaaatcttctagaattgnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnntcttatgtatcagcttgattcgttgcacattgttgagatgggcctctctttactcgctaatggacaataccgctcaagttttgggaccaagcgttcctcacctcaacacatcttatcaatagaactcctactaagcttcttgattatgacacatcgctccaccgtctcttaggtgctaccccagattactctaatctacgcgtctttggctatgcatgttagccaaatttgcggccatacaacacccataaactctagtttcggtccatttggtgtgcttttctagtctatagcaaccttcacaagggttacaagtgtcttgacatctcaacgggccgtgtttatatttcacatgatgttgtttttgatgagacgcttttccctttgctgctctccatcccacagtcggtgctcgatatacctctgacgtgcttcttctacccgatcctaataattctcgggccaactcagatgatcttgtgactaattctcctgctgaatccagcatgcttgctccgattttgtggcctaaccagcttttgcagccaccaatgatccctgctgcaaattctgtcccggctggtggcctcaatcccggtgctgatctgttgctaggctccacgccacacccctccgacgcggctacaggtgcgcccagcaacgcggtgcttcccaccaccacggccgcatcaatagcagcagccacttcgggattgcctcgtgccgactctggcgcggctggtccctctctcactgacagccatctgccctcgccatcagcctcgtgtcctattccgcttcctgctaggcgcactcggctacagagtggtattgtgaagcccagaaagtttacagatggcacgatcaggtatggaaatttggcaatttgtgaagaaccctccagcttgtctgttgcattgtttgacccaaactggaaaagctgccatggacctagaattttctgcccttatgcggaataaaacatggcacttggttcctcccgcacctgacagaaatttgattgattgcaagtgggtttataaactcaagagaaaagctgatgagtctattgaccatcataaagctcgattggtggctaaaggttttaaacagcgctacgacattgactatgatgacacttttagcctagtagttaaatttgctactgtccgccttattttgtctcttgctgtctctcagggttggagcctctgccaactggatgtgcagaacgcgtttcttcatggtgttctagaggaagatgtgtgtcggcaccctaaaactagggtaccccttactactgtataaagacgcagtacccacacgactatctttagtcgcgtggtaaataagctgtatgtgggaccagaccatgactcgccctagcctcgggcgactactctgggccagcaacagcacctgaccccaccacatgggcgggttcggggccgccatgtgtccagagaaagtgatgtactccaaggcatcaacagtgagtccggaccccatgggagagtgccggaccagtgccagacccctgtatatacggtccaggcctccaagtttggtccaggacctccacgtgtacaaaccggacccctaggatgggatccgaaccccccgtatgggtctgggccacccatagtggggtcccagggttctaggacagaacatacccgggccttgattaggacccaggtgggggtccggagccgacacgtgtctagacctggtctggtgggatccggacctatccgcatacactccttctccctgctcaggcggagacccgatgctgccacgtggcatactgcgcgcggcataaaccaacgggtggaacctggcatgatgcctctgggctacgcgtgccttcgcattcattacggagaagatgtgcgcctgtccattccactgacaggcggcatgctcagtccacgatacgtgggccatgcagttactcacacgttaccatatcgagggcaatgactcaccattactcgtatgtttccaagaaaagggttactgtctatcaatgctgcatggactgcagccatcatgactcccgctgattactcatgtgttactctgtcagcattagttattcacataatgtatttcttccattatgctcctgggcccacatgtcggggctcagcatccttgtatgtgcctcccttaaactataaaagggaaggcacacaacgttacaagggacacgctgtacacactcaatacaacatacacacagtggaggtagtgtattacgctccggcggcctgaaccactataatccctcgtgtcctcttgtgttcatcccgaattcaccaaacaggcaaccgcttaggccccctcctcatcttaggattagggcgggtgcattccgccacccggccggaggattttcccttcgacatttggtgctccaggtagggggctttggctttaggtttttgcctgttttcttgctcgacacgatggttcagatcgtcgagcaccgtggcttgtctcccgaggacttcttgatggaggaaggggcattatcttccatgccacgaggctccaaccgcgctgtgcctggtgctgctgctatgcacgctgcgcagcaacacacgcccgcacagacctctaggactccgtcgagggctacctatggtgggccattgtctgcagccagggagttgctgcgtaacccaccaagttccacggcctccccgggggccatgaggcagtggcgtgaagatgtcgaccgtctcctcggcatggcccatcctagctcggccaggtccaggcctcgatcattccggcatcagcgcgaggcgtcaacgtctgtgcattcaccctcagtgagggggcacagactaacgacctgcgagcagaactcaaccacaggcgtgcaggcgaggatgctcgaatctctctggagagggcgcgtgagcgccggcaaaacttcgagggtcgcaacctcgaccaagacttcactgcaagggacgcccgaatccagatgggtgtcccattggtcggcgtgggctgcgccgcactagcagatcatctccgcgcggcgacttggccacccatgttccggccacacctgccggagaagtacgatgggacatcgaacctgtcgaaattcctgtaggtctatgtcaccgccattacggcagctggtgggaacactgctgtaatggtaagctatttccatgtagccttgaatgtgccggcacagacctggctcatgaacctcaccccggggtcgatctactcctgggaagagctctgtgcacggttcacaatgaacttcgccagtgcttatcagtagcatggcgtggaggctcatctccatgcagtgaggcaggaacccgaggagactctccgggctttcatctcccgcttcaccaaggtacaggggactatacctcgcatctccgatgcctccattatcactgctttccaacaggggggtgcgtgataagaagatgttggagaaattggcgacgcatgacgtggaaaccgtcactacgctcttcactctggccgacaaatgtgccagagctactgagggccgtgcatggcactcgacgctgcaaaccagagtcacccaaatgggtggctcaggtgctgccacccagggtggtggcaagaaaaagaagaagcaccgtgtcacgataggccgtagtctggtgctccagttgctgtagctacggctggggaccgggacgagcgcggcaagcatccacggcaacagggaagtgacattgggtcatgccctgtccaccccaacagtcgccacagtgcctcagaatgacgagagatcctgaagctcgtgaagcgcatcagtgagcggcgcgagcatgcctccagggatggctcgccgcctcggcgccggcctggcaaggagaaggtcgacgaaggtgacctggccacgggagaatgggacctcgagaattaggcccccgagcaagtcctcaaggatatcctcactggagactccgactccggtgatgacaacgaccgccgcaagaagctgtacgtaatgtatggtggaagctgggagctcacctcccgtaggaacgtgaagtccctgcgccgcgaggtccttttggcgaccccaggggtcccgaaggcagccccacatcagcggtggcggagcaccactatctccttcggggcacccgactgccccgaaaacatggcaggggctggtatactaccactcatcactgcccctgtcatcgccaacatgaagttgcatcatgtgctgattgatggtggggttgggctcaacgtcatcagccacgctgcgttcaagcagctgcagatcccaggatcccgactaggaccctctcgcacgttctctggagtgggccctaaaccggtgtatccccttgggagcatcacactcctggttacattcgggactgaggataacttccacactaagaatgtctagttcgatgttgcggaggttaacctccctttcaatgccatcattggcaggccggccctgtaccggttcatgtccattgcccattacaggtacttggtcctcaagatgccatcccctgctggggtcctcaccatgcggggcgaccgtcccgctgcgcttgcagctatcgagaagttgcatgccctagcggcagaagctgctcgcccggatgacgaggggagggacccctcgacttcctgtaccaagatgcctgctaaggtgcctaaggtgcaaccatctggggcagacggcgtccctgtcaagaccatccggctcaacggggattcctcccagaccactcgcatcacgggcgatctggaggagaaataggaaatcgcgctcatcgccttcctccaggcaaatgccaatgtattcgcatgggaactatcgcagatgcctgggatccctagggaggtgatcgagcaacatctgaagatccaccctgacgccaaaccggtgagtcagaagcctcaaagacagtccatcgagcggcaggatttcatccgtaaggaggtccggaagctgctggacgctggtttcatcgaagaggtccatcacccagtatggctggccaatctagtcatcgtccccaaggctaacgggaagctttggatgtgcatcgactacaccagcctcaataaggcctgtcccaaggacccatatccacttccacgaatagatcaaatcgtggattctacctctgggtgcaacctcctatccttcctggatgcttactctagtttccatcagatcgagatgtctaggcaagataggaagcataccgcttttgtaactgtggatggactttactgttatgttgtaatgccttacagtctgaaaaacgccttgccaacatttgtacgggcgatgagtaatacttttggtgacttgattagggacagggtagaggtatacgtcgatgacatcgtagtcaagactaagggagggtcgaccctagtggaagacttaaccctagtctttgacaagctgcaggcaacacgcatgaagctgaacccggacaagtgcgtctttggtgtctctgcagggaagttgctaggattcctggtttcacaccggggcattgaagcaaacccagagaagatcaaagcaatagagacaatgaggcctccggcctgaatcaaagacgtccagaagcttacggggtcactggccgcccttagtcgcttcatctcaagactggttgagagggcactacccttcttcaagctattgcggaagtccgacccattctcttggaccaaagagacagaacaagcctttcaagagttgaagcagcaccatgtgtccctatcaatactggtagctccagagccaggagagccattatactagtacattgcagcggctacagaggcggtgagcatggtgctggtcgtcgaaagtacgacacaacatccctaggggagtcataaagttcccctaggagaaggtggtggtctgaccaccacgatgttgacagaaggccaggagtttgaggactcgggactgaatgcaggggtccgaaccatccagaagccggtctactacgtcagcgaggtcctccatgaggcaaaagccaggtaccttgagacgcacaagcttatctatgctatacttgttgtgtccaggaaattgcgccactatttttaggcacacagagttgtggtggtgacctccttcccgttaagggccattctccacaactcaaacgccacaggcaacatcgccaagtgggccacggagcttgctgagttccaactggagttccagccccgccacgctgtcaagagccaggtcctggctgacttcatcgtggagtggaccccttccccgagcgctcctgggggtccagatcccgattcggacaccacacctgcggagccaagggcttcggtcttcactgagccccactggatgcttttcttcgacggatccgcctgccagcagggtggcagtgctggagttgtgacaccccaggtgtcagtttcgtgttacgtcgcgagatttatcctaatctcggatgctcagtaaaaatttctatttctcgctcgcgtatgtccctgattatccagattattcattcatgtttcaccgaattcggagttactcagtctcatagaaggccaattttggagcctgttaaaacttttatccttggcacaaatgcgaactcaaaaatcattctcgaattataaacctcatctgaagctcaataaatcaaactctcgacggctgttatttgatctgtgtccgaatccaatttctcgatgttcgatcgatgtccaactattttaatccgagtccatactcacaaacgaaataatcaatatgtcgtcctctgatcaaatcttactcgactcagcttagcatctctgtatccaatccgatttcaaaatcaacatcggcaacgatttttatatatcacgattcgctttctccgactaaaaatccaaaaccgatcaaatctcaggacggtttattttcgatttacgcgtagggaattattttcaagcaaaatctaaacagactctcggctgagttaatcgcgcaaccttccgttcgtccgaactcttttcgctctgtttctcagtagcgacgaattccgcaggaacatttttagtccggaaattatttagcgcgacccaatttagtgttttgggccaaatccagtccagcccgtttggcccataagaaaccctaccctaatttctcctctataaatatgggcttccctaccttgcattctgaaaattttccatttccaccccagccgccaacacccttctcttcctcctctaccattttccagccatgggctccttcaagcacgtagagctggagctccttccccagcgcgcaggggcttccatggccgggcgttccttccctccagcgcgtcgaagctcttctcgtagcgtcctctgcctttcttcttccccgcttcacggcagcaaggccaccagcaggctccctgctccccgcgcccccagccatggcatccttcactcccctactgtttttctcccagggcgcagcagcaaatccatgcagcggctccatggccgagcaccctgcccggtgctccagccggcctcctctgcccctgccattttccataggagtcgagctcctacctgcagcaggcgccccctgctctttcctgtccgcgaccagggagcttcagctggcgtgaaacttcacttgcgcacggcggccagcaccctctccttgggctccaacagcttggatgccgaacccctttcttccttcccctggccgagctcgagcttcccatggagccattcctccctctctctgttgtacatagtgccaagcagcaactccattttccctggccgcgcccaaggtcggtgaccagcctccccttccctgttcttgccgtggccgagccaccacttccccagccgtagccctctccccctccattgtttcagcgcctgaaacaaacacctggccgccatccacacttgtgctcgatgaaatgtgcagcagccccgacggctccgcgtgctgccggcttgctgttttgttgcgtagtgagcagcacgccgtgatgccgccgtgtgttcgctgtttttgcgcagccccaaacgtcgtcgtcgttcaccccggtgagaccgcgacgctccttgtttgattccgcatcgatgttattttcctatgattaattatgtatgtgtgttgctttgttttatttttgtggaggagagaaccccgtgttttgcgaggagaaagcaagtcgcttaacgctcgttggatgtttggagcgatgcacgaatcggaatcaccgtcattcttgcaaacatcatttgggtttgtttatggtgagccgatgcatgtcgctctcgatcgactcgattaatcattttgtatggatgtgtgtaaaatgttcgattatgcgcattggtaggatcacgtttgcgattggagaacaagaggttaattgatgtgcacgatttgtagttgtctaattatgttttggtcgatgatgtgcatgtggttatatgtgtgtaactgtataattttataaatggacgcgtgtagggaagaaattgaaatagaaaagaactcgagtatttttattttgataggaaaatatgcgatgcgttgtttgatgcgaaaactaagttacaaaatgtggattttgttttgggaaatgcatcgatgtgtttatgtgaaaagtgtatttgttttaagcaatgtgatgggattcataattttagaggggatatatttattgatgtgacgagtagtttagagaatgctagtttgcgtagaggatgtatcgttaagacatgagtgtcagagtccatttatactagtggtcgcgccacatggattgaagtgtctcgagtgcacgccataatatggttgtatgcgagacagggttatgcgtacgatgagtttagtaaaaattccatcggtgtcagttgtgttaagttgaagtttatttgtgcgtataaagtagtaaggtatttaatgcttacgactcttaatcgatggtagaaattgtcttgacttaaatagagaggtggtgacatgccagagtagtcatcgctttctctatatttataggtcaagtcatgacgatgcgtattatgcgttcgttaaaattatgtttcgtatatagtgtatgattgtgctcacgatttcgagtagacacttcaaataagtcaagtagctttgtaatgcaagatgtgtgatgaagttagtttgttttaggatatgtgttgaaatgctccattcctgtgatagacatgtagggttatttcaaaacgggtcgatgtgtgtgatgatgatattcatgatttaagtagatgtcctgaaattatgtggcgaagcttaggttaagttgcaagcgatgtggaaatgttttcgtaaagatatatgtggaatgtgaacgagtcattcaatgtattcggtatgtcatgtagtggtggtatgaaaaatgggttaggaatcgatcggctaaatgccaagttcggttagagttattgtcggcgtttcgagaccggggggtccctcaggtcgacgagtgagtgccgcgtgcgccagcccagatgggtcgagcgcgtgggcgagcgcgaaggggggaaaggagcgaggcggccggagaccggcgtgagagaggtgggaatcccgcggccttcgtgttcgtcccgcgcccaggtcgggtgcgcttgcagtagggggttacaagcgtccacacgggtgagggaagcgagcggccccaagagagcgcctgtcccgtcctcgtcccgcgcggccaaccctctctaagaggaccctggtccttccttttatagacgcaaggagaggatccaggtgtacaatgggggtgtagcagagtgctacgtgtctagcgagggagagctagtgccctgagtacatgccaatgtggcagccgaagagatcttggaacccagctagtgtgatgtcgtggccgtcggaggagcggcggagcctggcggagggacagctgtcggagcggttgtgtccttgctgacgtcctcctgcttccgtaagagagctgagagctgccgtcgtcacagggcatgcggggcgccatcattgcctatctggtggagacagccagatgggacaccggtcttgttctctacggcccgagtcagctcggggtagggtgatgatggcgcttcctgttgacgtggctggcctgcgccctaggttgggcgacgtggaggctcctccgaagccgaggtcgagtctgtcttccatggccgaggacgagtccgagcccctgggtcgggcgaggcggaggtcgtcggcagaggccagggcggtgtccgagccctggggtcgggcgaagcggagttcgtcgtcttctggggctgagcccgagcccgagccctggggtcgggcgaagcggagttcgtcgtcttccgggtcttagcccgagtccgagccctgggtcggttggagcggagttcgccgtcttccgggtcttagcccgagtccgagccctgggtcggacggagcggagttcgccgtcttccgggtcttagcccgagtccgagccctgggtcgggcggagcggagttcgccgtcttccggggctgagcccgagtccgagccctgggtcgggcggagcggagttcgccgtcttccggggctgagcccgagtccgagccctgggtcgggcggagcttcctatggcgcctttggcagggcctggcttcctgtcagtatctctctgtcaagtggcactgcagtcgaagtggcgcaggcggcgctgtccttctgtcagaccggtcagtggagcggcgaagtgacggcggtcacttcggctctgccggagggcgcgcgtcaggataaaggtgtcaggtcacgtttgcgttaaatgctcctgcgacttggtcggtcggtgcggcgatttagtcagggttgcttcttagcgaaggcagggcctcgggcgagccgaagatgtgtccgccgttagaggggggcctcaggcgagacggaaatcctccggggtcggctgcccttgtccgaggctaggctcgggcgaggcgtgatcgagtcgctcgaatggactgatccctgacttaatcgcacccatcaggcctttgcagctttatgctgatgggggttaccagctgagaattaggagtcttgagggtacccctaattatggtccccgacagtagcccccgagcctcgaaaggagtgttagcactcgcttggaggctttcgtcgcacttttttgcaagggaccagcctttctcggttgcattttgttccggtgggtgcgcgcgagcgcacccgccgggtgtagcccccgaggcctcggaggagtggtttcactccttcgaggtcttaatgccttgcgtaatgcttcggctggtctggttgttccctcatgcgagctggccgtagcccgggtgtacggtcggggcccaagttctcgggctggtatgttgacgctgtcaacggtttggccggagccgggtttgcgagagcagcccctgagcctctgcacagggcaagagggcgatcagggacagactcggcttttttacatatgcccctgcgtcgcctttccgcaaggaggactagggggagggcgccatgttaccctcgatgggcgccgaacatggtgtctccggtgagctgcaagcaggtaatccgagtggacgtccgtgccccgttcgttaggggtcggctaggggcccagaggcacgcccaaaagtacctgcgggtgatctgccggacccggtcccctggcgacggggtccgagggctcgatgcctccctccgatgggattccattacaagatcgctcccgctggtctcggaaatgtcctagggtacctcaggagcgcagcccgagccttggttatgtatcgaacgtacccctggtcatccctcgctcggcgtctgaggcggctgtgaacccttcgggggccagccttcgaacccctgatcagtaatgggcacggagcccgagtagcctgaggcgaccgtggaacccttcggggggccggccttcgaacctctgaccagtagtgggtgtagggcccacgcgatctgaggcggctgttgaacccttcggggggccagccttcgaacctctgatcagtaaggaggctcggagcctggttccttcacggggaaggatccctttcggggtatccccctttcccggtccctgtcgcaagagatagagaaagaggaaaaagggaaaaggatacgaaaccgaacgacgcggcgtaccttttttggcgcggttatttcggcgaaggcgaagtgtcgcccgctgctcctgccagaagcgccgcctgtccagccgcggagttaatgcgacgaggcgagtagttggcggggcagccgttgcgcgtgcgcgagccgttcgaggaacggatcacgggcgcgttgtcttcacgccgtgagagggggttctcttgctgcccccggatgggacgtgagcttggctgacgacgtgaccgctgctcccacgcgcctgccaccgtcattactgccggcccacttttggccgtgttgaccgccgcgtcaggctggcgctgctgggtcgcacgctgggtcgcctcgagtcgcggtattggttccgcaatcgaggaggcgcggtggtggcgcaagtggcggtgcagttgcttgcatgtcgtcgtagtcagagcgggcggcggcgagccgctcgtcagtcttctgttgctccgtaggcccacccctatcgagtggggctgttcgtacctgcggaggggggaaccggagttccgtttgtaatggcacttcgaatgccggtgtttttgttcattgcggctttcggggcctgaacatgtatgtaattccggcacggagccgtgtttttcctcatttttgagcgctaagactcgtctgttgattatctgaaccgcttcaccaagcatgagtcgccccgtgtcaaggtgacgagtgaggtatccgtatcccggaggcgtaggagtccctcggctcggtcggccttgctgtccgaggctcctctagcttagttaaagggacccctcggccgctcttcgacgagccgaggccaggggtagcgatatcagtgtgaacagaggcggagttggctcgaaaatgaaacctggttggtcggagcctagccgggttgtccgttggcgggaccgacgtcggggctgatcagccgaggcctcaggtcgggctggcgcccttgggagatggtcggccgaggccccaggggtaaccggccgagccgcctgctcgggccggattcccggagaagtccctggcagcgattgcccgggcgtggtgatgacatcgtccttcggagcggagatcctcggaccgcgtcgccgtccgaggctaggtcgggcctcgctgaaggtgtcatcgatgccgagggtgttgctgcccccttccagcgtcaagacccgagcctgtagggtcagattgtcttgtagcgtgtgccttctgcagccgccgaggccagaatacacgccctcgctgtgttgtaaagctgcgtctcctttcctcttgtttcgagtatcttgacttttttgtcggtaacagggatgtttgtgtgagtgggagttgcttctcgcggaaggtgatgagtgaggtatccgtatcccggaggcgtggaagtccctcggctcggtcggccttgccgcttacacgtactttcactcgtccatgaggccctgccaccgactcagtcgagaaggctcgaaggattgcttcggcagaagaacttccgaacatgaagacttgttcggtccgcggaatcactttatccgaacgcgagttacttatcgcagaaggtgatgagtgaggtatccgtatcccggaggcgtaggagtccctcggctcggtccgccttgactgcttacgtgtactccgtcgttttcaggatccacttttcgaagtagtcaaaaagcacgaaagatattctggcagaagagaccttttttcgaggaaaatttcgacgcagagggggttcccccccttttagcccccgagggagggtcgggctttgccgaggcgaggccgacccttccttgatgactaaactttgcgtgggtgcgaggtatatgaacgacctgaaaacatcttaagggtagaagcgacgtagctgttggatgttccaagcgttgccgtagacctcgccttgactgttggccagcttgtacgttccgggcttcagaactttggcgatgacgaatggcccctcccaggggggcgtgagcttgtgcctccctcgggcgtcttgccgcagccgaagcaccaggtcgcccacctggaggtctcggggtcggacccctcgggcgtggtagcgccgcagggactgctgataccgcgccgagtgtagtaaggccttgtcccgagcctcttccagctggtccagcgagtcttctcggctagcttggttgctttgatcgtcgtaggccctcgtcctcggggagccgtattctaggtcagtgggcaagacggcctcagccccgtagaccaggaagaacggcgtgaaaacccgtggcccggctcggcgtcgtcctcaggctccagaccaccgaggggagttccttcatccatcgcttgccgaacttgttgaggtcgttgtaaatccgaggcttgagcccttgtagaatcatgccgctggcacactctacttgcccattcgacatgggatgagctacggcggcccagtccacccggatgtggtgatcctcgcagaagtccaagaattttctgccggtgaactgggtgccgttgtcggtgatgatggagttcaggaccccgaagcgatggatgatgttggtgaagaacgtcaccgcctgctcggacctgatgctgttcagaggtcggacctcgacccacttggagaatttgtcgatggcgaccagcaggtgcgtgtagcccccgggcgccttctgcaaagggccgacgaggtccagaccccacacagcgaagggccaggtgatgggtattgtctgcagagcctgagcgggcaggtgggtctactttgcatagaattgacacccttcgcaggtgcggacaattctagtggcgtcagccaccgccgttggccagtagaagccttgccggaaagcattcccaacgagggctcgaggcgctgcgtgatggccgcaagcccccgagtgtatctcttgcaggagttcctgaccttcggcgatggagatgcatcgctggaggatgcccgagggattgcggtggtagagctcctgctcatcgcccagcaagacgaacgacttggcgcgtcgcgctatccgtcgagcctcggctcggtcgaggggtagctctccttggcggagatattgcaggtacggggtctgccaatttcgatcaggcatggccccacttcgctcctcctcgatgcgcgatgcctcgccctcggagaccgagggtacctcgggttgagctgagggtgcctcgggccgtgccgagcgtacctcgggctggtccgagggcgcctcgggctcgggagggtcatcgatcttgacggagggctaatgcagatcccgggagaagacgtccggggaaccgttgttcgccccgaggctatttttgccagctcgtctgcagtctcgttgtagcgccgagcgatgtgattaagctcgagcccgtagaacttgtcttccaggcgccgaacctcatcgcaataggcctccatcttcgagtcgcgatagtgggagttcttcatgacttggtcgatgacgagctgcgagtcgccgcgagcgtcgaggcgtcggacccctagctcgatggcgattcgcaatccgttggtcagagcttcgtactcagccacattgttcgacgccgggaaatggaggcgtagcacatagcgtaggtgtttcccgaggggtgagacgaagagtaggcccgcgccggctcctgtcttcatcaatgacccgtcgaaaaacatggtccagagctccggttggatcggagccgtcggtagctgggtgtcgacccattcggctacgaagtccgccaagacctgggacttgatggccttccgaggcgcgaacgagatggtctcgcccatgatttccaccgcccacttcgcaatcctgcccgaggcctctcggcactggatgatctcccccagggggaaggatgacaccacagttaccgggtgagactcaaagtagtgtcgcaacttccgcctcgtcaggatcactgcatacagcagcttctgaacttgtgggtagcggatcttggtttcggacagtacctcactgacgaagtaaactagcctctgaatgggcaatgcatgcccctcttcttgcctctcgaccacaatcgcggcgctaaccacctgagtggtcgcggcgacgtagaccaagagggctttttctccatcagctgggggcaccaagataggcaccttggtgaggagcgccttcaggtctacgagagcttcctcggcctcaggggtccaagtgaagcactcggccttccttaagaggcggtacagaggcaggcctctttcgccgaggcgtgagatgaagcggctcagggccgcgagacatcccatgaccctctgtacaccttttaagtccttgatgggccccatgctggtgatggctgcgatcttctccaggttggcttcgatgccccgctcggagacgatgaaccccaagagcatgccccggggcaccccgaagacacacttctcgggattgagcttgacgccttttgccttgagacaccggaatgtcacttcaaggtcggagaggaggtcggaagctttccttgtcttgactatgatgtcatcgacgtaggcctcgaccgtgcgaccgatgtgttcgccgaacacatggttcatgcaccgctggtacgtcgcacccgcattcctcaaaccgaacggcatggtgacatagcagtacatgccgaagggcgtgatgaaagaagtcgcgagctggtcggactctttcatcctgatttgatgataccctgagtaggcatcgaggaaagacagggtttcgcacccagcagtggaatccacgatttgatcgatgcgaggcagagggtaaggaaccttcggacatgctttgttgagaccagtgtagtctacacacatccgccatttcccccctttctttctcacaagcacagggttggcgagccattcgggatggaatacctctttgatgaacccggctgccattagcttgtggatctcctcgcctatcgctctgcgcttctcctcgtcgaatcggcgcagaggctgcttgaccggtcgggctccggcccgaatatccagcgagtgctcggcgacatccctcggtatgctaggcatgtctgagggactccacgcgaagacgtcggcgttcgcgcggagaaagtcgacgagcactgcttcctatttgggctcgagcccggaaccgatccggatctgcttggaggcgtcgccactggggtcgagggggacggccttagccgtctccactggctcgaagttgccggcatgacgcttcacgtctggcacctctttggagaggctctccaggtcggcgatgagggcctcggactcggcgagggcctcggcgtactccacgcactccacgtcgcattcgaacgcgtgtttgtacgtggggccgacggtgatgaccccgttggggcccggcatcttgagcttcaggtaggtgtagttggggacggccatgaacttcgcgtagcatggcctccccagcaccgcgtggtaggttcctcggaacccgaccacctcgaacgtcagagtctcccttccgaagttggagggtgttccgaaacagacagggaggtcgagtcgtccgaggggctggacgcgcttcccgggaatgatcccgtggaagggcgcagcgcctgctcggacggaggacagatcgacgcgcaggagcccgagggtctcggcgttgatgatgttgaggctgctgcccccgtccataaggaccttggtgagcctgacgtcaccgatgacagggtcgacgacgagtgggtatttccccgggctcggcacgtggtcggggtgatcagcttggtcgaaggtgatgggcttgtcggaccagtctaggtaggctggcgccgccaccttcaccgagcagacctcccggcgctcttgcttgcgatgctgagccgaggcattcgccacatgcccgccgtagatcatgaagcagtcgcggacctcgtggaactctcctacttggtgatcttccttcttgtcgtcgtcgcgggccctgccaccctccgcgggtggcccggccctgtggaagtggcgccgaagcatgacgcactcctcaagggtgtgcttgacgggcccctgatgataggggcacggctccttgagcatcttgtcaaagaggttggcacctccggggggctttcgagggttcttgtactcggcggcggcgacaaggtccgcgtcggcggcgtcgcgtttcgcttacgacttcttcttgcctttcttcttggcgccgcacggagtagacgcctcgggagcatcttccgacgggcggccctggggctgcttgtcctttcggaagatagcctcgaccgcctcctggccagaggcgaacttggtggcgatgtccatcagctcgctcgccctggtgggggtcttgcgacccaacttgctcaccaggtcgcggcaggtggtgccggcaaggaacgcgccgatgacatctgagtcggtgatgttgggcagctcggtgcgctgcttcgagaatcgccggatgtagtcccgaagagactctcccggctgctgtcagcagcttcggaggtcccaggaattcccagggcgcacatacgtgccctggaaatttccggcgaaggcttggaccaggtcatcccagttggagatctgccccggaggcaggtgctccaaccaggcgcgagcggtgtcggagaggaacagggggaggttgcggatgatgaggttgtcgtcgtctgttccacccagttggcaggccaggcggtagtccgcgagccacaaatccggcctcgtttcccccgagtactttgtgatagtagtcgggggtcggaaccgggtcgggaacggtgcccgccggatggcccggctgaaggcctgcggaccgggtggttcgggcgagggactccgatcctccccgctgtcgtagcgtcccccacgcctggggtgatagcctcagcgcaccctctcgtcgaggtgggctcgacggtcgcagtgatggcgctcgttgccgaggtggcccggggccgcaggcgcggtgttgcgcgtgcgcccggtgtagaccgaggcttcccgcatgaatcgggaagtcgcggcatgaggttccgaggggtatccttgccttcgggaggcagtgctctcggcccgtcggaccgtggcgccttccaggagatttttgagctctccctagattcgccgaccctcggtggtggatggctccggcatcgcgcggaggagcatcgctgctgcgaccaggttctgaccgaccccactggatgcaggtggtggcctgaccctgacgacatcggcgacgcggtgctggagaccctggggcaggtgacgtatttctccggccgggggttggcccgcccatgcctgcccgacgtcccggcggatcggctcaagcgctcctgctccctcgtcgatcctggcctgcgccccgcggacttgctcgagctgtgggtcgtaaccccccgccggaacagggaccacaactagctcccgcgggatgtcagcgcgaggcaccggcccagggggagcaccgtcctccggcatgccgagatgattgccttcggagggaccccctagatcgacgtggaaacattcgcggcttgggccgcagtcctcgtcgtcgaggctgcggctaccgtcggaacagtcggagaggcagtagtcacatgcggtcatgaagttccgctggcactagggttgccaaatccagagaaatcccaacagatgttggggtcgtcatcttcctcggacccagagggcccgtaggtcgagacgtccgtcagccggtcccaaggcgaccgcaagcgaaaccccagagggtttgtactcgcctctacaagggcgcccgccaaagcaagattgctagacgggttgaggctgagtacaaatgacgtaggatgggaatcggttggtaccttttggtcgtcgagcggcgatgaagtcacgtcgaggactgaccgcatcgtcgcctcaggtacgagggcgatgtcctgcaagcttttcgcaagcgcgctggcgtcgtccacttgctcgggattggcgtgtcgcggggagacggcgctcgcctttgtctcaaacgcgaggtcgacgcccaacgcgccccccgttggggtgctagggacgtcgactcgctcgacagccgacgaggcgcggcctcctgcttggcctttgttgccccgcctcctcctccgttggcgggggagaggacggggcgagctcgaatgttgttcttccgccacgcggggaagacgtcgtcgattccgccgccggcgggcgggctgtcggccgccatcgtcgttgtcgcgcggcggtggaaggagtatcatgtcgtagctgccgtcgagggacatgaactcaagactcccgaaacggagcaccgtcccgggttggagaggttgttggagactgcccatctggagctcgacgggaagctgttcgtcaacacgcagcaggcccctacctggcgcgccaactgtaggcgtttcgagaccggggggtccctcaggccgacgagtgagtgccgcgtgccccagcccagatgggtcgagcgcgtgggcaagcgtgaaggggggaaaggagcgaggcggccggagaccggcgtgagagaggtgggaatcacgcggccttcgtgttcgtcccgcgcccaggtcgggtgcgcttgcagtagggggttacaagtgtccacgcgggtgagggaagcgagcggccccaagagagcgcctgtcccgtcctcgtcccgcgcggccaaccctctctaagagggccctggtccttccttttatagacgcaaggagaggatccatgtgtacaatgggggtgtagcagagtgctacgtgtctagcgagggagagctagtgccctgagtacatgccaatgtggcagccggagagatcttggaacccagctagtgtgatgtcgtggccgtcggaggagcggcggagcctggcggagggacagctgtcggagcggttgtgtccttgccgacgtcctcctgcttccgtaagagagctgagagctgccgtcgtcacagggcatgcggggcgccatcattgcctatctggtggagacagccagatgggacaccggtcttgttctctacggtccgagtcagctcggggtagggtaatgatggcgcttcctgttgacgtggctggcctgcgccctagtctgggggtacgtggaggctcctccgaagccgaggtggagtggatcttccatggccgagggtcgagtccgaagcccactgggtcgggccaaggcggaaggtcgtcggcaaaagtccagggcggtgtccgagccctgggctcgggtgaagcggaattcgtcgtcttctggggctgagctcgagcccgagccctggggtcgggcgaagcggagttcgtcgtcttccgggtcttagcccgagtccgagccctgggtcgggcggagcggagttcgccgtcttccgggtcttagcccgagtccgagccctgggtcgggcagagcggagttcgccgtcttccgggtcttagcccgagtccgagccctgggtcgggcggagcggagttcgccgtcttccggggctgagcccgagtccgagccctgggtcgggcggagcggagttcgccgtcttccggggctgagcccgagtccgagccctgggtcgggcggagcttcctatggcgcctttggcagggcctggcttcctgtcaatatcactctgtcaagtggcactgcagtcgaagtggcgcaggcggcgctgtccttctgtcagaccggtcagtggagcggcgaagtgacggcggtcacttcggctctgccggagggcgcgcgtcaggataaaggtgtcaggccacctttgcgttaaatgctcctgcgacttggtcggtcggtgcggcgatttagtcagggttgcttcttagcgaaggcagggcctcgggcgagccgaagatgtgtccgccgttagaggggggcctcgggcgagacggaaatcctctggggtcggctgcccttgtccgaggctaggctcgggcgaggcgtgatcgagtcgctcgaatggactgatccctgacttaatcgcacctatcaggcctttgcagctttatgctggtgggggttaccagctgagaattaggagtcttgagggtacccctaattatggtctccgacagttattttgatagttgggattgtggggtgaagtgatggcatgactacgtagccgtcacgtcatctattgcgtggctatgcttaagcgtgccttgatataatttagaataagtcgagtctctagaacgcggcaatttttaaaagtaaatagaagctgaatttattgattgctgttttgggctgcacgcactgttttagttgtgctgtttgtttgataaaccaaatcatgttttctatagaaaagtcatatagaagagttgtagatgacatgattatcttgcttgtactaaaatttgacagccataaacctgattgtttaggagttgtgcttttcacaagcccagcacctgaatctgtcaaatttctgaacatatttcagaaattgcaatgattgcttaagttaatgttgaaattagttattggtggtcacaaaaaagttgtagataactttattatcgtacttgtgttaaaatttgacaggcataagtctgattgtttaggagttatgttttttacaaattcagtaactgaatctgtccactttctgtacagatttcaaaagctgcattgtttgcttaagttaatgttagaatcagcccttgtcaattataagaaagttgtagaggcttttcttgtcttgcttgtgttaaaatttcataactataggcctgacggtttaagagttatgaattttacaaactggttgctgtgttctgtccaccgtcagaacagatttcgaaaactgtaatatttgatttagttaaacctggaatcacttcttggtgattataaaagttgtgtagtacttttgctaagcttttcaaaaagtcttagatcactctttttggtggtctgaagattaagttacatgtgtttgaagtgtgaagactgaatctgtccagttttggacagcacagccttcatagtatattttaaccttgatacatgctaaaccagcctgggatgtttataaataatttgtagaacatttaattagctttccagaaagtctaggatcaatttgtttggatgtctgaatcttcagttatgaatttttaaaatcacaagtctgaatctgtccaaatctggacagagctgctgtgattgcactttttgaccttgctaagtgtttaatcatgctgtgatgaaaataccaaaattgtagagcactttctaaactttccagaaagttttagtttgctatttttggattaatattttaaaagttatgattaaaacaagtagctgctgtgctgctgtcctaaaaatctgcacgtgctcaaatgaatatttagttcaccattttggctaaaaacgctttagtaagcacttaacggacatagacttgtgatggctaaacttaggttaacatgtgttccataattaatgtgtttgcttgctgtagttgattgtgatagaggagtccatcgacattgatgcatcggtccttttattaaacttgtgtttgtgatgtttttgtgtgatcaatataagaattaatgaaaagccgtagcaactaaataaatgcttgtacatatgatatcgtgttgcgttggttaattgtaggtagtgatcattgtctttccagtggtagtgtttacgtgtgcccaatgacacataaataactagtgtttgcgtatagttgttgcagtgtcttactaattaatgtttagttcgccactgtgtcttggtatatcttatgttacttttattatattcatacatatgcatcttgcacctcatataggaccgagagatgatgatcgagccagtgatgtggtgccaaccacaagatgccgttgatggacgacctgaagaatggacttaaccagtggatgctcaccaagcgagtacctcccccagcaaacactacctaagtgttaaattaaaggcaagccccggttttatgcataaccattatatatatgctattttactgcacttaatgtttgtaggcttgtaccgtgcacttaagtgtaggagttgaatgaaaccctagttgcatgaactcaggattccctttgagatggatactagtatgctaggtcgagtagctnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnacagtgcacagtgcaccggactttccggtgagcctaggcagaggtgattttgaaaattttcaaatttttcgatctaaattttaaccaaaccaaatcccaacttataatcatacaaaagaacacctattgggataggtattggccccctcatatattttcccataattttcaaaaatattttgccataggctagtcaatttttagagaaaatagtcaaatggtgagatttgcattttagctttgaactaggggttttcatgaataatttgagttttgaatactccccccctaagtgtagtactacatgcatatctcaagaaccaacaatggcatagtaaataagaatttaagtactaaaagcttaaagctaagacttgtcaagtttgagcccgagttaagcttttttcactcgctttgttggcggttatcttaactaggttagacaagccctagatgcaatacaagaaatttaaatatgcaatgcaggcttgacaacactattttgagatctttaaataaaatttctgagatcaagtatgtttaattcatttctcaacatgcaaaagcgggttttatcaagaggcttagtgaaaatatcctctaattgatcttccgacctcacttcttctaaaataatgtctcctttagcaatatgatctctaaggaagtgatgacgaatatcaatgtgcttggtgcgagagtgttgtacaagattattagcaattttaacagcactctcattgtcacacaacaaaggtaccttttctagaactacaccatagtctagaagagtttgtttcatatataaaatctgtgtgcaacaagcaccagcggcaatgtattccgcttcggcggttgacaaggcaacactatttttctttttggatgtccataatagtagtgatctcccaagcaaattacacccctagaagtactttttctatcaattttgcaaccggcataatccgaatcggaatagccaattaaatcaaaagtagctcctttgggataccaaaggccaacacttggggtgtgcttgagatacctaagaattcttttaagagcgcaaatatgagctttcttaggatttgattgaaatctagcacacatgcatacactaaacatgatatcgggcctagatgcaataagatacaataaactaccaatcatagaacggtagagagttaccatcttttcaacaagtgatctccatgttgaaccttttcaacaagtctttggtatacttctcttgtgagaggaagttaccatctttcatttgcttcacttgaaagccgaggaaatacatcagctcaccaatcattgacatctcgaactccttcgacatcaactcaccaaattccttgcaatgatagcgatttatcgagccaaagattatgtcatcaacatatacttgacaaatgaaaatatcaccgttatgtttctttgtgaatagagttgtgtcgacggtcttgatcttgaagccctttttgatgaggaagtcgcgaagacgctcataccaagcccttggagcttgctttaacccatatagcgccttggacaacctataagcatggttaggatatctagggtcttcaaacccgggtggttgctcaacatatacaagttcatttatgaagccatttaaaaatgcactttttacatccatttgataaagctttttatcatagcatgatgcatatgcgagtaggatacagatggcttcaagtcgagcaaccggtgcaaaggtctctccaaaatctaaaccttcaacttgagagaagccctttgcaacaagtcttgccttgttcctcacaatcacgccttgatcatcttgtttgtttctgaataaccactttgttccaatgatccttgcatcttgtggaggcttctccagggtccaaacttggttacgggtgaagttgtttagttgttcatgcatggcattcacccagtccggatcctgtagcgcctcatctatacagtaggctcaacacaagaaacaaaggagtgatgttcaataaaagaagcatgtttatgtgatcgagtaataaccccttgtgaaggacttccaatgatttgatcttgtgggtgtgcttgaagcagtgatgagtttctcctatcaaccacttaggaagaagatcctggagcatcaacatcttcggcttgtatccttgcttgttcatgagagacaaatgtatcttcatttgcatgcctctcatctttttcatcatcttgtggtacacttgatgaagaaggcctattaatgttttgcacctcttcttcatcttcttttggtttgatagctccaattggcatgttcttcatggcttccttaagtggctcatcacctacatcatcaagattttcaagtgctccttgggagccattagtctcatcaaactccacatcatatgtttattctaccacgccagtggcatgattgaatactcgatatgctttggactttaatgaataacccagaagaaaaccaatatcacaacgtctttgaaacttccctaggtgatggcgtttcttgtaaatgtagcatttgcatccaaacacccaaaagaatgagacgtctggctttttcccatttagcagttcatagagagtcttcgcaagtagccagtgaggaaatagcctgtttgatgcataacatgcagtgttgatagcttcggcccaaaacctctccggtgtgttatactcatcaatcattgtccttgcaagtgtgatcaaggtcctatttttcctttcaacaactccattttgttgaggtgtatatgttgctgatacttcatgcttgatcccaatctcatcacagtattcatgaatgttggtgttgtcaaattcttttccattatcacttctaatcttcttgatcttgtaatcaaattcattttgagctttcttggcaaacttcttgaatatagatgcaacttcagatttatcatggagaaaaacacccaagtgtatcttgagaaatcatcaactatcaccagacagtagaggttgccaccagcacttgcataagttgttggtccaaatagatccatgtgaagtagttccagtggccttgatgttgacatgaaagcttttgtaggatgtgtgttagcaacttgctttccagcttgataagcactacaaggcttgtccttttcaaatataacatcctttagtcctctaaccatgtccttctttaatactttcttcagtgtgctcattccaacatgtgcaagccttctatgccatagtcatccaagagatgctttggtaaagaggcaagttcttaagtctgcatcttcagaggtgaaatccactaagtagagattgttgtatctaaatcctttgagcaccatttattcatcatccatttttgatacaataacctctgttggagtgaataagcattgaagtccaagaacacagagttgacccactgataataagttgaatcttaaaggtgcaaccaagagaacatttgaaattgatagatcatttgaaattgccaccttgccaagtccttgaacttttccctttgaattgtccccaaatgtgattttgtcttgtccatcaacattatcatcaagtgaggtgaacatccgtgggttgcctatcatatgttatgtgcatccactatcaataacccaatggctcccaccggtcttgtagttcacctacatccacagacaaatcaagcctaagttttgagggccctatattgcataggaccagtgactttctcaatcaaggactttgcaacccaaatttgtctaggtctactcttgcttggtggacctaggaatgtaacttcgacttttccatttgctacttcttaaaacataatgagcattgaaggcaaatggtcttgagtgctttggcaggggagttggtggtttggctttgcagttgtgggaaaaatgaccttcttttccacactcaaagcatttgatatgcttatgagtctgatttggcttgtattgagttgtagctttcttttgcacaaaggagttatatccaataccactcttgttgtttttcatgacagtgttcatgagcaattcattttagaggtattggcccttgttgaacttttgcacacttgttgcaagatgttcattttcacgcttaagtttcttgacctcattttaagcctttctttatccactgccaactcattgttgaagtcattgttctcaatggcaacctttcctctagtagttgcatcagtcaagtatctcttcaatttttggttgtctaatgtcaagacttcaactttttcagtcaattcatcatgtagactagtttgatcaccttgactcaaatcatcacatgaggttgctacatcaatattaacaacagggttaatagcctcatgtgtattgcaagataaaagttcattttcaacaagaagattatcatgatcaaatttaatcttagtatagtcttccttattttttactagtctatctttcaactccctattagcttcatttaacttgtcacatttatccttagcatcttttagggaggatgtaagctcatttacagtggatgacatagttttgttttcttctttcatttcattactagctttcataactatgtcatatttagcatttaaaaattcattttcatctttcaacttatcacatttagcttttgacttcctaatgagttgagtgtattcattaagcaagtcaactagttcatcataggaaggtgaagcaaattcttcatcactatcactatcactatcatcaataatatcattatcattttgtacttttcgttcacctctagccatgaggcataggtgagaagtcgatgatggagatggtggtggtgaagagaagtccccagcgatggcggtaactttttcatcattttcttcttcacttgaagaagatccacttgacgactcaatgtcagtgagccaatcaccaacaatgtatgcctttacatttttctttttgtggaacctcttatgctttccatccttcctcttgaagaatctcttttcatttttctcatcatcactgtcatcttctttcttgcccttgaacttgttcttcttggacttgttacattgatgagcaagatgaccaagctctccacagttgtagcaatccatttaagaaatgggctttcttttgctggaaaagaatttcttctttcttgagtcaaatttgatgccttctctgttgagcttctttaatatcttggtggtcttcctcaccatcaaggcaatgttagcattaagatcatcgtcacttgaggattcctcctcaacttgtactttagcttttccttctctttcttgattttctttgagagccaaatcctttctcttgtaagatgactcatccttgtcattgatgtgcatgtacatctcatgtgcattgatctttcccaaaatttgtgtaggagtgacaactgaaagatccatctgatgcagcacagtgacaatgtgtccatatttatcaattgggaggacactgagaatcttcctcacaacatccggttgtgaaatttgtgtaagccccaagccatttacttcctctacaagaatattgagacgtgagtacatagcattggcattttcattagcaagcatttcaaaagaatttaattttctcatagcaatgtgatatctctcctcacgctcaattctagttccttcatgtagagcacatatgtccatccacaaatcatgacaatttttatggtttctaactctattaaacacatctttgcaaaggcctctaaaaagggtgtttttggccttagcattccatttctcatagttcaactcttcacctacaagatttgtgggatctctaggttcggggaatctttgtgtggcggctttgtagacaccaatgtctatagcctctaaatatgcttccatacgaattttccaatatggaaaatcgtcaccataaaaaacgggagaaggtccatccccaccggacatcgttactctagcggttaagctaatctaagagcaacaaggctcttataccaattgaaaggatcacgatgcccaagagggggggttgaattgggcttttctaaaaatcaacactaactaaaatctaagcaagagcccaacttcaccccgacaactagcactaagagaataatactagaaatacaacaatgctaagataatacttcaaatacttgctaaacaaatacacaatgtaaaatacttgaattaagtgcggaatgtaaagcaaggtttagaagactcctccaatttttctagaggtatcaaagagtcggcactctcccctagtcctcgttggagcacctgcgtaagggtatcgctctcccttggtcatcgcaagaaccaagtgctcacaacgagatgatcctttgccactccggcgcggtggatccctcacgaccgcttacaaacttgagtcgggtcaccaacaagatctccacggtgatcaccgagctcccaacgccaccaagccgtctaggtgatgccgatcaccaagagtaataagccatagactttcacttgaccaagagaagcctaatgcatgcggtgtgtgctctaggtggctctcgctagcgttaatgaggtccaaatgcgggattaagattctcaagtcacctcactaggctttgtggtgcttgcaatgctctaccaatgtgtaggagtaaatgtgggcagcaagaccatcaatatggtaggtggatggggtataaatagccctcacccaccaactagccattaccaggaatctgctgcgcatgggcgcaccggacagtccggtgtgccaccggtgcgccaacggtcgactcaaacggctagttctgacagctagccgttggacagatggcataccggacagtccgatacgctgtccggtgtgcctctaaaattcaactcacgaacagcgcgctctcgggtttctgcgcgcagggaaccctcttccctgggccaggctgggcccactggcaaagggtgcaccggacagtccggtgccccaaagccagaaaccctagcttctgttttgtgctgttttttcaatttggtttttgttctaacttgtgagtatgttctagagttacacctagcactatatgtgagtgtgaatatgcaccaacactacactagaactcttttggtcaaactacttatcgacaacccctctttatagtacggctaaaacaaaataaaagacctaactatatcacgagtgtccgcaactccttgacactcggaatacgaagaccttcactttttgtttcgtcgctttagccgttgcttcaagtttttatctccgggattgttttcaccattgtagtacatctacctgtaatgcgacctaacttaccatttgcctctgcaaaacacatgttagtcacatataaaattacgttgtcattaatcactaaaaccaaccaggggcctagatgctttctagtttaaatccccaacaagtcaaaattctttctatttttttttgcaagttccaattgacatctgaaaggttgtaaggtacacgtttggctctcattgataacgggggaaagatacagtgcaaaccaccatataatgacccacttctaatcgaatggacctgtaacgacgaaataccctgtgagaactatggttcactcatgttaattcattgaaattgttgtagtgaattgacatggttgggagcctgcttagagagtatagattgtcacttttttttggaccgcaacttatttttaaaagatattgcgatcgcttgtttagtagctgtttcaggccccaatgcagtttctatcgtgatccatttaagtcactcaacattctcatacttctcattttgcattaattcattccaatctccactactataaaatactagcttcgatggtcgtcatacgccatgcacgaagcatgtagatcaatccgcataccagtgggcatctatagataggctgtgaaaaccacccaaatccctactagtggacattttatctatagatggaccgtgagaaaccacacaagtctaacacgacagggaagccaaacgcagcgcagcgctcccacatagaaccacctcactacctaaaggaggacaagccatcgagcaagctttaaaaaagtagtcaggcttctttcaactcatacctttcctgatattttagctaagataaaagcgtaatatttgtttttatcagtttagtatctgatatatggaccatatgttcactttgatatttgatattatttttttattggtatcaaatatgattgtatgtcgtcgcagcgcacatgtgttgtactagttattttataagataatcaagtatttcttaatcatttaagacattttgatgattatttaaaacattctatttttttctcagtcattcactcgttaggtcattcagtacatattatgttaaattaagtcattctgttacaattctagtcatcacatgtcatttagtcattttatgacttatttaaaatatttcatattgtcaacagttgttacaagactttcttacaaatattttaagtcatccaatagtttattcatccagagactcataatatgtttttaagtcattcctttctattaaattgatgtaattatttttatcacgattggacttctttcttttatcacttagaagccgtgcgagatgaaagtctcatgcacggttttgcatgagagaaagaagcgaggaattctctttttgactctgactcccccactccaatcgttgcttttctttctgttacttcgaaagtagttgcttcagctttagccacgcgaattctcgatattcctttttatttctcatcaaacgaatgacatcttcttctggaaatcctagctattcttagcatgatattggagaatctccttgctattagtcaaacaagcatctgattggagcacaggcgtgtggggggagggatgctcaatgggttattgaggtgtgatggatagagcatccggttagagcgcagggcacgcagtggatactatttggcaccacgctcagcgagtatgcgtgtatgcagtcatgcaacccgcatatataggcataaaaaaccaaaatccctttttttgttatattcgtgtttatgagattttcgaacaaaactagacactcatgctatatctttttcaattttttatttaatcgcaatgtccgaccctaataaatacaatgattggtcctaataaatacgatgactggctctaataaaaaatacaatgacttatcttgatagctataatgagtgaccctgataaaatacaatgattgaacctaataatacaataactaaccctgataaaaatatcctgctaaatacaatgactgaccctaataaaaaaatacaatgaccgaccttgataactataatgagtgactctgatataaatacaatgactgatcctaataatacaatgattgaccttaataaatacattgactgacactgattaaaatataatgattgatcctgataactacaataactgaccttgataaaatgtagaccctaatagaagaagtacaatgactgatcctgataaaatacaatgactggccctggtaaaaaataaaatgaccctaataattacaatgaatgaccctgataaatacacgactgatcctagtaactataatgattgaccttgataaaagtacaagtgattcaccttgataactacaaatgattgatcctaataacataaagataaaggagaacaaatgagaggttggttatgaaataattggggaaatttgggctagccagttgcatgggtccgacctagtcacgaaccagccagccaggcgcgtggaataaccacacaaaaaataggacgtggggattcaaaccatgctctttcgatacaagcgagcgtcttctaccactataacttatgtctgtttatgttatataaaggagagatattgtatgtgtgcacacatatatacacacatacactataaaactgatgtcagccattcacattttgttcaaccatccattatcttttgttgagccatttctaatcaataccacttgtcgggtatcataattaggggtacccagattatgcccctaaaacacacttaacccttagaccaccttcaagacacattccccgagatcaaaggatcataaaccgcgcttcgcccgaggccccgctcaggggtcaccataggtccgcttcgctcaagcctgccctcggacatggtgtgctctagggagaattctcgtcccggccgaggctccatctcccagaacaaaagtctttgcctcgcccgagcacatctcgggtaaggaagacaaccccaatgcaagactcaaccaaagtctgcagggggcaggagcattcaatatgcatacctaccccacgtagagttgcaggtgaacaggagcaacaagaccgcggtcctgtcaagcttcaccaactacgatgacgcatgcgaccactattcccacatgccatctgtcaacccctgatgggacgtacaatacgacaagagtgcaggatggctctcggacgtgaactctgcctcgctgaaggcgacctcggcctcgggacaaacttcgcctcgcctgagcccggcctcgtttacctgctccccgcgaatactggagcgggctcggtcgtgacctcgggcggacttctgcctcgcccgagcccgactctagcctcaatatccacaacggaaaggcgcccaacgtcaccatatactgcagagctgacatattacttagggactttttgccatactcagtactgtgtcaaccactacggcatgggcaacccccttgtcaggggggctcgggtacgtgaccaagcgctcagcccttgcctcggctctcagcagaaatcaagcgggcacaagtcaccaaacaagtacaagaccatgcttcttgaagatctttgagtgatttctgcagatttgaacttttttcaacttcagcttcgagttttgtttcgaaatctttcttctcttgctcaatgctttttgacttcatggaaagttcactattcagtctggcgatctcggcttgagcttctgccagtgaaccttccattgtttgaattatgaagtctttcttttctagggcagcttcatgatctttaatcttgttctctaagccctcaattataacttcgtttttcttgtcctcgaggtcttgttgcatcctcaaggttttgcttagtagtaggctctgacaaaataaccttcatcagaaaacatcttcatatcaaaacaataaaaagttaagggaagaattttaccttaaagttagaataaaataggctaccgacgatatgctgtcgtcggtatctgctgagatcggcttcgagtttcggaaaaccaacacttttcgataaagtcctgacaactttctccccagtccggtctcaaaggcaacctaagctctcttcgtctataccaccgaagagcagtgcccctggctggtacccgcaagatatagcaaagtccctcagctcttcttttttagccttagacaactcttgtccaattatgttttgaaagttgatatttctttcttccgaagcatcttcggcaagctccttctccttttcaggcactgtagccggggtttcctcagcagctgcagcagtttcttcctcagccatattcaaaattatttcatcaatgtcagtaagcgtgttttccaaatttgtggcttcggctgctgcaacttcggaagtagaagcttcggctggagcagctttggctgctgctgtttttagcactgaggccgacgatggtgtttcttcaatagcctcaatgatagtaataatccttcgcttttttggttcagcgggcttctcggcaaccgaaggttccttcttcttcttctgtaaaagcttcatcagttccggtcccagcgggcttagcttattaggtagagattcggtcattacctttaaaatttcttctgcgtcagtggcagaaggtgttgagggaacttcttctaaatcagcttttggcttcggagctgtagcttttcttttcttcgaaacggccaccttcggctcagggctggattttttttcttttttgctaaattttcatcttcttttatcattctggcagcttgtctttgcataacactgacagctcttttttgttttggcccttcggcacctttacttaaccgttcatagtctgggtattcaaatttcagagtgttcattactcggtttagccttcgtttcggtcgggtgccgaaggctgccgtcatcaattgatcttctttcttcgtataattgcccaatatttcattgcacataacttcgatcgtatccaaccattcttggcagggttctttgaagtgtttcttgaacttaaaatgatagggcagtcgaacaagttcattctttttcttctctcctttaagcttcggcatactccattcctttaacgttgggaatactctattggctaagtattcctgaaccaaatccctagttccgatatgctcggacacaactctaaattcacccacaacatctgggcatgatgatcccagcgtcatgcgacactggggcctagttaacccgaaggttaggcccagtgggctctaaactagcttctccttcttctcatcaaccttaacataaaaccattcagttttccaaccggttgtccatttggtgcggtagctaaccaacggtgtcttcatgtctttgcggtaggcaaaattatagcagccgaagttctcgtgcagtccatcttctctagccttcgtctgatagtgaagttcgtgcacccggtagaaggcttcggcaagcggctccactccttggcttcgaagagcccagataaagacgctaagcctaacgatagcgttaggagtcagctgatgaaaataaatttcgaaattttccaaaacatccacaatcatcccatgcagaggaaacctcagtcctgctttaaagaaacttctgaaaactaccacctcatcattttctagcttcggagtgatttattctccgccaaaacgaattagcttcttctcggcttccccgaagtagcctagcttcgtcatcatgggcatatcggcctcagagacggtagactttccaaattccaagtggctgggtttagatggcatgacgaaataatctatctcctcttcatcagcctcaccttcttcaatgtcagcctgctcggtttcggcagcacgtgcaccttcgtcagaaacaccctctagcacaaccaagcctgattgtctcattacttcggagattggggcggtctcggcagcttcggcctcctccccgtcgcgtgtgactctagcagttgaacgcaccctggccatttgatgctgaatttctcgcggttttgacaaagttgattactttttgattttgccgaagctccctcttttgacgaagctaaagaacaagacgatgctctaattgagaatacgaagaataagcttcggctatggtcaaatttttcagcagcacaacaatacgatagtaatgaatgctgtggtaacttcacacctacccgtctgtttatatagtgctacaggtgggaaggtgaatcatcaagccacctgcacccgccgaacagtcgctcgcattcactgaacggtggaccgcatggcgcgagaaggagaatcaccagatcgtgcgtacccgtcctatggtgggaccacctcgcactaggaatacttaaatcgtttctcgacaacgagctcagggaaggtgtttttcggaccttcggcattccgaagcctaaaagaatttttcacgggtcgagctcgttacaaaaaatgatctggcaccgtgaaggggctactgttgggggtctgtttcgtcgccgaaggtcctgtgagaaaaaacaccttcggaaggccagaacaggaatgatgccgaagctaccaatcagagagcttcgtagcgtatttccagatgcaccgacttaaagatgaaatgacgaattgggcccatgataatctatgttatgattgtaatcatttgtagaggacatgaatgtaaatttacacaggctgcgccctgtgcctataaataggtgaacagtaccctcgtactgttcacgctttcgcatcttacttttatctttgccttctatcaagctcaaggtataaatgtaatttgatattattcttatgttcttatgattatttaataataaatatttatgttaagatgttatataattgtttatgttgtcttcctatgtttcataagcttcatcctttgtttatacatgtcatacttatgaaggtatgtccttcataaccttcgtccgaagatcgttatctcctaagggaaataatgcttcgaaggacgaaggacattaacatttaacattttgtgttgccttgttcttaactcatagcatttgagaacaagtccccaacaattattatgatatcctcgccactaacaagtgaatttttgggagaaggactaaaatgcagtcaacgataatgtataagactttggagcaaaaacaaagacaagagacataaatatccaatacaaaaggaaaccagagaggtagtggtatttttttctttcttggtggctaagcatcgctcaccctgtgatgcaaaaatctaccagagacaagtatagccaagaccatcaaataaagagacaatttagcaaacaatccaaatcaagatcagtgtttttatgtaaaatagagcatttttatcatctccaattgcattgacaattataaatatgatgaaattgagaaatagataggctgagtaccctagctcagcctcatctttggcagaggcatcaccatcaacatcttcaaagtcacaatcttggaagagtttctttgcccttttttggcaggggaagggtgggtaagtcctatcagtagattgcaatcaacaataggataagatctcatatgtattatggaaacaaataagtagatttttgcgttacaaaggttaccttttttataccactcttctgtgtcccggttatagaaccaccccaggttcacactagcatcaaagatctgtagcaacccgcgatcaagcacatagattaccattatattttagacatggtgtctcatgttattttattttcaagtactatgtaaattcaatgaaatgctaagattaatatggcaagaacatttgacagaaattagcatcatactgctggtgacattggaatgagagaatttccatcatctcttagtattagctaaaggaatgagttccaaggcgaaaagaggcttcagttagaagaaaaatttaccttaggtataagggcatcaccatcagttttctgtgtttctgttgacctcgcaaagcaacttgcagaaactgcactcatgatgtgcagattcatatcatcttccacagatttaagaataaaatatgagtgtacaaaaaatcaaattggtagtcaaacatgcgaactgtattctgttgtttgagtgatttcacaaattactgtcaaatgtgagttagaatataccttagaagtggtcctggcattcctgctttgtggtgtcactgttggttcagtgattttcatcaacattttgttgttggtattctcgaagcatggctagcctctgaagctggtagttaaggcatcacttttttgaagagtcccttgcatattgcttgttgtaacttgagagaccatgatcagtgttgattgtgatcctgctggtgacatttccataatctagctcaaccccctagctgatataaaacagatcaaccataaatcaaatataacatattgcaacaaacaattacacaatcatgatttctatagcagaatattatattgtgttcatgagttgtaactgttagatgaagttacgatattctagaagtttcttgtgcatgtaatctttagccaaccgaatcaatctcctatagatagaaaggatatattctaggctgtgcatagatagaaactccaacaatagattgattcggttaccttattgtataagttgttgcacccagccttgtgcctatataaacatgcaatccttggccacctagtgtggtagaacgcttcaactgtgacaccccagtgtcacgtagggtttttcctagagttgactccaaccattatcacatgtgaaccaaaaagaggaatgaacataaaaaaattaagaacaaggtttaagtgagtctttttcatcttaagaaattctccttaatcatgccatgcacctcaaggtaagaagaactctcaaaccctaattaatcctaagtggaccatttaagcacataaagggaatttgggaaaagacttgggaaaatacaaaattttggtaagaaccaaataacaaagttttagtgcactaaataaccaacaaaatatagtaagaaagttttgccatttgaattttccaaaatcccaaatcagcccatgaaccaatgccctatggggaaattcagaaattcagaaaactgaatttcaaaccctttcccaaagttcagatgtgttccctgttttccaaaactcgaatccacaaagtccaaatatcaaagtggcgccaaaataccctaggaacactttggagaagtttgagatcaaacccgaatcgtttgacacgacttgacataagttttgtctcggtttggacagtgctaacagagctatcttcaggccatcatatcttctcacctaggccatatcttcactcgggactcacacacgacaggaagaccttggcacggtgaagagacgctacacaggatccttggcaagatatgcacgttttggtcggccaacaggcgtttgaactcgggcagaatcacacttccacgtgttcgatcgcgtgctcaagcgcttggccgcgcactggctgccctctgatcgcgcgccatgcacggtcggcttctgtcccccgcgcctgcactcagccatgcctgagggcgcctataagtaccctggatgcacaatggtctgcccttcactccgcctcacgcctcgagcaagaactccaactccgcgagctctcccccgcccgccatcaccgcccgagcctcggccaccgcggccagctccctccagccacttccaagctgcaccagtcactcggttagcttcgccagtggcccgtgaagctttccaagtcctcggacccaacagagtttcaccagagacccaggatcgacctcgctggacttcggtcacccgcagccgcgcgtagaccgagcaatccggtgattcattctcaaattcctcgcgcgcatgtcttccttgacctctggtgaagctccctaacctgttcaattggactatcgcgccgtgagcaggccggatccctcgccgccgacgagctccccgcctgtgcacgtggaccaacctactccgaccaccaccgccgacgatccgcacctcgacgtgatcgccagagaccccggacctcacccgacccctcaccggagcaacctcgccgccggtaagcccctccgcccttttcttccactgcggtcactattccattaggggaaggatcgcgggttcgatttcgcaaaaccctaggggttttctgcagagtcatagactcagataaatagtgaaccaaggacctgtctgtaatacacttaaaacctttcgccagggaccccagtgcaaaaccctttttcctttatccatttctgtttattctttttaaattcagtaaaggacttaggaaatttgtatcttgagaaatattcaaccaaatttagtcaaaccaattttactagattcaaaatattatgaactatcacataaaaatattgaaccctgtgctttctgttttaaattttggagtttagaattaattaaagaaactgaccaaaccttattaaaatgaagaaaattagttatgcttctgtgctgaacttaagaaaatttgtagaagttcaaaccccacttagacactgtttaaaaatattgagcaccctagtattgaagatttaaacagggttatctattaaaagccataattgtccaaaacttaggaaaataagaaaggtactagaaaataatgaacagtggatgcaaatatttttcctagcccacttaagtaatgaagaacctagaaaaaataaaaggaacactagtccagagcaaattcaaggtgaaatgttttattaggcactaataaagctagaagggcaattattagaaatatgagaacaatttcaaaattggtaagaaaaattcagtagacttgtaaccactaggacaccactacaaaaatgataaatacctagcccatcattttaagtgggttgaacaaataaaacttgatattgagccatattccaattaaatcataagcaagccaaaaagtgtgcaacaatgggcgaataaatttttactagattattaatggaatagatcaccagagcaaaatgcaaaacctattcaaactacaaagtaatacccattgcccctacttcatgaaaaaggccatttaattcaagaaattcctaccacccttcccttaagaaaaaggttaccaaattttagaatgattgctcttgcgcaaagaagaagataggaaaaattggaaatctgttgtttgatatttttcaagtatagtggtagtagaaagcacccctttggctagaaactttagaaaatcataataaaataactaataaatattagtggctgaaaatttgtacaaaatcatgttataacatctaaatgccagcaaaaataagtcttaaagaataacccactgttaaaagagagttgtagttcaaaacatcccctttgccctaacacttgctaattttgtacagagagaacccctcactttttaagccccaaattttgagacagaaaattatacaccagtaagaagctactgtaatgtttgcagaatttctggaaatttattaagctatcttgtagttcaaacccaccttaaaagcataaaaggaataaagaagggaggaattagaaagattaataagtattaccccaacatggcagctaagaatcttgttaaaatatccataagatataaagaagaaaatcagtagaacactaaaaatgggttaaccattcagtaatcaacttgaccctaagttggtgagtgtaccaccaaaaatctccagtagtgagaatgaggtctaccctattaaattgatcatcctccatcaaattttaattgctaaattaaatatcatgccatgcatatatcttactcattgcattcattagattgcaacctcgctgatggagagtacgtgctcatccctgagcaaggagctgtccacgaggaagaccaggagcaagctcccgagactgccatcgaggatctccccgcagccccatcatttggaggcaagccccggttttatgcataaccaatttatatatgctactttactacacttagtgtttgtaggcttgtaatgtgcacttaagtgtaggagttgcttgaaacccttagttgcatgaactcaggattcctttttgagatggatactagtatgctaggtcgagtagctgctttactaattaggatctcggtagaagtcgagtgatttttctagcaatcgcgcgaggtcaggaattgattgtattcatcttgataatgggatctatgatggtctatggtcttggatccagggtggatgccttgtccatgagacaggaaaatgaattaaggattaatgtgtggatacctgagtcaagcgtttgaacgtactaaacacatgtcgggaaatatggtaaccggtaaacctagtacctgattgaagctgggcgcggacttttctcctcactcgtcctgagactgggtctcctatgctagctttggtgggtacaagtgcggtcactgcacggcggcagcccgggtcagtggagcattgtatgccaaggcggtgagccctggccgcgaaaggggaatcgatggggacggagtgccctgacatgtcgtgtgtttaggtttaccttgcaaggttaatactcgatttgaatcgtctgcttctcgcagctaatgagactgcttgaccccttgtactacattgagtaagaagtgaaatgaggattacatgagataacttgttgattgtattaaatgattgttaccatgtatgcttagaaagagcaaacttagctacaataatgatactagaaatggaaaagataaagttgaccttagatacaactagtgcttttggcaaaccaaacccctcaaccaaacagctacatggtctagaggtagaagagtagattcctcacaccgggtaagtctagctgagtattagtatacttagccttgcttgtggcataatttttgcaggtacgctctaggatatggttgacggtgtaacttggcctacaaccctgtcaccgggttggacggtcgagtgggatgctgctccggcaggagaggagcaggagaagtagtgggccaggccttgccctattcctcgcttttgacgacatcgattatccgctgcagtttattttgtgaacttttctcagctacttgaaaaactctgatttatgtaataactccagtactttaatttgaggttttcctgttttattgtatttcttctgtgactcaccttcgagtgagcttgtggtatttgatcctggataagtggctttattagactagatctgagggactgatggcttattccgatttaagtgcattgcggcctttaaggcgtgacttgggcacttaaactggaataatccgggcggttctgccacatcaaccattccaatctacatggtaccatagccaggtcctctacaacacatccatcatggcgagtagattctcaaattccaccaccatcccctcctccttctcgatcccggtcaccgaaaaactcaccaaaaccaactaccgcctatggagtgcccaaatcctaccgcccatccaatctgcacagctctacggtctgctcatcggcaaagaaaagatgctggttaagactgtctctgtgatgactaacgacgcctatatggagacgcccaatcccgagtacatcaactgggtgactcacgatcaagcgctgctgggatatatcctctcctctctgatgcgtgaggtcttgatgggtgtcacgacagccacgacctcggccgacgtctggagctccctcgcggctatgtacggatcttgcacacgtgcgcgttctgtcaacacgcgcattgcgctcgccaccacgaagaaaggcacgaccacaatggccggattctaatccaagatgaagagttatgccgatgagatgtcggcgtccggccaacctctgggcgatgaggagttcgtcgcctatgtcctcaccgaccttgatgaagaaatctacaacccgcttgtgtcgtccatcgtcacttgcgtcgagccaatctcctctgccaagttatactcgcagatgctcagctatgagcttcggcttgcgaagcagtccggcggcaggtacgctgctcatggatcagccaatacggctactcgtggccgtggtggctcctggcatgatggttctccaaaatcacggtcgcggacgctcgcgcggaaatggccatggctatccttcgtcgtcttcgcgcggcaactacagcaacaacaactacttcaggcgcagttccggtccaccgacagatcaatccggtggccagtcttgtccacgctgctaggtctaccttaaagtcggtcacagagctaatatctgttggtaccgcttttatgaagaattcactcctgatgatcgggttgcggccatggcatcatcctccactgctgctgatccaaactggtaccttgacttcggtgtgactgatcacatcaccgacgagctggaaaagctaacagcatgatcgttacaatggcaatgatcagattcgggcggctaatggtgcaggtatggagattactcacattggttattctgttttgcccacttccttccgccctctgcacctaaatcatgtccttcgtgtccctcatacccataaaaatcttgtttccattcatcgtttcaatcttgataataacacctttattgagttccatccgttctttttcttgattaaggatcaggccacgaggcaagtgctggtgcgcggaccatgtaggggtggcctctacccattgacatctcttgcacacctacccagaagcacgaccttgccgcaataaagccatcctatgagcgttggcattgcagattaggtcatccatcgcgtgatattgtcgctcgtgtcattagaaataataatttagtgtgttcaggcttagattcctcggagtatgtttgtgatgcctgccttcgtgctaaggcccatcagttgccttatcctaagtcgaccagtcagtctgctgctcctttagatctggtgtttttcgatgtctggggacccgccattgattctttttgtaataaaaggtattatgtcagcttcattgatgattatagtaaatttacttggatctatcttcttcgccataagtctgaggtgtttcagttcttcaaagaatttcaaagccttgttgagcgcttgctcaatagaaaaatcattgctatgcaaaccgattggggtggcgaatttgagcggcttatctccttttttcttatcactcggcgtccctcatcgtgtctactgcccccatgctctgcaacaaatgaggactcctatcgtgaattaatcgcgcttgtttatatgatcctttctttatttctgaacatagtcataaactttattctctttggacgaccggtcctaccgctcttggcaatattgctcagcnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnngaaatctagagtaatcgttctcatcgcctaatttatgttttaaaaaattaggcatgtgagttttaacaaatgcatgtgtcatcctctctatatcctccgtgatacttttaatccgattatcaaaagaaattttaatagatggaatatcatcggctgacctggcatcacctattgtggggagctgttgcagcacggctaacatatactcggcgtttatctccctctcttggacggtcttctggtggcagtctaccttgaagtgcgagaggtaccacttgtccgccaccttgagcacctgatccttttgtcggtgggcctcctcgtctattttcttcatgtcatcttctaattttttatgctcagcggccgataaattagtcagccttgtgttgctgtttggagaagcactgttgagatctttagaatcggccatgtaagcctgattttgtagatctgcaacttcttccccagcggagtcgccaaaaagtatgttgacgcctttttggagcgccaaacactcaacaagaaccgtggcggtgccctctggtcaggcgcggacggtctgcagccttgggccggacgatccgcagccttgggccggacggtccgcgacctgggcgcaggagtggtgtcttccctgcgtcacaccggacggtccgcagctctgggccggacggtccgcgacctggcgacagggtcgtcttcctactccttgctggaatctagatctcgtcccctggggggaaagatcttaaggtgctccgggtcgacaggtcacccggggcgtccccagacgacgtggagtcgcctaggaattaagagatcaaatcgaggaagaagtcttggatggacaactagatcttgccccccgggaggggtgagatcctagggtcgtcttgggatcggcaggccacccaagacggatctagacgacgtagagtcgaataggggtggaggtggatatgtggaagactacaactagaactatgctacatctactcctagggcaggaaaagtaaataaggtaattggttcgattggaatgtgttcgggggttctcaatcggccgtacccctttatatttataggggaggaggtctggaccttttcctaagagatagccaacaaactcccacgtgattagatggataaccacgcacgagataaggataaacatccgagttaatctaatctcgggacacgcggaccgtccgggcccatgggccggaccgtccgctcattttggtgtccaacagctacgtagtcatgccatcacttcaccccacaatcccaactatcaaaataactctaaccgaacttggcatttagccgatcgattcctaactcatttttcataccaccactacacgacataccgaatacattgaatgactcgttcacattccacatatatctttacgaaaacatttccacatcgcttgcaacttaacctaagcttcgccacataatttcaggacatctacttaaatcatgaatatcatcatcacacacatcgacccgttttgaaataaccctacatgtctatcacaggaatggagcatttcaacacatatcctaaaacaaactaacttcatcacacatcttgcattacaaagctacttgacttatttgaagtgtctactcgaaatcgtgagcacaatcatacactatatacgaaacataattttaacgaacgcataatacgcatcgtcatgacttgacctataaatatagagaaagcgatgactactctggcatgtcaccacctctctatttaagtcaagacaatttctaccatcgattaagagtcgtaagcattaaataccttactactttatacgcacaaataaacttcaacttaacacaactgacaccgatggaatttttactaaactcatcgtacgcataaccctgtctcgcatacaaccatattatggcgtgcactcgagacacttcaatccatgtggcgcgaccactagtataaatggactccgacactcatgtcttaacgatacatcctctacgcaaactagcattctctaaactactcgtcacatcaataaatatatcccctctaaaattacgaatcccatcacattgcttaaaacaaatacacttttcacataaacacatcgatgcattttccaaaacaaaatccacattttgtaacttagttttcgcatcaaacaacgcatcgcatattttcctatcaaaataaaaatactcgagttcttttgtatttcattttcttccctacacgcgtccatttataaaattatacttttacacacatataaccacatgcacatcatcgaccaaaacataattagacaactacaaatcgcgcacatcaattaacctcttgttctccaatcgcaaacatgatcctaccaatgcgcataatcgaacattttacacacatccatacaaaatgattaatcgagtcgatcgagagcgacatgcatcggctcaccataaacaaacccaaacgatgtttgcaagaatgacggtgattccgattcgtgcatcgctccaaacatccgacgagcgttaagcgacttgctttctcctcgcaaaacacggggttctctcctccacaaaaataaaacaaagcaacacacatacataattaatcataggaaaataacatcgatgcggaatcgaacaaggagcgtcgcggtctcaccggggtgaacgacgacgacgtttggggctgcgcaaaaacagcgaacacacggcggcatcacggcgtgctgctcactgcgcaacaaaacagcaagccgtcagcgcgcggagccgtcggggctgctgcacatttcatcgagcacaagtgtggatggcggccaggtgtttgtttcaggcgctgaaacaatggagggggagagggctacggctggggaagtggtggctcggccacagcaagaacagggaaggggaggctggtcgccgaccttgggcgcgggcagggaaaatggagttgctgcttggcgctatgtacaacagagagagggaggaatggcgccatgggaagctcgagctcggccaggggaaggaagaaaggggttcggcatccaagctgttggagcccaaggagagggtgctggccgccgtgcgcaagtgaagtttcacgccagctgaagctccctggtcgcggataggaaagagcagggggcgcctgctgcaggtaggagctcggctcctgtggaaaatggcaggggcagaggaggccggctggagcaccgggcagggcgctcggccatggagccgctgcatgggatttgctgctgcgccctgggagaaaaacagtaggggagtgaaggatgccatggctgggggcgcggggagcagggagcctgctggtggccttgctgctgtgaagcagggaagaagaaaggcagaggacgccacgggaagagcttcggcgcgctggagggaaggaacgcccggccatggaagcccctgcgcgctggggaaggagctccagctctacgtgcttgaaggagcccacggctggaaaatggtagaggaggaagagaagggtgttggcggctggggtggaaatggaaaattttcagaatgcaagggagggaagcccatatttatagaggagaaattagggtagggtttcttatgggccgaatgggctggactggatttggcccaaaacactaaattgggtcgcgctaaatattttccggactaaaaatgttcctgcggaattcgtcgctactgagaaacagagcgaaaagagttcggacgaacggaaggttgcgcgattaactcggccgagagtctgtttagatttcgcttgaaaataattccctacgcgtaaatcgaaaataaatcgtcctgagatttgatcggttttggatttttagtcggagaaagcgaatcgtgatatataaaaatcgttgccgatgttgattttgaaatcggattggatacagagatgctaagctgagtcgagtaagatttgattagaggacgacatattgattatttcgtttgtgagtatggactcggattaaaatagttggacatcgatcgaacatcgagaaattggattcggacacagatcaaataacagtcgtcgagagtttgatttaatgagcttcagatgaggtttataattcgagaatgatttttgagttcgcatttgtgccgacgataaaagttttaacaggctccaaaattggccttctgtgagactgagtaactccgaattcggtgaaacgtgaatgaataatctggataatcagggacatacgcgagcgagaaatagaaatttttactgagcatccgagattaggataaatctcgcgacgtaacacgaaactgacacctggggtgtcacagccttccccccttaaaaagaatctcgtcccgagattcgaatgaggatatttatgggtggagaagcatgtaactcccagactgaagatagatgcaaattcatgagagggtatctgacaagatactggagacagatttggttagaatatcgcgacatatcgagacaaaatgcagcgatcattctgagagtgtccacaaaaaaatagcacatcagtatagtctcgtaatggatcacgactattaaccgcgatactagcgcgtgccgagcagctcaaccatgtgtgcaccatagtaggctctcggtttcgtcgcggcaccatcagtcgttagtcatgacatcattaccaaacgcaaccaataagaaattcacatagcactgatagttggagcccatgagagtatggctcagaaaataagaatgtgatcagagttgaagcagagattattggcaaaagatcatcacatgagaattttcttcaactcatagagttattttatgatcatcacggggattagcaggccagcgattagtacgagatttgatatgagaaggaagcactccagagatcatgttgatgaacttgtagagacatgagagaaccacaagatgacaacaacatcccttgaaccaaatggatacactgtttagagataaagttgataaacatcgtcatgatcctcagagaacgagtatgagaatgaccagaattgagagacttaggtagatcaacattcgatacttgagaacgggttatagtagataacaagataatagggcagaatcatgaaagatcagagattcggatgataaggtcacaacatgattcacaaggaaaaagatcactagatccatgcgaaaggagaggtaggcaacaagatcagctggatgatcaacaggaatgctatgaagttttaggggcaaggaatttatggaaagaaacatggccttgatagggtttgcgcaactagacaccaaacaacaaattttttttgacgtaaccagtgcacaaggaagctttggtcgatctaggagtcaagctatgggaatctacaagctgtgcaggtgtaacttcaagggtaaaacccacaagggctagaaaacgccaacacaagcatttttttaaaagcgggttcacttgctaaactcaaggttgtttggaggagtctttttatgaacagaacaagcaacaaaatgttttgcaaaaagggttgaacaattacaatactacctagatagcaagacaagagaagcacataacataacctagtaaagactatcatgacacacaagataagacattttttttgcagttcctagcaatacagcacattattcacaattttttttattatttgaataaaggtgagagaagcatgttggtgcacaaaagacaattataatgcgacaatcatgatgcatgctcattctagtcgtcttctcagacctaactactttttcggttgcttctacagcatccttattaatagtagtagtagcctttatggcctatataaatagccacctagctacccatctatttcctaaggcttcacgtcctaagtctatccttatcgtcctgacatctatccaacattggtttctagcaagttttacttttagaaaaggttggtaatcatgacttattgacttctctgtgatggtattcgctccgataccagctgtggcggaaccgcccgaattattcaaacttaagtgcccaagtcccgccttagaggctagaccacacttaaataggaataaaccgtcagtccctcggatctagtccgataaagccacttatccaggatcgaataccactagctcactcgaaggtgagacacagagaaatacaataaaacataataccacaaatttaataagtatcattagtgattacattatcggagtttcagaaataataaccataaattttaatgcagcagaaataactaacggagaagaaccgagtaacatggcgaagcctggccactctactcctcctggtcctctcttgcggaagcagtaacccactcgaccatctatcccggtggtagggatggaggccaagtcacaccatcaaccaatcatcctaatgaatatctgcaaaaattatgccacaagcaaggctgagtatacattactcaactagacttacccggtgtgaggagtctacttctctacctctagacatgcagctgtttggctgaggggtttggtttgccaaaagcactagctgagtctaaaatcaagttttagcttttcaagttttagtatgatcctttttgactagatgtgtacctagctaatcatacatgatatcaagaatttttatcaaacaacatcttttgccaatcacctcatttccacttattactcaatgcagtacaatggatcaagaagtctcattagctgcgagaagcagacgattcgaatcaagtttttaaaccttgcaaggtaaacctaaacacacgacatgtaggggcactccgtccccacacacatcaaccgtccccatcgattccctggcaacagaaaggggctcaccgccttggcgtacaatgcctcactgaccccgactgccgtcgtgcagtgaccgcacttgtacccaccataaccggaatgggagaccacgtctcaggtcgcctgaggagggcaatctgcgggcaggttcactcaggtactaggcttaccgatttaccatatttctcggcatgtgtttagtacgttcaaacgcttgacacaggtatccgcacgttaatccttattccaatttcatctcgtagaccacgcgtccccatggacccgtgtccacagaccatcaccattatgttatcaaagtggatacaaccaattcctgacctcgcgcgagtgctagaaaaatcactcgacttctaccgagatccctaattagcaaagcagctactcaacctagcatactagtatccatctcaaagggaatcctgagttcatgcaactagggtttcattcaactcctacacttaagtgcatggtacaagcctacaaacattaagtgcagtaaaatagcatatatataacagttatgcataaaaccggggcttgcctttaatttaacacttaggtagtgtttgctgggggaggtactcgcttggcgagcatccactggttaagtccattctttaggtcgtccatcaacggcatcttgtggttggcaccacatcactggctcgatcatcatctctcggtcctatatgaggtgcaagatgcatatgtatgaatataataaaagtaacataagatataccaagacacagtggcgaactaaacattaattagtaagacactgcaacaactatacgcaaacactagttatttatgtgtcattgggcacacgtaaacactaccactggaaagacaatgatcactacctacaattaaccaacgcaacacgatatcatatgtacaagcatttatttagttgctacggcttttcattagttcttctattgatcacacaaaagcatcacaaacacaagtttaataaaggaccgatgcatcaatgtcgatggactcctctatcacaatcaactatagcaagcaagcacattaatcatggaacacatgttaacctaagtttagccatcacaagtctatgtccgttaagtgctaactaagcgtttttagccaaaatggtgaactaaatattcatttgagcacgtgcagattttttggacagcagcacagcagttacttgttttaataataacttttcaaatattaatccaaaaatagcaaactaaaactttctggaaagtttagaaagtgctctacaattttggtattttcatcacagcatgattaaacacttagcaaggtcaaaaagtgcaatcacaacagctctgtccagatttggacagattcagacttgtgattttaaaaattcataactgaagattcagacatccaaacaaattgatcctagactttctggaaagctaattaaatgttctacaaattatttataaacatcccaggctggtttagcatgtatcaaggttaaaatatactatgaaggctgtgctgtccaaaactggacagattcagtcttcacacttcaaacacatgtaacttaatcttcagaccaccaaaaagagtgatctaagactttttgaaaatcttagcaaaagtactacacaactttcataatcaccaagaagtgattccaggtttaactaaatcaaatattacagttttcgaaatctgttctgacggtggacagaacacagcaaccagtttgtaaaattcataactcttaaaccgtcaggcctatagttatgaaattttaacacaagcaagataagaaaagcctctacaacttttcttataatctacaagggctgattctaacattaacttaagcaaacaatgcagcttctgaaatctgtacagaaagtggacagattcagttactgaatttgtaaaaaacataactcctaaacaattagacttatgcctgtcaaattttaacacaagtacgataataaagttatctacaactttcttgtgaccaccaataactaatttcaacattaacttaagcaaccattgcaatttctgaaatatgttcagaaatttgacagattcaggtgctgggcttgtgaaaagcacaactcctaaacaatcaggtttatggctgtcaaattttagtacaagcaagataatcatgtcatctacaactcttctatatgacttttctacagaaaacatgatttggtttatcaaacaaacagcacaactaaaacagtgcgtgcagcccaaaacagcaatcaataaattcagcttctgtttacttttaaaaattgccgcgttctagagactcgacttattctaaattatatcaaggcacacttaagcatagccacacaatagatgatgtgacggctactgttgacgcctttttggagcgccaaacactcaacaagaaccgtggcggtgccctctggtcaggcgcggacggtccgcagccttgggccggacggtccgcagccttgggtcggacggtccgcgacctgggcgcaggagcggtgtcttccctgcgtcacaccggacggtccgcagctctgggccggacggtccgcgacctggcgacagggtcgtcttcctactccttgctggaatctagatctcgtcccctggggggaaagatcttaaggtgctccgggtcgacaggtcacccggggcgtccccagacgacgtggagtcgcctaggaattaagagatcaaatcgaggaagaagtcttggatggacaactagatcttgccccccgggaggggtgagatcctagggtcgtcttgggatcggcaggccacccaagacggatctagacgacgtagagtcgaataggggtggaggtggatatgtggaagactacaactagaactatgctacatctactcctagggcaggaaaagtaaataaggtaattggttcgattgacaagttttcggggtttctctcactgccgaccctttttatcataactgagcaccaggtctgaaactcaaacctctccgaaagggaagcgtatcacctgatccgagctggataagctccgactatcgacggatgacatagcatcacaactgatctcgggacagcaggtgctgccggttccctggaccaagcaagcccatatcatttgatgtccaccagatgccccctgccgcaagcgcgcaaaaagctgcacccgggagcctgaattacactccgaaaagcgtgagcccgtgattgccttttcatgtcaaaggatcgatacggatcgatgggagatcacgcccgatgggcctggattgcttctgttaccttggcgagcgtttggtgcagaggccatcctctggaacggattccactgcaccatggctgatggaatatcctgcgtcatgcagaccattgatggaggtgggtccccagcccagatagtgaagcgcgaactcgcatggtgtccacatggattgaccgcaccgtcccgcagcttgaaatagaagcccggtcccgaaggagacatgtcggggagctcggcggctgtccctactggacggctgctagctgcaaaatggggggttggccgctcctacgggacgttccgcacgcgtctctcgtgcgaccggacgcaatggccatcggatagtgttgctctggactggttcatgattagcacccnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnngaattctttattcctaagttaatttgatcctcatgcttactttggttcacataaaataatggttcttggtttggcatttttaagagaaaccgtaggtgacactagggtgtcacagccttccccccttaaaggaatctcgtcccgagattcgggccagagtcctcccagggtgaagcgaagggtgagacttataggaaaagggtggggattgttatgcttcaaatcatggtacatcttggtgcttcccggatgcatagagaatttggagagatgagcctcatccaaaattttcttcttgagatcctggtccttaggaattaccaatctgcttttgaaccataacacacccttctcatcctggcggaaacaattatacttctcaaccttctgatggagattcttcttgataatttgcactcccttgtcactgagctgggccatgataatctggtcttgcaaggctggctcaacagcaatgtgagacaaagaaccagaaggaatcacttcaatttgcatcttgctcaactcatcacacaaggtgttaacacgagaatccatcagaatacagttgcactgcaacttccgactcaaggcatctgctaccacattagctttccctgggtgataatgtacctccaggtcataatccttgatcagctctagccatcttctctgcctcatgttgagatcagcctgagtaaaaatgtacttaaggctcttatgatcagtgaagatgttgcagtgggttcccattagatagtgcctccacatcttcaatgcatgaaccactgctgctaactcaaggtcatgagtaggataattttgctcatgaggcctgagtgctcttgaggcataagcaatgactcggttgtcttgcatcaagacacaacctagcccggtgccagaggcatcacaatatacatcaaaaggcttgctgctgtcgggttgcgccaatactggtgctgtggtcagatgctgccttaatgcatggaaggcatcttcgcacttctgactccacacaaatttgacttctttcttcagcaactcagtaataggcttcgcaattcgagagaagtccggaataaatcttcggtaataaccagccaatcccagaaaactccgaatctggcgaacagtcgttggtggcctccagttcatcacctcttgcactttatcaggatcaacagctattccagcctgagagatagtgtgacccaagaatttgatttcctttagccaaaaatcacatttggataacttggcataaaggtggtgatctcgcagacgttgaagtactacatgcaaatgcccggcatgttcttcttcgttccttgagtacaccagaatatcatcgatgaaaaccaccacgaacttgtccaattctggcatgaaaacagaattcatcagatacatgaaatatgctggtgcattcgtcagcccgaatgacatcaccaagaattcatatagcccatatctggttgagaatgccgtcttcggaatatcacttgctcgtattttgatctgatggtagccagagcgaaggtctatcttggaaaacaccttggccccgaccaactggtcaaagagaacatcaatacgaggcaaaggatacttgttcttgatagttaccgcattaagagggcggtaatctatacacaacctcaagctttcatccttcttcttcacaaacagtgctggacagccccaaggcgaagtgcttgggcgaataaatcccttatccagcaactcttgcaactgcttcttcaactctgccaactcagcgggtggcattcggtagggcctcttggaaattggggccgttcccggttgcaactcgatggcgaactcaatatcccggtccagtggcattcttggcaattcatcaggaaagacatctgcatactcacagaccactgggatcttcttcaggggtaattccgtcatagagaaagcacatgactgagaagaaccctgactaggcagaatcaaagtgaaattcccgcagaagggagaattaacttccacggtacgactggctacgtcgagcacaacttggtgcaaggtcatccaatttgctcctagaataatgtccacattttccaatcccaacacaagaagagtggttttgataatgtggcttcccagttgaataggcacactttggtttaattgattagttgcaattttacccccaggtgtgactatcatgaatgacccttttgagtgagagaatggaagtttgcaattagcactgaacttttggctaatgaaactatgagatgcaccagaatcaaacagaattaaagcaggttgattataaactgaaaaggtaccggtcatgatgggagctccttctggcacttcctctagagcagtgaagttgagcttcccttgcctgacttgtaccttctgctttcttcccttgtcttgatttggtgctggcatctgcctctgctggttcctgggacaattcttggcatagtggcccacattgccacaagtgaaacacttgttcccattgccctggcggaactgctgctgctgcggaggctgattgtttcttggggcgggagctggatagcggttgggtgccggctgctgctgctgctgaggtggcctgatcacccatctgcctgcctgctgctgaaaacccctgctctgattgtgagaaacaatccggaacctctgagcctgagcggatggtgctgccattggtgcctttctcttcttctctgcccggtgagcaacaatgcaatcctcctgagagatggccatgttgaccaactcattgaagctatcggcccggacagtgttgagtcgttcccgcagcttggtattgagacccctgcggaagcgatccctcttcttttcatcagaatcagcatgataccctgcatactggcataagtcgttgaaggcttgcgcatactgcagtaccgtgcgggttccttgattgagggccaggaattcgttcaacttccgatcaagaatgccagctggaatgtggtgccctctgaaggcagtcttgaattcctcccaagatacttcacgatcaccggggagcatagcacggaagtgatcccaccaagtccgagcagggccgcgaagctgctgtgcggcgaagcgagccttggcctcatcagggcagtctcctgtgaggaggggaaacttggactcgacgacgcgaagccacacgtcggcgtccaatggatcctctgccttggtgaacaagggcggctgcgtgctcagaaactcctggtatgttgccatagccggaggtcgctgatgctggcctccaccaggatgctgagggtggggctggcgctgcaagagctgtcgcagaatctcattctgctgggccatcagctcctgcactgtgggagctggaggaggtggcgggggagcttgctcattttgcccgcgacgctgcctcgctgccatctgaaaacagagattgtcgccattgttatcccaattcacatttccgaacgacaagatatcatctcatatggaaggaaaatgccataatcataatattaggttcgaatgaagataacatggtgacaaggatcccacagatatcaaaagtttacagggttacattaatcaggggaaggtacccacaagcctagtccaaaatgtgataccactaagctcgcataggtttctatccgcctaaaaatgtcaaagcgactgcttaaccctgagcggtggaagcgacactggatacgggtgaaggaggtatcgcggaggtagtcccattggcaccaggggctggtcctagctcctcgggagcctcttctccctcgcttcctgcttcattggcctccatctccaggtggtgcatgtccaagtggtcatttgcttcctcgagttccctctgcacgtcgtgaagctggttctccaagacatcaatagtgttatctcggatctccacttgctgctccagggtggtaatacgctggttcagcctctccacctgcagatccttttccaccaactctgtggataggtcgaccacaaaatcttcccgactgtcgagggtgagcttggctgcctgagcggtattggcaagaagtgtcatagcatcgctctgaagggcctgaaggcggtacagcgcactcatgcactgaacagtgaccctcccaaccaagtcaggatacattgcccacacatccttcacatggctcacgcggttacaccacatgggatcatccttcttctcagcggggaagagtcccaaggggtgcatcaccatctccaggggatggtagccacaaaaagtcgtcagagtcttcatggctgctgcctcaacggtgtcgtccgtcctgagtccaatcgtctcagagtcaagagaacgccaacccggctgaaggggatgagcctccaaagttagccagacccgacaacgaggtacccgatgctcctcatacaactgcaccgtgtacaaagggggcgtagggtaaccggcggaattaagcacttcccacaaaatggaagggaagccatcgcgagaaaggaagtcagaactgaaacgagagtctcctccactggcgggggtgggtgaattcatctgcggaagggaatcaaagataaagattatggtggaaggaaaaagaaaaagagagcccggatgatttcgaagaaaagggggttagctcaattttaattcctctttatgttttataatgcatgcatgcggaaagaaacgttgcctctcaaaaggaaaatagggtgcctttttagggcatcctaaaatataagtattggcccacagggcctaattagttagccacctatttctccctctatgcctaaggcctttcgtcctaggtctagcggtctagtcctgacgatccgtagtagcttctaggcaggttttagattttgaaaattggtattcatggtttattgcccttctctgtggtggaatttgctctgataccagctgtggcagaaccgcccgaattattccagcttaagtgcctaagtcacgcctcaggggccgtaacacacttaaatcggaataacccgtcagtccctcagatctagtctgatgaagccacttaaccaggatcaaatcccacaatctcactcgaaggtgagtcacagaagaaatacaataaaacaggaaacctcaaattaagtactgagttattacataaatcggagtttttgagtagcgaataaagttcataaattaaagtgcagcggataatcgatgtcgtcggtaatgaggaaatgggcaaggcctagcccactactcctcatgctcctctcctgccggagcaacatcccactcgaccgtccaacccggtggcagggtggtaggccaagtcacaccatcaactacatcctgcatggtacctgcaaaaatggtgccacaagcaaggctgagtatactaatactcagctagacttaaccggtgtgaggagtctactcctctacctctagactatgcagctgtttggctgaggggtttggtttgccaaaagcactagctgtttctaaaatcaacttttagcttttcaaattctaccatcattaacttagctagatttgctccttctaagcatacatggtaacaatcaattagttcagtcaacaagttatctcatataatccacatttcacttcttactcgatgcagtacaaggaatcaagcagtctcattagctgcgagaagcagacgattcgaatcgagtttttaaaccttgcaaggtaaacctaaacacacggcatgtcagggtactccgaccccacacatgacaaccgtccccatcgattccccgttcgcgtccaggcctcaccgccttggcatacaatgctccactgaccccgactgccgtcatgcagtggccgcacttgtacccaccatagctagcatgggagaccctgtctcaggtcgcatgagggataaagtccgcgcccggcttcactcaggtactaggtttaccggttaccatttttcccggcatgtgcttagtacgttcaaaagcttgactcaggtatccacacattaatccttaattcatttttcccgtctcatggacatggcatcctccctggacccaagtccacggactaacatataccccattatcaagatgaatacaatcaattcctgacctcgcgcgagtgctagaaaaatcactcgacttctaccgagatcctgattagcaagcagctactcgacctagcatactagtattcatctcaaaaaggaatcctaagttcatgcaactagaggtttcaagcaactcctacacttaagtgcacattgcaatcctacaagcattaagtgtagtaaagtagcatataataacatggttatgcataaaaccggggcttgccttcaattgctggggctgcggggagatcctcaatagcagcctctgaagcctgctcctggtcctcctcttggataggtccttgctcagggatgagcacgtactctccgtcggcaagattacaatctaatgaatgcaatgcgtaagatatatgcatgatatgatatgtgctttagaatttataactttaaagatgtatgatcttttgatttaaaaccagttaactttacttatgtaaaaccctttagtggtatacttggtaaattgggttagtcttattgggatgaggtttatttcttcttctcttttcttttattctctttaatgttttggagtaggtttgaactacaagttgcttttataaaattccaaaaattctgcaaaaattacagtggcttgttactggtgtatggttctctgtctcaaaatttggggttcagaaagtgaatggttttctctggacaaaattaccaaattttagggcagaaggggtactttgaactacaactattatttaatagtgggtaattctcaaaaacttatttttgctggcttttaggtgttataacatgacttgatacaaatttctagtcattaataccctttaattctttccctaagattttcttaaggtttctagccaaaggggtgctttctactaccactatacttgaaaaacatcaaacaacagagttcttatttttcttagctagtattttgtgcaagagcaatcattctggagtttggcttccttttgcctaagggaaggggtggtttgcattatttgagctaaatggccttcctcacaaattactagcaaaaggcatgggttcacttctttttcatgggtttgtatttttctctggtggtttatctcatcatggacttagcaaaattttggttgcccattatcacattatttggggttgctcatgatttagtgggaaaatgccttattatcattctgtatttattttccctacttaaaaagttaggctggggtgctctgtatttttgtagtggggctctggtggttataagttcactggatttttgttaaccactttggttatagttttgcaattctaataattgattttcagtctacataatgctaattaaagcatcttaattagaaactggtccaaattaatggtctctgcatttttcctaggttctctgctgcataagtaatctaggaaaaatattactaatcactgttcattaatctctaaggcctttctgattttctctaagttttggacaaaatggctttaaatgaataactacatcataatctctaatgctagggctcctactatttttaaacagtgtctaattaaggtataagcatctacaaattttcttaagctcagcacaaaagaaaaactaattttccttaattaaacaaggtttagggggtttctgtttttaattttaaactctaaaatttagaacagaaagcatatggttcactatttttaaatgataggtcataaaattccagagctagcaaaattggtttgacagcttttcattaagatttcatcaagttatggattttctaagttctctggtcattttaaaaagaaataacaaaattgattaaatggaaatccactttgcactggggtccctggcggttttctaagttttcctcgcaattcagtccttaggttactattctcatgagtcgctgacattacgaaaaacccctcgggttctacagaacctaacccgaggtccttcttctaccttaaacagtagccgcggcgaagaaaagggcggaggggcttaccggcggcgagactgttccggtgaagtggccgagggtgaaggggaggtcgcggggatcacaacggtgtgcggaacaccgtcggagatggccggagtcggtcggtccacgcgcgcaggcggggatgctcgtcggcggcgaggagaccggcctggtcgcggcgagatagttcaatcaaataggtcatggaggtccacgggatgccagagaagacatgagcgaaaggaatcgggcgggagactcactggatagcttggtccacgcgcggcggcggaagaccgaagtccggtgaggttgattcttcgggcctcccggtgaagttccggtcgggtccgagggcttggcaagcttcacgggctactggcggagctagccgagcactggttgggctggagggtggctggagtgggctggccacggcggccgtagttctggcggcaatggcgggcggaaatgagctcgccggagctaaggaacagtggctggccggtgagggtgagtgcggggcgaagagaggtgcgcccggggaggctttataggcgcgggcgggcacggccgagggcgtgggcgcgcggcggacttgaccggacgccggggcgagcgcgcgcgcgggttgggcgagctctggcgtgccgaccagggtcgaacacgtgtgcccgtgcgttctgcccaagctctggcgcgtgtggtcgctcatccgagcctgctctcgccttggtcagtgcacaaaacctcttctcctccctacaagctaccattcttgtgtggaggtcataggattttgcctactggttgcagagatatggagccaggaaatctggtctgtctccctgcccaaacccgaggcaaatcccaagttttgtcgtgtctagggctcgcgtcccaatgccatcttctggcacaagacagaggggttagttagacacaattttgtcaatggggccattaggattcgagttagggatcaaggtgaacatccctgatctttggctcaaggtctgaatttcagaattctgaaattcagaattcccaatgagtcccaacaaaagaagcttgatttgggggttttcttgaattattttggctaagctttctcaatctatcttgttgcttatcaaatatactttaacttatataattggctcaactcaaaattttaaacttttcattcccttttgcttattttcttgaattttgttcatggggttcacttagggttcttaattagggttgcacattcttatcctttaagagactcaattgtcttgatcatgacacttttaagcatatacttggtgaattctttcttacttaagttattttgatgctcatgcttactttggttcacataaaataatggtccttggtttggctttttaagagaaaccctaggtgacactggggtgtcacaggaggcacatacaaggatgctgagcctcgacatgcgggcctaggagcataatggaagaaatagattatgtaaataactaatgctgacagagtaacgcatgaccaaacttggaggcctggaccgtatatacaggggtctggcatgggttcggcactctcctatgggggtccggactcactattgatgccttggagtacatcactttctctggacacatggcggccccggacccgcccatgtggtggggtcaggtgctgttgctggcctagagtagtcgcccgaggctagggcgagtcatggtttggtcccacatacagctcttttaccacgcgactaaagatagtcgcgtgggtactgcgtatttatacagtagtaaggggtacccttgtttcagggtgccgaaagtggcccccggacccaccttaggggaggatgcgagcctgcatgtggggccaaagcttgtactttgcttcaacgtgacctgatcggtgattggcatgccgttttagcgcgtctgcagacacgcccgctgtcaatccgccttcagtcacgtcaactgccatatctgtctctgcagctgactgacccatggccccatgcctggtggtttcgtcgggccacgcgtgggacgcctcgttgccgctgcataaccttttgtcttctgcagcggccccgaggaggtgcgctatcgtgcgcggcagttcgcatggcgattcgctctttccgcactcgaaatccagcacacaatctgtatgacttgtggacccgggccaccgtgtcatagagtgggctgcctgggtcctatgtgcgcatcgggcgagatttcctgtggcaattcaagggcgcacggaagggtttccctgaacaaggactcaggtttccttgaaaaaggattcaccccgcgtgcagcagttaccttttcgcattctctcccaatcgcctgcacccctttgccttcgtgctcctctgttccacgctcgcgccgccgcacacgccatggcctcgcttggtcatcctgactgctttcagtctaaggaggcgctcaacctggtgcgcggcctgcttggatggagcgcgccagggctcgccggaagttccgcgccggcgccgtccctcatggcgatctcaccgccggggagttcgtgctgttcacctcctacatcttctacgggttggcgttgccgattctcgcccttcttcttgctgctgctggaggagtttgggcttcagcttcaacacctcacaccccactccgtcctccaggcagccatcttcgtccacctctgtgagatgttcgtaggtgtggccccctgtacttccctcttccgctgcttcttcgtgctggtcaagttcgggaagactagggaccacatcggtgcctactacttccagacgaggccagatccagccgtcgtatacatccccacctttggcggtgcgaggtgggaaaactggcgcaacgattgggtgattgccagcgccgaggccaacgaccgcctcgtcctgccgagcgatgggccagcgctcgaccgcaagcagtggaggactaagccgtccctcttgctagagttcctgcctgtattggacagaatcaagggcttggctacgggcggcctgccatcaatgcacgtggtcggcgatctcctgaagcaccggatcgcgccgctgcagaggagaccgcgtatgtgctgttggttcaccggcccaaacgacatcgataggatccaacgcaggccgggcaccgttctgtcctgggacgagctagcagtcctgatgggagggattattggggaaacttttgtccctgagtccctgatactcccccagaacatccctgcgctctgcgacgatccaggcctgaggatggtgatcttggccacgttgccgaccctcgacgagagcggcatggcggttcgctagaccggtggccgggaccccctccgtgggatccagatttctaatgcaccgattggaggttcccagcccactggtgcggctcccagcaccaaccccgccgtggcccctagccccttggacaaaggcaaaggggctgcgagcagtgcctccgccccaggtagctccgagggggtcggaggaggagaggcaacgcaggccatgtcgcgctgatgggtcgctcatttcggagccccccccagaagcgtcagagggctgcaggtggggccgaggaagctagctcccaggcccacggcgcgcagaggcgcgtcagtcctcacccccaggggcaccagcagcagcaacagcaacagcaacagcgatagcaacagcaggagcggtgatcgccccgcttccagggtcactagaaagtctagggccccaagtaagcgtagccccttttccatgagtctaatcatcatgccgaccagttttaacccatcatctgttcgctagggcttcttccttcgccgctcccaaggtcatgcctcctccaccagataccaggcccaccgacgggtctggctctcaacagcaggaacctgctgagagtggtgccggcggcccacccccagctgctgccaagacagcaccagcggcttctcatgccccagccgggggtccggtggcagcgtcaggcggcgtcgcagtggcgaaggaggtcccagctgggggatccgcgcccgctctcgacactgggggtgacgcagtaggcatgtccagctccaaccccccgcctgctccggaggagatggaggtggtgtttgggcggcgactccggtcgggtgccgagcaagaagcggcgccagtccccctccctcgcataatgtctcgtgcccactaggtccttagtgacactggggcagcaatcttgcgggagtgggaggcgcttgaggctgagcaccagcgcctaagtgactggcgcacccaactggaggagcgcaccagaacggcgtcccaacaattcatctccgagcggtcccaactcgagcaggaccataaggagtacaagagggacctccagagggtgtgcgccagggagctggaggcgtcccggagggagaagaaggtgaccaggaaggaggaggtcgtgacccagcgggagaccctcacaacagagtaccaggccaagctgagtgccctggaccagactctggaagcccagcgggcccagcaggtcagggtcgtggagaggctgcaaaagtggtagcaggagctcgagggcaaggctagcaatgccaccctcgccgaggaaaatcttaaggcgaaggagcagtccttggaccggtgggagacggacctcgccaggcaagagacggatctcagcttcagggaagaaatgctcacccggcgaggcgagttgctggccaagcacaagctcgaggcagaggagaaagagaggaagctggaggagcagatccgctagttcaatgcagcgcaggcggcaccgggtccccaagcgatggaggccaccaggaaggcccttgaagatctccaagcggagcaccgcgtcgaggtccagtgtattgtcgcgtgggccggcgaggcaagcacggcactagtgccactagggatgagccccatcccaatgtcggagctaccagcgtcgatctctgatgcgctcccggtgctggactctaccgccgatcgcctccgtcgcctggatcagatcctcggggcccgcctagaggcagagggcagcaagctctgtcgggcagtggttgaataagtcctaacatgcttccggagtcacgaccccaccatatccttggcgctagtgatcgctggtccggtagccgccatagaagacgccgcctgggagagtgtacaagacgccgtggagctggtggccgagcgcttccagcacgatcctgctgacgacctatagagacaaagcaagggttccactgggaagcggttgtaataacttttgattttgtaagatattataagaaccgctaatgaggtagcattggaacttaaacttatttgtatgttatttgtccttgttatgtgtagtgtcatcaacttccccttggtacttggccccctgggaggtaggctcgacgtgtcgaggctggataccagtataccaaagataaaattggtggtccggcccctaggaggtagtctctacagtttgagactacctactactggactgggacctggacttgtacacagcttcggctttaaagtgttaggagcacaccataggatccatcgtctggtatctgccatcctttgatttatgcaacaggacctgcaggatttagcctgggaagccaagccgtatgcctggacccataggatcacagttccaaatactagggcacccgttatagagtggtggagcatgcaggcttagggtacggaaccatgctaagcggctacacaactccggacccctccaggaggctagcgcccattctctagaactggtccgcagtttgccggaccccctgtagcagtaaaggggtcttgaactgcaagcctgtctactcaattcggatgtcatcataccaacaagggtgggaaactatatgggtgggttagataaaaaataatgcatgtaaaccgaagtagaataaaaccatcacaaaatcacatctagggggtaaatcctttccttataactcgatatacatgggtgtagaccaacagatgggcttacgagggcgggcctcaccgaattgacatacacatatgcgtaacctagttacaaaggaagaaaactcaaccccccagttttgctattatggatagaacttacagagatgctctatattccagggattgggaagaggcactccttctgttgtggcaaggcggacacaccatggtcggcatatttctgtcaccttgaagggtccttcccaactgggggagagtttgtggagcccttctcggttcagtactcgccttaggactaggtccccgaccctgagctccctactatgcacaaaccgttggtggtagcgcctgagcgcttggttgtaccgtgcatttcggatcaccgcttgccatctgcgttcgtcgatgaagtccatgtcctcacgtcgtagctattcctgcatagactcatcgaaagactggactcatggggagcccataatgatttccgggagaaggcaggcttcggccccgtagaccaagaagaacggggtctccccggtagctcggctgggtgtggtccggttcccccatagtacggacggaagctcattggcccaattggcaccatgcttttttaagcagtcgtaggtgtgtgccttgagtcccctaaggatttctgtgtttgccctctcagcctggtcgttgctcctgggatgagacacagatgtaaagcagagctgggtgccaatgccctcgcaatactcttggaagagtcgacttttgaactgggtcccattgtccgtaatgatatggcttgggaccccaaatctgcatacaatcgaattgaggaaggcaacagcagcaccttgggtgatactgaccataggggtggcctccgaccactttatgaatttgtagatggcgacaaagagaaaacggtacccgccgacagccctaggaaatggtcccaggatatccaccccccatacggcgaatggccaagagggtggaatcatttgcagagcctgagctggtgtgtgtgtgtgtctgctttgcatgaaactgacatgcttcgcaggacttcaccaactcggctccctcctagagagcagttggccagtagaagccatgccagaagaacgaatcaagctgattctcagagttgaaaannnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnaattcttagaaaattcgtcctcaaacccaagagacattaaagggattcttgagacgggctcaaaatgagttcggcttaagggtcaagaaaataagaagcgacaacggaacggagttcaagaactctcaaattgaaagctttcttgaggaagagggaatcaagcatgagttctcttctccctacacccctcaacaaaatggtgtagtggagaggaagaatcgaactctattggacatggcaaggaccatgctcgatgagtacaaaacttcggatcggttttgggccgaggcggtcaacaccgcctgctacgccatcaaccgattgtatctacaccgaatcctcaagaagacatcatatgaactcctaaccggtaaaaagcccaacatttcatactttagagtttttggtagcaaatgctttattcttgttaaaagaggtagaaaatctaaatttgctcctaaaactgtagaaggttttttacttggttatgactcaaacacaagggcatatagggtctttaacaagtccactggactagttgaagtctcatgtgacgttgtgtttgatgaaactaacggctctcaagtagagcaagttgatcttgatgagataggtgatgaagaggctccatgcatcgcattaaggaacatgtccattggggatgtgtgtcctaaggaatccgaagagcctccaaatgcacaagatcaaccatcctcctccatgcaagcatctccaccaactcaagatgaggaagaagctcaagtcgatgaagaagaagatcaatcaaatgagccacctcaagatgatggcaatgatcaagggggagatgcaaataatcaagaaaaggaggatgagcaagaaccaagggcgccacacccaagagtccaccaagcaatacaacgagatcaccccgtcgacaccatcctcggcgacattcataagggggtaacaactagatctcgtattgcacatttttgtgaacattactcgtttgtttcctctattgagccacacagggtagaggaagcactacaagattcggattgggtggtggcaatgcaagaggagctcaacaacttcacaaggaatgaggtatggcatttggttccacgtcctaaccaaaatgttgtaggaaccaaatgggtcttccgcaacaagcaagatgagcatggtgtggtgacaaggaacaaagctcgacttgtggccaagggatactcccaagtcgaaggtttggatttcggtgaaacctatgcacccgtagctaggcttgagtcaattcgcattttattggcatatgctacttaccatggctttaagctttatcaaatggacgtgaaaagtgccttcctcaatggaccaatcaaggaagaggtctatgttgagcaacctcccggctttgaagacagtgagtaccctaaccatgtctataggctctctaaggcgctttatgggctcaagcaagccccaagagcatggtatgaatgcctaagagatttccttatttctaatagcttcaaagtcggcaaggccgatcctacactctttactaaaactcttgaaaatgacttgtttgtatgccaaatttatgttgatgatattatatttgggtctactaacgagtctacatgtgaagagtttagtaggattatgacacagaaattcgagatgtctatgatgggggagttgaagtatttcttaagatttcaagtaaagcaactccaagagggcactttcattagccaaacaaagtacactcaagacatcctaagcaagtttggaatgaaggatgccaagcccatcaaaacacccatgggaaccaatgggcatctcgacctcgacacgggaggtaagtccgtggatcaaaaggtataccggtcgatgattggttcattgctttatttatgtgcatctcgaccggacattatgctctccgtttgcatgtgtgcaagattccaatccgaccctaaggaatcccaccttacggccgtaaaacgaatcttgagatatttggcttatacacctaagtttgggctttggtaccctcggggatccacgtttgatttgattggttattcggatgccgattgggcggggtgcaaaattaataggaagagcacatcggggacttgccagttcttgggaagatccttggtgtcttgggcttcaaagaagcaaaactcggtcgctctttccaccgccgaagccgagtacattgccgcaggacattgttgcgcgcaattgctctggatgaggcaaaccctgcgggactatggttacaaattaaccaaagtccctttgctatgtgataatgagagtgcaatcaaaatggccgacaatcccgtcgagcatagccgcactaagcacatagccattcggtatcattttcttagggatcaccaacaaaagggggatatcgagatttcttacattaatactaaagatcaattagccgatatctttaccaagccacttgatgaacaatcttttaccagacttaggcatgagctcaatattcttgattctagaaatttcttttgctagcttgcacacatagctcatttgaatacccttgatcatatctcttttatatgctatgactaatgtgttttcaagtctatttcaaaccaagtcataggtatattggaagggaattggagtcttcggcgaagacaaaggcttccactccgtaactcatccttcgccatcactccaaccatctctctattctttgggggagaaatgagcatcaaagaaaaggacttcgtctttggtataatcttaactcatttacttatgaccaaaggagaagaaattacttcgagggctctaatgattccgtttttggcgattcatgccaaaaagggggagaaaggagcccaaagcaaaaggaccgcaccaccaccaatttcaaaaacttagtgttttccaagaaatatttatcaattggcatcctatcgtgttcaaaagggggagaaagtagtatttcaaaaatgatatatcaaaaccctcttgaacactaagaggaggatttaatttagggggagttttgtttagtcaaaggaaaagcatttgaaacagggggagaaaacttcaaaatcttgaaaatgctttgcaaaaatcttattcattcacctttgactatttgcaaaagatctttgaaatggacttacaaaagaatttgcaaaaacaaaacatgtggtgcaaacgtggtccaaaatgctaaataaagaaagaaacattccatgcatatcttgtaagtagttatattggctcaattccaagcaacctttacacttacattatgcaaactagttcaattatgcacttctatatttgctttggtttgtgttggcatcaatcaccaaaaagggggagattgaaagggaattaggcttacacctagttcctaaataattttggtggttgaattgcccaacacaaatcttttggactaacttgtttgcccaagtgtatagtgtatacaggagtaaaaggttcacactcagccaataaaaagaccaagttttggattcaacaaaagagcaaaggggcaaccgaaggcacccctggtctggcgcaccggactgtccggtgtgccaccggacagtgaacagtacctgtccggtgcaccaggggactcagactcaaactcgccaccttcgggaatttctaaggcgactcggctataattcaccggactgtccggtgtacaccggacagtgtccggtgcgccaagggaggtcggcctcaggaactcgctagcctcgggttcgcgcggcagccgctccgctaaaattcaccggactgtccggtgtgcaccggactgtccggtgtgccagcggagcaacggctccctgcggcgccaacggctccctgcggtgcatttaatgcgcgcgcagcgcgcgcagacgccaggcacgcccataccggtgcaccggacatcaaacagtacatgtccggtgtgcaccggacacccaggcgggcccacaagtcggaagcttcaacggctagaatccaacggcagtgatgacgtggcaggggcaccggactgtccggtgtgcaccggactgtccggtgcgccatcgagcagacgcctccagccaacggtcaagtttggtggttggggctataaataccccaaccaccccaccattcatagcatccaagttttccacttcccaactactacaagagctaggcattcaattctagacacatacaaagagatcaaatcctctccaattcatcacaaagccctagtgactagtgagagtgatttgtcgtgttcatttgagctcttgcgcttggattgcttcttttctttctcacttgttcttgagatcaaaactccattgtaatcaaggcaagaggcaccaattgtgtggtggcccttgcggggaagttttgttcccggctttgatttgagaagagaagctcactcgatccgtggatcgtttgagagagggaagggttgaaagagacccggcctttgtggcctcctcaacggggagtaggtttgcaagaaccgaacctcggtaaaacaaatctccgtgtctcacttgctcattcgcttgggatttgttttgcgccctctcttgcggactcattccttattactaacgctaaccccggcttgtagttgtgtttatatttgcaaatttcagtttcgccctattcacccccctctaggcgactatcaattggtatcggagcccggtgcttcattagagcctaaccgctcgaagtgatgtcgggagatcacgccaagaaggagatggagaccggcgaaaggcccactacaagccacgggagcacttcatcggaagagtctcgcaccaaaaggagggagaagaagaagagctcctccaacaaagggaaggagaagaaatcttcttctcaccacaaagagaagaaggaaaaatcttcttcccacaagccgcatcggaaaggcgacaagcacaaaaggatgaggaaggtggtctactacgagaccgacacttcatcaacatcgacctccgactccgatgcgccctccgtcacttctaagcgccaagagcgcaagaagtatagtaagatccccctacgctaccctcgcatttccaaacatacacctttactttccgtcccattaggcaaaccaccaacttttgatggtgaagattacgctaggtggagcgatttaatgcgatttcatctaacctcgctccacaaaagcatatgggatgttgttgagtttggcgcgcaggtaccatccgtaggggatgaggactatgatgaggatgaggtggcccaaatcgagcacttcaactctcaagcaacaacaatactcctcgcctctctaagtagagaggagtataacaaagtacaagggttgaagagcgccaaggagatttgggatgtactcaaaaccgcgcacgagggagacgagctcaccaagatcaccaagcgggaaacgatcgagggggagctcggtcggttccggcttcacaaaggagaggagccacaacacatgtacaaccggctcaagactttggtgaaccaagtgcgcaacctcgggagcaagaagtgggacgatcacgaagtggtaaatgttattttaagatctctcatttttcttaatcccactcaagttcaattgattcgtggtaatcctagatatactaaaatgacccccgaggaagttatcgggcattttgtaagttttgagtgcatgatagaaggctcgaggaaaatcaacgagcttggcgactcatccgaagcccaacccgttgcattcaaggcaacggaggagaagaaggaggagtctacaccaagtcgacaaccaatagacgcctccaagcttgacaatgaggagatggcgctcgtcattaagagcttccgccaaatcctcaaacaaaggagggggaaagactacaagtcccgctccaagaaggtttgctacaaatgtggtaagcccggtcattttattgctaaatgtccaatatctagtgacagtgaccgaggcgacgacaagaaggggagaagaaaggagaagaagaggtattacaagaagaagggcggcgatgcccatgtttgtcgcaaatgggactccgacgagagctcaagcgactcctccgacgacgaggatgccgccaacatcgccgtcaccaagggacttctcttccccaacgtcggccacaagtgcctcatggcaaaggacggcaaaaagaagaaggttaaatccaactcctccactaaatatgaatcgtctagtgatgataatgctagtgatgaggaggaaaatttgcgtatcctctttgccaaccttaacatagctcaaaaggaaaaattaaatgaattagtcagtgctattcatgaaaaggatgaccttttggattcccaagaggattgtctaattaaagaaaacaagaaacatgttaaggttagaaaggcttatgctctagaagttgagaaatgtgaaaaattgtctagtgagctaagcacttgccgtgagatgattgacaaccttagaaatgaaaatgctagtttaaatgctaaggttgattctcatatttgtaatgtttcaattcccaatcctagagataataatgatgagttgcttgctaggattgaagaattaaacatttctcttgctagccttagattagagaatgaaaatttgattgctaaggctaaagattttgatgtttgcaaagttacaatttccgatcttagagataagaatgatattcttcatgctaagattgttgaacttaattcttgcaaaccctctacatctattgatgagcatgtatctatttgtactagatgtagagatgttgatgttaatgctattcttgatcatatggctttaattaaacaacaaaatgatcatatagcaaaattagatgctaaaattgccgagcacaacctagagaatgagaaatttaaatttgctcgtagcatgctttataatgggagacgccctgacattaaggatggcattggcttccaaaggggagacaatgtcaaacttaatgcccctcttaaaaacttgtctaactttgttaagggcaaggctcccatgcctcaggataacgagggttacattttgtaccctgccggttatcccgagagcaaaattaggaaaattcattctaggaagtctcactctggccctaatcatgcttttatgtataagggtgagacatctagctctaggcaaccaacccgtgccaagttgcctagaaagaaaactcctattgcatcaaatgatcatgctatttcatttaaaacttttgatgcttcttatgtgcttacaaacaaatccggcaaagtagttgccaaatatgttgggggcaagcacaaggggtcaaagacttgtgtttgggtacccaaagttattgtgtctaatgccaaaggacccaaaaccatttgggtacctaaagtcaagaactaaatttgtttttgtaggtttatgcatccgggggctcaagttggatactcgacagcgggtgcacaaacccacatgaccggggagaaaaggatgttctcctcatatgagaaaaaccaagatccccaacgagctatcacattcggggatggaaatcgaggtttggtcaaaggattgggtaaaattgctatatcacctgaccatactatttccaatgtttttcttgttgattcattagattacaacttgctttctgtttcccaattgtgtcaaatgggctacaactgtctttttactgatgtaggtgtcactgtctttagaagaagtgacgattcaatagcatttaagggtgtgttagagggtcagctatacttagtagattttgatagagctgaactcgacacatgcttaattgccaagactaacatgggttggctctggcaccgccgactagcccatgttgggatgaagaatcttcataagcttctaaagggagaacacattttaggattaacaaatgttcattttgagaaagacaggatttgtagcgcatgccaagccgggaagcaagttggcactcatcatccacacaagaacataatgacaagtgacaggccactggagctcctccacatggatttattcggcccgatcgcttacataagtatcggcgggagtaagtactgtctagttattgtggatgattattctcgcttcacttgggtattctttttacaggaaaaatctctaacccaagagacattaaagggattcttgagacgggctcaaaatgagacgaatctcagatcgtctgtatagattannnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnngcatcttgcaacctcacagaccgtggcgtgctctggtcaggcgcggacggtcccagccttgggcggacgtccgagccttgggtcggacggtccgcgacctgggcgaggagcggtgtcttccctgcgtcacaccggacgtccgcagctctgggccggacgtccgcgacctggcgacagggtcgtcttcctactccttgctggaatctagatctcgtcccctgggggaaagatcttaaggtgctccgggtcgacaggtcacccggggcgtccccagacgacgtggagtcgcctaggaattaagagatcaaatcgaggaagaagtcttggatggacaactagatcttgccccccggaggggtgagatcctagggtcgtcttgggatcggcaggccacccaagacggatctagacgacgtagagttgaataggggtggaggtggatatgtggaagactacaactagaactatgctacatctactcctagggcaggaaaagtaaataaggtaattggttcgattggaatgtgttcgggggttctcaatcggccgtacccctttatatttataggggaggaggtctggaccttttcctaagagatagccaacaaactcccacgtgattagatggataaccacgcacgagataaggataaacatccgagttaatctaatctcgggacacgcggaccgtccgggcccatgggccggaccgtccgctcattttggtgtccaacatatgcccccctgccttttggtggagcatggcgaaccaaaagcattagcgaaaacttcggaaacaattgacctcatgaggtttttttttccgaagtaaggactcagctcgatgcaagtcatcggctcttgcgatcagataatataaatacttgatgggactttaatgcacagaggccgtttcggatcgcatcctcttcagccatgtctatctgatcaacctgtcaataggcaaaaacttgtggtgccccccagcccaaataagcaaacggattgggccagtaatacaaattcatcgccgtaccaccccacacatgagtaggacaacacatcggcgatggatagaatgggacgcaccatgctatccctggaggaggatgataaggcgatattggttgtgctaccctttgggtccgtttagtcggcttttgctttcgcacagatcgccctattgactttgtttgttttattggccggttgtgtggaacggccttcttcatatatttggcaagcaactgaccaaaagtagggccgactctactgagtcgtccagacgtcttagtagtgttttgtttcctaacacttgtgttggaacgttgtggtccgatggtctgaggttgctgcttctgaccatctgcggaccgtccggccatcatagccggactgtccgcgcctgtctcggactgttcggccttagtacccggatcgtccggcgtacgcatgacaggcgaccgtgatcgggtgtccgatcgtgcttgccccccggtgcctccggtctttcttttgtccggagccttcagagtaaccattctgcgtgacatatttggtgtgcgaggatcaccaatgacgatatttttatttttacttttatcggccgcacaaggccgaattatggcctttttgctcatgggctctaatgtggtgacaggaacaggtggcctgtcaattttcacctctttttgaaacctcaaccggccttcgtttatagccgattgtatttgccgacggaagacggcacaatcattggtgttatggagaaaggagccatgccatttgcaataaacacgcccttttaattgttcaaccggaggaattacatgtgacaatttaatattaccatgtttaagcaactcatcaaatattttatcacatttagtaatattaaatgtgaacttaaccttttccttttgtttcgagtgcgggtaagagcgaacagaaggtttggccttagtgggccaaacaagctcagggacatgtgacttttttagttcttggggcttgggtggccgattatatttatgtcggccttccgcactaggtggatcacaggtgacttctggtgccccggacggtccgacttgcacagtcggacggtctgcgggtggatcggacggtccggtactatcctcggacagtccggtcacgtcaggcaacacctgtgacccttgtggtgggctctgtgtaactccagactgtccggcgtagggtgccggacggtccgacagagggccggacggtccgcaattgtgtgcggacggtccggctgtgcccagggttgactcaccatttagcaaagatggtgatgacggtcgtcctagatatgagtccatcggcataccagaatatggctggggaaacccatttgccgccgatgtgtttggcgcaattgtttcatcgcctaatttatgtgataaaaaattaggcatgtgagtttttcctaatgcatgtgtcatcctctctatatcctccgtgatacttttaatccgattatcaaaagaaattttaatagatggaatatcatcggctgacctggcatcacctattgtggggagctgttgcagcacggctaacatatactcggcgtttatctccctctcttggacggtcttctggtggcagtctaccttgaagtgcgagaggtaccacttgtccgccaccttgagcacctgatccttttgtcggtgggcctcctcgtctattttcttcatgtcatcttctaattttttatgctcagcggccgataaattagtcagccttgtgttgctgtttggagaagcactgttgagatctttagaatcggccatgtaagcctgattttgtagatctgcaacttcttccccagcggagtcgccaaaaagtatgttgacgcctttttggagcgccaaacactcaacaagaaccgtggcggtgccctctggtcaggcgcggacggtccgcagccttgggccggacggtccgcagccttgggccggacggtccgcgacctgggcgcaggagcggtgtcttccctgcgtcacaccggacggtccgcagctctgggccggacggtccgcgacctggcgacagggtcgtcttcctactccttgctggaatctagatctcgtcccctggggggaaagatcttaaggtgctccgggtcgacaggtcacccggggcgtccccagacgacgtggagtcgcctaggaattaagagatcaaatcgaggaagaagtcttggatggacaactagatcttgccccccgggaggggtgagatcctagggtcgtcttgggatcggcaggccacccaagacggatctagacgacgtagagttgaataggggtggaggtggatatgtggaagactacaactagaactatgctacatctactcctagggcaggaaaagtaaataaggtaattggttcgattggaatgtgttcgggggttctcaatcggccgtacccctttatatttataggggaggaggtctggaccttttcctaagagatagccaacaaactcccacgtgattagatggataaccacgcacgagataaagaaaaacccccnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnngaattccaagatttaaatagaagtcttttataatgagagattaaataaaagaccctcatataatttaaaccaacccttgttgaataacatgattagagatattctccaaaagaattaagcttaaaaaaccttaataaatactatacacacaaaaaaatcctctatcttaaaaattatgaacataattttaaatggactatacattcaaagaagtaatttttactctatgtgtgtgcattgcatacttaaaatatttggataaaataaacaaaactaaacagatatatgtaattattgcatatcatgccggagttttggattgagcatttagattagagtttaaaataagggaaagaaatatgaaagggaagataaaacagaaaatcattaaagaataaagaaaaaggggaagctttctgcgctatgggccggatctctggcttctcggcccagtttctttccttcgttagcgggcccaactctatttccctgctccggcgcagcccgctcctgcccactctcgcgcctgcagccgcgtctggcatgtgggccatggccgtcaagtctatcctccccatggcgatcctgctcgtccgctgcaagctcgcctcctgtaaactgtgcaacgaccttcgtgccatggtgcacccgcccactgctagccgtacccctggccatatataacggacgctccaacctcggccatgggtgcagctctagtttcctctccttcagcatcgtgggctacgctcggtctgccgatcgggagagaaggcgccatcaccatcgtcgtaagggagaaggagaacacagggggtgaattgccaccgacgggggttcccgggcacgccggtattgcggtctcggcgtcgggttgggtcatccgtgggacgcgtgcaggattctagaaggcacctcgtgcgagaacaacgaccagtgcatgcttcgctggtgacccgcggcgccacggagcaactgcgtggtggggtcaacacttgaaacaccgtgatccttggtaagaacagccctagcatacttggagcctcctcctctccgtgattcacgtacccacgctcgatactaggaaatggggagccgggcgggatatcactggtggtgtggtggggcatggccgcggcgtgcccgcaccagtgctctgctttccgtcgtgaggtggaaggaaatgcagcagccgttagatcatgggtgagcgatcacgatcagggcatggctgggcctcgcgtgaaccgtggatctgggaggtatcggctgtgattagatcacacgtaacgtttcatccgaatcgatccgggtcgtctgatctggatcttgcatatgaggatcgatctctattattttaagcgtgggccgtttatcgtagatccgacgatctaggatgcgtaccggttcggcgggcaaatcttctactctgggcgcttggctgatgatccaaggaattagtcacgtgtaccccttcaccgtgactaacttataaaagagaccccagacttcttgcaaatcagcccgcagtccgggtataggtagaaatcattgcggataagtcctaaatattatatggagccccctgatcttttatagaatagtgtccccaatccagaaatatttaataattatagaattaaatcctaaaacttaataaatacatatctctttcattttaactctgatttaatgtattcatgttgcgttagcttcgtaataattttgcctacgcttctgtaaaattattttagcaaatagcatgtttccaaaaaataaatattcatttaatatatgcttagtagattattcctactaatcaaagttagtttgtctatgattataaggtaactaaaatattatgtctactctagtatgatgtagattaaagttatttctttaatatctttatcacataatttataaaatcaacataaagacctagtctcatatttaatcacataggtcttccgaaaaccacatcttgttaaccgtaactccgaatttagtggttctcgaacctaggatctcgttgtggtgcgtagatcattattatgcagtttgttctttatgtttggtgtgatgttaattttgcctataccatgtttgtttgtattgctatgattagcagcgaggttacgagaatcttgaagaccaagctggtacctaggaatcttgagtctcagccaagttgtgcccttgatcacttttctttacctaataatgttcctattaatcactgtgacatgctcaggttaatttgatgggacccaataggttttcctagtattgtttatcccctaccttgcaaacaaaagcactattgggtagtattgctattgctctacctggttttgggaaattaatgttacattatgatcatgttacaattcttttgttattttaattattgttcatgataagattgctatgttaattggaacatggagcaaccacccaggaaaacagtgctaccacaagggtggtatgggacgcccttggctgactaattaagaaagctagtggaagactaccttacccgaaaggggcaagggcggtagaggagcatgcgtatagggaggttctcgagtcgatcatgctgcgatggctttttggacgagggattcctatattttccttcttagaaaccgtagcgggttttcggaagctagtggaagtttgtaaaggcctcgtagtggtaacctaccttgtcttctcggtagagatgaatgagaagtcgcgatcccttggcaaataggtaacatgacttgtgggtaaagatgtgcaacctgtgcagactgtaaaactgttatatcagccgtgctcacggtcatgagcagctcggaccctcacatgagtaaattatggaactaaacttaaattgtcatatgcattgcattgtgggtgttgttattaatttaatctcttatttatttgggtcggtatctacttatacttagtaactgctaataaaattttgaccaactttaaaagtcatgctcatctttacccatctcctttggtaagccttacacttcacatgagctcccacctttggtgagttcatacacattattccccacaacttgttgagcgatgaacgtatgtgagctcacccttgctgtactcaaatccccctggtcaagaacaggtaccgcaagatgaggagcatgaaggatgtcgcgatgagttcatgagaggtctaggccgtcgtctcacagtaaactttgggttgatggatcgtcgtcatcgtatgatgtaattatttagttattttgtgcagaacttctattatatagtaaagatgtgacatttgtttctataccatgagtcatcatatgtgtgagactcgatcccagcacttggtgaatttcgcgcctgggttttggacccctaaaacccgggtgtgacatgctgctgttgagggaactgcctctggaattgctactggtgcgaacattggttctggtgttggtatccctgagggtggatctacttgaactgctagggtggattgccagaaacgggagacgactgctgctcctggcctagggtccaccaatcttgcgcttttggtcttccatctcctggcgcttcctctcagtcattattgccctatcaatcagatgttggaaggtagggaatgtgtggttcatcaactagtagtgcaggggtcaaccaaccctctcggaaacctgtagtacctcttagcatcaatgttgacatcctcgggtgcattgtgagatagttgcaggaatttgtccatgtactcactgacagacaggggcccttgcttcagtgccagaaattcttccttcctcactatcatcaaaccttgtagaacgtggtacccgcagnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnncgcgcggaggccggtttgtcggtgccggtttctttcacgcaacacgcccgctcctttttgcctcggtgggtcggcctgtcagcgcagaaccgctcgttcgcgtattcaccctcgctggcaagcggaccccacctgtcagccacctcccctttccctaaccacccgctcgcgcaccccgccgtggatgcacacatgtcgcgtgtttttcggccactccccccacgcgcctgacttttttggagcccacactcactcgctcactcccctcgctcagtagcgtcccacagccgaccccctcgcacctctctctcgcaccgagcgcacagccgtggagcactgccgtagtccaccgtccgttccgtggccgtcgtcgagttcctgtcgcgtccattgccctactgatcttcgcctcctcgccagcaacacgagacaccctctggttttccccagcccctctatttcccttggttcgctcaccggacctatcaccatgcagccgagtctccgccaccgtccaccagggccctcgcggtgtcctcgccgttgctcaagcgctctagagtcatctctcgacgtaaccaacccacccatgcccttaatttcccatttactgccctgttgtccatgcaatcgctcgccagagttaagctgcgccgccgtggggctgctttgcctcggaccgtgctctctggtgcctctacgccggtgtcgtgcccatggctgagcccgccgtgtcaccctgagctcgcctgagccttttcccagcgcccagaccctcaccatggccgcgccacgccgcgaaattgggcggcggcgccatgagcagcctagcaaccccgcccgagcttgccatcagatttcaggcatccatctgagatctaacgacctggcttcaattaaactcgatctgatcccagctgtccgatggagatctggccactcggatccgccacctcacccgcgccctgcagctaggcccggccagacagtccgcctcgcccctaggtcgctgactatcctggcccacctgttagctcgtgctcgtgctcgcgctcaaatctaatcctggccgttgatctgtgatcatgcagtcgagatcagctgatacccctttgcgtggtagttttgttaaaaaggccctcggctttctgagaatcaacccatcgtccctggttttcgcacgcatgcccctgtacttttgcagaaaggcccctaatcttttaggttatcacataattagacctagttttgtattttgaattccaaaacttgtttatttcatatcttttgcatatgaactccaaattgagtgattcaaattgcaaaatgtttgtaaggttattctctacctgtttaaattataaccttttactgtctgcatgtgctaattttatgcctagactataggttagtgtaactgatggcttatttattaataagaaggataaaaggaaaaccataatggtagttagatgtttaactttgtgggttaataatatgtaatatatgaacctatccctggtataattcttttgtctcattaagataaatgaaattaagttatgtaatctattgagataagtaatacttagagaaccacaaacctatatgtgtattggtccaccctagaccctaggcttcgcttgagtttgttactttcttttgaattagtgttcacttgattgtatatttttggtgtattgtttctttatcattatcgaaatgtgttgaatgcatgatcgctttgcgtagacaacaagcagtctatggttcctgagtgtgttgccgaagatcttcctgggcaacaacctggtgaaggcaagtgtcctctgacctattatgtcctacttacttcataattcactgtccccctttacttaattgaaacctaaggtttgactagtctgtatttatcttgtccttgtttaccttttgggttattatggtaagcttcaagctattgctccactttaatcaacaaacatgatgcgaatatttatgatatgatgttgttattatgattacgatgatgttcttatggcactttaggagactcaggctattttcctgagtacctttcctttggacctgctcgttgagtgaccacccgtgataacagaacgaatcaagctgattcatcagcggccggg

SEQ ID NO: 111 - DNA, 1348 bp, Zea mays
  source 1..1348: mol_type = genomic DNA
tacaagaatattgagacgtgagtacatagcattggcattttcattagcaagcatttcaaaagaatttaattttctcatagcaatgtgatatctctcctcacgctcaattctagttccttcatgtagagcacatatgtccatccacaaatcatgacaatttttatggtttctaactctattaaacacatctttgcaaaggcctctaaaaagggtgtttttggccttagcattccatttctcatagttcaactcttcacctacaagatttgtgggatctctaggttcggggaatctttgtgtggcggctttgtagacaccaatgtctatagcctctaaatatgcttccatacgaattttccaatatggaaaatcgtcaccataaaaaacgggagaaggtccatccccaccggacatcgttactctagcggttaagctaatctaagagcaacaaggctcttataccaattgaaaggatcacgatgcccaagagggggggttgaattgggcttttctaaaaatcaacactaactaaaatctaagcaagagcccaacttcaccccgacaactagcactaagagaataatactagaaatacaacaatgctaagataatacttcaaatacttgctaaacaaatacacaatgtaaaatacttgaattaagtgcggaatgtaaagcaaggtttagaagactcctccaatttttctagaggtatcaaagagtcggcactctcccctagtcctcgttggagcacctgcgtaagggtatcgctctcccttggtcatcgcaagaaccaagtgctcacaacgagatgatcctttgccactccggcgcggtggatccctcacgaccgcttacaaacttgagtcgggtcaccaacaagatctccacggtgatcaccgagctcccaacgccaccaagccgtctaggtgatgccgatcaccaagagtaataagccatagactttcacttgaccaagagaagcctaatgcatgcggtgtgtgctctaggtggctctcgctagcgttaatgaggtccaaatgcgggattaagattctcaagtcacctcactaggctttgtggtgcttgcaatgctctaccaatgtgtaggagtaaatgtgggcagcaagaccatcaatatggtaggtggatggggtataaatagccctcacccaccaactagccattaccaggaatctgctgcgcatgggcgcaccggacagtccggtgtgccaccggtgcgccaacggtcgactcaaacggctagttctgacagctagccgttggacagatggcataccggacagtccgatacgctgtccggtgtgcctctaaaattcaactcacga